SEQUENCE LISTING (77 sequences)

SEQ ID NO: 1 - DNA, 103 bp, Artificial Sequence
  Description of Artificial Sequence Synthetic polynucleotide
cgcgctcgctcgctcactgaggccgcccgggcaaagcccgggcgtcgggcgacctttggtcgcccggcctcagtgagcgagcgagcgcgcagagagggagtgg

SEQ ID NO: 2 - DNA, 119 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 3 - DNA, 590 bp, Homo sapiens
ttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccga

SEQ ID NO: 4 - DNA, 183 bp, Artificial Sequence
  Description of Artificial Sequence Synthetic polynucleotide
aactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatcc

SEQ ID NO: 5 - DNA, 6 bp, Artificial Sequence
  Description of Artificial Sequence Synthetic oligonucleotide
gccacc

SEQ ID NO: 6 - DNA, 954 bp, Homo sapiens
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctga

SEQ ID NO: 7 - protein, 317 aa, Homo sapiens
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDLRKMVDMLQDSFPARFKAIHFIHQPWYFTTTYNVVKPFLKSKLLERVFVHGDDLSGFYQEIDENILPSDFGGTLPKYDGKAVAEQLFGPQAQAENTAF

SEQ ID NO: 8 - DNA, 236 bp, Simian virus 40
gatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 9 - DNA, 130 bp, Adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 3157 bp, Homo sapiens
ttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacctggcaggtaagtcaatacctggggcttgcctgggccagggagcccaggactggggtgaggactcaggggagcagggagaccacgtcccaagatgcctgtaaaactgaaaccacctggccattctccaggttgagccagaccaatttgatggcagatttagcaaataaaaatacaggacacccagttaaatgtgaatttcagatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggtaaccaagcattgcttatgtgtccattacatttcataacacttccatcctactttacagggaacaaccaagactggggttaaatctcacagcctgcaagtggaagagaagaacttgaacccaggtccaacttttgcgccacagcaggctgcctcttggtcctgacaggaagtcacaacttgggtctgagtactgatccctggctattttttggctgtgttaccttggacaagtcacttattcctcctcccgtttcctcctatgtaaaatggaaataataatgttgaccctgggtctgagagagtggatttgaaagtacttagtgcatcacaaagcacagaacacacttccagtctcgtgattatgtacttatgtaactggtcatcacccatcttgagaatgaatgcattggggaaagggccatccactaggctgcgaagtttctgagggactccttcgggctggagaaggatggccacaggagggaggagagattgccttatcctgcagtgatcatgtcattgagaacagagccagattctttttttcctggcagggccaacttgttttaacatctaaggactgagctatttgtgtctgtgccctttgtccaagcagtgtttcccaaagtgtagcccaagaaccatctccctcagagccaccaggaagtgctttaaattgcaggttcctaggccacagcctgcacctgcagagtcagaatcatggaggttgggacccaggcacctgcgtttctaacaaatgcctcgggtgattctgatgcaattgaaagtttgagatccacagttctgagacaataacagaatggtttttctaacccctgcagccctgacttcctatcctagggaaggggccggctggagaggccaggacagagaaagcagatcccttctttttccaaggactctgtgtcttccataggcaac

SEQ ID NO: 11 - DNA, 1585 bp, Homo sapiens
tacgtaatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggt

SEQ ID NO: 12 - DNA, 628 bp, Homo sapiens
tacgtaattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 13 - DNA, 2454 bp, Homo sapiens
gggccccggtgttatctcattcttttttctcctctgtaagttgacatgtgatgtgggaacaaaggggataaagtcattattttgtgctaaaatcgtaattggagaggacctcctgttagctgggctttcttctatttattgtggtggttactggagttccttcttctagttttaggatatatatatatattttttttttttctttccctgaagatataataatatatatacttctgaagattgagatttttaaattagttgtattgaaaactagctaatcagcaatttaaggctagcttgagacttatgtcttgaatttgtttttgtaggctccaaaaccaaggagggagtggtgcatggtgtggcaacaggtaagctccattgtgcttatatccaaagatgatatttaaagtatctagtgattagtgtggcccagtattcaagattcctatgaaattgtaaaacaatcactgagcattctaagaacatatcagtcttattgaaactgaattctttataaagtatttttaaaaaggtaaatattgattataaataaaaaatatacttgccaagaataatgagggctttgaattgataagctatgtttaatttatagtaagtgggcatttaaatattctgaccaaaaatgtattgacaaactgctgacaaaaataaaatgtgaatattgccataattttaaaaaaagagtaaaatttctgttgattacagtaaaatattttgaccttaaattatgttgattacaatattcctttgataattcagagtgcatttcaggaaacacccttggacagtcagtaaattgtttattgtatttatctttgtattgttatggtatagctatttgtacaaatattattgtgcaattattacatttctgattatattattcatttggcctaaatttaccaagaatttgaacaagtcaattaggtttacaatcaagaaatatcaaaaatgatgaaaaggatgataatcatcatcagatgttgaggaagatgacgatgagagtgccagaaatagagaaatcaaaggagaaccaaaatttaacaaattaaaagcccacagacttgctgtaattaagttttctgttgtaagtactccacgtttcctggcagatgtggtgaagcaaaagatataatcagaaatataatttatatgatcggaaagcattaaacacaatagtgcctatacaaataaaatgttcctatcactgacttctaaaatggaaatgaggacaatgatatgggaatcttaatacagtgttgtggataggactaaaaacacaggagtcagatcttcttggttcaacttcctgcttactccttaccagctgtgtgttttttgcaaggttcttcacctctatgtgatttagcttcctcatctataaaataattcagtgaattaatgtacacaaaacatctggaaaacaaaagcaaacaatatgtattttataagtgttacttatagttttatagtgaactttcttgtgcaacatttttacaactagtggagaaaaatatttctttaaatgaatacttttgatttaaaaatcagagtgtaaaaataaaacagactcctttgaaactagttctgttagaagttaattgtgcacctttaatgggctctgttgcaatccaacagagaagtagttaagtaagtggactatgatggcttctagggacctcctataaatatgatattgtgaagcatgattataataagaactagataacagacaggtggagactccactatctgaagagggtcaacctagatgaatggtgttccatttagtagttgaggaagaacccatgaggtttagaaagcagacaagcatgtggcaagttctggagtcagtggtaaaaattaaagaacccaactattactgtcacctaatgatctaatggagactgtggagatgggctgcatttttttaatcttctccagaatgccaaaatgtaaacacatatctgtgtgtgtgtgtgtgtgtgtgtgtgtgtgtgagagagagagagagagagagagagactgaagtttgtacaattagacattttataaaatgttttctgaaggacagtggctcacaatcttaagtttctaacattgtacaatgttgggagactttgtatactttattttctctttagcatattaaggaatctgagatgtcctacagtaaagaaatttgcattacatagttaaaatcagggttattcaaactttttgattattgaaacctttcttcattagttactagggttgaatgaaactagtgttccacagaaaactatgggaaatgttgctaggcagtaaggacatggtgatttcagcatgtgcaatatttacagcgattgcacccatggaccaccctggcagtagtgaaataaccaaaaatgctgtcataactagtatggctatgagaaacacattggg

SEQ ID NO: 14 - DNA, 1503 bp, Homo sapiens
attctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctgg

SEQ ID NO: 15 - DNA, 2619 bp, Artificial Sequence
  Description of Artificial Sequence Synthetic polynucleotide
ctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 16 - DNA, 130 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 145 bp, Adeno-associated virus
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 18 - DNA, 2208 bp, Adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 19 - protein, 735 aa, Adeno-associated virus [sequence omitted]

SEQ ID NO: 20 - DNA, 2217 bp, Adeno-associated virus
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 21 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 22 - DNA, 1616 bp, Unknown
  Description of Unknown CVM enhancer and CBA promoter polynucleotide
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggc

SEQ ID NO: 23 - DNA, 1503 bp, Homo sapiens
ccagaacaggtcccattcatggcccacatgacaacctgcttccccagtgggtatttttggagacagctcttctgtttccaggttttctctcctgcctaaatgtcctgcctaagtgccttcaagaacccttcaccatcctgctcctgcatgtgaccaggttccatggtcagttcaatcacctagtcacagttggtaagtgacagagttgggacttgaacctatgcctgcctgacaccaagtctttttttgacacctagagccaagacatctgaagacaaactccctaggagagctggcgtcatagaaaccttaaaggttagggagacctgggtttgaatcaggctttgtcagttatgacttgtgtgaccctagcaagttatttaacctttctgggtctcagtttcctcatctgcaaactgaggataataacagtacctaccaaaaagaactgtcgtgaaaaccatataatttctgcaatgctcctggcacagtgtcctgttctaaagcatagttccccttctctttcttagctccatattgattattaccctaacttgcacaaagagacttggaggacccccatagagtatcggagggtcccccatttcctgctctttccactccacacccccagcaagcacagggaagttctgggggccataatccacccacaggaaccaaatctaagccacctttctggctggtagacatccaggtatgtgggcacagaggtagacaggctgaaatgctgctgtgctatcagttgggttttgctggaacaggaatggaaatggagaggctgacagaactgccctggggagcccaggcaagagggacagtggctggacacccccagccagttgtgcagaccatcagaacaagatcctagattttaggaatacagggttcaagtccgtgcggcaactcttttctaaatatgcccaagccattaactttgagttttaaaaatactgatttacaagctgtacacaatgaaaaaatgcctatccctcacaccatgctgatgctgttccctgccatctcagattaccaattaaatacagaatgcccagttaaatgtgaactttttttttttttttttttttgagatggagttttgttcttgtcgcccaggctagagtgcaatggtgcgatctcagctcactgcaacctctgcctcccaggttcaagcaattctcctgccttagcctcctgagtagctggaactacaggtgcccaccagcacgcctggctaatttttggtatttttagtggagatggggtttcaccatgttggccaggctggtctcgaactcctgacctcaggtgatctgcctgcctcggcctcccaaagtgctgggattacaggcgtgagcctaaatgtgaacttttttaatactaaaaaagtatttgctgttcatcggaaattcacatttaactgggtgtcctgtatttttatttgctaaatctaccatcaaattggtctggctcaacctggagaat

SEQ ID NO: 24 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 25 - protein, 239 aa, Artificial Sequence
  Description of Artificial Sequence Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 26 - DNA, 4949 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggtaccacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 27 - DNA, 7330 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcagcttttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacctggcaggtaagtcaatacctggggcttgcctgggccagggagcccaggactggggtgaggactcaggggagcagggagaccacgtcccaagatgcctgtaaaactgaaaccacctggccattctccaggttgagccagaccaatttgatggcagatttagcaaataaaaatacaggacacccagttaaatgtgaatttcagatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggtaaccaagcattgcttatgtgtccattacatttcataacacttccatcctactttacagggaacaaccaagactggggttaaatctcacagcctgcaagtggaagagaagaacttgaacccaggtccaacttttgcgccacagcaggctgcctcttggtcctgacaggaagtcacaacttgggtctgagtactgatccctggctattttttggctgtgttaccttggacaagtcacttattcctcctcccgtttcctcctatgtaaaatggaaataataatgttgaccctgggtctgagagagtggatttgaaagtacttagtgcatcacaaagcacagaacacacttccagtctcgtgattatgtacttatgtaactggtcatcacccatcttgagaatgaatgcattggggaaagggccatccactaggctgcgaagtttctgagggactccttcgggctggagaaggatggccacaggagggaggagagattgccttatcctgcagtgatcatgtcattgagaacagagccagattctttttttcctggcagggccaacttgttttaacatctaaggactgagctatttgtgtctgtgccctttgtccaagcagtgtttcccaaagtgtagcccaagaaccatctccctcagagccaccaggaagtgctttaaattgcaggttcctaggccacagcctgcacctgcagagtcagaatcatggaggttgggacccaggcacctgcgtttctaacaaatgcctcgggtgattctgatgcaattgaaagtttgagatccacagttctgagacaataacagaatggtttttctaacccctgcagccctgacttcctatcctagggaaggggccggctggagaggccaggacagagaaagcagatcccttctttttccaaggactctgtgtcttccataggcaacgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 28 - DNA, 7264 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttacgtaatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggtgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatctaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 29 - DNA, 7321 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggcatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtactagggttaccccagaacaggtcccattcatggcccacatgacaacctgcttccccagtgggtatttttggagacagctcttctgtttccaggttttctctcctgcctaaatgtcctgcctaagtgccttcaagaacccttcaccatcctgctcctgcatgtgaccaggttccatggtcagttcaatcacctagtcacagttggtaagtgacagagttgggacttgaacctatgcctgcctgacaccaagtctttttttgacacctagagccaagacatctgaagacaaactccctaggagagctggcgtcatagaaaccttaaaggttagggagacctgggtttgaatcaggctttgtcagttatgacttgtgtgaccctagcaagttatttaacctttctgggtctcagtttcctcatctgcaaactgaggataataacagtacctaccaaaaagaactgtcgtgaaaaccatataatttctgcaatgctcctggcacagtgtcctgttctaaagcatagttccccttctctttcttagctccatattgattattaccctaacttgcacaaagagacttggaggacccccatagagtatcggagggtcccccatttcctgctctttccactccacacccccagcaagcacagggaagttctgggggccataatccacccacaggaaccaaatctaagccacctttctggctggtagacatccaggtatgtgggcacagaggtagacaggctgaaatgctgctgtgctatcagttgggttttgctggaacaggaatggaaatggagaggctgacagaactgccctggggagcccaggcaagagggacagtggctggacacccccagccagttgtgcagaccatcagaacaagatcctagattttaggaatacagggttcaagtccgtgcggcaactcttttctaaatatgcccaagccattaactttgagttttaaaaatactgatttacaagctgtacacaatgaaaaaatgcctatccctcacaccatgctgatgctgttccctgccatctcagattaccaattaaatacagaatgcccagttaaatgtgaactttttttttttttttttttttgagatggagttttgttcttgtcgcccaggctagagtgcaatggtgcgatctcagctcactgcaacctctgcctcccaggttcaagcaattctcctgccttagcctcctgagtagctggaactacaggtgcccaccagcacgcctggctaatttttggtatttttagtggagatggggtttcaccatgttggccaggctggtctcgaactcctgacctcaggtgatctgcctgcctcggcctcccaaagtgctgggattacaggcgtgagcctaaatgtgaacttttttaatactaaaaaagtatttgctgttcatcggaaattcacatttaactgggtgtcctgtatttttatttgctaaatctaccatcaaattggtctggctcaacctggagaatggttaccctaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 30 - DNA, 6492 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtactagggttaccccagaacaggtcccattcatggcccacatgacaacctgcttccccagtgggtatttttggagacagctcttctgtttccaggttttctctcctgcctaaatgtcctgcctaagtgccttcaagaacccttcaccatcctgctcctgcatgtgaccaggttccatggtcagttcaatcacctagtcacagttggtaagtgacagagttgggacttgaacctatgcctgcctgacaccaagtctttttttgacacctagagccaagacatctgaagacaaactccctaggagagctggcgtcatagaaaccttaaaggttagggagacctgggtttgaatcaggctttgtcagttatgacttgtgtgaccctagcaagttatttaacctttctgggtctcagtttcctcatctgcaaactgaggataataacagtacctaccaaaaagaactgtcgtgaaaaccatataatttctgcaatgctcctggcacagtgtcctgttctaaagcatagttccccttctctttcttagctccatattgattattaccctaacttgcacaaagagacttggaggacccccatagagtatcggagggtcccccatttcctgctctttccactccacacccccagcaagcacagggaagttctgggggccataatccacccacaggaaccaaatctaagccacctttctggctggtagacatccaggtatgtgggcacagaggtagacaggctgaaatgctgctgtgctatcagttgggttttgctggaacaggaatggaaatggagaggctgacagaactgccctggggagcccaggcaagagggacagtggctggacacccccagccagttgtgcagaccatcagaacaagatcctagattttaggaatacagggttcaagtccgtgcggcaactcttttctaaatatgcccaagccattaactttgagttttaaaaatactgatttacaagctgtacacaatgaaaaaatgcctatccctcacaccatgctgatgctgttccctgccatctcagattaccaattaaatacagaatgcccagttaaatgtgaactttttttttttttttttttttgagatggagttttgttcttgtcgcccaggctagagtgcaatggtgcgatctcagctcactgcaacctctgcctcccaggttcaagcaattctcctgccttagcctcctgagtagctggaactacaggtgcccaccagcacgcctggctaatttttggtatttttagtggagatggggtttcaccatgttggccaggctggtctcgaactcctgacctcaggtgatctgcctgcctcggcctcccaaagtgctgggattacaggcgtgagcctaaatgtgaacttttttaatactaaaaaagtatttgctgttcatcggaaattcacatttaactgggtgtcctgtatttttatttgctaaatctaccatcaaattggtctggctcaacctggagaatggttaccctaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 31 - DNA, 4741 bp, Artificial Sequence
  Description of Artificial Sequence Synthetic polynucleotide
cgcgctcgctcgctcactgaggccgcccgggcaaagcccgggcgtcgggcgacctttggtcgcccggcctcagtgagcgagcgagcgcgcagagagggagtggggtaccacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctg

SEQ ID NO: 32 - DNA, 7122 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcagcttttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacctggcaggtaagtcaatacctggggcttgcctgggccagggagcccaggactggggtgaggactcaggggagcagggagaccacgtcccaagatgcctgtaaaactgaaaccacctggccattctccaggttgagccagaccaatttgatggcagatttagcaaataaaaatacaggacacccagttaaatgtgaatttcagatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggtaaccaagcattgcttatgtgtccattacatttcataacacttccatcctactttacagggaacaaccaagactggggttaaatctcacagcctgcaagtggaagagaagaacttgaacccaggtccaacttttgcgccacagcaggctgcctcttggtcctgacaggaagtcacaacttgggtctgagtactgatccctggctattttttggctgtgttaccttggacaagtcacttattcctcctcccgtttcctcctatgtaaaatggaaataataatgttgaccctgggtctgagagagtggatttgaaagtacttagtgcatcacaaagcacagaacacacttccagtctcgtgattatgtacttatgtaactggtcatcacccatcttgagaatgaatgcattggggaaagggccatccactaggctgcgaagtttctgagggactccttcgggctggagaaggatggccacaggagggaggagagattgccttatcctgcagtgatcatgtcattgagaacagagccagattctttttttcctggcagggccaacttgttttaacatctaaggactgagctatttgtgtctgtgccctttgtccaagcagtgtttcccaaagtgtagcccaagaaccatctccctcagagccaccaggaagtgctttaaattgcaggttcctaggccacagcctgcacctgcagagtcagaatcatggaggttgggacccaggcacctgcgtttctaacaaatgcctcgggtgattctgatgcaattgaaagtttgagatccacagttctgagacaataacagaatggtttttctaacccctgcagccctgacttcctatcctagggaaggggccggctggagaggccaggacagagaaagcagatcccttctttttccaaggactctgtgtcttccataggcaacgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 33 - DNA, 7162 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacgtcgtttaaacgggccccggtgttatctcattcttttttctcctctgtaagttgacatgtgatgtgggaacaaaggggataaagtcattattttgtgctaaaatcgtaattggagaggacctcctgttagctgggctttcttctatttattgtggtggttactggagttccttcttctagttttaggatatatatatatattttttttttttctttccctgaagatataataatatatatacttctgaagattgagatttttaaattagttgtattgaaaactagctaatcagcaatttaaggctagcttgagacttatgtcttgaatttgtttttgtaggctccaaaaccaaggagggagtggtgcatggtgtggcaacaggtaagctccattgtgcttatatccaaagatgatatttaaagtatctagtgattagtgtggcccagtattcaagattcctatgaaattgtaaaacaatcactgagcattctaagaacatatcagtcttattgaaactgaattctttataaagtatttttaaaaaggtaaatattgattataaataaaaaatatacttgccaagaataatgagggctttgaattgataagctatgtttaatttatagtaagtgggcatttaaatattctgaccaaaaatgtattgacaaactgctgacaaaaataaaatgtgaatattgccataattttaaaaaaagagtaaaatttctgttgattacagtaaaatattttgaccttaaattatgttgattacaatattcctttgataattcagagtgcatttcaggaaacacccttggacagtcagtaaattgtttattgtatttatctttgtattgttatggtatagctatttgtacaaatattattgtgcaattattacatttctgattatattattcatttggcctaaatttaccaagaatttgaacaagtcaattaggtttacaatcaagaaatatcaaaaatgatgaaaaggatgataatcatcatcagatgttgaggaagatgacgatgagagtgccagaaatagagaaatcaaaggagaaccaaaatttaacaaattaaaagcccacagacttgctgtaattaagttttctgttgtaagtactccacgtttcctggcagatgtggtgaagcaaaagatataatcagaaatataatttatatgatcggaaagcattaaacacaatagtgcctatacaaataaaatgttcctatcactgacttctaaaatggaaatgaggacaatgatatgggaatcttaatacagtgttgtggataggactaaaaacacaggagtcagatcttcttggttcaacttcctgcttactccttaccagctgtgtgttttttgcaaggttcttcacctctatgtgatttagcttcctcatctataaaataattcagtgaattaatgtacacaaaacatctggaaaacaaaagcaaacaatatgtattttataagtgttacttatagttttatagtgaactttcttgtgcaacatttttacaactagtggagaaaaatatttctttaaatgaatacttttgatttaaaaatcagagtgtaaaaataaaacagactcctttgaaactagttctgttagaagttaattgtgcacctttaatgggctctgttgcaatccaacagagaagtagttaagtaagtggactatgatggcttctagggacctcctataaatatgatattgtgaagcatgattataataagaactagataacagacaggtggagactccactatctgaagagggtcaacctagatgaatggtgttccatttagtagttgaggaagaacccatgaggtttagaaagcagacaagcatgtggcaagttctggagtcagtggtaaaaattaaagaacccaactattactgtcacctaatgatctaatggagactgtggagatgggctgcatttttttaatcttctccagaatgccaaaatgtaaacacatatctgtgtgtgtgtgtgtgtgtgtgtgtgtgtgtgagagagagagagagagagagagagactgaagtttgtacaattagacattttataaaatgttttctgaaggacagtggctcacaatcttaagtttctaacattgtacaatgttgggagactttgtatactttattttctctttagcatattaaggaatctgagatgtcctacagtaaagaaatttgcattacatagttaaaatcagggttattcaaactttttgattattgaaacctttcttcattagttactagggttgaatgaaactagtgttccacagaaaactatgggaaatgttgctaggcagtaaggacatggtgatttcagcatgtgcaatatttacagcgattgcacccatggaccaccctggcagtagtgaaataaccaaaaatgctgtcataactagtatggctatgagaaacacattgggcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 34 - DNA, 7057 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttacgtaatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggtgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 35 - DNA, 6100 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttacgtaattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgaaatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcag

SEQ ID NO: 36 - DNA, 130 bp, Artificial Sequence; identical to SEQ ID NO: 16
  Description of Artificial Sequence Synthetic polynucleotide

SEQ ID NO: 37 - DNA, 954 bp, Macaca mulatta
atgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaagatgagctgaatgagagagaggagacccgggaggaggcagtgcgagagctgcaggagatggtgcaggcgcaggcggcctcgggggaggagctggccgtggccgtggcggagagggtgcaagagaaggacagcggcttcttcctgcgcttcatccgcgcgcgaaagttcaacgtgggccgtgcctatgagctgctcagaggctatgtgaatttccggctgcagtaccctgagctctttgacagcctgtccccagaggctgtccgctgtaccattgaagctggctaccctggtgtcctctctagtcgggacaagtatggccgagtggtcatgctcttcaacattgagaactggcaaagtcaagaaatcaccttcgatgagatcttgcaggcatattgcttcatcctggagaagctgctggagaatgaggaaactcaaattaatggattctgcatcattgagaacttcaagggctttaccatgcagcaggctgctagtctccgcacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggaggacctctctggtttctaccaggagattgatgagaacatcctgccctctgactttgggggcacgctgcccaagtatgatggcaaagctgttgctgagcagctctttggcccccgggcccaagctgagaacacagccttctga

SEQ ID NO: 38 - protein, 317 aa, Macaca mulatta
MSEGVGTFRMVPEEEQELRAQLEQLTTKDHGPVFGPCSQLPRHTLQKAKDELNEREETREEAVRELQEMVQAQAASGEELAVAVAERVQEKDSGFFLRFIRARKFNVGRAYELLRGYVNFRLQYPELFDSLSPEAVRCTIEAGYPGVLSSRDKYGRVVMLFNIENWQSQEITFDEILQAYCFILEKLLENEETQINGFCIIENFKGFTMQQAASLRTSDLRKMVDMLQDSFPARFKAIHFIHQPWYFTTTYNVVKPFLKSKLLERVFVHGEDLSGFYQEIDENILPSDFGGTLPKYDGKAVAEQLFGPRAQAENTAF

SEQ ID NO: 39 - DNA, 954 bp, Bos taurus
atgtcagagggggcgggcacgttccgcatggtccctgaagaggaacaggagctccgtgcccaactggagaggcttacgaccaaagaccatggacctgtctttggcccgtgcagccagctgccccgccacaccttgcagaaggccaaggacgagctgaatgaaaaggaagagacccgggaagaggcagtgcgggagctacaggagctggtgcaggcggaggccgcctcggggcaggagctggccgtggccgtggcggagagggtgcagggaaaagacagtgccttcttcctgcgcttcatccgcgcgcgcaagttccacgtggggcgcgcctacgagctgctcagaggctacgtgaacttccggctgcagtacccagagctcttcgacagcctgtccccagaggctgtccgctgcaccgttgaggctggctaccctggtgtcctctccacgcgggacaagtatggccgagtggtcatgctcttcaatattgagaactgggactctgaagaaatcacctttgatgagatcttgcaggcatactgcgtcatcctggagaagctactggagaatgaggagactcaaattaatggcttttgcatcattgagaacttcaagggcttcaccatgcagcaggctgccggacttcggccttccgatctcagaaagatggtggacatgctccaggattccttcccagctcggttcaaagccatccacttcatctaccagccctggtacttcaccaccacctacaacgtggtcaagcccttcttgaagagcaaattgctccagagggtatttgtccatggagaagacctctccagcttctaccaggagtttgacgaggacatcctgccctccgactttgggggtacactgcccaagtatgatggcaaggccgttgctgagcagctctttggtcctcgggaccaaactgagaacacagccttctga

SEQ ID NO: 40 - protein, 317 aa, Bos taurus
MSEGAGTFRMVPEEEQELRAQLERLTTKDHGPVFGPCSQLPRHTLQKAKDELNEKEETREEAVRELQELVQAEAASGQELAVAVAERVQGKDSAFFLRFIRARKFHVGRAYELLRGYVNFRLQYPELFDSLSPEAVRCTVEAGYPGVLSTRDKYGRVVMLFNIENWDSEEITFDEILQAYCVILEKLLENEETQINGFCIIENFKGFTMQQAAGLRPSDLRKMVDMLQDSFPARFKAIHFIYQPWYFTTTYNVVKPFLKSKLLQRVFVHGEDLSSFYQEFDEDILPSDFGGTLPKYDGKAVAEQLFGPRDQTENTAF

SEQ ID NO: 41 - DNA, 954 bp, Canis lupus
atgtcagaaggcgtgggcacattccgtgtggtccctgaagaggaacaggagctccgtgcccagctggagcggcttacaaccaaggaccatgggcctgtctttggcccttgcagccagctccctcgtcataccttacagaaggccaaggacgagctgaacgagagggaggagacccgggaggaggtggtgcgagagctgcaggagctggtgcaggcacaggctgccaccgggcaggagctggccagggcggtggctgagagggtgcagggaagggacagtgccttcttcctgcgcttcatccgcgcgcggaagttccatgtggggcgtgcctacgagctgcttcgaggctacgtgaacttccggctgcagtacccagagctcttcgacagcctgtccctggaggctgtccgttgcaccgtcgaggccggctatcctggggtcctccccagtcgggacaagtatggccgagtggtcatgctcttcaacatcgagaactgggactccgaagaaatcaccttcgatgagatcttgcaggcatattgtttcatcctggagaagctactagagaatgaggaaactcaaattaatggcttctgcattattgagaactttaagggctttaccatgcagcaggctgctggacttcgggcttccgatctcaggaagatggtggacatgctccaggattccttcccagcgcggttcaaagccatccacttcattcaccaaccatggtacttcaccaccacctacaacatggtcaagcccctcctgaagaacaagctgctccaaagagtctttgtccatggagatgacctctctggcttcttccaggagattgatgaagacatactgcccgctgactttgggggcacactgcccaagtatgatggcaaggtggttgctgagcagctctttggcccccgggcccaagctgagaacacagccttctga

SEQ ID NO: 42 - protein, 317 aa, Canis lupus
MSEGVGTFRVVPEEEQELRAQLERLTTKDHGPVFGPCSQLPRHTLQKAKDELNEREETREEVVRELQELVQAQAATGQELARAVAERVQGRDSAFFLRFIRARKFHVGRAYELLRGYVNFRLQYPELFDSLSLEAVRCTVEAGYPGVLPSRDKYGRVVMLFNIENWDSEEITFDEILQAYCFILEKLLENEETQINGFCIIENFKGFTMQQAAGLRASDLRKMVDMLQDSFPARFKAIHFIHQPWYFTTTYNMVKPLLKNKLLQRVFVHGDDLSGFFQEIDEDILPADFGGTLPKYDGKVVAEQLFGPRAQAENTAF

SEQ ID NO: 43 - DNA, 954 bp, Rattus norvegicus
atgtcagagggggtgggcacattccgaatggtccctgaagaggagcaggagctccgggcacagctagaacagctcacaaccaaggatcatggtcctgtctttggcccatgcagccagctgccccgccacactttgcagaaggctaaggatgagctgaatgaaagggaggaaacccgggatgaggcggtgagggagctacaggagctggtccaggcacaggcagcttctggggaagagttggccgtggcagtggctgagagggtgcaggcaagagacagcgccttcctcctgcgcttcatccgtgcccgaaagtttgatgtgggccgggcttatgagctgctcaaaggctatgtgaacttccggctccagtaccctgaactcttcgatagcctatctatggaggctctccgctgcactatcgaggccggttaccctggtgtcctttccagtcgggacaagtatggtcgagtggttatgctcttcaacattgaaaactggcactgtgaagaagtcacctttgatgagatcttacaggcatattgtttcattctggagaaactgctggagaacgaggaaacccaaatcaacggcttctgtattgtggagaacttcaagggcttcaccatgcagcaggccgcgggactccgcccctccgatctcaagaagatggtggacatgctccaggattcattcccagccaggttcaaagctatccacttcatccaccaaccatggtacttcaccaccacttacaatgtggtcaagcccttcttgaagaacaagttgctacagagggtcttcgttcatggagatgacctggacggcttcttccaggagattgatgagaatatcttgcctgctgactttgggggtacactgcccaagtatgacggcaaagttgtcgctgagcagctcttcggtccccgggttgaggttgagaacacagccttgtga

SEQ ID NO: 44 - protein, 317 aa, Rattus norvegicus
MSEGVGTFRMVPEEEQELRAQLEQLTTKDHGPVFGPCSQLPRHTLQKAKDELNEREETRDEAVRELQELVQAQAASGEELAVAVAERVQARDSAFLLRFIRARKFDVGRAYELLKGYVNFRLQYPELFDSLSMEALRCTIEAGYPGVLSSRDKYGRVVMLFNIENWHCEEVTFDEILQAYCFILEKLLENEETQINGFCIVENFKGFTMQQAAGLRPSDLKKMVDMLQDSFPARFKAIHFIHQPWYFTTTYNVVKPFLKNKLLQRVFVHGDDLDGFFQEIDENILPADFGGTLPKYDGKVVAEQLFGPRVEVENTAL

SEQ ID NO: 45 - DNA, 954 bp, Mus musculus
atgtcagacggggtgggcactttccgcatggttcctgaagaggagcaggagctccgagcacaactggagcagctcacaaccaaggatcatggtcctgtctttggcccatgcagccagctgccccgccacactttgcagaaggccaaggatgagctgaatgaaaaggaggagacccgggaggaagcggtgagggagctacaggagctggtacaggcacaggcagcttctggcgaggaattggccctggcagtggctgagagggtgcaggcaagagacagcgccttcctcctgcgcttcatccgtgcccgcaagttcgatgtgggtcgtgcttatgagctgctcaaaggctatgtgaacttccgcctccagtaccctgaactcttcgatagtctctccatggaggctctccgctgcactatcgaggccggataccctggtgtcctttccagtcgggacaagtatggtcgagtggttatgctcttcaacatcgaaaactggcactgtgaagaagtgacctttgatgagatcttacaggcatattgtttcattttggagaaactgctggaaaatgaggaaacccaaatcaacggcttctgtattgttgagaacttcaagggcttcaccatgcagcaggcagcagggctccgcccctcggatctcaagaagatggtggacatgctccaggattcattcccagccaggttcaaagctatccacttcatccaccagccatggtacttcaccaccacctataatgtggtcaagcccttcttgaagaacaagctgctacagagggtctttgttcacggagatgacctggatggcttcttccaggagattgatgagaacatcctgcctgctgactttgggggtacactgcccaagtacgacggcaaagttgttgctgagcagctctttggtccccgggctgaagttgagaacacagccttatga

SEQ ID NO: 46 - protein, 317 aa, Mus musculus
MSDGVGTFRMVPEEEQELRAQLEQLTTKDHGPVFGPCSQLPRHTLQKAKDELNEKEETREEAVRELQELVQAQAASGEELALAVAERVQARDSAFLLRFIRARKFDVGRAYELLKGYVNFRLQYPELFDSLSMEALRCTIEAGYPGVLSSRDKYGRVVMLFNIENWHCEEVTFDEILQAYCFILEKLLENEETQINGFCIVENFKGFTMQQAAGLRPSDLKKMVDMLQDSFPARFKAIHFIHQPWYFTTTYNVVKPFLKNKLLQRVFVHGDDLDGFFQEIDENILPADFGGTLPKYDGKVVAEQLFGPRAEVENTAL

SEQ ID NO: 47 - DNA, 951 bp, Gallus gallus
atgtctgctgttacgggcaccttccgcattgtctcggaagaggagcaggcgctgcgcaccaaactggagcgcctcaccaccaaggaccacggccctgtttttgggaggtgccagcagatcccccctcacaccctgcagaaggcaaaagatgagctgaatgagacggaggagcagagggaggcagcggtcaaagcgctgcgggagctggtgcaggagcgggccggcagcgaggatgtctgcaaggcagtggcagagaagatgcaggggaaggacgattccttcttcctccgcttcatccgtgcccgcaagtttgacgtgcacagggcctacgacctgctgaaaggctatgtgaactttcgccagcaataccctgaactctttgacaacctgacccccgaggccgtgcgcagcaccatcgaggcgggctaccccggcatcctggccagcagggacaaatacgggcgggtagtgatgctcttcaacatcgagaactgggactacgaggagatcacctttgatgagatccttcgtgcctactgcgttatcttggagaagctgctggaaaacgaagagacccagatcaatgggttctgcatcattgagaacttcaagggcttcaccatgcagcaggcatcagggatcaaaccctccgagctcaagaagatggtggacatgctacaggactccttcccagcgcggttcaaagctgtccacttcatccaccagccctggtacttcaccactacctacaacgtggtcaaaccgttcctgaagagcaagctgctggagagggtgtttgtgcacggcgaggagctggagtccttctaccaggagatcgatgctgacatactgccagcagacttcggtggcaacctgcccaagtacgacggcaaagcaactgcagagcagctctttgggccccgcattgaggctgaagacacggcactttaa

SEQ ID NO: 48 - protein, 316 aa, Gallus gallus
MSAVTGTFRIVSEEEQALRTKLERLTTKDHGPVFGRCQQIPPHTLQKAKDELNETEEQREAAVKALRELVQERAGSEDVCKAVAEKMQGKDDSFFLRFIRARKFDVHRAYDLLKGYVNFRQQYPELFDNLTPEAVRSTIEAGYPGILASRDKYGRVVMLFNIENWDYEEITFDEILRAYCVILEKLLENEETQINGFCIIENFKGFTMQQASGIKPSELKKMVDMLQDSFPARFKAVHFIHQPWYFTTTYNVVKPFLKSKLLERVFVHGEELESFYQEIDADILPADFGGNLPKYDGKATAEQLFGPRIEAEDTAL

SEQ ID NO: 49 - DNA, 2659 bp, Artificial Sequence
  Description of Artificial Sequence Synthetic polynucleotide
ctgcctgcagggttccatcccaatggcgcgtcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggcgcgccattgggatggaaccctgcaggcag

SEQ ID NO: 50 - DNA, 4989 bp, Artificial Sequence
  Description of Artificial Sequence Synthetic polynucleotide
ctgcgcgctcgctcgctcactgaggccgcccgggcaaagcccgggcgtcgggcgacctttggtcgcccggcctcagtgagcgagcgagcgcgcagagagggagtggggtaccacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagggttccatcccaatggcgcgtcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggcgcgccattgggatggaaccctgcaggcag

SEQ ID NO: 51 - DNA, 2327 bp, Artificial Sequence
  Description of Artificial Sequence Synthetic polynucleotide
cgcgctcgctcgctcactgaggccgcccgggcaaagcccgggcgtcgggcgacctttggtcgcccggcctcagtgagcgagcgagcgcgcagagagggagtggggtaccacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 52 - DNA, 4711 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcagcttttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacctggcaggtaagtcaatacctggggcttgcctgggccagggagcccaggactggggtgaggactcaggggagcagggagaccacgtcccaagatgcctgtaaaactgaaaccacctggccattctccaggttgagccagaccaatttgatggcagatttagcaaataaaaatacaggacacccagttaaatgtgaatttcagatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggtaaccaagcattgcttatgtgtccattacatttcataacacttccatcctactttacagggaacaaccaagactggggttaaatctcacagcctgcaagtggaagagaagaacttgaacccaggtccaacttttgcgccacagcaggctgcctcttggtcctgacaggaagtcacaacttgggtctgagtactgatccctggctattttttggctgtgttaccttggacaagtcacttattcctcctcccgtttcctcctatgtaaaatggaaataataatgttgaccctgggtctgagagagtggatttgaaagtacttagtgcatcacaaagcacagaacacacttccagtctcgtgattatgtacttatgtaactggtcatcacccatcttgagaatgaatgcattggggaaagggccatccactaggctgcgaagtttctgagggactccttcgggctggagaaggatggccacaggagggaggagagattgccttatcctgcagtgatcatgtcattgagaacagagccagattctttttttcctggcagggccaacttgttttaacatctaaggactgagctatttgtgtctgtgccctttgtccaagcagtgtttcccaaagtgtagcccaagaaccatctccctcagagccaccaggaagtgctttaaattgcaggttcctaggccacagcctgcacctgcagagtcagaatcatggaggttgggacccaggcacctgcgtttctaacaaatgcctcgggtgattctgatgcaattgaaagtttgagatccacagttctgagacaataacagaatggtttttctaacccctgcagccctgacttcctatcctagggaaggggccggctggagaggccaggacagagaaagcagatcccttctttttccaaggactctgtgtcttccataggcaacgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 4645 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttacgtaatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggtgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatctaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 54 - DNA, 4702 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggcatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtactagggttaccccagaacaggtcccattcatggcccacatgacaacctgcttccccagtgggtatttttggagacagctcttctgtttccaggttttctctcctgcctaaatgtcctgcctaagtgccttcaagaacccttcaccatcctgctcctgcatgtgaccaggttccatggtcagttcaatcacctagtcacagttggtaagtgacagagttgggacttgaacctatgcctgcctgacaccaagtctttttttgacacctagagccaagacatctgaagacaaactccctaggagagctggcgtcatagaaaccttaaaggttagggagacctgggtttgaatcaggctttgtcagttatgacttgtgtgaccctagcaagttatttaacctttctgggtctcagtttcctcatctgcaaactgaggataataacagtacctaccaaaaagaactgtcgtgaaaaccatataatttctgcaatgctcctggcacagtgtcctgttctaaagcatagttccccttctctttcttagctccatattgattattaccctaacttgcacaaagagacttggaggacccccatagagtatcggagggtcccccatttcctgctctttccactccacacccccagcaagcacagggaagttctgggggccataatccacccacaggaaccaaatctaagccacctttctggctggtagacatccaggtatgtgggcacagaggtagacaggctgaaatgctgctgtgctatcagttgggttttgctggaacaggaatggaaatggagaggctgacagaactgccctggggagcccaggcaagagggacagtggctggacacccccagccagttgtgcagaccatcagaacaagatcctagattttaggaatacagggttcaagtccgtgcggcaactcttttctaaatatgcccaagccattaactttgagttttaaaaatactgatttacaagctgtacacaatgaaaaaatgcctatccctcacaccatgctgatgctgttccctgccatctcagattaccaattaaatacagaatgcccagttaaatgtgaactttttttttttttttttttttgagatggagttttgttcttgtcgcccaggctagagtgcaatggtgcgatctcagctcactgcaacctctgcctcccaggttcaagcaattctcctgccttagcctcctgagtagctggaactacaggtgcccaccagcacgcctggctaatttttggtatttttagtggagatggggtttcaccatgttggccaggctggtctcgaactcctgacctcaggtgatctgcctgcctcggcctcccaaagtgctgggattacaggcgtgagcctaaatgtgaacttttttaatactaaaaaagtatttgctgttcatcggaaattcacatttaactgggtgtcctgtatttttatttgctaaatctaccatcaaattggtctggctcaacctggagaatggttaccctaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 55 - DNA, 3873 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattcgccaccatgtcagaaggggtgggcacgttccgcatggtacctgaagaggaacaggagctccgtgcccaactggagcagctcacaaccaaggaccatggacctgtctttggcccgtgcagccagctgccccgccacaccttgcagaaggccaaggatgagctgaacgagagagaggagacccgggaggaggcagtgcgagagctgcaggagatggtgcaggcgcaggcggcctcgggggaggagctggcggtggccgtggcggagagggtgcaagagaaggacagcggcttcttcctgcgcttcatccgcgcacggaagttcaacgtgggccgtgcctatgagctgctcagaggctatgtgaatttccggctgcagtaccctgagctctttgacagcctgtccccagaggctgtccgctgcaccattgaagctggctaccctggtgtcctctctagtcgggacaagtatggccgagtggtcatgctcttcaacattgagaactggcaaagtcaagaaatcacctttgatgagatcttgcaggcatattgcttcatcctggagaagctgctggagaatgaggaaactcaaatcaatggcttctgcatcattgagaacttcaagggctttaccatgcagcaggctgctagtctccggacttcagatctcaggaagatggtggacatgctccaggattccttcccagcccggttcaaagccatccacttcatccaccagccatggtacttcaccacgacctacaatgtggtcaagcccttcttgaagagcaagctgcttgagagggtctttgtccacggggatgacctttctggtttctaccaggagatcgatgagaacatcctgccctctgacttcgggggcacgctgcccaagtatgatggcaaggccgttgctgagcagctctttggcccccaggcccaagctgagaacacagccttctgaggatcgtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtactagggttaccccagaacaggtcccattcatggcccacatgacaacctgcttccccagtgggtatttttggagacagctcttctgtttccaggttttctctcctgcctaaatgtcctgcctaagtgccttcaagaacccttcaccatcctgctcctgcatgtgaccaggttccatggtcagttcaatcacctagtcacagttggtaagtgacagagttgggacttgaacctatgcctgcctgacaccaagtctttttttgacacctagagccaagacatctgaagacaaactccctaggagagctggcgtcatagaaaccttaaaggttagggagacctgggtttgaatcaggctttgtcagttatgacttgtgtgaccctagcaagttatttaacctttctgggtctcagtttcctcatctgcaaactgaggataataacagtacctaccaaaaagaactgtcgtgaaaaccatataatttctgcaatgctcctggcacagtgtcctgttctaaagcatagttccccttctctttcttagctccatattgattattaccctaacttgcacaaagagacttggaggacccccatagagtatcggagggtcccccatttcctgctctttccactccacacccccagcaagcacagggaagttctgggggccataatccacccacaggaaccaaatctaagccacctttctggctggtagacatccaggtatgtgggcacagaggtagacaggctgaaatgctgctgtgctatcagttgggttttgctggaacaggaatggaaatggagaggctgacagaactgccctggggagcccaggcaagagggacagtggctggacacccccagccagttgtgcagaccatcagaacaagatcctagattttaggaatacagggttcaagtccgtgcggcaactcttttctaaatatgcccaagccattaactttgagttttaaaaatactgatttacaagctgtacacaatgaaaaaatgcctatccctcacaccatgctgatgctgttccctgccatctcagattaccaattaaatacagaatgcccagttaaatgtgaactttttttttttttttttttttgagatggagttttgttcttgtcgcccaggctagagtgcaatggtgcgatctcagctcactgcaacctctgcctcccaggttcaagcaattctcctgccttagcctcctgagtagctggaactacaggtgcccaccagcacgcctggctaatttttggtatttttagtggagatggggtttcaccatgttggccaggctggtctcgaactcctgacctcaggtgatctgcctgcctcggcctcccaaagtgctgggattacaggcgtgagcctaaatgtgaacttttttaatactaaaaaagtatttgctgttcatcggaaattcacatttaactgggtgtcctgtatttttatttgctaaatctaccatcaaattggtctggctcaacctggagaatggttaccctaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 56 - DNA, 2119 bp, Artificial Sequence
  Description of Artificial Sequence Synthetic polynucleotide
cgcgctcgctcgctcactgaggccgcccgggcaaagcccgggcgtcgggcgacctttggtcgcccggcctcagtgagcgagcgagcgcgcagagagggagtggggtaccacgcgtttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgaatcgatggatcgggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgccccgggatccatcgattgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 57 - DNA, 4503 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcagcttttgtcctctccctgcttggccttaaccagccacatttctcaactgaccccactcactgcagaggtgaaaactaccatgccaggtcctgctggctgggggaggggtgggcaataggcctggatttgccagagctgccactgtagatgtagtcatatttacgatttcccttcacctcttattaccctggtggtggtggtgggggggggggggtgctctctcagcaaccccaccccgggatcttgaggagaaagagggcagagaaaagagggaatgggactggcccagatcccagccccacagccgggcttccacatggccgagcaggaactccagagcaggagcacacaaaggagggctttgatgcgcctccagccaggcccaggcctctcccctctcccctttctctctgggtcttcctttgccccactgagggcctcctgtgagcccgatttaacggaaactgtgggcggtgagaagttccttatgacacactaatcccaacctgctgaccggaccacgcctccagcggagggaacctctagagctccaggacattcaggtaccaggtagccccaaggaggagctgccgacctggcaggtaagtcaatacctggggcttgcctgggccagggagcccaggactggggtgaggactcaggggagcagggagaccacgtcccaagatgcctgtaaaactgaaaccacctggccattctccaggttgagccagaccaatttgatggcagatttagcaaataaaaatacaggacacccagttaaatgtgaatttcagatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggtaaccaagcattgcttatgtgtccattacatttcataacacttccatcctactttacagggaacaaccaagactggggttaaatctcacagcctgcaagtggaagagaagaacttgaacccaggtccaacttttgcgccacagcaggctgcctcttggtcctgacaggaagtcacaacttgggtctgagtactgatccctggctattttttggctgtgttaccttggacaagtcacttattcctcctcccgtttcctcctatgtaaaatggaaataataatgttgaccctgggtctgagagagtggatttgaaagtacttagtgcatcacaaagcacagaacacacttccagtctcgtgattatgtacttatgtaactggtcatcacccatcttgagaatgaatgcattggggaaagggccatccactaggctgcgaagtttctgagggactccttcgggctggagaaggatggccacaggagggaggagagattgccttatcctgcagtgatcatgtcattgagaacagagccagattctttttttcctggcagggccaacttgttttaacatctaaggactgagctatttgtgtctgtgccctttgtccaagcagtgtttcccaaagtgtagcccaagaaccatctccctcagagccaccaggaagtgctttaaattgcaggttcctaggccacagcctgcacctgcagagtcagaatcatggaggttgggacccaggcacctgcgtttctaacaaatgcctcgggtgattctgatgcaattgaaagtttgagatccacagttctgagacaataacagaatggtttttctaacccctgcagccctgacttcctatcctagggaaggggccggctggagaggccaggacagagaaagcagatcccttctttttccaaggactctgtgtcttccataggcaacgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 58 - DNA, 4543 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacgtcgtttaaacgggccccggtgttatctcattcttttttctcctctgtaagttgacatgtgatgtgggaacaaaggggataaagtcattattttgtgctaaaatcgtaattggagaggacctcctgttagctgggctttcttctatttattgtggtggttactggagttccttcttctagttttaggatatatatatatattttttttttttctttccctgaagatataataatatatatacttctgaagattgagatttttaaattagttgtattgaaaactagctaatcagcaatttaaggctagcttgagacttatgtcttgaatttgtttttgtaggctccaaaaccaaggagggagtggtgcatggtgtggcaacaggtaagctccattgtgcttatatccaaagatgatatttaaagtatctagtgattagtgtggcccagtattcaagattcctatgaaattgtaaaacaatcactgagcattctaagaacatatcagtcttattgaaactgaattctttataaagtatttttaaaaaggtaaatattgattataaataaaaaatatacttgccaagaataatgagggctttgaattgataagctatgtttaatttatagtaagtgggcatttaaatattctgaccaaaaatgtattgacaaactgctgacaaaaataaaatgtgaatattgccataattttaaaaaaagagtaaaatttctgttgattacagtaaaatattttgaccttaaattatgttgattacaatattcctttgataattcagagtgcatttcaggaaacacccttggacagtcagtaaattgtttattgtatttatctttgtattgttatggtatagctatttgtacaaatattattgtgcaattattacatttctgattatattattcatttggcctaaatttaccaagaatttgaacaagtcaattaggtttacaatcaagaaatatcaaaaatgatgaaaaggatgataatcatcatcagatgttgaggaagatgacgatgagagtgccagaaatagagaaatcaaaggagaaccaaaatttaacaaattaaaagcccacagacttgctgtaattaagttttctgttgtaagtactccacgtttcctggcagatgtggtgaagcaaaagatataatcagaaatataatttatatgatcggaaagcattaaacacaatagtgcctatacaaataaaatgttcctatcactgacttctaaaatggaaatgaggacaatgatatgggaatcttaatacagtgttgtggataggactaaaaacacaggagtcagatcttcttggttcaacttcctgcttactccttaccagctgtgtgttttttgcaaggttcttcacctctatgtgatttagcttcctcatctataaaataattcagtgaattaatgtacacaaaacatctggaaaacaaaagcaaacaatatgtattttataagtgttacttatagttttatagtgaactttcttgtgcaacatttttacaactagtggagaaaaatatttctttaaatgaatacttttgatttaaaaatcagagtgtaaaaataaaacagactcctttgaaactagttctgttagaagttaattgtgcacctttaatgggctctgttgcaatccaacagagaagtagttaagtaagtggactatgatggcttctagggacctcctataaatatgatattgtgaagcatgattataataagaactagataacagacaggtggagactccactatctgaagagggtcaacctagatgaatggtgttccatttagtagttgaggaagaacccatgaggtttagaaagcagacaagcatgtggcaagttctggagtcagtggtaaaaattaaagaacccaactattactgtcacctaatgatctaatggagactgtggagatgggctgcatttttttaatcttctccagaatgccaaaatgtaaacacatatctgtgtgtgtgtgtgtgtgtgtgtgtgtgtgtgagagagagagagagagagagagagactgaagtttgtacaattagacattttataaaatgttttctgaaggacagtggctcacaatcttaagtttctaacattgtacaatgttgggagactttgtatactttattttctctttagcatattaaggaatctgagatgtcctacagtaaagaaatttgcattacatagttaaaatcagggttattcaaactttttgattattgaaacctttcttcattagttactagggttgaatgaaactagtgttccacagaaaactatgggaaatgttgctaggcagtaaggacatggtgatttcagcatgtgcaatatttacagcgattgcacccatggaccaccctggcagtagtgaaataaccaaaaatgctgtcataactagtatggctatgagaaacacattgggcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 59 - DNA, 4438 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttacgtaatatttattgaagtttaatattgtgtttgtgatacagaagtatttgctttaattctaaataaaaattttatgcttttattgctggtttaagaagatttggattatccttgtactttgaggagaagtttcttatttgaaatattttggaaacaggtcttttaatgtggaaagatagatattaatctcctcttctattactctccaagatccaacaaaagtgattataccccccaaaatatgatggtagtatcttatactaccatcattttataggcatagggctcttagctgcaaataatggaactaactctaataaagcagaacgcaaatattgtaaatattagagagctaacaatctctgggatggctaaaggatggagcttggaggctacccagccagtaacaatattccgggctccactgttgaatggagacactacaactgccttggatgggcagagatattatggatgctaagccccaggtgctaccattaggacttctaccactgtccctaacgggtggagcccatcacatgcctatgccctcactgtaaggaaatgaagctactgttgtatatcttgggaagcacttggattaattgttatacagttttgttgaagaagacccctagggtaagtagccataactgcacactaaatttaaaattgttaatgagtttctcaaaaaaaatgttaaggttgttagctggtatagtatatatcttgcctgttttccaaggacttctttgggcagtaccttgtctgtgctggcaagcaactgagacttaatgaaagagtattggagatatgaatgaattgatgctgtatactctcagagtgccaaacatataccaatggacaagaaggtgaggcagagagcagacaggcattagtgacaagcaaagatatgcagaatttcattctcagcaaatcaaaagtcctcaacctggttggaagaatattggcactgaatggtatcaataaggttgctagagagggttagaggtgcacaatgtgcttccataacattttatacttctccaatcttagcactaatcaaacatggttgaatactttgtttactataactcttacagagttataagatctgtgaagacagggacagggacaatacccatctctgtctggttcataggtggtatgtaatagatatttttaaaaataagtgagttaatgaatgagggtgagaatgaaggcacagaggtattagggggaggtgggccccagagaatggtgccaaggtccagtggggtgactgggatcagctcaggcctgacgctggccactcccacctagctcctttctttctaatctgttctcattctccttgggaaggattgaggtctctggaaaacagccaaacaactgttatgggaacagcaagcccaaataaagccaagcatcagggggatctgagagctgaaagcaacttctgttccccctccctcagctgaaggggtggggaagggctcccaaagccataactccttttaagggatttagaaggcataaaaaggcccctggctgagaacttccttcttcattctgcagttggtgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 60 - DNA, 3481 bp, Artificial Sequence
  Description of Artificial Sequence Synthetic polynucleotide
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttacgtaattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccgaattccccggggatcctctagagtcgaaattcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagggtaccggtcgacctgcagaagcttgcctcgagcagcgctgctcgagagatctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggtaaccattctccaggttgagccagaccaatttgatggtagatttagcaaataaaaatacaggacacccagttaaatgtgaatttccgatgaacagcaaatacttttttagtattaaaaaagttcacatttaggctcacgcctgtaatcccagcactttgggaggccgaggcaggcagatcacctgaggtcaggagttcgagaccagcctggccaacatggtgaaaccccatctccactaaaaataccaaaaattagccaggcgtgctggtgggcacctgtagttccagctactcaggaggctaaggcaggagaattgcttgaacctgggaggcagaggttgcagtgagctgagatcgcaccattgcactctagcctgggcgacaagaacaaaactccatctcaaaaaaaaaaaaaaaaaaaaagttcacatttaactgggcattctgtatttaattggtaatctgagatggcagggaacagcatcagcatggtgtgagggataggcattttttcattgtgtacagcttgtaaatcagtatttttaaaactcaaagttaatggcttgggcatatttagaaaagagttgccgcacggacttgaaccctgtattcctaaaatctaggatcttgttctgatggtctgcacaactggctgggggtgtccagccactgtccctcttgcctgggctccccagggcagttctgtcagcctctccatttccattcctgttccagcaaaacccaactgatagcacagcagcatttcagcctgtctacctctgtgcccacatacctggatgtctaccagccagaaaggtggcttagatttggttcctgtgggtggattatggcccccagaacttccctgtgcttgctgggggtgtggagtggaaagagcaggaaatgggggaccctccgatactctatgggggtcctccaagtctctttgtgcaagttagggtaataatcaatatggagctaagaaagagaaggggaactatgctttagaacaggacactgtgccaggagcattgcagaaattatatggttttcacgacagttctttttggtaggtactgttattatcctcagtttgcagatgaggaaactgagacccagaaaggttaaataacttgctagggtcacacaagtcataactgacaaagcctgattcaaacccaggtctccctaacctttaaggtttctatgacgccagctctcctagggagtttgtcttcagatgtcttggctctaggtgtcaaaaaaagacttggtgtcaggcaggcataggttcaagtcccaactctgtcacttaccaactgtgactaggtgattgaactgaccatggaacctggtcacatgcaggagcaggatggtgaagggttcttgaaggcacttaggcaggacatttaggcaggagagaaaacctggaaacagaagagctgtctccaaaaatacccactggggaagcaggttgtcatgtgggccatgaatgggacctgttctggggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 61 - DNA, 2327 bp, Artificial Sequence; identical to SEQ ID NO: 51
  Description of Artificial Sequence Synthetic polynucleotide

SEQ ID NO: 62 - DNA, 236 bp, Simian virus 40
agacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatc

SEQ ID NO: 63 - DNA, 954 bp, Homo sapiens
tcagaaggctgtgttctcagcttgggcctgggggccaaagagctgctcagcaacggccttgccatcatacttgggcagcgtgcccccgaagtcagagggcaggatgttctcatcgatctcctggtagaaaccagaaaggtcatccccgtggacaaagaccctctcaagcagcttgctcttcaagaagggcttgaccacattgtaggtcgtggtgaagtaccatggctggtggatgaagtggatggctttgaaccgggctgggaaggaatcctggagcatgtccaccatcttcctgagatctgaagtccggagactagcagcctgctgcatggtaaagcccttgaagttctcaatgatgcagaagccattgatttgagtttcctcattctccagcagcttctccaggatgaagcaatatgcctgcaagatctcatcaaaggtgatttcttgactttgccagttctcaatgttgaagagcatgaccactcggccatacttgtcccgactagagaggacaccagggtagccagcttcaatggtgcagcggacagcctctggggacaggctgtcaaagagctcagggtactgcagccggaaattcacatagcctctgagcagctcataggcacggcccacgttgaacttccgtgcgcggatgaagcgcaggaagaagccgctgtccttctcttgcaccctctccgccacggccaccgccagctcctcccccgaggccgcctgcgcctgcaccatctcctgcagctctcgcactgcctcctcccgggtctcctctctctcgttcagctcatccttggccttctgcaaggtgtggcggggcagctggctgcacgggccaaagacaggtccatggtccttggttgtgagctgctccagttgggcacggagctcctgttcctcttcaggtaccatgcggaacgtgcccaccccttctgacat

SEQ ID NO: 64 - DNA, 6 bp, Artificial Sequence
  Description of Artificial Sequence Synthetic oligonucleotide
ggtggc

SEQ ID NO: 65 - DNA, 183 bp, Artificial Sequence
  Description of Artificial Sequence Synthetic polynucleotide
ggatcccggggcgggtacaattccgcagcttttagagcagaagtaacacttccgtacaggcctagaagtaaaggcaacatccactgaggagcagttctttgatttgcaccaccaccggatccgggacctgaaataaaagacaaaaagactaaacttaccagttaactttctggtttttcagtt

SEQ ID NO: 66 - DNA, 590 bp, Homo sapiens
tcggcagctcctccttggggctacctggtacctgaatgtcctggagctctagaggttccctccgctggaggcgtggtccggtcagcaggttgggattagtgtgtcataaggaacttctcaccgcccacagtttccgttaaatcgggctcacaggaggccctcagtggggcaaaggaagacccagagagaaaggggagaggggagaggcctgggcctggctggaggcgcatcaaagccctcctttgtgtgctcctgctctggagttcctgctcggccatgtggaagcccggctgtggggctgggatctgggccagtcccattccctcttttctctgccctctttctcctcaagatcccggggtggggttgctgagagagcacccccccccccccaccaccaccaccagggtaataagaggtgaagggaaatcgtaaatatgactacatctacagtggcagctctggcaaatccaggcctattgcccacccctcccccagccagcaggacctggcatggtagttttcacctctgcagtgagtggggtcagttgagaaatgtggctggttaaggccaagcagggagaggacaa

SEQ ID NO: 67 - DNA, 720 bp, Artificial Sequence
  Description of Artificial Sequence Synthetic polynucleotide
ttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccat

SEQ ID NO: 68 - protein, 598 aa, Adeno-associated virus
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 69 - protein, 533 aa, Adeno-associated virus
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 70 - protein, 601 aa, Adeno-associated virus
MAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 71 - protein, 535 aa, Adeno-associated virus
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 72 - DNA, 1602 bp, Homo sapiens
atgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttga

SEQ ID NO: 73 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 74 - DNA, 693 bp, Homo sapiens
atgaagaaccccatgctggaggtggtgtctttactactggagaagctgctcctcatctccaacttcacgctctttagttcgggcgccgcgggcgaagacaaagggaggaacagtttttatgaaaccagctctttccaccgaggcgacgtgctggaggtgccccggacccacctgacccactatggcatctacctaggagacaaccgtgttgcccacatgatgcccgacatcctgttggccctgacagacgacatggggcgcacgcagaaggtggtctccaacaagcgtctcatcctgggcgttattgtcaaagtggccagcatccgcgtggacacagtggaggacttcgcctacggagctaacatcctggtcaatcacctggacgagtccctccagaaaaaggcactgctcaacgaggaggtggcgcggagggctgaaaagctgctgggctttaccccctacagcctgctgtggaacaactgcgagcacttcgtgacctactgcagatatggcaccccgatcagtccccagtccgacaagttttgtgagactgtgaagataattattcgtgatcagagaagtgttcttgcttcagcagtcttgggattggcgtctatagtctgtacgggcttggtatcatacactacccttcctgcaatttttattccattcttcctatggatggctggctaa

SEQ ID NO: 75 - protein, 230 aa, Homo sapiens
MKNPMLEVVSLLLEKLLLISNFTLFSSGAAGEDKGRNSFYETSSFHRGDVLEVPRTHLTHYGIYLGDNRVAHMMPDILLALTDDMGRTQKVVSNKRLILGVIVKVASIRVDTVEDFAYGANILVNHLDESLQKKALLNEEVARRAEKLLGFTPYSLLWNNCEHFVTYCRYGTPISPQSDKFCETVKIIIRDQRSVLASAVLGLASIVCTGLVSYTTLPAIFIPFFLWMAG

SEQ ID NO: 76 - DNA, 957 bp, Homo sapiens
atgtggctgcctcttctgctgggtgccttactctgggcagtgctgtggttgctcagggaccggcagagcctgcccgccagcaatgcctttgtcttcatcaccggctgtgactcaggctttgggcgccttctggcactgcagctggaccagagaggcttccgagtcctggccagctgcctgaccccctccggggccgaggacctgcagcgggtggcctcctcccgcctccacaccaccctgttggatatcactgatccccagagcgtccagcaggcagccaagtgggtggagatgcacgttaaggaagcagggctttttggtctggtgaataatgctggtgtggctggtatcatcggacccacaccatggctgacccgggacgatttccagcgggtgctgaatgtgaacacaatgggtcccatcggggtcacccttgccctgctgcctctgctgcagcaagcccggggccgggtgatcaacatcaccagcgtcctgggtcgcctggcagccaatggtgggggctactgtgtctccaaatttggcctggaggccttctctgacagcctgaggcgggatgtagctcattttgggatacgagtctccatcgtggagcctggcttcttccgaacccctgtgaccaacctggagagtctggagaaaaccctgcaggcctgctgggcacggctgcctcctgccacacaggcccactatgggggggccttcctcaccaagtacctgaaaatgcaacagcgcatcatgaacctgatctgtgacccggacctaaccaaggtgagccgatgcctggagcatgccctgactgctcgacacccccgaacccgctacagcccaggttgggatgccaagctgctctggctgcctgcctcctacctgccagccagcctggtggatgctgtgctcacctgggtccttcccaagcctgcccaagcagtctactga

SEQ ID NO: 77 - protein, 318 aa, Homo sapiens
MWLPLLLGALLWAVLWLLRDRQSLPASNAFVFITGCDSGFGRLLALQLDQRGFRVLASCLTPSGAEDLQRVASSRLHTTLLDITDPQSVQQAAKWVEMHVKEAGLFGLVNNAGVAGIIGPTPWLTRDDFQRVLNVNTMGPIGVTLALLPLLQQARGRVINITSVLGRLAANGGGYCVSKFGLEAFSDSLRRDVAHFGIRVSIVEPGFFRTPVTNLESLEKTLQACWARLPPATQAHYGGAFLTKYLKMQQRIMNLICDPDLTKVSRCLEHALTARHPRTRYSPGWDAKLLWLPASYLPASLVDAVLTWVLPKPAQAVY

(19) United States
(12) Patent Application Publication
(10) Pub. No.: US 2024/0060989 A1
(43) Pub. Date: Feb. 22, 2024

(54) METHODS FOR MEASURING CRALBP ACTIVITY

(71) Applicant: NOVARTIS AG, Basel (CH)

(72) Inventor: Konrad MUELLER, Emmendingen (DE)

(21) Appl. No.: 17/766,437

(22) PCT Filed: Oct. 2, 2020
(86) PCT No.: PCT/IB2020/059296

§ 371 Date: Apr. 4, 2022

Related U.S. Application Data
(60) Provisional application No. 62/910,746, filed on Oct. 4, 2019.

(51) Int. Cl. G01N 33/68; C12N 15/86; C12N 5/071

(52) U.S. Cl. CPC G01N 33/6872; G01N 33/6845; C12N 15/86; C12N 5/0686; C12N 2750/14143

(57) ABSTRACT

The present disclosure provides methods for measuring activity of cellular retinaldehyde-binding protein (CRALBP) or potency of a composition comprising an AAV vector comprising a CRALBP coding sequence for expressing a CRALBP protein. Also provided are kits for use in measuring activity of CRALBP.

### CROSS-REFERENCE TO RELATED APPLICATION AND INCORPORATION OF SEQUENCE LISTING

This application claims priority to U.S. Provisional Patent Appln. No. 62/910,746 filed Oct. 4, 2019, and is incorporated into this application by reference in its entirety. The sequence listing that is contained in the filed named “PAT058721-WO-PCT_ST25,” which is 267,180 bytes (measured in operating system MS-Windows) and was created on Sep. 30, 2020, is filed herewith and incorporated herein by reference.

### FIELD

The present disclosure relates to assays and methods for measuring activity of cellular retinaldehyde-binding protein (CRALBP) or potency of a composition comprising AAV vectors comprising a CRALBP coding sequence for expressing a CRALBP protein. Also provided is a kit for use in measuring activity of CRALBP.

### BACKGROUND

Retinitis pigmentosa (RP) refers to a group of inherited degenerations of the photoreceptor cells (rods and cones) of the retina leading to visual loss and blindness. RLBP1-associated retinal dystrophy is a rare form of RP caused by mutations in the retinaldehyde binding protein 1 (RLBP1) gene on chromosome 15. RLBP1-associated retinal dystrophy is characterized by early severe night blindness and slow dark adaptation, followed by progressive loss of visual acuity, visual fields, and color vision, leading to legal blindness typically around middle adulthood. The fundus appearance is characterized by yellow or white spots in the retina. The reduction in visual acuity and visual field significantly impacts patients' quality of life.

Mutations in the RLBP1 gene cause the absence of or dysfunction of the cellular retinaldehyde-binding protein (CRALBP), a protein that is important in the visual cycle (FIG. 1). CRALBP is expressed in retinal pigment epithelium (RPE) and Midler cells, ciliary epithelium, iris, cornea, pineal gland, and a subset of oligodendrocytes of the optic nerve and brain. CRALBP accepts 11-cis retinol from the isomerase retinal pigment epithelium-specific protein 65-KD (RPE65) and acts as a carrier for 11-cis retinol dehydrogenase 5 (RDH5) to convert 11-cis retinol to 11-cis retinal. The rate of chromophore regeneration is severely reduced in the absence of functional CRALBP.

The use of recombinant adeno-associated viral (AAV) vectors to express CRALBP proteins for treating subjects suffering from retinal diseases and blindness is described in U.S. Pat. No. 9,163,259 B2 and U.S. Pat. No. 9,803,217 B2, which are incorporated by reference in their entirety. There is a need for assays and methods to measure the potency of viral vector batches of recombinant AAVs for expressing CRALBP proteins for gene therapy for RP.

### BRIEF DESCRIPTION OF THE DRAWINGS

FIG. 1 shows the visual cycle.

FIG. 2 shows binding between 11-cis-retinol and human CRALBP under ambient light (2A) or dark (2B) condition.

### SUMMARY

The present disclosure provides assays and methods for measuring activity of cellular retinaldehyde-binding protein (CRALBP). In specific aspects, the present disclosure provides a method for measuring activity of cellular retinaldehyde-binding protein (CRALBP) comprising: a) contacting a cell with an adeno-associated viral (AAV) vector comprising a heterologous gene encoding a CRALBP protein, whereby a transduced cell expressing the CRALBP protein is generated; b) lysing the transduced cell to produce a cell extract thereof; c) incubating the cell extract with a composition comprising a substrate of the vision cycle, under conditions wherein the substrate is converted to a reaction product in the presence of CRALBP protein; and d) determining the reaction product, whereby the amount of the reaction product reflects the activity of the CRALBP protein.

The present disclosure also provides a method for measuring potency of a composition comprising an AAV vector comprising a CRALBP coding sequence for expressing a CRALBP protein, the method comprising: a) contacting a cell with the AAV vector, whereby a transduced cell expressing the CRALBP protein is generated; b) lysing the transduced cell to produce a cell extract thereof; c) incubating the cell extract with a composition comprising a substrate of the vision cycle, wherein the substrate is converted to a reaction product in the presence of CRALBP protein; and d) determining the reaction product, whereby the amount of the reaction product reflects the activity of the CRALBP protein.

In one aspect, the cell expresses a protein having lecithin retinol acyltransferase (LRAT) activity. In one aspect, the composition further comprises a protein having LRAT activity.

In one aspect, the substrate in step (c) is all-trans retinyl ester or all-trans retinol. In one aspect, the reaction product is 11-cis retinol.

In one aspect, the composition in step (c) further comprises a protein having retinal pigment epithelium-specific protein 65-KD (RPE65) activity. In one aspect, the protein having RPE65 activity is a mammalian RPE65. In one aspect, the protein having RPE65 activity is a human RPE65. In one aspect, the protein having RPE65 activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 72. In one aspect, the protein having RPE65 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 73.

In one aspect, the reaction product comprises 11-cis retinal. In one aspect, the composition in step (c) further comprises a protein having RPE65 activity and a protein having 11-cis retinol dehydrogenase 5 (RDH5) activity. In one aspect, the protein having RDH5 activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 76. In one aspect, the protein having RDH5 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 77.

In one aspect, the AAV vector comprises in the 5′ to 3′ direction: a) a 5′ inverted terminal repeat (ITR); b) a recombinant CRALBP-coding sequence; and c) a 3′ ITR.

In one aspect, the recombinant CRALBP-coding sequence is operably linked to a promoter sequence selected from the group consisting of SEQ ID NOs: 3, 10, 11, 12, and 22. In one aspect, the recombinant CRALBP-coding sequence comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6. In one aspect, the recombinant CRALBP-coding sequence encodes a protein that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7. In one aspect, the recombinant CRALBP-coding sequence comprises a nucleic acid sequence selected from the group consisting of SEQ ID NOs: 37, 39, 41, 43, 45, and 47. In one aspect, the recombinant CRALBP-coding sequence encodes a protein that comprises an amino acid sequence selected from the group consisting of SEQ ID NOs: 38, 40, 42, 44, 46, and 48.

In one aspect, the 5′ ITR comprises a nucleic acid sequence set forth in SEQ ID NO: 2. In one aspect, the 5′ ITR comprises a nucleic acid sequence as set forth in SEQ ID NO: 16 or 17. In one aspect, the AAV vector comprises a nucleic acid sequence, in the 5′ to 3′ direction, selected from the group consisting of: SEQ ID NOs: 2, 10, 5, 6, 8, and 9; SEQ ID NOs: 2, 11, 5, 6, 8, 14, and 9; SEQ ID NOs: 2, 22, 5, 6, 8, 23, and 9; and SEQ ID NOs: 2, 3, 4, 5, 6, 8, 23, and 9.

In one aspect, the 5′ ITR comprises a non-resolvable ITR. In one aspect, the non-resolvable ITR comprises a nucleic acid sequence as set forth in SEQ ID NO: 1. In one aspect, the recombinant CRALBP-coding sequence comprises a nucleic acid sequence as set forth in SEQ ID NO: 6. In one aspect, the AAV vector comprises a nucleic acid sequence, in the 5′ to 3′ direction, of SEQ ID NOs: 1, 5, 6, 8, and 9. In one aspect, the AAV vector comprises a nucleic acid sequence, in the 5′ to 3′ direction, of SEQ ID NOs: 1, 3, 4, 5, 6, 8, and 9. In one aspect, the AAV vector comprises a nucleic acid sequence, in the 5′ to 3′ direction, of SEQ ID NOs: 36, 62, 63, 64, 65, 66, 1, 3, 4, 5, 6, 8, and 9.

In one aspect, the AAV vector comprises an AAV serotype 2 capsid. In one aspect, the AAV serotype 2 capsid is encoded by a nucleic acid sequence of SEQ ID NO: 18. In one aspect, the AAV vector comprises an AAV serotype 8 capsid. In one aspect, AAV serotype 8 capsid is encoded by a nucleic acid sequence of SEQ ID NO: 20. In one aspect, the AAV vector comprises an AAV serotype 5 capsid.

In one aspect, the cell expressing a protein having LRAT activity is a mammalian cell. In one aspect, the cell expressing a protein having LRAT activity is a human cell. In one aspect, the cell expressing a protein having LRAT activity is a HeLa cell. In one aspect, the cell expressing a protein having LRAT activity is a human embryonic kidney (HEK) 293 cell. In one aspect, the cell expresses a protein having LRAT activity stably. In one aspect, the cell expresses a protein having LRAT activity transiently. In one aspect, the protein having LRAT activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 74. In one aspect, the protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75.

In one aspect, step (c) comprises adding a precursor of the substrate to the cell extract, whereby the precursor is converted to the substrate. In one aspect, the precursor comprises all-trans retinol. In one aspect, the precursor is mixed with an at least 10% solution of dimethylformamide (DMF). In one aspect, the all-trans retinol is added such that the final concentration is about 1 mM to about 20 mM.

In one aspect, the contacting in step (a) is with an amount of about 500 to about 5×10⁶ of the AAV vector per cell. In one aspect, the contacting in step (a) is with an amount of about 1,000 to about 1×10⁶ of the AAV vector per cell. In one aspect, the contacting in step (a) is with an amount of about 2,000 to about 5×10⁵ of the AAV vector per cell. In one aspect, the lysing in step (b) comprises freeze-thawing, sonication, or a combination thereof.

In one aspect, after the lysing in step (b) the transduced cell is diluted in a salt buffer. In one aspect, the salt buffer is a sodium chloride buffer. In one aspect, steps (c) and (d) are performed in the dark, under dim light, or under dim yellow light. In one aspect, the incubating in step (c) is from about 30 minutes to about 240 minutes. In one aspect, the incubating in step (c) is from about 6 hours to about 96 hours. In one aspect, the incubating in step (c) is at a temperature from about 30° C. to about 40° C.

In one aspect, after step (c) but before step (d) the reaction is quenched or stopped. In one aspect, after step (c) but before step (d) an alcohol is added. In one aspect, the reaction product is extracted with an organic solvent. In one aspect, the organic solvent is hexane.

In one aspect, the determining in step (d) comprises subjecting the reaction product to column chromatography, thereby producing a column chromatography purified reaction product. In one aspect, the column chromatography comprises a reverse-phase chromatography. In one aspect, the column chromatography comprises a reverse-phase stationary phase. In one aspect, step (d) comprises subjecting the column chromatography purified reaction product to mass spectrometry, thereby quantifying the reaction product.

Also provided in the present disclosure is a kit for use in measuring activity of CRALBP comprising: a) an AAV-ITR-containing plasmid comprising a heterologous gene encoding a CRALBP protein; b) an AAV-Rep-Cap-containing plasmid; c) a helper plasmid; and d) a composition comprising a substrate of the vision cycle. In one aspect, the kit further comprises a composition of cells that can be transduced with a viral vector to express CRALBP protein.

In one aspect, the kit further comprises cell expressing a protein having LRAT activity. In one aspect, the kit further comprises a protein having LRAT activity. In one aspect, the composition further comprises a protein having RPE65 activity. In one aspect, the helper plasmid is an Adeno-helper plasmid.

In one aspect, the cell expressing a protein having LRAT activity is a human embryonic kidney (HEK) 293 cell. In one aspect, the protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75. In one aspect, the recombinant CRALBP-coding sequence comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6.

In one aspect, the recombinant CRALBP-coding sequence comprises a nucleic acid sequence selected from the group consisting of SEQ ID NOs: 37, 39, 41, 43, 45, and 47.

In one aspect, the AAV-ITR-containing plasmid comprises a nucleic acid sequence selected from the group consisting of SEQ ID NOs: 26, 27, 28, 29, 30, and 50. In one aspect, the AAV-ITR-containing plasmid comprises a nucleic acid sequence in the 5′ to 3′ direction, selected from the group consisting of: SEQ ID NOs: 2, 10, 5, 6, 8, and 9; SEQ ID NOs: 2, 11, 5, 6, 8, 14, and 9; SEQ ID NOs: 2, 22, 5, 6, 8, 23, and 9; SEQ ID NOs: 2, 3, 4, 5, 6, 8, 23, and 9; and SEQ ID NOs: 1, 5, 6, 8, and 9.

In one aspect, the AAV-Rep-Cap-containing plasmid encodes an AAV serotype 2 capsid. In one aspect, the 2 capsid is encoded by a nucleic acid sequence of SEQ ID NO: 18. In one aspect, the AAV-Rep-Cap-containing plasmid encodes an AAV serotype 8 capsid. In one aspect, the AAV serotype 8 capsid is encoded by a nucleic acid sequence of SEQ ID NO: 20.

In one aspect, the substrate comprises all-trans retinyl ester or all-trans retinol.

In one aspect, the protein having RPE65 activity is a human RPE65. In one aspect, the protein having RPE65 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 73.

The present disclosure further provides a cell for use in a method for measuring activity of CRALBP, wherein the cell recombinantly expresses a protein having LRAT activity and a protein having CRALBP activity. In one aspect, the protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75. In another aspect, the protein having CRALBP activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7. In one aspect, the cell comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 74. In another aspect, the cell comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6. In one aspect, the cell is an HEK293 cell. In another aspect, the cell is a HeLa cell.

### DETAILED DESCRIPTION

### Definitions

Unless defined otherwise, all technical and scientific terms used herein have the same meaning as commonly understood by those of ordinary skill in the art to which this present disclosure pertains. Any references cited herein, including, e.g., all patents, published patent applications, and non-patent publications, are incorporated by reference in their entirety. To facilitate understanding of the disclosure, several terms and abbreviations as used herein are defined below as follows:

The term “about” as used herein, is intended to qualify the numerical values that it modifies, denoting such a value as variable within a margin of error. When no particular margin of error, such as a standard deviation to a mean value, is recited, the term “about” should be understood to mean that range which would encompass the recited value and the range which would be included by rounding up or down to that figure, taking into account significant figures.

The term “gene cassette” refers to a manipulatable fragment of DNA carrying, and capable of expressing, one or more genes, or coding sequences, of interest between one or more sets of restriction sites. A gene cassette can be transferred from one DNA sequence (often in a plasmid vector) to another by ‘cutting’ the fragment out using restriction enzymes and ligating it back into a new context, for example into a new plasmid backbone.

The term “heterologous gene” or “heterologous nucleotide sequence” in the context of a viral vector will typically refer to a gene or nucleotide sequence that is not naturally-occurring in the virus. Alternatively, a heterologous gene or nucleotide sequence may refer to a viral sequence that is placed into a non-naturally occurring environment (e.g., by association with a promoter with which it is not naturally associated in the virus).

The terms “ITR” or “inverted terminal repeat” refer to the stretch of nucleic acid sequences that exist in Adeno-Associated Viruses (AAV) and/or recombinant Adeno-Associated Viral Vectors (rAAV) that can form a T-shaped palindromic structure, that is required for completing AAV lytic and latent life cycles (Muzyczka and Berns 2001).

The term “non-resolvable ITR” refers to a modified ITR such that the resolution by the Rep protein is reduced. A non-resolvable ITR can be an ITR sequence without the terminal resolution site (TRS) which leads to low or no resolution of the non-resolvable ITR and would yield 90-95% of self-complementary AAV vectors (McCarty et al 2003). A specific example of a non-resolvable ITR is “ΔITR”, having a sequence of SEQ ID NO: 1.

As commonly understood in the art, a “mutation” refers to any alteration of a nucleotide sequence of the genome, extrachromosomal DNA, or other genetic element of an organism (e.g., a gene or regulatory element operably linked to a gene in an organism), such as a nucleotide insertion, deletion, inversion, substitution, duplication, etc.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” can also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

It is recognized that residue positions of proteins that are not identical often differ by conservative amino acid substitutions, where amino acid residues are substituted for other amino acid residues with similar size and chemical properties (e.g., charge, hydrophobicity, polarity, etc.), and therefore may not change the functional properties of the molecule. When sequences differ in conservative substitutions, the percent sequence similarity can be adjusted upwards to correct for the conservative nature of the non-identical substitution(s). Sequences that differ by such conservative substitutions are said to have “sequence similarity” or “similarity.” Thus, “percent similarity” or “percent similar” as used herein in reference to two or more protein sequences is calculated by (i) comparing two optimally aligned protein sequences over a window of comparison, (ii) determining the number of positions at which the same or similar amino acid residue occurs in both sequences to yield the number of matched positions, (iii) dividing the number of matched positions by the total number of positions in the window of comparison (or the total length of the reference or query protein if a window of comparison is not specified), and then (iv) multiplying this quotient by 100% to yield the percent similarity. Conservative amino acid substitutions for proteins are known in the art.

For optimal alignment of sequences to calculate their percent identity or similarity, various pair-wise or multiple sequence alignment algorithms and programs are known in the art, such as ClustalW, or Basic Local Alignment Search Tool® (BLAST®), etc., that can be used to compare the sequence identity or similarity between two or more nucleotide or protein sequences. Although other alignment and comparison methods are known in the art, the alignment between two sequences (including the percent identity ranges described above) can be as determined by the ClustalW or BLAST® algorithm, see, e.g., Chenna R. et al., “Multiple sequence alignment with the Clustal series of programs,” Nucleic Acids Research 31: 3497-3500 (2003); Thompson J D et al., “Clustal W: Improving the sensitivity of progressive multiple sequence alignment through sequence weighting, position-specific gap penalties and weight matrix choice,” Nucleic Acids Research 22: 4673-4680 (1994); and Larkin M A et al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

The term “operably linked” refers to a functional relationship between two or more polynucleotide (e.g., DNA) segments. Typically, the term refers to the functional relationship of a transcriptional regulatory sequence to a sequence to be transcribed. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribable sequence are contiguous to the transcribable sequence, i.e., they are cis-acting. However, some transcriptional regulatory sequences, such as enhancers, need not be physically contiguous or located in close proximity to the coding sequences whose transcription they enhance.

The term “promoter” refers to a sequence that regulates transcription of an operably-linked gene, or nucleotide sequence encoding a protein or an RNA transcript, etc. Promoters provide the sequence sufficient to direct transcription, as well as, the recognition sites for RNA polymerase and other transcription factors required for efficient transcription and can direct cell specific expression. In addition to the sequence sufficient to direct transcription, a promoter sequence of the present disclosure can also include sequences of other regulatory elements that are involved in modulating transcription (e.g., enhancers, kozak sequences and introns). Examples of promoters known in the art and useful in the viral vectors described herein, include, but are not limited to, the CMV promoter, CBA promoter, smCBA promoter and those promoters derived from an immunoglobulin gene, SV40, or other tissue specific genes (e.g: RLBP1, RPE, VMD2). Specific promoters may also include those described in Table 1, for example, the “RLBP1 (short)” promoter (SEQ ID NO: 3), the “RLBP1 (long)” promoter (SEQ ID NO: 10), RPE65 promoter (SEQ ID NO: 11), VMD2 promoter (SEQ ID NO: 12), and the CMV enhancer and CBA promoter (SEQ ID NO: 22). In addition, standard techniques are known in the art for creating functional promoters by mixing and matching known regulatory elements. “Truncated promoters” may also be generated from promoter fragments or by mix and matching fragments of known regulatory elements; for example the smCBA promoter is a truncated form of the CBA promoter.

As used herein, a “functional portion” of a promoter sequence refers to a part of the promoter sequence that provides essentially the same or similar expression pattern of an operably linked coding sequence or gene as the full promoter sequence. For this definition, “essentially the same or similar” means that the pattern and level of expression of a coding sequence operably linked to the functional portion of the promoter sequence closely resembles the pattern and level of expression of the same coding sequence operably linked to the full promoter sequence.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc.

As used herein, an “encoding region” or “coding region” refers to a portion of a polynucleotide that encodes a functional unit or molecule (e.g., without being limiting, a mRNA, protein, or non-coding RNA sequence or molecule).

The term “RLBP1” refers to the “Retinaldehyde Binding Protein 1.” The human RLBP1 gene is found on chromosome 15, and an exemplary nucleic acid coding sequence of human RLBP1 is set out in SEQ ID NO: 6. The “RLBP1 gene product” is also known as, “cellular retinaldehyde-binding protein” or “CRALBP” and is the protein encoded by the RLBP1 gene. One of skill in the art would understand that an RLBP1 coding sequence may include any nucleic acid sequence that encodes an RLBP1 gene product. The RLBP1 coding sequence may or may not include intervening regulatory elements (e.g., introns, enhancers, or other non-coding sequences).

As used herein, “CRALBP,” “a CRALBP protein,” or “a protein having CRALBP activity,” refers to a protein having the activity of CRALBP to act as a carrier for 11-cis retinol for its conversion to 11-cis retinal in the presence of 11-cis retinol dehydrogenase 5 (RDH5) in a eukaryotic cell. In one aspect, a “CRALBP,” “a CRALBP protein,” or “a protein having CRALBP activity” comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7. In one aspect, a “CRALBP,” “a CRALBP protein,” or “a protein having CRALBP activity” is encoded by a CRALBP-coding sequence comprising a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6. In another aspect, a CRALBP,” “a CRALBP protein,” or “a protein having CRALBP activity” encodes a protein that comprises an amino acid sequence selected from the group consisting of SEQ ID NOs: 38, 40, 42, 44, 46, and 48.

As used herein, “LRAT,” “a LRAT protein,” or “a protein having LRAT activity,” refers to a protein having the activity of lecithin retinol acyltransferase to convert all-trans retinol to retinyl ester in a eukaryotic cell. In one aspect, a “LRAT,” “a LRAT protein,” or “a protein having LRAT activity” comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75. In one aspect, a “LRAT,” “a LRAT protein,” or “a protein having LRAT activity” is encoded by a LRAT-coding sequence comprising a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 74.

As used herein, “RPE65,” “an RPE65 protein,” or “a protein having RPE65 activity,” refers to a protein having the activity of retinal pigment epithelium-specific protein 65-KD to convert retinyl ester to 11-cis retinol in a eukaryotic cell. In one aspect, an “RPE65,” “an RPE65 protein,” or “a protein having RPE65 activity” comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 73. In one aspect, an “RPE65,” “an RPE65 protein,” or “a protein having RPE65 activity” is encoded by an RPE65-coding sequence comprising a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 72.

As used herein, “RDH5,” “an RDH5 protein,” or “a protein having RDH5 activity,” refers to a protein having the activity of 11-cis retinol dehydrogenase 5 to convert 11-cis retinol to 11-cis retinal in a eukaryotic cell. In one aspect, an “RDH5,” “an RDH5 protein,” or “a protein having RDH5 activity” comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 77. In one aspect, an “RDH5,” “an RDH5 protein,” or “a protein having RDH5 activity” is encoded by an RDH5-coding sequence comprising a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 76.

The term “subject” includes human and non-human animals. Non-human animals include all vertebrates (e.g., mammals and non-mammals) such as, non-human primates (e.g., cynomolgus monkey), mice, rats, rabbits, sheep, dogs, cows, chickens, amphibians, and reptiles. Except when noted, the terms “patient” or “subject” are used herein interchangeably.

As used herein, the term “treating” or “treatment” of any disease or disorder (e.g., retinitis pigmentosa, RBLP1-associated retinal dystrophy) refers, to ameliorating the disease or disorder such as by slowing or arresting or reducing the development of the disease or at least one of the clinical symptoms thereof. “Treating” or “treatment” can also refer to alleviating or ameliorating at least one physical parameter including those which may not be discernible by the patient. “Treating” or “treatment” can also refer to modulating the disease or disorder, either physically, (e.g., stabilization of a discernible symptom), physiologically, (e.g., stabilization of a physical parameter), or both. More specifically, “treatment” of RLBP1-associated retinal dystrophy means any action that results in the improvement or preservation of visual function and/or regional anatomy in a subject having RLBP1-associated retinal dystrophy.

The term “AAV vector” or “viral vector” refers to a non-wild-type recombinant AAV viral particle that functions as a gene delivery vehicle and which comprises a recombinant AAV viral genome packaged within an AAV capsid. The recombinant viral genome packaged in the a viral vector is also referred to herein as the “vector genome.”

The term “capsid” refers to the protein coat of the virus or viral vector. The term “AAV capsid” refers to the protein coat of the adeno-associated virus (AAV), which is composed of a total of 60 subunits; each subunit is an amino acid sequence, which can be viral protein 1 (VP1), VP2 or VP3.

### DETAILED DESCRIPTION

### The Visual Cycle

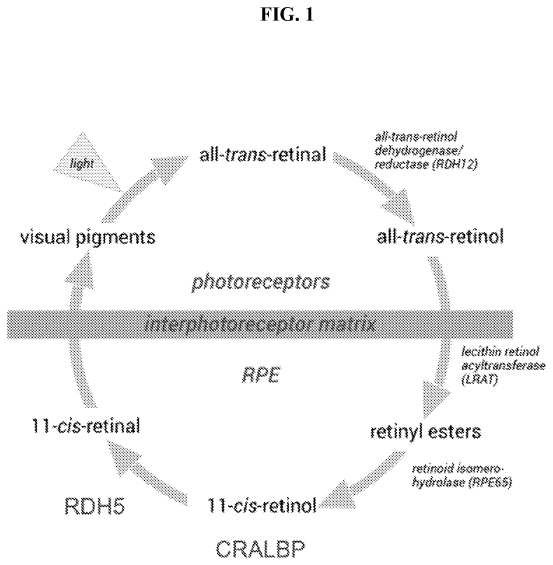

The visual cycle (FIG. 1) regenerates 11-cis retinal through a series of steps involving specialized enzymes and retinoid binding proteins, and the importance of each step is underscored by the fact that each has been identified as sources of visual impairment or blindness in humans.

The visual cycle begins in the rod outer segment with the absorption of a photon by a visual pigment molecule. Rod outer segments contain stacks of membranous discs made of a lipid bi-layer. All-trans retinal is released from the activated opsin into inner leaflet of the disc bi-layer and is believed to complex with phosphatidylethanolamine. The resulting N-retinylidine-phosphatidylethanolamine is transported to the cytoplasmic disc surface by the retina specific ATP binding cassette transporter (ABCR), and released into the cytoplasm as all-trans retinal. Once in the cytoplasm, all-trans retinal is reduced to all-trans-retinol (Vitamin A) by all-trans retinol dehydrogenase/reductase (RDH12) in an NADPH-dependent reaction. All-trans retinol then exits the photoreceptor, crosses the sub-retinal space bound to the interphotoreceptor retinoid binding protein (IRBP), and enters the retinal pigment epithelium (RPE).

In the RPE, at least three enzymes associated with the smooth endoplasmic reticulum convert all-trans retinol to 11-cis retinal. After entering an RPE cell, all-trans retinol is transferred to the cellular retinoid binding protein (CRBP) and delivered to the first visual cycle enzyme in the RPE, lecithin retinol acyl transferase (LRAT). LRAT links all-trans retinol to phosphatidyl choline in the membrane to generate all-trans retinyl esters. Additionally, all-trans retinol from systemic circulation can enter the visual cycle through the basal surface of RPE cells for esterification by LRAT. The esters generated by LRAT are the primary storage form of retinoids in the eye, and their accumulation is thought to be an important force driving subsequent reactions in the visual cycle. More importantly, they serve as the substrate for the next step of the visual cycle and are required for 11-cis retinal regeneration.

The next step of the visual cycle involves the simultaneous hydrolysis and isomerization of all-trans retinyl esters to yield 11-cis retinol. The coupling of isomerization and hydrolysis is facilitated by a single enzyme, an isomerohydrolase, named retinal pigment epithelium-specific protein 65-KD (RPE65). 11-cis retinol binds the cellular retinaldehyde-binding protein (CRALBP), a retinoid binding protein with high affinity for 11-cis retinoids.

CRALBP delivers the 11-cis retinol to 11-cis retinol dehydrogenase 5 (RDH5) for the third and final enzymatic step in the RPE. RDH5 oxidizes 11-cis retinol to 11-cis retinal using NAD as a cofactor, and newly generated 11-cis retinal crosses the sub-retinal space and re-enters the photoreceptors. After entering the outer segment, the newly generated 11-cis retinal can bind with opsin and regenerate functional visual pigment to complete the cycle.

### Viral Vectors

The present disclosure provides a method for measuring CRALBP activity in which an AAV vector comprising a heterologous gene encoding a CRALBP protein is used. In one aspect, an AAV vector of the present disclosure comprises in the 5′ to 3′ direction: a) a 5′ inverted terminal repeat (ITR); b) a recombinant CRALBP-coding sequence; and c) a 3′ ITR.

AAVs are small, single-stranded DNA viruses which require helper virus to facilitate efficient replication. A viral vector comprises a vector genome and a protein capsid. The viral vector capsid may be supplied from any of the AAV serotypes known in the art, including presently identified human and non-human AAV serotypes and AAV serotypes yet to be identified. Virus capsids can be mixed and matched with other vector components to form a hybrid viral vector. For example, the ITRs and capsid of the viral vector may come from different AAV serotypes. In one aspect, the ITRs can be from an AAV2 serotype while the capsid is from, for example, an AAV2 or AAV8 serotype. In addition, one of skill in the art would recognize that the vector capsid may also be a mosaic capsid (e.g., a capsid composed of a mixture of capsid proteins from different serotypes), or even a chimeric capsid (e.g., a capsid protein containing a foreign or unrelated protein sequence for generating markers and/or altering tissue tropism). It is contemplated that the viral vector of the present disclosure may comprise an AAV2 capsid. It is further contemplated that the present disclosure provides methods and assays to measure the activity of CRALBP produced by a viral vector comprising an AAV8 capsid. In certain aspects, the present disclosure provides methods and assays for measuring the activity of CRALBP produced by a viral vector comprising an AAVS capsid, AA6 capsid, or AAV9 capsid.

The SEQ ID NOs in the present disclosure are summarized in Table 1 below.

TABLE 1
Summary of SEQ ID NOS

| SEQ | | |
|---|---|---|
| ID NO | Description | Sequence |
| 1 | ΔITR | cgcgctcgctcgctcactgaggccgcccgggcaaagcccgggcgtcgggcgacctttggtcgcccggcct |
| | | cagtgagcgagcgagcgcgcagagagggagtgg |
| 2 | 5′ ITR | ctgcgcgctcgctcgctcactgaggccgcccgggcgtcgggcgacctttggtcgcccggcctcagtgagc |
| | | gagcgagcgcgcagagagggagtggccaactccatcactaggggttcct |
| 3 | Human RLBP1 | ttgtcctctccctgcttggccttaaccagccacatttctcaactgaccccactcactgcagaggtgaaaactacc |
| | promoter (short) | atgccaggtcctgctggctgggggaggggtgggcaataggcctggatttgccagagctgccactgtagatg |
| | NT_010274.17 | tagtcatatttacgatttcccttcacctcttattaccctggtggtggtggtgggggggggggggtgctctctcag |
| | | caaccccaccccgggatcttgaggagaaagagggcagagaaaagagggaatgggactggcccagatcc |
| | | cagccccacagccgggcttccacatggccgagcaggaactccagagcaggagcacacaaaggagggctt |
| | | tgatgcgcctccagccaggcccaggcctctcccctctcccctttctctctgggtcttcctttgccccactgagg |
| | | gcctcctgtgagcccgatttaacggaaactgtgggggtgagaagttccttatgacacactaatcccaacctg |
| | | ctgaccggaccacgcctccagcggagggaacctctagagctccaggacattcaggtaccaggtagcccca |
| | | aggaggagctgccga |
| 4 | Modified SV40 | aactgaaaaaccagaaagttaactggtaagtttagtctttttgtcttttatttcaggtcccggatccggtggtggtg |
| | intron (modified | caaatcaaagaactgctcctcagtggatgttgcctttacttctaggcctgtacggaagtgttacttctgctctaaa |
| | EF579804) | agctgcggaattgtacccgccccgggatcc |
| 5 | Kozak sequence | gccacc |
| 6 | Human RLBP1 | atgtcagaaggggtgggcacgttccgcatggtacctgaagaggaacaggagctccgtgcccaactggagc |
| | gene CDS | agctcacaaccaaggaccatggacctgtctttggcccgtgcagccagctgccccgccacaccttgcagaag |
| | NM_000326.4 | gccaaggatgagctgaacgagagagaggagacccgggaggaggcagtgcgagagctgcaggagatgg |
| | | tgcaggcgcaggcggcctcgggggaggagctggcggtggccgtggcggagagggtgcaagagaagga |
| | | cagcggcttcttcctgcgcttcatccgcgcacggaagttcaacgtgggccgtgcctatgagctgctcagagg |
| | | ctatgtgaatttccggctgcagtaccctgagctctttgacagcctgtccccagaggctgtccgctgcaccattg |
| | | aagctggctaccctggtgtcctctctagtcgggacaagtatggccgagtggtcatgctcttcaacattgagaac |
| | | tggcaaagtcaagaaatcacctttgatgagatcttgcaggcatattgcttcatcctggagaagctgctggagaa |
| | | tgaggaaactcaaatcaatggcttctgcatcattgagaacttcaagggctttaccatgcagcaggctgctagtc |
| | | tccggacttcagatctcaggaagatggtggacatgctccaggattccttcccagcccggttcaaagccatcca |
| | | cttcatccaccagccatggtacttcaccacgacctacaatgtggtcaagcccttcttgaagagcaagctgcttg |
| | | agagggtctttgtccacggggatgacctttctggtttctaccaggagatcgatgagaacatcctgccctctgac |
| | | ttcgggggcacgctgcccaagtatgatggcaaggccgttgctgagcagctctttggcccccaggcccaagc |
| | | tgagaacacagccttctga |
| 7 | Human RLBP1 | MSEGVGTFRMVPEEEQELRAQLEQLTTKDHGPVFGPCSQLPRHTLQ |
| | gene product | KAKDELNEREETREEAVRELQEMVQAQAASGEELAVAVAERVQEK |
| | (CRALBP) | DSGFFLRFIRARKFNVGRAYELLRGYVNFRLQYPELFDSLSPEAVRC |
| | | TIEAGYPGVLSSRDKYGRVVMLFNIENWQSQEITFDEILQAYCFILEK |
| | | LLENEETQINGFCIIENFKGFTMQQAASLRTSDLRKMVDMLQDSFPA |
| | | RFKAIHFIHQPWYFTTTYNVVKPFLKSKLLERVFVHGDDLSGFYQEI |
| | | DENILPSDFGGTLPKYDGKAVAEQLFGPQAQAENTAF |
| 8 | SV40 poly A | gatcataatcagccataccacatttgtagaggttttacttgctttaaaaaacctcccacacctccccctgaacctg |
| | (EF579804) | aaacataaaatgaatgcaattgttgttgttaacttgtttattgcagcttataatggttacaaataaagcaatagcatc |
| | | acaaatttcacaaataaagcatttttttcactgcattctagttgtggtttgtccaaactcatcaatgtatcttatcatgt |
| | | ct |
| 9 | 3′ ITR | aggaacccctagtgatggagttggccactccctctctgcgcgctcgctcgctcactgaggccgggcgacca |
| | | aaggtcgcccgacgcccgggctttgcccgggcggcctcagtgagcgagcgagcgcgcag |
| 10 | Human RLBP1 | ttgtcctctccctgcttggccttaaccagccacatttctcaactgaccccactcactgcagaggtgaaaactacc |
| | promoter (long) | atgccaggtcctgctggctgggggaggggtgggcaataggcctggatttgccagagctgccactgtagatg |
| | (NT_010274.17) | tagtcatatttacgatttcccttcacctcttattaccctggtggtggtggtgggggggggggggtgctctctcag |
| | | caaccccaccccgggatcttgaggagaaagagggcagagaaaagagggaatgggactggcccagatcc |
| | | cagccccacagccgggcttccacatggccgagcaggaactccagagcaggagcacacaaaggagggctt |
| | | tgatgcgcctccagccaggcccaggcctctcccctctcccctttctctctgggtcttcctttgccccactgagg |
| | | gcctcctgtgagcccgatttaacggaaactgtgggcggtgagaagttccttatgacacactaatcccaacctg |
| | | ctgaccggaccacgcctccagcggagggaacctctagagctccaggacattcaggtaccaggtagcccca |
| | | aggaggagctgccgacctggcaggtaagtcaatacctggggcttgcctgggccagggagcccaggactg |
| | | gggtgaggactcaggggagcagggagaccacgtcccaagatgcctgtaaaactgaaaccacctggccatt |
| | | ctccaggttgagccagaccaatttgatggcagatttagcaaataaaaatacaggacacccagttaaatgtgaa |
| | | tttcagatgaacagcaaatacttttttagtattaaaaaagttcacatttaggctcacgcctgtaatcccagcacttt |
| | | gggaggccgaggcaggcagatcacctgaggtcaggagttcgagaccagcctggccaacatggtgaaacc |
| | | ccatctccactaaaaataccaaaaattagccaggcgtgctggtgggcacctgtagttccagctactcaggag |
| | | gctaaggcaggagaattgcttgaacctgggaggcagaggttgcagtgagctgagatcgcaccattgcactct |
| | | agcctgggcgacaagaacaaaactccatctcaaaaaaaaaaaaaaaaaaaaagttcacatttaactgggcat |
| | | tctgtatttaattggtaatctgagatggcagggaacagcatcagcatggtgtgagggataggcattttttcattgt |
| | | gtacagcttgtaaatcagtatttttaaaactcaaagttaatggcttgggcatatttagaaaagagttgccgcacg |
| | | gacttgaaccctgtattcctaaaatctaggatcttgttctgatggtctgcacaactggctgggggtgtccagcca |
| | | ctgtccctcttgcctgggctccccagggcagttctgtcagcctctccatttccattcctgttccagcaaaaccca |
| | | actgatagcacagcagcatttcagcctgtctacctctgtgcccacatacctggatgtctaccagccagaaagg |
| | | tggcttagatttggttcctgtgggtggattatggcccccagaacttccctgtgcttgctgggggtgtggagtgg |
| | | aaagagcaggaaatgggggaccctccgatactctatgggggtcctccaagtctctttgtgcaagttagggtaa |
| | | taatcaatatggagctaagaaagagaaggggaactatgctttagaacaggacactgtgccaggagcattgca |
| | | gaaattatatggttttcacgacagttctttttggtaggtactgttattatcctcagtttgcagatgaggaaactgaga |
| | | cccagaaaggttaaataacttgctagggtcacacaagtcataactgacaaagcctgattcaaacccaggtctc |
| | | cctaacctttaaggtttctatgacgccagctctcctagggagtttgtcttcagatgtcttggctctaggtgtcaaaa |
| | | aaagacttggtgtcaggcaggcataggttcaagtcccaactctgtcacttaccaactgtgactaggtgattgaa |
| | | ctgaccatggaacctggtcacatgcaggagcaggatggtgaagggttcttgaaggcacttaggcaggacatt |
| | | taggcaggagagaaaacctggaaacagaagagctgtctccaaaaatacccactggggaagcaggttgtcat |
| | | gtgggccatgaatgggacctgttctggtaaccaagcattgcttatgtgtccattacatttcataacacttccatcc |
| | | tactttacagggaacaaccaagactggggttaaatctcacagcctgcaagtggaagagaagaacttgaaccc |
| | | aggtccaacttttgcgccacagcaggctgcctcttggtcctgacaggaagtcacaacttgggtctgagtactg |
| | | atccctggctattttttggctgtgttaccttggacaagtcacttattcctcctcccgtttcctcctatgtaaaatggaa |
| | | ataataatgttgaccctgggtctgagagagtggatttgaaagtacttagtgcatcacaaagcacagaacacact |
| | | tccagtctcgtgattatgtacttatgtaactggtcatcacccatcttgagaatgaatgcattggggaaagggcca |
| | | tccactaggctgcgaagtttctgagggactccttcgggctggagaaggatggccacaggagggaggagag |
| | | attgccttatcctgcagtgatcatgtcattgagaacagagccagattctttttttcctggcagggccaacttgtttt |
| | | aacatctaaggactgagctatttgtgtctgtgccctttgtccaagcagtgtttcccaaagtgtagcccaagaacc |
| | | atctccctcagagccaccaggaagtgctttaaattgcaggttcctaggccacagcctgcacctgcagagtca |
| | | gaatcatggaggttgggacccaggcacctgcgtttctaacaaatgcctcgggtgattctgatgcaattgaaag |
| | | tttgagatccacagttctgagacaataacagaatggtttttctaacccctgcagccctgacttcctatcctaggg |
| | | aaggggccggctggagaggccaggacagagaaagcagatcccttctttttccaaggactctgtgtcttccat |
| | | aggcaac |
| 11 | Human RPE65 | tacgtaatatttattgaagtttaatattgtgtttgtgatacagaagtatttgctttaattctaaataaaaattttatgctttt |
| | promoter | attgctggtttaagaagatttggattatccttgtactttgaggagaagtttcttatttgaaatattttggaaacaggtc |
| | | ttttaatgtggaaagatagatattaatctcctcttctattactctccaagatccaacaaaagtgattatacccccca |
| | | aaatatgatggtagtatcttatactaccatcattttataggcatagggctcttagctgcaaataatggaactaactc |
| | | taataaagcagaacgcaaatattgtaaatattagagagctaacaatctctgggatggctaaaggatggagctt |
| | | ggaggctacccagccagtaacaatattccgggctccactgttgaatggagacactacaactgccttggatgg |
| | | gcagagatattatggatgctaagccccaggtgctaccattaggacttctaccactgtccctaacgggtggagc |
| | | ccatcacatgcctatgccctcactgtaaggaaatgaagctactgttgtatatcttgggaagcacttggattaattg |
| | | ttatacagttttgttgaagaagacccctagggtaagtagccataactgcacactaaatttaaaattgttaatgagtt |
| | | tctcaaaaaaaatgttaaggttgttagctggtatagtatatatcttgcctgttttccaaggacttctttgggcagtac |
| | | cttgtctgtgctggcaagcaactgagacttaatgaaagagtattggagatatgaatgaattgatgctgtatactct |
| | | cagagtgccaaacatataccaatggacaagaaggtgaggcagagagcagacaggcattagtgacaagca |
| | | aagatatgcagaatttcattctcagcaaatcaaaagtcctcaacctggttggaagaatattggcactgaatggta |
| | | tcaataaggttgctagagagggttagaggtgcacaatgtgcttccataacattttatacttctccaatcttagcac |
| | | taatcaaacatggttgaatactttgtttactataactcttacagagttataagatctgtgaagacagggacaggga |
| | | caatacccatctctgtctggttcataggtggtatgtaatagatatttttaaaaataagtgagttaatgaatgagggt |
| | | gagaatgaaggcacagaggtattagggggaggtgggccccagagaatggtgccaaggtccagtggggtg |
| | | actgggatcagctcaggcctgacgctggccactcccacctagctcctttctttctaatctgttctcattctccttgg |
| | | gaaggattgaggtctctggaaaacagccaaacaactgttatgggaacagcaagcccaaataaagccaagca |
| | | tcagggggatctgagagctgaaagcaacttctgttccccctccctcagctgaaggggggggaagggctcc |
| | | caaagccataactccttttaagggatttagaaggcataaaaaggcccctggctgagaacttccttcttcattctg |
| | | cagttggt |
| 12 | Human VMD2 | tacgtaattctgtcattttactagggtgatgaaattcccaagcaacaccatccttttcagataagggcactgagg |
| | promoter | ctgagagaggagctgaaacctacccggcgtcaccacacacaggtggcaaggctgggaccagaaaccag |
| | | gactgttgactgcagcccggtattcattctttccatagcccacagggctgtcaaagaccccagggcctagtca |
| | | gaggctcctccttcctggagagttcctggcacagaagttgaagctcagcacagccccctaacccccaactct |
| | | ctctgcaaggcctcaggggtcagaacactggtggagcagatcctttagcctctggattttagggccatggtag |
| | | agggggtgttgccctaaattccagccctggtctcagcccaacaccctccaagaagaaattagaggggccat |
| | | ggccaggctgtgctagccgttgcttctgagcagattacaagaagggactaagacaaggactcctttgtggag |
| | | gtcctggcttagggagtcaagtgacggcggctcagcactcacgtgggcagtgccagcctctaagagtgggc |
| | | aggggcactggccacagagtcccagggagtcccaccagcctagtcgccagacc |
| 13 | Synuclein | gggccccggtgttatctcattcttttttctcctctgtaagttgacatgtgatgtgggaacaaaggggataaagtca |
| | intronic sequence | ttattttgtgctaaaatcgtaattggagaggacctcctgttagctgggctttcttctatttattgtggtggttactgga |
| | as stuffer | gttccttcttctagttttaggatatatatatatattttttttttttctttccctgaagatataataatatatatacttctgaag |
| | sequence | attgagatttttaaattagttgtattgaaaactagctaatcagcaatttaaggctagcttgagacttatgtcttgaatt |
| | | tgtttttgtaggctccaaaaccaaggagggagtggtgcatggtgtggcaacaggtaagctccattgtgcttata |
| | | tccaaagatgatatttaaagtatctagtgattagtgtggcccagtattcaagattcctatgaaattgtaaaacaatc |
| | | actgagcattctaagaacatatcagtcttattgaaactgaattctttataaagtatttttaaaaaggtaaatattgatt |
| | | ataaataaaaaatatacttgccaagaataatgagggctttgaattgataagctatgtttaatttatagtaagtggg |
| | | catttaaatattctgaccaaaaatgtattgacaaactgctgacaaaaataaaatgtgaatattgccataattttaaa |
| | | aaaagagtaaaatttctgttgattacagtaaaatattttgaccttaaattatgttgattacaatattcctttgataattc |
| | | agagtgcatttcaggaaacacccttggacagtcagtaaattgtttattgtatttatctttgtattgttatggtatagct |
| | | atttgtacaaatattattgtgcaattattacatttctgattatattattcatttggcctaaatttaccaagaatttgaaca |
| | | agtcaattaggtttacaatcaagaaatatcaaaaatgatgaaaaggatgataatcatcatcagatgttgaggaa |
| | | gatgacgatgagagtgccagaaatagagaaatcaaaggagaaccaaaatttaacaaattaaaagcccacag |
| | | acttgctgtaattaagttttctgttgtaagtactccacgtttcctggcagatgtggtgaagcaaaagatataatcag |
| | | aaatataatttatatgatcggaaagcattaaacacaatagtgcctatacaaataaaatgttcctatcactgacttct |
| | | aaaatggaaatgaggacaatgatatgggaatcttaatacagtgttgtggataggactaaaaacacaggagtca |
| | | gatcttcttggttcaacttcctgcttactccttaccagctgtgtgttttttgcaaggttcttcacctctatgtgatttag |
| | | cttcctcatctataaaataattcagtgaattaatgtacacaaaacatctggaaaacaaaagcaaacaatatgtatt |
| | | ttataagtgttacttatagttttatagtgaactttcttgtgcaacatttttacaactagtggagaaaaatatttctttaaa |
| | | tgaatacttttgatttaaaaatcagagtgtaaaaataaaacagactcctttgaaactagttctgttagaagttaattg |
| | | tgcacctttaatgggctctgttgcaatccaacagagaagtagttaagtaagtggactatgatggcttctaggga |
| | | cctcctataaatatgatattgtgaagcatgattataataagaactagataacagacaggtggagactccactatc |
| | | tgaagagggtcaacctagatgaatggtgttccatttagtagttgaggaagaacccatgaggtttagaaagcag |
| | | acaagcatgtggcaagttctggagtcagtggtaaaaattaaagaacccaactattactgtcacctaatgatcta |
| | | atggagactgtggagatgggctgcatttttttaatcttctccagaatgccaaaatgtaaacacatatctgtgtgtg |
| | | tgtgtgtgtgtgtgtgtgtgtgtgagagagagagagagagagagagagactgaagtttgtacaattagacattt |
| | | tataaaatgttttctgaaggacagtggctcacaatcttaagtttctaacattgtacaatgttgggagactttgtatac |
| | | tttattttctctttagcatattaaggaatctgagatgtcctacagtaaagaaatttgcattacatagttaaaatcagg |
| | | gttattcaaactttttgattattgaaacctttcttcattagttactagggttgaatgaaactagtgttccacagaaaac |
| | | tatgggaaatgttgctaggcagtaaggacatggtgatttcagcatgtgcaatatttacagcgattgcacccatg |
| | | gaccaccctggcagtagtgaaataaccaaaaatgctgtcataactagtatggctatgagaaacacattggg |
| 14 | RLBP1 intronic | attctccaggttgagccagaccaatttgatggtagatttagcaaataaaaatacaggacacccagttaaatgtg |
| | sequence as | aatttccgatgaacagcaaatacttttttagtattaaaaaagttcacatttaggctcacgcctgtaatcccagcact |
| | stuffer sequence | ttgggaggccgaggcaggcagatcacctgaggtcaggagttcgagaccagcctggccaacatggtgaaac |
| | (NT_010274.17) | cccatctccactaaaaataccaaaaattagccaggcgtgctggtgggcacctgtagttccagctactcaggag |
| | | gctaaggcaggagaattgcttgaacctgggaggcagaggttgcagtgagctgagatcgcaccattgcactct |
| | | agcctgggcgacaagaacaaaactccatctcaaaaaaaaaaaaaaaaaaaaagttcacatttaactgggcat |
| | | tctgtatttaattggtaatctgagatggcagggaacagcatcagcatggtgtgagggataggcattttttcattgt |
| | | gtacagcttgtaaatcagtatttttaaaactcaaagttaatggcttgggcatatttagaaaagagttgccgcacg |
| | | gacttgaaccctgtattcctaaaatctaggatcttgttctgatggtctgcacaactggctgggggtgtccagcca |
| | | ctgtccctcttgcctgggctccccagggcagttctgtcagcctctccatttccattcctgttccagcaaaaccca |
| | | actgatagcacagcagcatttcagcctgtctacctctgtgcccacatacctggatgtctaccagccagaaagg |
| | | tggcttagatttggttcctgtgggtggattatggcccccagaacttccctgtgcttgctgggggtgtggagtgg |
| | | aaagagcaggaaatgggggaccctccgatactctatgggggtcctccaagtctctttgtgcaagttagggtaa |
| | | taatcaatatggagctaagaaagagaaggggaactatgctttagaacaggacactgtgccaggagcattgca |
| | | gaaattatatggttttcacgacagttctttttggtaggtactgttattatcctcagtttgcagatgaggaaactgaga |
| | | cccagaaaggttaaataacttgctagggtcacacaagtcataactgacaaagcctgattcaaacccaggtctc |
| | | cctaacctttaaggtttctatgacgccagctctcctagggagtttgtcttcagatgtcttggctctaggtgtcaaaa |
| | | aaagacttggtgtcaggcaggcataggttcaagtcccaactctgtcacttaccaactgtgactaggtgattgaa |
| | | ctgaccatggaacctggtcacatgcaggagcaggatggtgaagggttcttgaaggcacttaggcaggacatt |
| | | taggcaggagagaaaacctggaaacagaagagctgtctccaaaaatacccactggggaagcaggttgtcat |
| | | gtgggccatgaatgggacctgttctgg |
| 15 | AMP bacterial | ctgcctgcaggggcgcctgatgcggtattttctccttacgcatctgtgcggtatttcacaccgcatacgtcaaa |
| | backbone | gcaaccatagtacgcgccctgtagcggcgcattaagcgcggcgggtgtggtggttacgcgcagcgtgacc |
| | | gctacacttgccagcgccttagcgcccgctcctttcgctttcttcccttcctttctcgccacgttcgccggctttcc |
| | | ccgtcaagctctaaatcgggggctccctttagggttccgatttagtgctttacggcacctcgaccccaaaaaac |
| | | ttgatttgggtgatggttcacgtagtgggccatcgccctgatagacggtttttcgccctttgacgttggagtcca |
| | | cgttctttaatagtggactcttgttccaaactggaacaacactcaactctatctcgggctattcttttgatttataag |
| | | ggattttgccgatttcggtctattggttaaaaaatgagctgatttaacaaaaatttaacgcgaattttaacaaaata |
| | | ttaacgtttacaattttatggtgcactctcagtacaatctgctctgatgccgcatagttaagccagccccgacacc |
| | | cgccaacacccgctgacgcgccctgacgggcttgtctgctcccggcatccgcttacagacaagctgtgacc |
| | | gtctccgggagctgcatgtgtcagaggttttcaccgtcatcaccgaaacgcgcgagacgaaagggcctcgt |
| | | gatacgcctatttttataggttaatgtcatgataataatggtttcttagacgtcaggtggcacttttcggggaaatg |
| | | tgcgcggaacccctatttgtttatttttctaaatacattcaaatatgtatccgctcatgagacaataaccctgataa |
| | | atgcttcaataatattgaaaaaggaagagtatgagtattcaacatttccgtgtcgcccttattcccttttttgcggc |
| | | attttgccttcctgtttttgctcacccagaaacgctggtgaaagtaaaagatgctgaagatcagttgggtgcacg |
| | | agtgggttacatcgaactggatctcaacagcggtaagatccttgagagttttcgccccgaagaacgttttccaa |
| | | tgatgagcacttttaaagttctgctatgtggcgcggtattatcccgtattgacgccgggcaagagcaactcggt |
| | | cgccgcatacactattctcagaatgacttggttgagtactcaccagtcacagaaaagcatcttacggatggcat |
| | | gacagtaagagaattatgcagtgctgccataaccatgagtgataacactgcggccaacttacttctgacaacg |
| | | atcggaggaccgaaggagctaaccgcttttttgcacaacatgggggatcatgtaactcgccttgatcgttggg |
| | | aaccggagctgaatgaagccataccaaacgacgagcgtgacaccacgatgcctgtagcaatggcaacaac |
| | | gttgcgcaaactattaactggcgaactacttactctagcttcccggcaacaattaatagactggatggaggcg |
| | | gataaagttgcaggaccacttctgcgctcggcccttccggctggctggtttattgctgataaatctggagccgg |
| | | tgagcgtgggtctcgcggtatcattgcagcactggggccagatggtaagccctcccgtatcgtagttatctac |
| | | acgacggggagtcaggcaactatggatgaacgaaatagacagatcgctgagataggtgcctcactgattaa |
| | | gcattggtaactgtcagaccaagtttactcatatatactttagattgatttaaaacttcatttttaatttaaaaggatct |
| | | aggtgaagatcctttttgataatctcatgaccaaaatcccttaacgtgagttttcgttccactgagcgtcagaccc |
| | | cgtagaaaagatcaaaggatcttcttgaaatcctttttttctgcgcgtaatctgctgcttgcaaacaaaaaaacca |
| | | ccgctaccagcggtggtttgtttgccggatcaagagctaccaactctttttccgaaggtaactggcttcagcag |
| | | agcgcagataccaaatactgttcttctagtgtagccgtagttaggccaccacttcaagaactctgtagcaccgc |
| | | ctacatacctcgctctgctaatcctgttaccagtggctgctgccagtggcgataagtcgtgtcttaccgggttgg |
| | | actcaagacgatagttaccggataaggcgcagcggtcgggctgaacggggggttcgtgcacacagcccag |
| | | cttggagcgaacgacctacaccgaactgagatacctacagcgtgagctatgagaaagcgccacgcttcccg |
| | | aagggagaaaggcggacaggtatccggtaagcggcagggtcggaacaggagagcgcacgagggagct |
| | | tccagggggaaacgcctggtatctttatagtcctgtcgggtttcgccacctctgacttgagcgtcgatttttgtga |
| | | tgctcgtcaggggggcggagcctatggaaaaacgccagcaacgcggcctttttacggttcctggccttttgct |
| | | ggccttttgctcacatgtcctgcaggcag |
| 16 | 5′ ITR - | ctgcgcgctcgctcgctcactgaggccgcccgggcaaagcccgggcgtcgggcgacctttggtcgcccg |
| | Stratagene | gcctcagtgagcgagcgagcgcgcagagagggagtggccaactccatcactaggggttcct |
| 17 | 5′ ITR - NCBI | ttggccactccctctctgcgcgctcgctcgctcactgaggccgggcgaccaaaggtcgcccgacgcccgg |
| | (AF043303) | gctttgcccgggcggcctcagtgagcgagcgagcgcgcagagagggagtggccaactccatcactaggg |
| | | gttcct |
| 18 | AAV2 capsid | atggctgccgatggttatcttccagattggctcgaggacactctctctgaaggaataagacagtggtggaagc |
| | coding sequence | tcaaacctggcccaccaccaccaaagcccgcagagcggcataaggacgacagcaggggtcttgtgcttcc |
| | | tgggtacaagtacctcggacccttcaacggactcgacaagggagagccggtcaacgaggcagacgccgc |
| | | ggccctcgagcacgacaaagcctacgaccggcagctcgacagcggagacaacccgtacctcaagtacaa |
| | | ccacgccgacgcggagtttcaggagcgccttaaagaagatacgtcttttgggggcaacctcggacgagcag |
| | | tcttccaggcgaaaaagagggttcttgaacctctgggcctggttgaggaacctgttaagacggctccgggaa |
| | | aaaagaggccggtagagcactctcctgtggagccagactcctcctcgggaaccggaaaggcgggccagc |
| | | agcctgcaagaaaaagattgaattttggtcagactggagacgcagactcagtacctgacccccagcctctcg |
| | | gacagccaccagcagccccctctggtctgggaactaatacgatggctacaggcagtggcgcaccaatggc |
| | | agacaataacgagggcgccgacggagtgggtaattcctcgggaaattggcattgcgattccacatggatgg |
| | | gcgacagagtcatcaccaccagcacccgaacctgggccctgcccacctacaacaaccacctctacaaaca |
| | | aatttccagccaatcaggagcctcgaacgacaatcactactttggctacagcaccccttgggggtattttgactt |
| | | caacagattccactgccacttttcaccacgtgactggcaaagactcatcaacaacaactggggattccgaccc |
| | | aagagactcaacttcaagctctttaacattcaagtcaaagaggtcacgcagaatgacggtacgacgacgattg |
| | | ccaataaccttaccagcacggttcaggtgtttactgactcggagtaccagctcccgtacgtcctcggctcggc |
| | | gcatcaaggatgcctcccgccgttcccagcagacgtcttcatggtgccacagtatggatacctcaccctgaac |
| | | aacgggagtcaggcagtaggacgctcttcattttactgcctggagtactttccttctcagatgctgcgtaccgg |
| | | aaacaactttaccttcagctacacttttgaggacgttcctttccacagcagctacgctcacagccagagtctgg |
| | | accgtctcatgaatcctctcatcgaccagtacctgtattacttgagcagaacaaacactccaagtggaaccacc |
| | | acgcagtcaaggcttcagttttctcaggccggagcgagtgacattcgggaccagtctaggaactggcttcctg |
| | | gaccctgttaccgccagcagcgagtatcaaagacatctgcggataacaacaacagtgaatactcgtggactg |
| | | gagctaccaagtaccacctcaatggcagagactctctggtgaatccgggcccggccatggcaagccacaa |
| | | ggacgatgaagaaaagttttttcctcagagcggggttctcatctttgggaagcaaggctcagagaaaacaaat |
| | | gtggacattgaaaaggtcatgattacagacgaagaggaaatcaggacaaccaatcccgtggctacggagca |
| | | gtatggttctgtatctaccaacctccagagaggcaacagacaagcagctaccgcagatgtcaacacacaag |
| | | gcgttcttccaggcatggtctggcaggacagagatgtgtaccttcaggggcccatctgggcaaagattccac |
| | | acacggacggacattttcacccctctcccctcatgggggattcggacttaaacaccctcctccacagattctc |
| | | atcaagaacaccccggtacctgcgaatccttcgaccaccttcagtgcggcaaagtttgcttccttcatcacaca |
| | | gtactccacgggacaggtcagcgtggagatcgagtgggagctgcagaaggaaaacagcaaacgctggaa |
| | | tcccgaaattcagtacacttccaactacaacaagtctgttaatgtggactttactgtggacactaatggcgtgtat |
| | | tcagagcctcgccccattggcaccagatacctgactcgtaatctgtaa |
| 19 | AAV2 capsid | MAADGYLPDWLEDTLSEGIRQWWKLKPGPPPPKPAERHKDDSRGL |
| | protein sequence | VLPGYKYLGPFNGLDKGEPVNEADAAALEHDKAYDRQLDSGDNPY |
| | (VP1) | LKYNHADAEFQERLKEDTSFGGNLGRAVFQAKKRVLEPLGLVEEPV |
| | | KTAPGKKRPVEHSPVEPDSSSGTGKAGQQPARKRLNFGQTGDADSV |
| | | PDPQPLGQPPAAPSGLGTNTMATGSGAPMADNNEGADGVGNSSGN |
| | | WHCDSTWMGDRVITTSTRTWALPTYNNHLYKQISSQSGASNDNHY |
| | | FGYSTPWGYFDFNRFHCHFSPRDWQRLINNNWGFRPKRLNFKLFNI |
| | | QVKEVTQNDGTTTIANNLTSTVQVFTDSEYQLPYVLGSAHQGCLPPF |
| | | PADVFMVPQYGYLTLNNGSQAVGRSSFYCLEYFPSQMLRTGNNFTF |
| | | SYTFEDVPFHSSYAHSQSLDRLMNPLIDQYLYYLSRTNTPSGTTTQSR |
| | | LQFSQAGASDIRDQSRNWLPGPCYRQQRVSKTSADNNNSEYSWTGA |
| | | TKYHLNGRDSLVNPGPAMASHKDDEEKFFPQSGVLIFGKQGSEKTN |
| | | VDIEKVMITDEEEIRTTNPVATEQYGSVSTNLQRGNRQAATADVNTQ |
| | | GVLPGMVWQDRDVYLQGPIWAKIPHTDGHFHPSPLMGGFGLKHPPP |
| | | QILIKNTPVPANPSTTFSAAKFASFITQYSTGQVSVEIEWELQKENSKR |
| | | WNPEIQYTSNYNKSVNVDFTVDTNGVYSEPRPIGTRYLTRNL |
| 20 | AAV8 capsid | atggctgccgatggttatcttccagattggctcgaggacaacctctctgagggcattcgcgagtggtgggcgc |
| | coding sequence | tgaaacctggagccccgaagcccaaagccaaccagcaaaagcaggacgacggccggggtctggtgcttc |
| | | ctggctacaagtacctcggacccttcaacggactcgacaagggggagcccgtcaacgcggcggacgcag |
| | | cggccctcgagcacgacaaggcctacgaccagcagctgcaggcgggtgacaatccgtacctgcggtataa |
| | | ccacgccgacgccgagtttcaggagcgtctgcaagaagatacgtcttttgggggcaacctcgggcgagca |
| | | gtcttccaggccaagaagcgggttctcgaacctctcggtctggttgaggaaggcgctaagacggctcctgga |
| | | aagaagagaccggtagagccatcaccccagcgttctccagactcctctacgggcatcggcaagaaaggcc |
| | | aacagcccgccagaaaaagactcaattttggtcagactggcgactcagagtcagttccagaccctcaacctc |
| | | tcggagaacctccagcagcgccctctggtgtgggacctaatacaatggctgcaggcggtggcgcaccaatg |
| | | gcagacaataacgaaggcgccgacggagtgggtagttcctcgggaaattggcattgcgattccacatggct |
| | | gggcgacagagtcatcaccaccagcacccgaacctgggccctgcccacctacaacaaccacctctacaag |
| | | caaatctccaacgggacatcgggaggagccaccaacgacaacacctacttcggctacagcaccccctggg |
| | | ggtattttgactttaacagattccactgccacttttcaccacgtgactggcagcgactcatcaacaacaactggg |
| | | gattccggcccaagagactcagcttcaagctcttcaacatccaggtcaaggaggtcacgcagaatgaaggc |
| | | accaagaccatcgccaataacctcaccagcaccatccaggtgtttacggactcggagtaccagctgccgtac |
| | | gttctcggctctgcccaccagggctgcctgcctccgttcccggcggacgtgttcatgattccccagtacggct |
| | | acctaacactcaacaacggtagtcaggccgtgggacgctcctccttctactgcctggaatactttccttcgcag |
| | | atgctgagaaccggcaacaacttccagtttacttacaccttcgaggacgtgcctttccacagcagctacgccc |
| | | acagccagagcttggaccggctgatgaatcctctgattgaccagtacctgtactacttgtctcggactcaaaca |
| | | acaggaggcacggcaaatacgcagactctgggcttcagccaaggtgggcctaatacaatggccaatcagg |
| | | caaagaactggctgccaggaccctgttaccgccaacaacgcgtctcaacgacaaccgggcaaaacaacaa |
| | | tagcaactttgcctggactgctgggaccaaataccatctgaatggaagaaattcattggctaatcctggcatcg |
| | | ctatggcaacacacaaagacgacgaggagcgtttttttcccagtaacgggatcctgatttttggcaaacaaaat |
| | | gctgccagagacaatgcggattacagcgatgtcatgctcaccagcgaggaagaaatcaaaaccactaaccc |
| | | tgtggctacagaggaatacggtatcgtggcagataacttgcagcagcaaaacacggctcctcaaattggaac |
| | | tgtcaacagccagggggccttacccggtatggtctggcagaaccgggacgtgtacctgcagggtcccatct |
| | | gggccaagattcctcacacggacggcaacttccacccgtctccgctgatgggcggctttggcctgaaacatc |
| | | ctccgcctcagatcctgatcaagaacacgcctgtacctgcggatcctccgaccaccttcaaccagtcaaagct |
| | | gaactctttcatcacgcaatacagcaccggacaggtcagcgtggaaattgaatgggagctgcagaaggaaa |
| | | acagcaagcgctggaaccccgagatccagtacacctccaactactacaaatctacaagtgtggactttgctgt |
| | | taatacagaaggcgtgtactctgaaccccgccccattggcacccgttacctcacccgtaatctgtaa |
| 21 | AAV8 capsid | MAADGYLPDWLEDNLSEGIREWWALKPGAPKPKANQQKQDDGRG |
| | protein sequence | LVLPGYKYLGPFNGLDKGEPVNAADAAALEHDKAYDQQLQAGDNP |
| | (VP1) | YLRYNHADAEFQERLQEDTSFGGNLGRAVFQAKKRVLEPLGLVEEG |
| | | AKTAPGKKRPVEPSPQRSPDSSTGIGKKGQQPARKRLNFGQTGDSES |
| | | VPDPQPLGEPPAAPSGVGPNTMAAGGGAPMADNNEGADGVGSSSG |
| | | NWHCDSTWLGDRVITTSTRTWALPTYNNHLYKQISNGTSGGATND |
| | | NTYFGYSTPWGYFDFNRFHCHFSPRDWQRLINNNWGFRPKRLSFKL |
| | | FNIQVKEVTQNEGTKTIANNLTSTIQVFTDSEYQLPYVLGSAHQGCLP |
| | | PFPADVFMIPQYGYLTLNNGSQAVGRSSFYCLEYFPSQMLRTGNNFQ |
| | | FTYTFEDVPFHSSYAHSQSLDRLMNPLIDQYLYYLSRTQTTGGTANT |
| | | QTLGFSQGGPNTMANQAKNWLPGPCYRQQRVSTTTGQNNNSNFAW |
| | | TAGTKYHLNGRNSLANPGIAMATHKDDEERFFPSNGILIFGKQNAAR |
| | | DNADYSDVMLTSEEEIKTTNPVATEEYGIVADNLQQQNTAPQIGTVN |
| | | SQGALPGMVWQNRDVYLQGPIWAKIPHTDGNFHPSPLMGGFGLKH |
| | | PPPQILIKNTPVPADPPTTFNQSKLNSFITQYSTGQVSVEIEWELQKEN |
| | | SKRWNPEIQYTSNYYKSTSVDFAVNTEGVYSEPRPIGTRYLTRNL |
| 22 | CVM enhancer | actagttattaatagtaatcaattacggggtcattagttcatagcccatatatggagttccgcgttacataacttac |
| | and CBA | ggtaaatggcccgcctggctgaccgcccaacgacccccgcccattgacgtcaataatgacgtatgttcccat |
| | promoter | agtaacgccaatagggactttccattgacgtcaatgggtggagtatttacggtaaactgcccacttggcagtac |
| | (GenBank Acc. | atcaagtgtatcatatgccaagtacgccccctattgacgtcaatgacggtaaatggcccgcctggcattatgcc |
| | DD215332 from | cagtacatgaccttatgggactttcctacttggcagtacatctacgtattagtcatcgctattaccatggtcgagg |
| | bp 1-1616) | tgagccccacgttctgcttcactctccccatctcccccccctccccacccccaattttgtatttatttattttttaatta |
| | | ttttgtgcagcgatgggggcggggggggggggggggcgcgcgccaggcggggcggggcggggcgag |
| | | gggcggggcggggcgaggcggagaggtgcggcggcagccaatcagagcggcgcgctccgaaagtttc |
| | | cttttatggcgaggcggcggcggcggcggccctataaaaagcgaagcgcgcggcgggcggggagtcgc |
| | | tgcgacgctgccttcgccccgtgccccgctccgccgccgcctcgcgccgcccgccccggctctgactgac |
| | | cgcgttactcccacaggtgagcggggggacggcccttctcctccgggctgtaattagcgcttggtttaatga |
| | | cggcttgtttcttttctgtggctgcgtgaaagccttgaggggctccgggagggccctttgtgcggggggagcg |
| | | gctcggggggtgcgtgcgtgtgtgtgtgcgtggggagcgccgcgtgcggctccgcgctgcccggcggct |
| | | gtgagcgctgcgggcgcggcgcggggctttgtgcgctccgcagtgtgcgcgaggggagcgcggccggg |
| | | ggcggtgccccgcggtgcggggggggctgcgaggggaacaaaggctgcgtgcggggtgtgtgcgtgg |
| | | gggggtgagcagggggtgtgggcgcgtcggtcgggctgcaaccccccctgcacccccctccccgagttg |
| | | ctgagcacggcccggcttcgggtgcggggctccgtacggggcgtggcgcggggctcgccgtgccgggc |
| | | ggggggggcggcaggtgggggtgccgggcggggggggccgcctcgggccggggagggctcggg |
| | | ggaggggcgcggcggcccccggagcgccggcggctgtcgaggcgcggcgagccgcagccattgccttt |
| | | tatggtaatcgtgcgagagggcgcagggacttcctttgtcccaaatctgtgcggagccgaaatctgggaggc |
| | | gccgccgcaccccctctagcgggcgcggggcgaagcggtgcggcgccggcaggaaggaaatgggcgg |
| | | ggagggccttcgtgcgtcgccgcgccgccgtccccttctccctctccagcctcggggctgtccgcgggggg |
| | | acggctgccttcgggggggacggggcagggcggggttcggcttctggcgtgtgaccggcggc |
| 23 | Reverse | ccagaacaggtcccattcatggcccacatgacaacctgcttccccagtgggtatttttggagacagctcttctg |
| | complement of | tttccaggttttctctcctgcctaaatgtcctgcctaagtgccttcaagaacccttcaccatcctgctcctgcatgt |
| | RLBP1 intronic | gaccaggttccatggtcagttcaatcacctagtcacagttggtaagtgacagagttgggacttgaacctatgcc |
| | sequence as | tgcctgacaccaagtctttttttgacacctagagccaagacatctgaagacaaactccctaggagagctggcg |
| | stuffer sequence | tcatagaaaccttaaaggttagggagacctgggtttgaatcaggctttgtcagttatgacttgtgtgaccctagc |
| | (NT_010274.17) | aagttatttaacctttctgggtctcagtttcctcatctgcaaactgaggataataacagtacctaccaaaaagaac |
| | | tgtcgtgaaaaccatataatttctgcaatgctcctggcacagtgtcctgttctaaagcatagttccccttctctttct |
| | | tagctccatattgattattaccctaacttgcacaaagagacttggaggacccccatagagtatcggagggtccc |
| | | ccatttcctgctctttccactccacacccccagcaagcacagggaagttctgggggccataatccacccacag |
| | | gaaccaaatctaagccacctttctggctggtagacatccaggtatgtgggcacagaggtagacaggctgaaa |
| | | tgctgctgtgctatcagttgggttttgctggaacaggaatggaaatggagaggctgacagaactgccctggg |
| | | gagcccaggcaagagggacagtggctggacacccccagccagttgtgcagaccatcagaacaagatcct |
| | | agattttaggaatacagggttcaagtccgtgcggcaactcttttctaaatatgcccaagccattaactttgagtttt |
| | | aaaaatactgatttacaagctgtacacaatgaaaaaatgcctatccctcacaccatgctgatgctgttccctgcc |
| | | atctcagattaccaattaaatacagaatgcccagttaaatgtgaactttttttttttttttttttttgagatggagttttgt |
| | | tcttgtcgcccaggctagagtgcaatggtgcgatctcagctcactgcaacctctgcctcccaggttcaagcaat |
| | | tctcctgccttagcctcctgagtagctggaactacaggtgcccaccagcacgcctggctaatttttggtattttta |
| | | gtggagatggggtttcaccatgttggccaggctggtctcgaactcctgacctcaggtgatctgcctgcctcgg |
| | | cctcccaaagtgctgggattacaggcgtgagcctaaatgtgaacttttttaatactaaaaaagtatttgctgttca |
| | | tcggaaattcacatttaactgggtgtcctgtatttttatttgctaaatctaccatcaaattggtctggctcaacctgg |
| | | agaat |
| 24 | EGFP sequence | atggtgagcaagggcgaggagctgttcaccggggtggtgcccatcctggtcgagctggacggcgacgtaa |
| | | acggccacaagttcagcgtgtccggcgagggcgagggcgatgccacctacggcaagctgaccctgaagtt |
| | | catctgcaccaccggcaagctgcccgtgccctggcccaccctcgtgaccaccctgacctacggcgtgcagt |
| | | gcttcagccgctaccccgaccacatgaagcagcacgacttcttcaagtccgccatgcccgaaggctacgtcc |
| | | aggagcgcaccatcttcttcaaggacgacggcaactacaagacccgcgccgaggtgaagttcgagggcga |
| | | caccctggtgaaccgcatcgagctgaagggcatcgacttcaaggaggacggcaacatcctggggcacaag |
| | | ctggagtacaactacaacagccacaacgtctatatcatggccgacaagcagaagaacggcatcaaggtgaa |
| | | cttcaagatccgccacaacatcgaggacggcagcgtgcagctcgccgaccactaccagcagaacaccccc |
| | | atcggcgacggccccgtgctgctgcccgacaaccactacctgagcacccagtccgccctgagcaaagacc |
| | | ccaacgagaagcgcgatcacatggtcctgctggagttcgtgaccgccgccgggatcactctcggcatggac |
| | | gagctgtacaagtaa |
| 25 | GFP amino acid | MVSKGEELFTGVVPILVELDGDVNGHKFSVSGEGEGDATYGKLTLK |
| | sequence | FICTTGKLPVPWPTLVTTLTYGVQCFSRYPDHMKQHDFFKSAMPEG |
| | | YVQERTIFFKDDGNYKTRAEVKFEGDTLVNRIELKGIDFKEDGNILG |
| | | HKLEYNYNSHNVYIMADKQKNGIKVNFKIRHNIEDGSVQLADHYQQ |
| | | NTPIGDGPVLLPDNHYLSTQSALSKDPNEKRDHMVLLEFVTAAGITL |
| | | GMDELYK |
| 26 | Plasmid TM017 | ctgcgcgctcgctcgctcactgaggccgcccgggcaaagcccgggcgtcgggcgacctttggtcgcccg |
| | | gcctcagtgagcgagcgagcgcgcagagagggagtggggtaccacgcgtttgtcctctccctgcttggcct |
| | | taaccagccacatttctcaactgaccccactcactgcagaggtgaaaactaccatgccaggtcctgctggctg |
| | | ggggaggggtgggcaataggcctggatttgccagagctgccactgtagatgtagtcatatttacgatttccctt |
| | | cacctcttattaccctggtggtggtggtgggggggggggggtgctctctcagcaaccccaccccgggatctt |
| | | gaggagaaagagggcagagaaaagagggaatgggactggcccagatcccagccccacagccgggcttc |
| | | cacatggccgagcaggaactccagagcaggagcacacaaaggagggctttgatgcgcctccagccaggc |
| | | ccaggcctctcccctctcccctttctctctgggtcttcctttgccccactgagggcctcctgtgagcccgatttaa |
| | | cggaaactgtgggcggtgagaagttccttatgacacactaatcccaacctgctgaccggaccacgcctccag |
| | | cggagggaacctctagagctccaggacattcaggtaccaggtagccccaaggaggagctgccgaatcgat |
| | | ggatcgggaactgaaaaaccagaaagttaactggtaagtttagtctttttgtcttttatttcaggtcccggatccg |
| | | gtggtggtgcaaatcaaagaactgctcctcagtggatgttgcctttacttctaggcctgtacggaagtgttactt |
| | | ctgctctaaaagctgcggaattgtacccgccccgggatccatcgattgaattcgccaccatgtcagaaggggt |
| | | gggcacgttccgcatggtacctgaagaggaacaggagctccgtgcccaactggagcagctcacaaccaag |
| | | gaccatggacctgtctttggcccgtgcagccagctgccccgccacaccttgcagaaggccaaggatgagct |
| | | gaacgagagagaggagacccgggaggaggcagtgcgagagctgcaggagatggtgcaggcgcaggc |
| | | ggcctcgggggaggagctggcggtggccgtggcggagagggtgcaagagaaggacagcggcttcttcct |
| | | gcgcttcatccgcgcacggaagttcaacgtgggccgtgcctatgagctgctcagaggctatgtgaatttccg |
| | | gctgcagtaccctgagctctttgacagcctgtccccagaggctgtccgctgcaccattgaagctggctaccct |
| | | ggtgtcctctctagtcgggacaagtatggccgagtggtcatgctcttcaacattgagaactggcaaagtcaag |
| | | aaatcacctttgatgagatcttgcaggcatattgcttcatcctggagaagctgctggagaatgaggaaactcaa |
| | | atcaatggcttctgcatcattgagaacttcaagggctttaccatgcagcaggctgctagtctccggacttcagat |
| | | ctcaggaagatggtggacatgctccaggattccttcccagcccggttcaaagccatccacttcatccaccagc |
| | | catggtacttcaccacgacctacaatgtggtcaagcccttcttgaagagcaagctgcttgagagggtctttgtc |
| | | cacggggatgacctttctggtttctaccaggagatcgatgagaacatcctgccctctgacttcgggggcacgc |
| | | tgcccaagtatgatggcaaggccgttgctgagcagctctttggcccccaggcccaagctgagaacacagcc |
| | | ttctgaggatcgtaccggtcgacctgcagaagcttgcctcgagcagcgctgctcgagagatctggatcataat |
| | | cagccataccacatttgtagaggttttacttgctttaaaaaacctcccacacctccccctgaacctgaaacataa |
| | | aatgaatgcaattgttgttgttaacttgtttattgcagcttataatggttacaaataaagcaatagcatcacaaattt |
| | | cacaaataaagcatttttttcactgcattctagttgtggtttgtccaaactcatcaatgtatcttatcatgtctggtaa |
| | | ccacgtgcggaccgagcggccgcaggaacccctagtgatggagttggccactccctctctgcgcgctcgct |
| | | cgctcactgaggccgggcgaccaaaggtcgcccgacgcccgggctttgcccgggcggcctcagtgagcg |
| | | agcgagcgcgcagctgcctgcaggggcgcctgatgcggtattttctccttacgcatctgtgcggtatttcaca |
| | | ccgcatacgtcaaagcaaccatagtacgcgccctgtagcggcgcattaagcgcggcgggtgtggtggttac |
| | | gcgcagcgtgaccgctacacttgccagcgccttagcgcccgctcctttcgctttcttcccttcctttctcgccac |
| | | gttcgccggctttccccgtcaagctctaaatcgggggctccctttagggttccgatttagtgctttacggcacct |
| | | cgaccccaaaaaacttgatttgggtgatggttcacgtagtgggccatcgccctgatagacggtttttcgcccttt |
| | | gacgttggagtccacgttctttaatagtggactcttgttccaaactggaacaacactcaactctatctcgggctat |
| | | tcttttgatttataagggattttgccgatttcggtctattggttaaaaaatgagctgatttaacaaaaatttaacgcg |
| | | aattttaacaaaatattaacgtttacaattttatggtgcactctcagtacaatctgctctgatgccgcatagttaagc |
| | | cagccccgacacccgccaacacccgctgacgcgccctgacgggcttgtctgctcccggcatccgcttacag |
| | | acaagctgtgaccgtctccgggagctgcatgtgtcagaggttttcaccgtcatcaccgaaacgcgcgagacg |
| | | aaagggcctcgtgatacgcctatttttataggttaatgtcatgataataatggtttcttagacgtcaggtggcactt |
| | | ttcggggaaatgtgcgcggaacccctatttgtttatttttctaaatacattcaaatatgtatccgctcatgagacaa |
| | | taaccctgataaatgcttcaataatattgaaaaaggaagagtatgagtattcaacatttccgtgtcgcccttattc |
| | | ccttttttgcggcattttgccttcctgtttttgctcacccagaaacgctggtgaaagtaaaagatgctgaagatca |
| | | gttgggtgcacgagtgggttacatcgaactggatctcaacagcggtaagatccttgagagttttcgccccgaa |
| | | gaacgttttccaatgatgagcacttttaaagttctgctatgtggcgcggtattatcccgtattgacgccgggcaa |
| | | gagcaactcggtcgccgcatacactattctcagaatgacttggttgagtactcaccagtcacagaaaagcatc |
| | | ttacggatggcatgacagtaagagaattatgcagtgctgccataaccatgagtgataacactgcggccaactt |
| | | acttctgacaacgatcggaggaccgaaggagctaaccgcttttttgcacaacatgggggatcatgtaactcgc |
| | | cttgatcgttgggaaccggagctgaatgaagccataccaaacgacgagcgtgacaccacgatgcctgtagc |
| | | aatggcaacaacgttgcgcaaactattaactggcgaactacttactctagcttcccggcaacaattaatagact |
| | | ggatggaggcggataaagttgcaggaccacttctgcgctcggcccttccggctggctggtttattgctgataa |
| | | atctggagccggtgagcgtgggtctcgcggtatcattgcagcactggggccagatggtaagccctcccgtat |
| | | cgtagttatctacacgacggggagtcaggcaactatggatgaacgaaatagacagatcgctgagataggtg |
| | | cctcactgattaagcattggtaactgtcagaccaagtttactcatatatactttagattgatttaaaacttcattttta |
| | | atttaaaaggatctaggtgaagatcctttttgataatctcatgaccaaaatcccttaacgtgagttttcgttccact |
| | | gagcgtcagaccccgtagaaaagatcaaaggatcttcttgaaatcctttttttctgcgcgtaatctgctgcttgca |
| | | aacaaaaaaaccaccgctaccagcggtggtttgtttgccggatcaagagctaccaactctttttccgaaggta |
| | | actggcttcagcagagcgcagataccaaatactgttcttctagtgtagccgtagttaggccaccacttcaagaa |
| | | ctctgtagcaccgcctacatacctcgctctgctaatcctgttaccagtggctgctgccagtggcgataagtcgt |
| | | gtcttaccgggttggactcaagacgatagttaccggataaggcgcagcggtcgggctgaacggggggttcg |
| | | tgcacacagcccagcttggagcgaacgacctacaccgaactgagatacctacagcgtgagctatgagaaa |
| | | gcgccacgcttcccgaagggagaaaggcggacaggtatccggtaagcggcagggtcggaacaggagag |
| | | cgcacgagggagcttccagggggaaacgcctggtatctttatagtcctgtcgggtttcgccacctctgacttg |
| | | agcgtcgatttttgtgatgctcgtcaggggggcggagcctatggaaaaacgccagcaacgcggcctttttac |
| | | ggttcctggccttttgctggccttttgctcacatgtcctgcaggcag |
| 27 | Plasmid TM037 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcagcttttgt |
| | | cctctccctgcttggccttaaccagccacatttctcaactgaccccactcactgcagaggtgaaaactaccatg |
| | | ccaggtcctgctggctgggggaggggtgggcaataggcctggatttgccagagctgccactgtagatgtag |
| | | tcatatttacgatttcccttcacctcttattaccctggtggtggtggtgggggggggggggtgctctctcagcaa |
| | | ccccaccccgggatcttgaggagaaagagggcagagaaaagagggaatgggactggcccagatcccag |
| | | ccccacagccgggcttccacatggccgagcaggaactccagagcaggagcacacaaaggagggctttga |
| | | tgcgcctccagccaggcccaggcctctcccctctcccctttctctctgggtcttcctttgccccactgagggcct |
| | | cctgtgagcccgatttaacggaaactgtgggcggtgagaagttccttatgacacactaatcccaacctgctga |
| | | ccggaccacgcctccagcggagggaacctctagagctccaggacattcaggtaccaggtagccccaagg |
| | | aggagctgccgacctggcaggtaagtcaatacctggggcttgcctgggccagggagcccaggactggggt |
| | | gaggactcaggggagcagggagaccacgtcccaagatgcctgtaaaactgaaaccacctggccattctcc |
| | | aggttgagccagaccaatttgatggcagatttagcaaataaaaatacaggacacccagttaaatgtgaatttca |
| | | gatgaacagcaaatacttttttagtattaaaaaagttcacatttaggctcacgcctgtaatcccagcactttggga |
| | | ggccgaggcaggcagatcacctgaggtcaggagttcgagaccagcctggccaacatggtgaaaccccatc |
| | | tccactaaaaataccaaaaattagccaggcgtgctggtgggcacctgtagttccagctactcaggaggctaa |
| | | ggcaggagaattgcttgaacctgggaggcagaggttgcagtgagctgagatcgcaccattgcactctagcct |
| | | gggcgacaagaacaaaactccatctcaaaaaaaaaaaaaaaaaaaaagttcacatttaactgggcattctgta |
| | | tttaattggtaatctgagatggcagggaacagcatcagcatggtgtgagggataggcattttttcattgtgtaca |
| | | gcttgtaaatcagtatttttaaaactcaaagttaatggcttgggcatatttagaaaagagttgccgcacggacttg |
| | | aaccctgtattcctaaaatctaggatcttgttctgatggtctgcacaactggctgggggtgtccagccactgtcc |
| | | ctcttgcctgggctccccagggcagttctgtcagcctctccatttccattcctgttccagcaaaacccaactgat |
| | | agcacagcagcatttcagcctgtctacctctgtgcccacatacctggatgtctaccagccagaaaggtggctt |
| | | agatttggttcctgtgggggattatggcccccagaacttccctgtgcttgctgggggtgtggagtggaaaga |
| | | gcaggaaatgggggaccctccgatactctatgggggtcctccaagtctctttgtgcaagttagggtaataatc |
| | | aatatggagctaagaaagagaaggggaactatgctttagaacaggacactgtgccaggagcattgcagaaa |
| | | ttatatggttttcacgacagttctttttggtaggtactgttattatcctcagtttgcagatgaggaaactgagaccca |
| | | gaaaggttaaataacttgctagggtcacacaagtcataactgacaaagcctgattcaaacccaggtctcccta |
| | | acctttaaggtttctatgacgccagctctcctagggagtttgtcttcagatgtcttggctctaggtgtcaaaaaaa |
| | | gacttggtgtcaggcaggcataggttcaagtcccaactctgtcacttaccaactgtgactaggtgattgaactg |
| | | accatggaacctggtcacatgcaggagcaggatggtgaagggttcttgaaggcacttaggcaggacatttag |
| | | gcaggagagaaaacctggaaacagaagagctgtctccaaaaatacccactggggaagcaggttgtcatgt |
| | | gggccatgaatgggacctgttctggtaaccaagcattgcttatgtgtccattacatttcataacacttccatccta |
| | | ctttacagggaacaaccaagactggggttaaatctcacagcctgcaagtggaagagaagaacttgaaccca |
| | | ggtccaacttttgcgccacagcaggctgcctcttggtcctgacaggaagtcacaacttgggtctgagtactgat |
| | | ccctggctattttttggctgtgttaccttggacaagtcacttattcctcctcccgtttcctcctatgtaaaatggaaat |
| | | aataatgttgaccctgggtctgagagagtggatttgaaagtacttagtgcatcacaaagcacagaacacacttc |
| | | cagtctcgtgattatgtacttatgtaactggtcatcacccatcttgagaatgaatgcattggggaaagggccatc |
| | | cactaggctgcgaagtttctgagggactccttcgggctggagaaggatggccacaggagggaggagagat |
| | | tgccttatcctgcagtgatcatgtcattgagaacagagccagattctttttttcctggcagggccaacttgttttaa |
| | | catctaaggactgagctatttgtgtctgtgccctttgtccaagcagtgtttcccaaagtgtagcccaagaaccat |
| | | ctccctcagagccaccaggaagtgctttaaattgcaggttcctaggccacagcctgcacctgcagagtcaga |
| | | atcatggaggttgggacccaggcacctgcgtttctaacaaatgcctcgggtgattctgatgcaattgaaagttt |
| | | gagatccacagttctgagacaataacagaatggtttttctaacccctgcagccctgacttcctatcctagggaa |
| | | ggggccggctggagaggccaggacagagaaagcagatcccttctttttccaaggactctgtgtcttccatag |
| | | gcaacgaattcgccaccatgtcagaaggggtgggcacgttccgcatggtacctgaagaggaacaggagct |
| | | ccgtgcccaactggagcagctcacaaccaaggaccatggacctgtctttggcccgtgcagccagctgcccc |
| | | gccacaccttgcagaaggccaaggatgagctgaacgagagagaggagacccgggaggaggcagtgcg |
| | | agagctgcaggagatggtgcaggcgcaggcggcctcgggggaggagctggcggtggccgtggcggag |
| | | agggtgcaagagaaggacagcggcttcttcctgcgcttcatccgcgcacggaagttcaacgtgggccgtgc |
| | | ctatgagctgctcagaggctatgtgaatttccggctgcagtaccctgagctctttgacagcctgtccccagagg |
| | | ctgtccgctgcaccattgaagctggctaccctggtgtcctctctagtcgggacaagtatggccgagtggtcat |
| | | gctcttcaacattgagaactggcaaagtcaagaaatcacctttgatgagatcttgcaggcatattgcttcatcct |
| | | ggagaagctgctggagaatgaggaaactcaaatcaatggcttctgcatcattgagaacttcaagggctttacc |
| | | atgcagcaggctgctagtctccggacttcagatctcaggaagatggtggacatgctccaggattccttcccag |
| | | cccggttcaaagccatccacttcatccaccagccatggtacttcaccacgacctacaatgtggtcaagcccttc |
| | | ttgaagagcaagctgcttgagagggtctttgtccacggggatgacctttctggtttctaccaggagatcgatga |
| | | gaacatcctgccctctgacttcgggggcacgctgcccaagtatgatggcaaggccgttgctgagcagctcttt |
| | | ggcccccaggcccaagctgagaacacagccttctgaggatcgtaccggtcgacctgcagaagcttgcctcg |
| | | agcagcgctgctcgagagatctggatcataatcagccataccacatttgtagaggttttacttgctttaaaaaac |
| | | ctcccacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtttgtc |
| | | caaactcatcaatgtatcttatcatgtctggtaaccacgtgcggaccgagcggccgcaggaacccctagtgat |
| | | ggagttggccactccctctctgcgcgctcgctcgctcactgaggccgggcgaccaaaggtcgcccgacgc |
| | | ccgggctttgcccgggcggcctcagtgagcgagcgagcgcgcagctgcctgcaggggcgcctgatgcgg |
| | | tattttctccttacgcatctgtgcggtatttcacaccgcatacgtcaaagcaaccatagtacgcgccctgtagcg |
| | | gcgcattaagcgcggcgggtgtggtggttacgcgcagcgtgaccgctacacttgccagcgccttagcgccc |
| | | gctcctttcgctttcttcccttcctttctcgccacgttcgccggctttccccgtcaagctctaaatcgggggctccc |
| | | tttagggttccgatttagtgctttacggcacctcgaccccaaaaaacttgatttgggtgatggttcacgtagtgg |
| | | gccatcgccctgatagacggtttttcgccctttgacgttggagtccacgttctttaatagtggactcttgttccaaa |
| | | ctggaacaacactcaactctatctcgggctattcttttgatttataagggattttgccgatttcggtctattggttaa |
| | | aaaatgagctgatttaacaaaaatttaacgcgaattttaacaaaatattaacgtttacaattttatggtgcactctca |
| | | gtacaatctgctctgatgccgcatagttaagccagccccgacacccgccaacacccgctgacgcgccctga |
| | | cgggcttgtctgctcccggcatccgcttacagacaagctgtgaccgtctccgggagctgcatgtgtcagagg |
| | | ttttcaccgtcatcaccgaaacgcgcgagacgaaagggcctcgtgatacgcctatttttataggttaatgtcatg |
| | | ataataatggtttcttagacgtcaggtggcacttttcggggaaatgtgcgcggaacccctatttgtttatttttctaa |
| | | atacattcaaatatgtatccgctcatgagacaataaccctgataaatgcttcaataatattgaaaaaggaagagt |
| | | atgagtattcaacatttccgtgtcgcccttattcccttttttgcggcattttgccttcctgtttttgctcacccagaaa |
| | | cgctggtgaaagtaaaagatgctgaagatcagttgggtgcacgagtgggttacatcgaactggatctcaaca |
| | | gcggtaagatccttgagagttttcgccccgaagaacgttttccaatgatgagcacttttaaagttctgctatgtgg |
| | | cgcggtattatcccgtattgacgccgggcaagagcaactcggtcgccgcatacactattctcagaatgacttg |
| | | gttgagtactcaccagtcacagaaaagcatcttacggatggcatgacagtaagagaattatgcagtgctgcca |
| | | taaccatgagtgataacactgcggccaacttacttctgacaacgatcggaggaccgaaggagctaaccgctt |
| | | ttttgcacaacatgggggatcatgtaactcgccttgatcgttgggaaccggagctgaatgaagccataccaaa |
| | | cgacgagcgtgacaccacgatgcctgtagcaatggcaacaacgttgcgcaaactattaactggcgaactact |
| | | tactctagcttcccggcaacaattaatagactggatggaggcggataaagttgcaggaccacttctgcgctcg |
| | | gcccttccggctggctggtttattgctgataaatctggagccggtgagcgtgggtctcgcggtatcattgcagc |
| | | actggggccagatggtaagccctcccgtatcgtagttatctacacgacggggagtcaggcaactatggatga |
| | | acgaaatagacagatcgctgagataggtgcctcactgattaagcattggtaactgtcagaccaagtttactcat |
| | | atatactttagattgatttaaaacttcatttttaatttaaaaggatctaggtgaagatcctttttgataatctcatgacc |
| | | aaaatcccttaacgtgagttttcgttccactgagcgtcagaccccgtagaaaagatcaaaggatcttcttgaaat |
| | | cctttttttctgcgcgtaatctgctgcttgcaaacaaaaaaaccaccgctaccagcggtggtttgtttgccggatc |
| | | aagagctaccaactctttttccgaaggtaactggcttcagcagagcgcagataccaaatactgttcttctagtgt |
| | | agccgtagttaggccaccacttcaagaactctgtagcaccgcctacatacctcgctctgctaatcctgttacca |
| | | gtggctgctgccagtggcgataagtcgtgtcttaccgggttggactcaagacgatagttaccggataaggcg |
| | | cagcggtcgggctgaacggggggttcgtgcacacagcccagcttggagcgaacgacctacaccgaactg |
| | | agatacctacagcgtgagctatgagaaagcgccacgcttcccgaagggagaaaggcggacaggtatccg |
| | | gtaagcggcagggtcggaacaggagagcgcacgagggagcttccagggggaaacgcctggtatctttata |
| | | gtcctgtcgggtttcgccacctctgacttgagcgtcgatttttgtgatgctcgtcaggggggcggagcctatgg |
| | | aaaaacgccagcaacgcggcctttttacggttcctggccttttgctggccttttgctcacatgtcctgcaggcag |
| 28 | Plasmid AG007 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcgttacgtaa |
| | | tatttattgaagtttaatattgtgtttgtgatacagaagtatttgctttaattctaaataaaaattttatgcttttattgctg |
| | | gtttaagaagatttggattatccttgtactttgaggagaagtttcttatttgaaatattttggaaacaggtcttttaatg |
| | | tggaaagatagatattaatctcctcttctattactctccaagatccaacaaaagtgattataccccccaaaatatg |
| | | atggtagtatcttatactaccatcattttataggcatagggctcttagctgcaaataatggaactaactctaataaa |
| | | gcagaacgcaaatattgtaaatattagagagctaacaatctctgggatggctaaaggatggagcttggaggct |
| | | acccagccagtaacaatattccgggctccactgttgaatggagacactacaactgccttggatgggcagaga |
| | | tattatggatgctaagccccaggtgctaccattaggacttctaccactgtccctaacgggtggagcccatcaca |
| | | tgcctatgccctcactgtaaggaaatgaagctactgttgtatatcttgggaagcacttggattaattgttatacagt |
| | | tttgttgaagaagacccctagggtaagtagccataactgcacactaaatttaaaattgttaatgagtttctcaaaa |
| | | aaaatgttaaggttgttagctggtatagtatatatcttgcctgttttccaaggacttctttgggcagtaccttgtctgt |
| | | gctggcaagcaactgagacttaatgaaagagtattggagatatgaatgaattgatgctgtatactctcagagtg |
| | | ccaaacatataccaatggacaagaaggtgaggcagagagcagacaggcattagtgacaagcaaagatatg |
| | | cagaatttcattctcagcaaatcaaaagtcctcaacctggttggaagaatattggcactgaatggtatcaataag |
| | | gttgctagagagggttagaggtgcacaatgtgcttccataacattttatacttctccaatcttagcactaatcaaa |
| | | catggttgaatactttgtttactataactcttacagagttataagatctgtgaagacagggacagggacaatacc |
| | | catctctgtctggttcataggtggtatgtaatagatatttttaaaaataagtgagttaatgaatgagggtgagaatg |
| | | aaggcacagaggtattagggggaggtgggccccagagaatggtgccaaggtccagtggggtgactggga |
| | | tcagctcaggcctgacgctggccactcccacctagctcctttctttctaatctgttctcattctccttgggaaggat |
| | | tgaggtctctggaaaacagccaaacaactgttatgggaacagcaagcccaaataaagccaagcatcaggg |
| | | ggatctgagagctgaaagcaacttctgttccccctccctcagctgaaggggggggaagggctcccaaagc |
| | | cataactccttttaagggatttagaaggcataaaaaggcccctggctgagaacttccttcttcattctgcagttgg |
| | | tgaattcgccaccatgtcagaaggggtgggcacgttccgcatggtacctgaagaggaacaggagctccgtg |
| | | cccaactggagcagctcacaaccaaggaccatggacctgtctttggcccgtgcagccagctgccccgccac |
| | | accttgcagaaggccaaggatgagctgaacgagagagaggagacccgggaggaggcagtgcgagagct |
| | | gcaggagatggtgcaggcgcaggcggcctcgggggaggagctggcggtggccgtggcggagagggtg |
| | | caagagaaggacagcggcttcttcctgcgcttcatccgcgcacggaagttcaacgtgggccgtgcctatga |
| | | gctgctcagaggctatgtgaatttccggctgcagtaccctgagctctttgacagcctgtccccagaggctgtcc |
| | | gctgcaccattgaagctggctaccctggtgtcctctctagtcgggacaagtatggccgagtggtcatgctcttc |
| | | aacattgagaactggcaaagtcaagaaatcacctttgatgagatcttgcaggcatattgcttcatcctggagaa |
| | | gctgctggagaatgaggaaactcaaatcaatggcttctgcatcattgagaacttcaagggctttaccatgcag |
| | | caggctgctagtctccggacttcagatctcaggaagatggtggacatgctccaggattccttcccagcccggt |
| | | tcaaagccatccacttcatccaccagccatggtacttcaccacgacctacaatgtggtcaagcccttcttgaag |
| | | agcaagctgcttgagagggtctttgtccacggggatgacctttctggtttctaccaggagatcgatgagaacat |
| | | cctgccctctgacttcgggggcacgctgcccaagtatgatggcaaggccgttgctgagcagctctttggccc |
| | | ccaggcccaagctgagaacacagccttctgaggatctaccggtcgacctgcagaagcttgcctcgagcagc |
| | | gctgctcgagagatctggatcataatcagccataccacatttgtagaggttttacttgctttaaaaaacctcccac |
| | | acctccccctgaacctgaaacataaaatgaatgcaattgttgttgttaacttgtttattgcagcttataatggttaca |
| | | aataaagcaatagcatcacaaatttcacaaataaagcatttttttcactgcattctagttgtggtttgtccaaactca |
| | | tcaatgtatcttatcatgtctggtaaccattctccaggttgagccagaccaatttgatggtagatttagcaaataaa |
| | | aatacaggacacccagttaaatgtgaatttccgatgaacagcaaatacttttttagtattaaaaaagttcacattta |
| | | ggctcacgcctgtaatcccagcactttgggaggccgaggcaggcagatcacctgaggtcaggagttcgag |
| | | accagcctggccaacatggtgaaaccccatctccactaaaaataccaaaaattagccaggcgtgctggtgg |
| | | gcacctgtagttccagctactcaggaggctaaggcaggagaattgcttgaacctgggaggcagaggttgca |
| | | gtgagctgagatcgcaccattgcactctagcctgggcgacaagaacaaaactccatctcaaaaaaaaaaaa |
| | | aaaaaaaaagttcacatttaactgggcattctgtatttaattggtaatctgagatggcagggaacagcatcagc |
| | | atggtgtgagggataggcattttttcattgtgtacagcttgtaaatcagtatttttaaaactcaaagttaatggcttg |
| | | ggcatatttagaaaagagttgccgcacggacttgaaccctgtattcctaaaatctaggatcttgttctgatggtct |
| | | gcacaactggctgggggtgtccagccactgtccctcttgcctgggctccccagggcagttctgtcagcctctc |
| | | catttccattcctgttccagcaaaacccaactgatagcacagcagcatttcagcctgtctacctctgtgcccaca |
| | | tacctggatgtctaccagccagaaaggtggcttagatttggttcctgtgggtggattatggcccccagaacttc |
| | | cctgtgcttgctgggggtgtggagtggaaagagcaggaaatgggggaccctccgatactctatgggggtcc |
| | | tccaagtctctttgtgcaagttagggtaataatcaatatggagctaagaaagagaaggggaactatgctttaga |
| | | acaggacactgtgccaggagcattgcagaaattatatggttttcacgacagttctttttggtaggtactgttattat |
| | | cctcagtttgcagatgaggaaactgagacccagaaaggttaaataacttgctagggtcacacaagtcataact |
| | | gacaaagcctgattcaaacccaggtctccctaacctttaaggtttctatgacgccagctctcctagggagtttgt |
| | | cttcagatgtcttggctctaggtgtcaaaaaaagacttggtgtcaggcaggcataggttcaagtcccaactctg |
| | | tcacttaccaactgtgactaggtgattgaactgaccatggaacctggtcacatgcaggagcaggatggtgaa |
| | | gggttcttgaaggcacttaggcaggacatttaggcaggagagaaaacctggaaacagaagagctgtctcca |
| | | aaaatacccactggggaagcaggttgtcatgtgggccatgaatgggacctgttctggggtaaccacgtgcg |
| | | gaccgagcggccgcaggaacccctagtgatggagttggccactccctctctgcgcgctcgctcgctcactg |
| | | aggccgggcgaccaaaggtcgcccgacgcccgggctttgcccgggcggcctcagtgagcgagcgagcg |
| | | cgcagctgcctgcaggggcgcctgatgcggtattttctccttacgcatctgtgcggtatttcacaccgcatacg |
| | | tcaaagcaaccatagtacgcgccctgtagcggcgcattaagcgcggcgggtgtggtggttacgcgcagcgt |
| | | gaccgctacacttgccagcgccttagcgcccgctcctttcgctttcttcccttcctttctcgccacgttcgccgg |
| | | ctttccccgtcaagctctaaatcgggggctccctttagggttccgatttagtgctttacggcacctcgaccccaa |
| | | aaaacttgatttgggtgatggttcacgtagtgggccatcgccctgatagacggtttttcgccctttgacgttgga |
| | | gtccacgttctttaatagtggactcttgttccaaactggaacaacactcaactctatctcgggctattcttttgattt |
| | | ataagggattttgccgatttcggtctattggttaaaaaatgagctgatttaacaaaaatttaacgcgaattttaaca |
| | | aaatattaacgtttacaattttatggtgcactctcagtacaatctgctctgatgccgcatagttaagccagccccg |
| | | acacccgccaacacccgctgacgcgccctgacgggcttgtctgctcccggcatccgcttacagacaagctg |
| | | tgaccgtctccgggagctgcatgtgtcagaggttttcaccgtcatcaccgaaacgcgcgagacgaaagggc |
| | | ctcgtgatacgcctatttttataggttaatgtcatgataataatggtttcttagacgtcaggtggcacttttcgggg |
| | | aaatgtgcgcggaacccctatttgtttatttttctaaatacattcaaatatgtatccgctcatgagacaataaccct |
| | | gataaatgcttcaataatattgaaaaaggaagagtatgagtattcaacatttccgtgtcgcccttattcccttttttg |
| | | cggcattttgccttcctgtttttgctcacccagaaacgctggtgaaagtaaaagatgctgaagatcagttgggtg |
| | | cacgagtgggttacatcgaactggatctcaacagcggtaagatccttgagagttttcgccccgaagaacgtttt |
| | | ccaatgatgagcacttttaaagttctgctatgtggcgcggtattatcccgtattgacgccgggcaagagcaact |
| | | cggtcgccgcatacactattctcagaatgacttggttgagtactcaccagtcacagaaaagcatcttacggatg |
| | | gcatgacagtaagagaattatgcagtgctgccataaccatgagtgataacactgcggccaacttacttctgac |
| | | aacgatcggaggaccgaaggagctaaccgcttttttgcacaacatgggggatcatgtaactcgccttgatcgt |
| | | tgggaaccggagctgaatgaagccataccaaacgacgagcgtgacaccacgatgcctgtagcaatggcaa |
| | | caacgttgcgcaaactattaactggcgaactacttactctagcttcccggcaacaattaatagactggatggag |
| | | gcggataaagttgcaggaccacttctgcgctcggcccttccggctggctggtttattgctgataaatctggagc |
| | | cggtgagcgtgggtctcgcggtatcattgcagcactggggccagatggtaagccctcccgtatcgtagttatc |
| | | tacacgacggggagtcaggcaactatggatgaacgaaatagacagatcgctgagataggtgcctcactgatt |
| | | aagcattggtaactgtcagaccaagtttactcatatatactttagattgatttaaaacttcatttttaatttaaaagga |
| | | tctaggtgaagatcctttttgataatctcatgaccaaaatcccttaacgtgagttttcgttccactgagcgtcagac |
| | | cccgtagaaaagatcaaaggatcttcttgaaatcctttttttctgcgcgtaatctgctgcttgcaaacaaaaaaac |
| | | caccgctaccagcggtggtttgtttgccggatcaagagctaccaactctttttccgaaggtaactggcttcagc |
| | | agagcgcagataccaaatactgttcttctagtgtagccgtagttaggccaccacttcaagaactctgtagcacc |
| | | gcctacatacctcgctctgctaatcctgttaccagtggctgctgccagtggcgataagtcgtgtcttaccgggtt |
| | | ggactcaagacgatagttaccggataaggcgcagcggtcgggctgaacggggggttcgtgcacacagcc |
| | | cagcttggagcgaacgacctacaccgaactgagatacctacagcgtgagctatgagaaagcgccacgcttc |
| | | ccgaagggagaaaggcggacaggtatccggtaagcggcagggtcggaacaggagagcgcacgaggga |
| | | gcttccagggggaaacgcctggtatctttatagtcctgtcgggtttcgccacctctgacttgagcgtcgatttttg |
| | | tgatgctcgtcaggggggggagcctatggaaaaacgccagcaacgcggcctttttacggttcctggcctttt |
| | | gctggccttttgctcacatgtcctgcaggcag |
| 29 | Plasmid TM039 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcgtactagtt |
| | | attaatagtaatcaattacggggtcattagttcatagcccatatatggagttccgcgttacataacttacggtaaat |
| | | ggcccgcctggctgaccgcccaacgacccccgcccattgacgtcaataatgacgtatgttcccatagtaacg |
| | | ccaatagggactttccattgacgtcaatgggtggagtatttacggtaaactgcccacttggcagtacatcaagt |
| | | gtatcatatgccaagtacgccccctattgacgtcaatgacggtaaatggcccgcctggcattatgcccagtac |
| | | atgaccttatgggactttcctacttggcagtacatctacgtattagtcatcgctattaccatggtcgaggtgagcc |
| | | ccacgttctgcttcactctccccatctcccccccctccccacccccaattttgtatttatttattttttaattattttgtg |
| | | cagcgatgggggcggggggggggggggggcgcgcgccaggcggggcggggcggggcgaggggcg |
| | | gggcggggcgaggcggagaggtgcggcggcagccaatcagagcggcgcgctccgaaagtttccttttat |
| | | ggcgaggcggcggcggcggcggccctataaaaagcgaagcgcgcggcgggcggggagtcgctgcga |
| | | cgctgccttcgccccgtgccccgctccgccgccgcctcgcgccgcccgccccggctctgactgaccgcgtt |
| | | actcccacaggtgagcggggggacggcccttctcctccgggctgtaattagcgcttggtttaatgacggctt |
| | | gtttcttttctgtggctgcgtgaaagccttgaggggctccgggagggccctttgtgcggggggagcggctcg |
| | | gggggtgcgtgcgtgtgtgtgtgcgtggggagcgccgcgtgcggctccgcgctgcccggcggctgtgag |
| | | cgctgcgggcgcggcgcggggctttgtgcgctccgcagtgtgcgcgaggggagcgcggccgggggcg |
| | | gtgccccgcggtgcggggggggctgcgaggggaacaaaggctgcgtgcggggtgtgtgcgtgggggg |
| | | gtgagcagggggtgtgggcgcgtcggtcgggctgcaaccccccctgcacccccctccccgagttgctgag |
| | | cacggcccggcttcgggtgcggggctccgtacggggcgtggcgcggggctcgccgtgccgggcgggg |
| | | ggtggcggcaggtgggggtgccgggcggggcggggccgcctcgggccggggagggctcgggggag |
| | | gggcgcggcggcccccggagcgccggcggctgtcgaggcgcggcgagccgcagccattgccttttatgg |
| | | taatcgtgcgagagggcgcagggacttcctttgtcccaaatctgtgcggagccgaaatctgggaggcgccg |
| | | ccgcaccccctctagcgggcgcggggcgaagcggtgcggcgccggcaggaaggaaatgggcgggga |
| | | gggccttcgtgcgtcgccgcgccgccgtccccttctccctctccagcctcggggctgtccgcggggggacg |
| | | gctgccttcgggggggacggggcagggcggggttcggcttctggcgtgtgaccggcggcatcgattgaat |
| | | tcgccaccatgtcagaagggggggcacgttccgcatggtacctgaagaggaacaggagctccgtgccca |
| | | actggagcagctcacaaccaaggaccatggacctgtctttggcccgtgcagccagctgccccgccacacct |
| | | tgcagaaggccaaggatgagctgaacgagagagaggagacccgggaggaggcagtgcgagagctgca |
| | | ggagatggtgcaggcgcaggcggcctcgggggaggagctggcggtggccgtggcggagagggtgcaa |
| | | gagaaggacagcggcttcttcctgcgcttcatccgcgcacggaagttcaacgtgggccgtgcctatgagctg |
| | | ctcagaggctatgtgaatttccggctgcagtaccctgagctctttgacagcctgtccccagaggctgtccgctg |
| | | caccattgaagctggctaccctggtgtcctctctagtcgggacaagtatggccgagtggtcatgctcttcaaca |
| | | ttgagaactggcaaagtcaagaaatcacctttgatgagatcttgcaggcatattgcttcatcctggagaagctg |
| | | ctggagaatgaggaaactcaaatcaatggcttctgcatcattgagaacttcaagggctttaccatgcagcagg |
| | | ctgctagtctccggacttcagatctcaggaagatggtggacatgctccaggattccttcccagcccggttcaa |
| | | agccatccacttcatccaccagccatggtacttcaccacgacctacaatgtggtcaagcccttcttgaagagc |
| | | aagctgcttgagagggtctttgtccacggggatgacctttctggtttctaccaggagatcgatgagaacatcct |
| | | gccctctgacttcgggggcacgctgcccaagtatgatggcaaggccgttgctgagcagctctttggccccca |
| | | ggcccaagctgagaacacagccttctgaggatcgtaccggtcgacctgcagaagcttgcctcgagcagcg |
| | | ctgctcgagagatctggatcataatcagccataccacatttgtagaggttttacttgctttaaaaaacctcccaca |
| | | cctccccctgaacctgaaacataaaatgaatgcaattgttgttgttaacttgtttattgcagcttataatggttacaa |
| | | ataaagcaatagcatcacaaatttcacaaataaagcatttttttcactgcattctagttgtggtttgtccaaactcat |
| | | caatgtatcttatcatgtctggtactagggttaccccagaacaggtcccattcatggcccacatgacaacctgct |
| | | tccccagtgggtatttttggagacagctcttctgtttccaggttttctctcctgcctaaatgtcctgcctaagtgcct |
| | | tcaagaacccttcaccatcctgctcctgcatgtgaccaggttccatggtcagttcaatcacctagtcacagttgg |
| | | taagtgacagagttgggacttgaacctatgcctgcctgacaccaagtctttttttgacacctagagccaagaca |
| | | tctgaagacaaactccctaggagagctggcgtcatagaaaccttaaaggttagggagacctgggtttgaatc |
| | | aggctttgtcagttatgacttgtgtgaccctagcaagttatttaacctttctgggtctcagtttcctcatctgcaaac |
| | | tgaggataataacagtacctaccaaaaagaactgtcgtgaaaaccatataatttctgcaatgctcctggcacag |
| | | tgtcctgttctaaagcatagttccccttctctttcttagctccatattgattattaccctaacttgcacaaagagactt |
| | | ggaggacccccatagagtatcggagggtcccccatttcctgctctttccactccacacccccagcaagcaca |
| | | gggaagttctgggggccataatccacccacaggaaccaaatctaagccacctttctggctggtagacatcca |
| | | ggtatgtgggcacagaggtagacaggctgaaatgctgctgtgctatcagttgggttttgctggaacaggaatg |
| | | gaaatggagaggctgacagaactgccctggggagcccaggcaagagggacagtggctggacaccccca |
| | | gccagttgtgcagaccatcagaacaagatcctagattttaggaatacagggttcaagtccgtgcggcaactct |
| | | tttctaaatatgcccaagccattaactttgagttttaaaaatactgatttacaagctgtacacaatgaaaaaatgcc |
| | | tatccctcacaccatgctgatgctgttccctgccatctcagattaccaattaaatacagaatgcccagttaaatgt |
| | | gaactttttttttttttttttttttgagatggagttttgttcttgtcgcccaggctagagtgcaatggtgcgatctcagct |
| | | cactgcaacctctgcctcccaggttcaagcaattctcctgccttagcctcctgagtagctggaactacaggtgc |
| | | ccaccagcacgcctggctaatttttggtatttttagtggagatggggtttcaccatgttggccaggctggtctcg |
| | | aactcctgacctcaggtgatctgcctgcctcggcctcccaaagtgctgggattacaggcgtgagcctaaatgt |
| | | gaacttttttaatactaaaaaagtatttgctgttcatcggaaattcacatttaactgggtgtcctgtatttttatttgcta |
| | | aatctaccatcaaattggtctggctcaacctggagaatggttaccctaggtaaccacgtgcggaccgagcgg |
| | | ccgcaggaacccctagtgatggagttggccactccctctctgcgcgctcgctcgctcactgaggccgggcg |
| | | accaaaggtcgcccgacgcccgggctttgcccgggggcctcagtgagcgagcgagcgcgcagctgcct |
| | | gcaggggcgcctgatgcggtattttctccttacgcatctgtgcggtatttcacaccgcatacgtcaaagcaacc |
| | | atagtacgcgccctgtagcggcgcattaagcgcggcgggtgtggtggttacgcgcagcgtgaccgctacac |
| | | ttgccagcgccttagcgcccgctcctttcgctttcttcccttcctttctcgccacgttcgccggctttccccgtcaa |
| | | gctctaaatcgggggctccctttagggttccgatttagtgctttacggcacctcgaccccaaaaaacttgatttg |
| | | ggtgatggttcacgtagtgggccatcgccctgatagacggtttttcgccctttgacgttggagtccacgttcttta |
| | | atagtggactcttgttccaaactggaacaacactcaactctatctcgggctattcttttgatttataagggattttgc |
| | | cgatttcggtctattggttaaaaaatgagctgatttaacaaaaatttaacgcgaattttaacaaaatattaacgttta |
| | | caattttatggtgcactctcagtacaatctgctctgatgccgcatagttaagccagccccgacacccgccaaca |
| | | cccgctgacgcgccctgacgggcttgtctgctcccggcatccgcttacagacaagctgtgaccgtctccggg |
| | | agctgcatgtgtcagaggttttcaccgtcatcaccgaaacgcgcgagacgaaagggcctcgtgatacgccta |
| | | tttttataggttaatgtcatgataataatggtttcttagacgtcaggtggcacttttcggggaaatgtgcgcggaa |
| | | cccctatttgtttatttttctaaatacattcaaatatgtatccgctcatgagacaataaccctgataaatgcttcaata |
| | | atattgaaaaaggaagagtatgagtattcaacatttccgtgtcgcccttattcccttttttgcggcattttgccttcc |
| | | tgtttttgctcacccagaaacgctggtgaaagtaaaagatgctgaagatcagttgggtgcacgagtgggttac |
| | | atcgaactggatctcaacagcggtaagatccttgagagttttcgccccgaagaacgttttccaatgatgagcac |
| | | ttttaaagttctgctatgtggcgcggtattatcccgtattgacgccgggcaagagcaactcggtcgccgcatac |
| | | actattctcagaatgacttggttgagtactcaccagtcacagaaaagcatcttacggatggcatgacagtaaga |
| | | gaattatgcagtgctgccataaccatgagtgataacactgcggccaacttacttctgacaacgatcggaggac |
| | | cgaaggagctaaccgcttttttgcacaacatgggggatcatgtaactcgccttgatcgttgggaaccggagct |
| | | gaatgaagccataccaaacgacgagcgtgacaccacgatgcctgtagcaatggcaacaacgttgcgcaaa |
| | | ctattaactggcgaactacttactctagcttcccggcaacaattaatagactggatggaggcggataaagttgc |
| | | aggaccacttctgcgctcggcccttccggctggctggtttattgctgataaatctggagccggtgagcgtggg |
| | | tctcgcggtatcattgcagcactggggccagatggtaagccctcccgtatcgtagttatctacacgacgggga |
| | | gtcaggcaactatggatgaacgaaatagacagatcgctgagataggtgcctcactgattaagcattggtaact |
| | | gtcagaccaagtttactcatatatactttagattgatttaaaacttcatttttaatttaaaaggatctaggtgaagatc |
| | | ctttttgataatctcatgaccaaaatcccttaacgtgagttttcgttccactgagcgtcagaccccgtagaaaaga |
| | | tcaaaggatcttcttgaaatcctttttttctgcgcgtaatctgctgcttgcaaacaaaaaaaccaccgctaccagc |
| | | ggtggtttgtttgccggatcaagagctaccaactctttttccgaaggtaactggcttcagcagagcgcagatac |
| | | caaatactgttcttctagtgtagccgtagttaggccaccacttcaagaactctgtagcaccgcctacatacctcg |
| | | ctctgctaatcctgttaccagtggctgctgccagtggcgataagtcgtgtcttaccgggttggactcaagacga |
| | | tagttaccggataaggcgcagcggtcgggctgaacggggggttcgtgcacacagcccagcttggagcgaa |
| | | cgacctacaccgaactgagatacctacagcgtgagctatgagaaagcgccacgcttcccgaagggagaaa |
| | | ggcggacaggtatccggtaagcggcagggtcggaacaggagagcgcacgagggagcttccaggggga |
| | | aacgcctggtatctttatagtcctgtcgggtttcgccacctctgacttgagcgtcgatttttgtgatgctcgtcagg |
| | | ggggcggagcctatggaaaaacgccagcaacgcggcctttttacggttcctggccttttgctggccttttgctc |
| | | acatgtcctgcaggcag |
| 30 | Plasmid TM040 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcgtttgtcct |
| | | ctccctgcttggccttaaccagccacatttctcaactgaccccactcactgcagaggtgaaaactaccatgcca |
| | | ggtcctgctggctgggggaggggtgggcaataggcctggatttgccagagctgccactgtagatgtagtcat |
| | | atttacgatttcccttcacctcttattaccctggtggtggtggtgggggggggggggtgctctctcagcaaccc |
| | | caccccgggatcttgaggagaaagagggcagagaaaagagggaatgggactggcccagatcccagccc |
| | | cacagccgggcttccacatggccgagcaggaactccagagcaggagcacacaaaggagggctttgatgc |
| | | gcctccagccaggcccaggcctctcccctctcccctttctctctgggtcttcctttgccccactgagggcctcct |
| | | gtgagcccgatttaacggaaactgtgggcggtgagaagttccttatgacacactaatcccaacctgctgaccg |
| | | gaccacgcctccagcggagggaacctctagagctccaggacattcaggtaccaggtagccccaaggagg |
| | | agctgccgaatcgatggatcgggaactgaaaaaccagaaagttaactggtaagtttagtctttttgtcttttatttc |
| | | aggtcccggatccggtggtggtgcaaatcaaagaactgctcctcagtggatgttgcctttacttctaggcctgt |
| | | acggaagtgttacttctgctctaaaagctgcggaattgtacccgccccgggatccatcgattgaattcgccacc |
| | | atgtcagaagggggggcacgttccgcatggtacctgaagaggaacaggagctccgtgcccaactggagc |
| | | agctcacaaccaaggaccatggacctgtctttggcccgtgcagccagctgccccgccacaccttgcagaag |
| | | gccaaggatgagctgaacgagagagaggagacccgggaggaggcagtgcgagagctgcaggagatgg |
| | | tgcaggcgcaggcggcctcgggggaggagctggcggtggccgtggcggagagggtgcaagagaagga |
| | | cagcggcttcttcctgcgcttcatccgcgcacggaagttcaacgtgggccgtgcctatgagctgctcagagg |
| | | ctatgtgaatttccggctgcagtaccctgagctctttgacagcctgtccccagaggctgtccgctgcaccattg |
| | | aagctggctaccctggtgtcctctctagtcgggacaagtatggccgagtggtcatgctcttcaacattgagaac |
| | | tggcaaagtcaagaaatcacctttgatgagatcttgcaggcatattgcttcatcctggagaagctgctggagaa |
| | | tgaggaaactcaaatcaatggcttctgcatcattgagaacttcaagggctttaccatgcagcaggctgctagtc |
| | | tccggacttcagatctcaggaagatggtggacatgctccaggattccttcccagcccggttcaaagccatcca |
| | | cttcatccaccagccatggtacttcaccacgacctacaatgtggtcaagcccttcttgaagagcaagctgcttg |
| | | agagggtctttgtccacggggatgacctttctggtttctaccaggagatcgatgagaacatcctgccctctgac |
| | | ttcgggggcacgctgcccaagtatgatggcaaggccgttgctgagcagctctttggcccccaggcccaagc |
| | | tgagaacacagccttctgaggatcgtaccggtcgacctgcagaagcttgcctcgagcagcgctgctcgaga |
| | | gatctggatcataatcagccataccacatttgtagaggttttacttgctttaaaaaacctcccacacctccccctg |
| | | aacctgaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgtatctt |
| | | atcatgtctggtactagggttaccccagaacaggtcccattcatggcccacatgacaacctgcttccccagtg |
| | | ggtatttttggagacagctcttctgtttccaggttttctctcctgcctaaatgtcctgcctaagtgccttcaagaacc |
| | | cttcaccatcctgctcctgcatgtgaccaggttccatggtcagttcaatcacctagtcacagttggtaagtgaca |
| | | gagttgggacttgaacctatgcctgcctgacaccaagtctttttttgacacctagagccaagacatctgaagac |
| | | aaactccctaggagagctggcgtcatagaaaccttaaaggttagggagacctgggtttgaatcaggctttgtc |
| | | agttatgacttgtgtgaccctagcaagttatttaacctttctgggtctcagtttcctcatctgcaaactgaggataat |
| | | aacagtacctaccaaaaagaactgtcgtgaaaaccatataatttctgcaatgctcctggcacagtgtcctgttct |
| | | aaagcatagttccccttctctttcttagctccatattgattattaccctaacttgcacaaagagacttggaggaccc |
| | | ccatagagtatcggagggtcccccatttcctgctctttccactccacacccccagcaagcacagggaagttct |
| | | gggggccataatccacccacaggaaccaaatctaagccacctttctggctggtagacatccaggtatgtggg |
| | | cacagaggtagacaggctgaaatgctgctgtgctatcagttgggttttgctggaacaggaatggaaatggag |
| | | aggctgacagaactgccctggggagcccaggcaagagggacagtggctggacacccccagccagttgtg |
| | | cagaccatcagaacaagatcctagattttaggaatacagggttcaagtccgtgcggcaactcttttctaaatatg |
| | | cccaagccattaactttgagttttaaaaatactgatttacaagctgtacacaatgaaaaaatgcctatccctcaca |
| | | ccatgctgatgctgttccctgccatctcagattaccaattaaatacagaatgcccagttaaatgtgaacttttttttt |
| | | ttttttttttttgagatggagttttgttcttgtcgcccaggctagagtgcaatggtgcgatctcagctcactgcaacc |
| | | tctgcctcccaggttcaagcaattctcctgccttagcctcctgagtagctggaactacaggtgcccaccagca |
| | | cgcctggctaatttttggtatttttagtggagatggggtttcaccatgttggccaggctggtctcgaactcctgac |
| | | ctcaggtgatctgcctgcctcggcctcccaaagtgctgggattacaggcgtgagcctaaatgtgaactttttta |
| | | atactaaaaaagtatttgctgttcatcggaaattcacatttaactgggtgtcctgtatttttatttgctaaatctaccat |
| | | caaattggtctggctcaacctggagaatggttaccctaggtaaccacgtgcggaccgagcggccgcaggaa |
| | | cccctagtgatggagttggccactccctctctgcgcgctcgctcgctcactgaggccgggcgaccaaaggtc |
| | | gcccgacgcccgggctttgcccgggggcctcagtgagcgagcgagcgcgcagctgcctgcaggggcg |
| | | cctgatgcggtattttctccttacgcatctgtgcggtatttcacaccgcatacgtcaaagcaaccatagtacgcg |
| | | ccctgtagcggcgcattaagcgcggcgggtgtggtggttacgcgcagcgtgaccgctacacttgccagcgc |
| | | cttagcgcccgctcctttcgctttcttcccttcctttctcgccacgttcgccggctttccccgtcaagctctaaatc |
| | | gggggctccctttagggttccgatttagtgctttacggcacctcgaccccaaaaaacttgatttgggtgatggtt |
| | | cacgtagtgggccatcgccctgatagacggtttttcgccctttgacgttggagtccacgttctttaatagtggac |
| | | tcttgttccaaactggaacaacactcaactctatctcgggctattcttttgatttataagggattttgccgatttcgg |
| | | tctattggttaaaaaatgagctgatttaacaaaaatttaacgcgaattttaacaaaatattaacgtttacaattttatg |
| | | gtgcactctcagtacaatctgctctgatgccgcatagttaagccagccccgacacccgccaacacccgctga |
| | | cgcgccctgacgggcttgtctgctcccggcatccgcttacagacaagctgtgaccgtctccgggagctgcat |
| | | gtgtcagaggttttcaccgtcatcaccgaaacgcgcgagacgaaagggcctcgtgatacgcctatttttatag |
| | | gttaatgtcatgataataatggtttcttagacgtcaggtggcacttttcggggaaatgtgcgcggaacccctattt |
| | | gtttatttttctaaatacattcaaatatgtatccgctcatgagacaataaccctgataaatgcttcaataatattgaa |
| | | aaaggaagagtatgagtattcaacatttccgtgtcgcccttattcccttttttgcggcattttgccttcctgtttttgc |
| | | tcacccagaaacgctggtgaaagtaaaagatgctgaagatcagttgggtgcacgagtgggttacatcgaact |
| | | ggatctcaacagcggtaagatccttgagagttttcgccccgaagaacgttttccaatgatgagcacttttaaagt |
| | | tctgctatgtggcgcggtattatcccgtattgacgccgggcaagagcaactcggtcgccgcatacactattctc |
| | | agaatgacttggttgagtactcaccagtcacagaaaagcatcttacggatggcatgacagtaagagaattatg |
| | | cagtgctgccataaccatgagtgataacactgcggccaacttacttctgacaacgatcggaggaccgaagga |
| | | gctaaccgcttttttgcacaacatgggggatcatgtaactcgccttgatcgttgggaaccggagctgaatgaa |
| | | gccataccaaacgacgagcgtgacaccacgatgcctgtagcaatggcaacaacgttgcgcaaactattaact |
| | | ggcgaactacttactctagcttcccggcaacaattaatagactggatggaggcggataaagttgcaggacca |
| | | cttctgcgctcggcccttccggctggctggtttattgctgataaatctggagccggtgagcgtgggtctcgcgg |
| | | tatcattgcagcactggggccagatggtaagccctcccgtatcgtagttatctacacgacggggagtcaggc |
| | | aactatggatgaacgaaatagacagatcgctgagataggtgcctcactgattaagcattggtaactgtcagac |
| | | caagtttactcatatatactttagattgatttaaaacttcatttttaatttaaaaggatctaggtgaagatcctttttgat |
| | | aatctcatgaccaaaatcccttaacgtgagttttcgttccactgagcgtcagaccccgtagaaaagatcaaagg |
| | | atcttcttgaaatcctttttttctgcgcgtaatctgctgcttgcaaacaaaaaaaccaccgctaccagcggtggttt |
| | | gtttgccggatcaagagctaccaactctttttccgaaggtaactggcttcagcagagcgcagataccaaatact |
| | | gttcttctagtgtagccgtagttaggccaccacttcaagaactctgtagcaccgcctacatacctcgctctgcta |
| | | atcctgttaccagtggctgctgccagtggcgataagtcgtgtcttaccgggttggactcaagacgatagttacc |
| | | ggataaggcgcagcggtcgggctgaacggggggttcgtgcacacagcccagcttggagcgaacgaccta |
| | | caccgaactgagatacctacagcgtgagctatgagaaagcgccacgcttcccgaagggagaaaggcgga |
| | | caggtatccggtaagcggcagggtcggaacaggagagcgcacgagggagcttccagggggaaacgcct |
| | | ggtatctttatagtcctgtcgggtttcgccacctctgacttgagcgtcgatttttgtgatgctcgtcaggggggcg |
| | | gagcctatggaaaaacgccagcaacgcggcctttttacggttcctggccttttgctggccttttgctcacatgtc |
| | | ctgcaggcag |
| 31 | Plasmid TM016 | cgcgctcgctcgctcactgaggccgcccgggcaaagcccgggcgtcgggcgacctttggtcgcccggcct |
| | | cagtgagcgagcgagcgcgcagagagggagtggggtaccacgcgtttgtcctctccctgcttggccttaac |
| | | cagccacatttctcaactgaccccactcactgcagaggtgaaaactaccatgccaggtcctgctggctgggg |
| | | gaggggtgggcaataggcctggatttgccagagctgccactgtagatgtagtcatatttacgatttcccttcac |
| | | ctcttattaccctggtggtggtggtgggggggggggggtgctctctcagcaaccccaccccgggatcttgag |
| | | gagaaagagggcagagaaaagagggaatgggactggcccagatcccagccccacagccgggcttccac |
| | | atggccgagcaggaactccagagcaggagcacacaaaggagggctttgatgcgcctccagccaggccca |
| | | ggcctctcccctctcccctttctctctgggtcttcctttgccccactgagggcctcctgtgagcccgatttaacgg |
| | | aaactgtgggcggtgagaagttccttatgacacactaatcccaacctgctgaccggaccacgcctccagcgg |
| | | agggaacctctagagctccaggacattcaggtaccaggtagccccaaggaggagctgccgaatcgatgga |
| | | tcgggaactgaaaaaccagaaagttaactggtaagtttagtctttttgtcttttatttcaggtcccggatccggtg |
| | | gtggtgcaaatcaaagaactgctcctcagtggatgttgcctttacttctaggcctgtacggaagtgttacttctgc |
| | | tctaaaagctgcggaattgtacccgccccgggatccatcgattgaattccccggggatcctctagagtcgaaa |
| | | ttcgccaccatggtgagcaagggcgaggagctgttcaccggggtggtgcccatcctggtcgagctggacg |
| | | gcgacgtaaacggccacaagttcagcgtgtccggcgagggcgagggcgatgccacctacggcaagctga |
| | | ccctgaagttcatctgcaccaccggcaagctgcccgtgccctggcccaccctcgtgaccaccctgacctacg |
| | | gcgtgcagtgcttcagccgctaccccgaccacatgaagcagcacgacttcttcaagtccgccatgcccgaa |
| | | ggctacgtccaggagcgcaccatcttcttcaaggacgacggcaactacaagacccgcgccgaggtgaagtt |
| | | cgagggcgacaccctggtgaaccgcatcgagctgaagggcatcgacttcaaggaggacggcaacatcct |
| | | ggggcacaagctggagtacaactacaacagccacaacgtctatatcatggccgacaagcagaagaacggc |
| | | atcaaggtgaacttcaagatccgccacaacatcgaggacggcagcgtgcagctcgccgaccactaccagc |
| | | agaacacccccatcggcgacggccccgtgctgctgcccgacaaccactacctgagcacccagtccgccct |
| | | gagcaaagaccccaacgagaagcgcgatcacatggtcctgctggagttcgtgaccgccgccgggatcact |
| | | ctcggcatggacgagctgtacaagtaatagggtaccggtcgacctgcagaagcttgcctcgagcagcgctg |
| | | ctcgagagatctggatcataatcagccataccacatttgtagaggttttacttgctttaaaaaacctcccacacct |
| | | ccccctgaacctgaaacataaaatgaatgcaattgttgttgttaacttgtttattgcagcttataatggttacaaata |
| | | aagcaatagcatcacaaatttcacaaataaagcatttttttcactgcattctagttgtggtttgtccaaactcatcaa |
| | | tgtatcttatcatgtctggtaaccacgtgcggaccgagcggccgcaggaacccctagtgatggagttggcca |
| | | ctccctctctgcgcgctcgctcgctcactgaggccgggcgaccaaaggtcgcccgacgcccgggctttgcc |
| | | cgggcggcctcagtgagcgagcgagcgcgcagctgcctgcaggggcgcctgatgcggtattttctccttac |
| | | gcatctgtgcggtatttcacaccgcatacgtcaaagcaaccatagtacgcgccctgtagcggcgcattaagc |
| | | gcggcgggtgtggtggttacgcgcagcgtgaccgctacacttgccagcgccttagcgcccgctcctttcgct |
| | | ttcttcccttcctttctcgccacgttcgccggctttccccgtcaagctctaaatcgggggctccctttagggttcc |
| | | gatttagtgctttacggcacctcgaccccaaaaaacttgatttgggtgatggttcacgtagtgggccatcgccc |
| | | tgatagacggtttttcgccctttgacgttggagtccacgttctttaatagtggactcttgttccaaactggaacaac |
| | | actcaactctatctcgggctattcttttgatttataagggattttgccgatttcggtctattggttaaaaaatgagctg |
| | | atttaacaaaaatttaacgcgaattttaacaaaatattaacgtttacaattttatggtgcactctcagtacaatctgc |
| | | tctgatgccgcatagttaagccagccccgacacccgccaacacccgctgacgcgccctgacgggcttgtct |
| | | gctcccggcatccgcttacagacaagctgtgaccgtctccgggagctgcatgtgtcagaggttttcaccgtca |
| | | tcaccgaaacgcgcgagacgaaagggcctcgtgatacgcctatttttataggttaatgtcatgataataatggtt |
| | | tcttagacgtcaggtggcacttttcggggaaatgtgcgcggaacccctatttgtttatttttctaaatacattcaaa |
| | | tatgtatccgctcatgagacaataaccctgataaatgcttcaataatattgaaaaaggaagagtatgagtattca |
| | | acatttccgtgtcgcccttattcccttttttgcggcattttgccttcctgtttttgctcacccagaaacgctggtgaa |
| | | agtaaaagatgctgaagatcagttgggtgcacgagtgggttacatcgaactggatctcaacagcggtaagat |
| | | ccttgagagttttcgccccgaagaacgttttccaatgatgagcacttttaaagttctgctatgtggcgcggtatta |
| | | tcccgtattgacgccgggcaagagcaactcggtcgccgcatacactattctcagaatgacttggttgagtact |
| | | caccagtcacagaaaagcatcttacggatggcatgacagtaagagaattatgcagtgctgccataaccatga |
| | | gtgataacactgcggccaacttacttctgacaacgatcggaggaccgaaggagctaaccgcttttttgcacaa |
| | | catgggggatcatgtaactcgccttgatcgttgggaaccggagctgaatgaagccataccaaacgacgagc |
| | | gtgacaccacgatgcctgtagcaatggcaacaacgttgcgcaaactattaactggcgaactacttactctagc |
| | | ttcccggcaacaattaatagactggatggaggcggataaagttgcaggaccacttctgcgctcggcccttcc |
| | | ggctggctggtttattgctgataaatctggagccggtgagcgtgggtctcgcggtatcattgcagcactgggg |
| | | ccagatggtaagccctcccgtatcgtagttatctacacgacggggagtcaggcaactatggatgaacgaaat |
| | | agacagatcgctgagataggtgcctcactgattaagcattggtaactgtcagaccaagtttactcatatatacttt |
| | | agattgatttaaaacttcatttttaatttaaaaggatctaggtgaagatcctttttgataatctcatgaccaaaatccc |
| | | ttaacgtgagttttcgttccactgagcgtcagaccccgtagaaaagatcaaaggatcttcttgaaatcctttttttc |
| | | tgcgcgtaatctgctgcttgcaaacaaaaaaaccaccgctaccagcggtggtttgtttgccggatcaagagct |
| | | accaactctttttccgaaggtaactggcttcagcagagcgcagataccaaatactgttcttctagtgtagccgta |
| | | gttaggccaccacttcaagaactctgtagcaccgcctacatacctcgctctgctaatcctgttaccagtggctg |
| | | ctgccagtggcgataagtcgtgtcttaccgggttggactcaagacgatagttaccggataaggcgcagcggt |
| | | cgggctgaacggggggttcgtgcacacagcccagcttggagcgaacgacctacaccgaactgagatacct |
| | | acagcgtgagctatgagaaagcgccacgcttcccgaagggagaaaggcggacaggtatccggtaagcgg |
| | | cagggtcggaacaggagagcgcacgagggagcttccagggggaaacgcctggtatctttatagtcctgtcg |
| | | ggtttcgccacctctgacttgagcgtcgatttttgtgatgctcgtcaggggggcggagcctatggaaaaacgc |
| | | cagcaacgcggcctttttacggttcctggccttttgctggccttttgctcacatgtcctgcaggcagctg |
| 32 | Plasmid TM035 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcagcttttgt |
| | | cctctccctgcttggccttaaccagccacatttctcaactgaccccactcactgcagaggtgaaaactaccatg |
| | | ccaggtcctgctggctgggggaggggtgggcaataggcctggatttgccagagctgccactgtagatgtag |
| | | tcatatttacgatttcccttcacctcttattaccctggtggtggtggtgggggggggggggtgctctctcagcaa |
| | | ccccaccccgggatcttgaggagaaagagggcagagaaaagagggaatgggactggcccagatcccag |
| | | ccccacagccgggcttccacatggccgagcaggaactccagagcaggagcacacaaaggagggctttga |
| | | tgcgcctccagccaggcccaggcctctcccctctcccctttctctctgggtcttcctttgccccactgagggcct |
| | | cctgtgagcccgatttaacggaaactgtgggcggtgagaagttccttatgacacactaatcccaacctgctga |
| | | ccggaccacgcctccagcggagggaacctctagagctccaggacattcaggtaccaggtagccccaagg |
| | | aggagctgccgacctggcaggtaagtcaatacctggggcttgcctgggccagggagcccaggactggggt |
| | | gaggactcaggggagcagggagaccacgtcccaagatgcctgtaaaactgaaaccacctggccattctcc |
| | | aggttgagccagaccaatttgatggcagatttagcaaataaaaatacaggacacccagttaaatgtgaatttca |
| | | gatgaacagcaaatacttttttagtattaaaaaagttcacatttaggctcacgcctgtaatcccagcactttggga |
| | | ggccgaggcaggcagatcacctgaggtcaggagttcgagaccagcctggccaacatggtgaaaccccatc |
| | | tccactaaaaataccaaaaattagccaggcgtgctggtgggcacctgtagttccagctactcaggaggctaa |
| | | ggcaggagaattgcttgaacctgggaggcagaggttgcagtgagctgagatcgcaccattgcactctagcct |
| | | gggcgacaagaacaaaactccatctcaaaaaaaaaaaaaaaaaaaaagttcacatttaactgggcattctgta |
| | | tttaattggtaatctgagatggcagggaacagcatcagcatggtgtgagggataggcattttttcattgtgtaca |
| | | gcttgtaaatcagtatttttaaaactcaaagttaatggcttgggcatatttagaaaagagttgccgcacggacttg |
| | | aaccctgtattcctaaaatctaggatcttgttctgatggtctgcacaactggctgggggtgtccagccactgtcc |
| | | ctcttgcctgggctccccagggcagttctgtcagcctctccatttccattcctgttccagcaaaacccaactgat |
| | | agcacagcagcatttcagcctgtctacctctgtgcccacatacctggatgtctaccagccagaaaggtggctt |
| | | agatttggttcctgtgggtggattatggcccccagaacttccctgtgcttgctgggggtgtggagtggaaaga |
| | | gcaggaaatgggggaccctccgatactctatgggggtcctccaagtctctttgtgcaagttagggtaataatc |
| | | aatatggagctaagaaagagaaggggaactatgctttagaacaggacactgtgccaggagcattgcagaaa |
| | | ttatatggttttcacgacagttctttttggtaggtactgttattatcctcagtttgcagatgaggaaactgagaccca |
| | | gaaaggttaaataacttgctagggtcacacaagtcataactgacaaagcctgattcaaacccaggtctcccta |
| | | acctttaaggtttctatgacgccagctctcctagggagtttgtcttcagatgtcttggctctaggtgtcaaaaaaa |
| | | gacttggtgtcaggcaggcataggttcaagtcccaactctgtcacttaccaactgtgactaggtgattgaactg |
| | | accatggaacctggtcacatgcaggagcaggatggtgaagggttcttgaaggcacttaggcaggacatttag |
| | | gcaggagagaaaacctggaaacagaagagctgtctccaaaaatacccactggggaagcaggttgtcatgt |
| | | gggccatgaatgggacctgttctggtaaccaagcattgcttatgtgtccattacatttcataacacttccatccta |
| | | ctttacagggaacaaccaagactggggttaaatctcacagcctgcaagtggaagagaagaacttgaaccca |
| | | ggtccaacttttgcgccacagcaggctgcctcttggtcctgacaggaagtcacaacttgggtctgagtactgat |
| | | ccctggctattttttggctgtgttaccttggacaagtcacttattcctcctcccgtttcctcctatgtaaaatggaaat |
| | | aataatgttgaccctgggtctgagagagtggatttgaaagtacttagtgcatcacaaagcacagaacacacttc |
| | | cagtctcgtgattatgtacttatgtaactggtcatcacccatcttgagaatgaatgcattggggaaagggccatc |
| | | cactaggctgcgaagtttctgagggactccttcgggctggagaaggatggccacaggagggaggagagat |
| | | tgccttatcctgcagtgatcatgtcattgagaacagagccagattctttttttcctggcagggccaacttgttttaa |
| | | catctaaggactgagctatttgtgtctgtgccctttgtccaagcagtgtttcccaaagtgtagcccaagaaccat |
| | | ctccctcagagccaccaggaagtgctttaaattgcaggttcctaggccacagcctgcacctgcagagtcaga |
| | | atcatggaggttgggacccaggcacctgcgtttctaacaaatgcctcgggtgattctgatgcaattgaaagttt |
| | | gagatccacagttctgagacaataacagaatggtttttctaacccctgcagccctgacttcctatcctagggaa |
| | | ggggccggctggagaggccaggacagagaaagcagatcccttctttttccaaggactctgtgtcttccatag |
| | | gcaacgaattccccggggatcctctagagtcgaaattcgccaccatggtgagcaagggcgaggagctgttc |
| | | accggggtggtgcccatcctggtcgagctggacggcgacgtaaacggccacaagttcagcgtgtccggcg |
| | | agggcgagggcgatgccacctacggcaagctgaccctgaagttcatctgcaccaccggcaagctgcccgt |
| | | gccctggcccaccctcgtgaccaccctgacctacggcgtgcagtgcttcagccgctaccccgaccacatga |
| | | agcagcacgacttcttcaagtccgccatgcccgaaggctacgtccaggagcgcaccatcttcttcaaggacg |
| | | acggcaactacaagacccgcgccgaggtgaagttcgagggcgacaccctggtgaaccgcatcgagctga |
| | | agggcatcgacttcaaggaggacggcaacatcctggggcacaagctggagtacaactacaacagccacaa |
| | | cgtctatatcatggccgacaagcagaagaacggcatcaaggtgaacttcaagatccgccacaacatcgagg |
| | | acggcagcgtgcagctcgccgaccactaccagcagaacacccccatcggcgacggccccgtgctgctgc |
| | | ccgacaaccactacctgagcacccagtccgccctgagcaaagaccccaacgagaagcgcgatcacatggt |
| | | cctgctggagttcgtgaccgccgccgggatcactctcggcatggacgagctgtacaagtaatagggtaccg |
| | | gtcgacctgcagaagcttgcctcgagcagcgctgctcgagagatctggatcataatcagccataccacatttg |
| | | tagaggttttacttgctttaaaaaacctcccacacctccccctgaacctgaaacataaaatgaatgcaattgttgt |
| | | tgttaacttgtttattgcagcttataatggttacaaataaagcaatagcatcacaaatttcacaaataaagcatttttt |
| | | tcactgcattctagttgtggtttgtccaaactcatcaatgtatcttatcatgtctggtaaccacgtgcggaccgag |
| | | cggccgcaggaacccctagtgatggagttggccactccctctctgcgcgctcgctcgctcactgaggccgg |
| | | gcgaccaaaggtcgcccgacgcccgggctttgcccgggcggcctcagtgagcgagcgagcgcgcagct |
| | | gcctgcaggggcgcctgatgcggtattttctccttacgcatctgtgcggtatttcacaccgcatacgtcaaagc |
| | | aaccatagtacgcgccctgtagcggcgcattaagcgcggcgggtgtggtggttacgcgcagcgtgaccgct |
| | | acacttgccagcgccttagcgcccgctcctttcgctttcttcccttcctttctcgccacgttcgccggctttcccc |
| | | gtcaagctctaaatcgggggctccctttagggttccgatttagtgctttacggcacctcgaccccaaaaaactt |
| | | gatttgggtgatggttcacgtagtgggccatcgccctgatagacggtttttcgccctttgacgttggagtccacg |
| | | ttctttaatagtggactcttgttccaaactggaacaacactcaactctatctcgggctattcttttgatttataaggg |
| | | attttgccgatttcggtctattggttaaaaaatgagctgatttaacaaaaatttaacgcgaattttaacaaaatatta |
| | | acgtttacaattttatggtgcactctcagtacaatctgctctgatgccgcatagttaagccagccccgacacccg |
| | | ccaacacccgctgacgcgccctgacgggcttgtctgctcccggcatccgcttacagacaagctgtgaccgtc |
| | | tccgggagctgcatgtgtcagaggttttcaccgtcatcaccgaaacgcgcgagacgaaagggcctcgtgat |
| | | acgcctatttttataggttaatgtcatgataataatggtttcttagacgtcaggtggcacttttcggggaaatgtgc |
| | | gcggaacccctatttgtttatttttctaaatacattcaaatatgtatccgctcatgagacaataaccctgataaatg |
| | | cttcaataatattgaaaaaggaagagtatgagtattcaacatttccgtgtcgcccttattcccttttttgcggcatttt |
| | | gccttcctgtttttgctcacccagaaacgctggtgaaagtaaaagatgctgaagatcagttgggtgcacgagt |
| | | gggttacatcgaactggatctcaacagcggtaagatccttgagagttttcgccccgaagaacgttttccaatga |
| | | tgagcacttttaaagttctgctatgtggcgcggtattatcccgtattgacgccgggcaagagcaactcggtcgc |
| | | cgcatacactattctcagaatgacttggttgagtactcaccagtcacagaaaagcatcttacggatggcatgac |
| | | agtaagagaattatgcagtgctgccataaccatgagtgataacactgcggccaacttacttctgacaacgatc |
| | | ggaggaccgaaggagctaaccgcttttttgcacaacatgggggatcatgtaactcgccttgatcgttgggaac |
| | | cggagctgaatgaagccataccaaacgacgagcgtgacaccacgatgcctgtagcaatggcaacaacgtt |
| | | gcgcaaactattaactggcgaactacttactctagcttcccggcaacaattaatagactggatggaggcggat |
| | | aaagttgcaggaccacttctgcgctcggcccttccggctggctggtttattgctgataaatctggagccggtga |
| | | gcgtgggtctcgcggtatcattgcagcactggggccagatggtaagccccccgtatcgtagttatctacacg |
| | | acggggagtcaggcaactatggatgaacgaaatagacagatcgctgagataggtgcctcactgattaagcat |
| | | tggtaactgtcagaccaagtttactcatatatactttagattgatttaaaacttcatttttaatttaaaaggatctaggt |
| | | gaagatcctttttgataatctcatgaccaaaatcccttaacgtgagttttcgttccactgagcgtcagaccccgta |
| | | gaaaagatcaaaggatcttcttgaaatcctttttttctgcgcgtaatctgctgcttgcaaacaaaaaaaccaccg |
| | | ctaccagcggtggtttgtttgccggatcaagagctaccaactctttttccgaaggtaactggcttcagcagagc |
| | | gcagataccaaatactgttcttctagtgtagccgtagttaggccaccacttcaagaactctgtagcaccgccta |
| | | catacctcgctctgctaatcctgttaccagtggctgctgccagtggcgataagtcgtgtcttaccgggttggact |
| | | caagacgatagttaccggataaggcgcagcggtcgggctgaacggggggttcgtgcacacagcccagctt |
| | | ggagcgaacgacctacaccgaactgagatacctacagcgtgagctatgagaaagcgccacgcttcccgaa |
| | | gggagaaaggcggacaggtatccggtaagcggcagggtcggaacaggagagcgcacgagggagcttc |
| | | cagggggaaacgcctggtatctttatagtcctgtcgggtttcgccacctctgacttgagcgtcgatttttgtgatg |
| | | ctcgtcaggggggcggagcctatggaaaaacgccagcaacgcggcctttttacggttcctggccttttgctg |
| | | gccttttgctcacatgtcctgcaggcag |
| 33 | Plasmid AG012 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcgtgacgtc |
| | | gtttaaacgggccccggtgttatctcattcttttttctcctctgtaagttgacatgtgatgtgggaacaaaggggat |
| | | aaagtcattattttgtgctaaaatcgtaattggagaggacctcctgttagctgggctttcttctatttattgtggtggt |
| | | tactggagttccttcttctagttttaggatatatatatatattttttttttttctttccctgaagatataataatatatatact |
| | | tctgaagattgagatttttaaattagttgtattgaaaactagctaatcagcaatttaaggctagcttgagacttatgt |
| | | cttgaatttgtttttgtaggctccaaaaccaaggagggagtggtgcatggtgtggcaacaggtaagctccattg |
| | | tgcttatatccaaagatgatatttaaagtatctagtgattagtgtggcccagtattcaagattcctatgaaattgtaa |
| | | aacaatcactgagcattctaagaacatatcagtcttattgaaactgaattctttataaagtatttttaaaaaggtaaa |
| | | tattgattataaataaaaaatatacttgccaagaataatgagggctttgaattgataagctatgtttaatttatagta |
| | | agtgggcatttaaatattctgaccaaaaatgtattgacaaactgctgacaaaaataaaatgtgaatattgccata |
| | | attttaaaaaaagagtaaaatttctgttgattacagtaaaatattttgaccttaaattatgttgattacaatattcctttg |
| | | ataattcagagtgcatttcaggaaacacccttggacagtcagtaaattgtttattgtatttatctttgtattgttatgg |
| | | tatagctatttgtacaaatattattgtgcaattattacatttctgattatattattcatttggcctaaatttaccaagaatt |
| | | tgaacaagtcaattaggtttacaatcaagaaatatcaaaaatgatgaaaaggatgataatcatcatcagatgttg |
| | | aggaagatgacgatgagagtgccagaaatagagaaatcaaaggagaaccaaaatttaacaaattaaaagcc |
| | | cacagacttgctgtaattaagttttctgttgtaagtactccacgtttcctggcagatgtggtgaagcaaaagatat |
| | | aatcagaaatataatttatatgatcggaaagcattaaacacaatagtgcctatacaaataaaatgttcctatcact |
| | | gacttctaaaatggaaatgaggacaatgatatgggaatcttaatacagtgttgtggataggactaaaaacaca |
| | | ggagtcagatcttcttggttcaacttcctgcttactccttaccagctgtgtgttttttgcaaggttcttcacctctatg |
| | | tgatttagcttcctcatctataaaataattcagtgaattaatgtacacaaaacatctggaaaacaaaagcaaaca |
| | | atatgtattttataagtgttacttatagttttatagtgaactttcttgtgcaacatttttacaactagtggagaaaaatat |
| | | ttctttaaatgaatacttttgatttaaaaatcagagtgtaaaaataaaacagactcctttgaaactagttctgttaga |
| | | agttaattgtgcacctttaatgggctctgttgcaatccaacagagaagtagttaagtaagtggactatgatggctt |
| | | ctagggacctcctataaatatgatattgtgaagcatgattataataagaactagataacagacaggtggagact |
| | | ccactatctgaagagggtcaacctagatgaatggtgttccatttagtagttgaggaagaacccatgaggtttag |
| | | aaagcagacaagcatgtggcaagttctggagtcagtggtaaaaattaaagaacccaactattactgtcaccta |
| | | atgatctaatggagactgtggagatgggctgcatttttttaatcttctccagaatgccaaaatgtaaacacatatc |
| | | tgtgtgtgtgtgtgtgtgtgtgtgtgtgtgtgagagagagagagagagagagagagactgaagtttgtacaatt |
| | | agacattttataaaatgttttctgaaggacagtggctcacaatcttaagtttctaacattgtacaatgttgggagac |
| | | tttgtatactttattttctctttagcatattaaggaatctgagatgtcctacagtaaagaaatttgcattacatagttaa |
| | | aatcagggttattcaaactttttgattattgaaacctttcttcattagttactagggttgaatgaaactagtgttccac |
| | | agaaaactatgggaaatgttgctaggcagtaaggacatggtgatttcagcatgtgcaatatttacagcgattgc |
| | | acccatggaccaccctggcagtagtgaaataaccaaaaatgctgtcataactagtatggctatgagaaacac |
| | | attgggcagaagcttgcctcgagcagcgctgctcgagagatctggatcataatcagccataccacatttgtag |
| | | aggttttacttgctttaaaaaacctcccacacctccccctgaacctgaaacataaaatgaatgcaattgttgttgtt |
| | | aacttgtttattgcagcttataatggttacaaataaagcaatagcatcacaaatttcacaaataaagcatttttttca |
| | | ctgcattctagttgtggtttgtccaaactcatcaatgtatcttatcatgtctggtaaccattctccaggttgagccag |
| | | accaatttgatggtagatttagcaaataaaaatacaggacacccagttaaatgtgaatttccgatgaacagcaa |
| | | atacttttttagtattaaaaaagttcacatttaggctcacgcctgtaatcccagcactttgggaggccgaggcag |
| | | gcagatcacctgaggtcaggagttcgagaccagcctggccaacatggtgaaaccccatctccactaaaaat |
| | | accaaaaattagccaggcgtgctggtgggcacctgtagttccagctactcaggaggctaaggcaggagaat |
| | | tgcttgaacctgggaggcagaggttgcagtgagctgagatcgcaccattgcactctagcctgggcgacaag |
| | | aacaaaactccatctcaaaaaaaaaaaaaaaaaaaaagttcacatttaactgggcattctgtatttaattggtaat |
| | | ctgagatggcagggaacagcatcagcatggtgtgagggataggcattttttcattgtgtacagcttgtaaatca |
| | | gtatttttaaaactcaaagttaatggcttgggcatatttagaaaagagttgccgcacggacttgaaccctgtattc |
| | | ctaaaatctaggatcttgttctgatggtctgcacaactggctgggggtgtccagccactgtccctcttgcctggg |
| | | ctccccagggcagttctgtcagcctctccatttccattcctgttccagcaaaacccaactgatagcacagcagc |
| | | atttcagcctgtctacctctgtgcccacatacctggatgtctaccagccagaaaggtggcttagatttggttcct |
| | | gtgggtggattatggcccccagaacttccctgtgcttgctgggggtgtggagtggaaagagcaggaaatgg |
| | | gggaccctccgatactctatgggggtcctccaagtctctttgtgcaagttagggtaataatcaatatggagcta |
| | | agaaagagaaggggaactatgctttagaacaggacactgtgccaggagcattgcagaaattatatggttttca |
| | | cgacagttctttttggtaggtactgttattatcctcagtttgcagatgaggaaactgagacccagaaaggttaaat |
| | | aacttgctagggtcacacaagtcataactgacaaagcctgattcaaacccaggtctccctaacctttaaggtttc |
| | | tatgacgccagctctcctagggagtttgtcttcagatgtcttggctctaggtgtcaaaaaaagacttggtgtcag |
| | | gcaggcataggttcaagtcccaactctgtcacttaccaactgtgactaggtgattgaactgaccatggaacctg |
| | | gtcacatgcaggagcaggatggtgaagggttcttgaaggcacttaggcaggacatttaggcaggagagaaa |
| | | acctggaaacagaagagctgtctccaaaaatacccactggggaagcaggttgtcatgtgggccatgaatgg |
| | | gacctgttctggggtaaccacgtgcggaccgagcggccgcaggaacccctagtgatggagttggccactc |
| | | cctctctgcgcgctcgctcgctcactgaggccgggcgaccaaaggtcgcccgacgcccgggctttgcccg |
| | | ggcggcctcagtgagcgagcgagcgcgcagctgcctgcaggggcgcctgatgcggtattttctccttacgc |
| | | atctgtgcggtatttcacaccgcatacgtcaaagcaaccatagtacgcgccctgtagcggcgcattaagcgc |
| | | ggcgggtgtggtggttacgcgcagcgtgaccgctacacttgccagcgccttagcgcccgctcctttcgctttc |
| | | ttcccttcctttctcgccacgttcgccggctttccccgtcaagctctaaatcgggggctccctttagggttccgat |
| | | ttagtgctttacggcacctcgaccccaaaaaacttgatttgggtgatggttcacgtagtgggccatcgccctga |
| | | tagacggtttttcgccctttgacgttggagtccacgttctttaatagtggactcttgttccaaactggaacaacact |
| | | caactctatctcgggctattcttttgatttataagggattttgccgatttcggtctattggttaaaaaatgagctgatt |
| | | taacaaaaatttaacgcgaattttaacaaaatattaacgtttacaattttatggtgcactctcagtacaatctgctct |
| | | gatgccgcatagttaagccagccccgacacccgccaacacccgctgacgcgccctgacgggcttgtctgct |
| | | cccggcatccgcttacagacaagctgtgaccgtctccgggagctgcatgtgtcagaggttttcaccgtcatca |
| | | ccgaaacgcgcgagacgaaagggcctcgtgatacgcctatttttataggttaatgtcatgataataatggtttct |
| | | tagacgtcaggtggcacttttcggggaaatgtgcgcggaacccctatttgtttatttttctaaatacattcaaatat |
| | | gtatccgctcatgagacaataaccctgataaatgcttcaataatattgaaaaaggaagagtatgagtattcaac |
| | | atttccgtgtcgcccttattcccttttttgcggcattttgccttcctgtttttgctcacccagaaacgctggtgaaagt |
| | | aaaagatgctgaagatcagttgggtgcacgagtgggttacatcgaactggatctcaacagcggtaagatcctt |
| | | gagagttttcgccccgaagaacgttttccaatgatgagcacttttaaagttctgctatgtggcgcggtattatccc |
| | | gtattgacgccgggcaagagcaactcggtcgccgcatacactattctcagaatgacttggttgagtactcacc |
| | | agtcacagaaaagcatcttacggatggcatgacagtaagagaattatgcagtgctgccataaccatgagtgat |
| | | aacactgcggccaacttacttctgacaacgatcggaggaccgaaggagctaaccgcttttttgcacaacatg |
| | | ggggatcatgtaactcgccttgatcgttgggaaccggagctgaatgaagccataccaaacgacgagcgtga |
| | | caccacgatgcctgtagcaatggcaacaacgttgcgcaaactattaactggcgaactacttactctagcttccc |
| | | ggcaacaattaatagactggatggaggcggataaagttgcaggaccacttctgcgctcggcccttccggctg |
| | | gctggtttattgctgataaatctggagccggtgagcgtgggtctcgcggtatcattgcagcactggggccaga |
| | | tggtaagccctcccgtatcgtagttatctacacgacggggagtcaggcaactatggatgaacgaaatagaca |
| | | gatcgctgagataggtgcctcactgattaagcattggtaactgtcagaccaagtttactcatatatactttagatt |
| | | gatttaaaacttcatttttaatttaaaaggatctaggtgaagatcctttttgataatctcatgaccaaaatcccttaac |
| | | gtgagttttcgttccactgagcgtcagaccccgtagaaaagatcaaaggatcttcttgaaatcctttttttctgcg |
| | | cgtaatctgctgcttgcaaacaaaaaaaccaccgctaccagcggtggtttgtttgccggatcaagagctacca |
| | | actctttttccgaaggtaactggcttcagcagagcgcagataccaaatactgttcttctagtgtagccgtagtta |
| | | ggccaccacttcaagaactctgtagcaccgcctacatacctcgctctgctaatcctgttaccagtggctgctgc |
| | | cagtggcgataagtcgtgtcttaccgggttggactcaagacgatagttaccggataaggcgcagcggtcgg |
| | | gctgaacggggggttcgtgcacacagcccagcttggagcgaacgacctacaccgaactgagatacctaca |
| | | gcgtgagctatgagaaagcgccacgcttcccgaagggagaaaggcggacaggtatccggtaagcggcag |
| | | ggtcggaacaggagagcgcacgagggagcttccagggggaaacgcctggtatctttatagtcctgtcgggt |
| | | ttcgccacctctgacttgagcgtcgatttttgtgatgctcgtcaggggggcggagcctatggaaaaacgccag |
| | | caacgcggcctttttacggttcctggccttttgctggccttttgctcacatgtcctgcaggcag |
| 34 | Plasmid AG004 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcgttacgtaa |
| | | tatttattgaagtttaatattgtgtttgtgatacagaagtatttgctttaattctaaataaaaattttatgcttttattgctg |
| | | gtttaagaagatttggattatccttgtactttgaggagaagtttcttatttgaaatattttggaaacaggtcttttaatg |
| | | tggaaagatagatattaatctcctcttctattactctccaagatccaacaaaagtgattataccccccaaaatatg |
| | | atggtagtatcttatactaccatcattttataggcatagggctcttagctgcaaataatggaactaactctaataaa |
| | | gcagaacgcaaatattgtaaatattagagagctaacaatctctgggatggctaaaggatggagcttggaggct |
| | | acccagccagtaacaatattccgggctccactgttgaatggagacactacaactgccttggatgggcagaga |
| | | tattatggatgctaagccccaggtgctaccattaggacttctaccactgtccctaacgggtggagcccatcaca |
| | | tgcctatgccctcactgtaaggaaatgaagctactgttgtatatcttgggaagcacttggattaattgttatacagt |
| | | tttgttgaagaagacccctagggtaagtagccataactgcacactaaatttaaaattgttaatgagtttctcaaaa |
| | | aaaatgttaaggttgttagctggtatagtatatatcttgcctgttttccaaggacttctttgggcagtaccttgtctgt |
| | | gctggcaagcaactgagacttaatgaaagagtattggagatatgaatgaattgatgctgtatactctcagagtg |
| | | ccaaacatataccaatggacaagaaggtgaggcagagagcagacaggcattagtgacaagcaaagatatg |
| | | cagaatttcattctcagcaaatcaaaagtcctcaacctggttggaagaatattggcactgaatggtatcaataag |
| | | gttgctagagagggttagaggtgcacaatgtgcttccataacattttatacttctccaatcttagcactaatcaaa |
| | | catggttgaatactttgtttactataactcttacagagttataagatctgtgaagacagggacagggacaatacc |
| | | catctctgtctggttcataggtggtatgtaatagatatttttaaaaataagtgagttaatgaatgagggtgagaatg |
| | | aaggcacagaggtattagggggaggtgggccccagagaatggtgccaaggtccagtggggtgactggga |
| | | tcagctcaggcctgacgctggccactcccacctagctcctttctttctaatctgttctcattctccttgggaaggat |
| | | tgaggtctctggaaaacagccaaacaactgttatgggaacagcaagcccaaataaagccaagcatcaggg |
| | | ggatctgagagctgaaagcaacttctgttccccctccctcagctgaaggggggggaagggctcccaaagc |
| | | cataactccttttaagggatttagaaggcataaaaaggcccctggctgagaacttccttcttcattctgcagttgg |
| | | tgaattccccggggatcctctagagtcgaaattcgccaccatggtgagcaagggcgaggagctgttcaccg |
| | | gggtggtgcccatcctggtcgagctggacggcgacgtaaacggccacaagttcagcgtgtccggcgaggg |
| | | cgagggcgatgccacctacggcaagctgaccctgaagttcatctgcaccaccggcaagctgcccgtgccct |
| | | ggcccaccctcgtgaccaccctgacctacggcgtgcagtgcttcagccgctaccccgaccacatgaagcag |
| | | cacgacttcttcaagtccgccatgcccgaaggctacgtccaggagcgcaccatcttcttcaaggacgacggc |
| | | aactacaagacccgcgccgaggtgaagttcgagggcgacaccctggtgaaccgcatcgagctgaagggc |
| | | atcgacttcaaggaggacggcaacatcctggggcacaagctggagtacaactacaacagccacaacgtcta |
| | | tatcatggccgacaagcagaagaacggcatcaaggtgaacttcaagatccgccacaacatcgaggacggc |
| | | agcgtgcagctcgccgaccactaccagcagaacacccccatcggcgacggccccgtgctgctgcccgac |
| | | aaccactacctgagcacccagtccgccctgagcaaagaccccaacgagaagcgcgatcacatggtcctgc |
| | | tggagttcgtgaccgccgccgggatcactctcggcatggacgagctgtacaagtaatagggtaccggtcga |
| | | cctgcagaagcttgcctcgagcagcgctgctcgagagatctggatcataatcagccataccacatttgtagag |
| | | gttttacttgctttaaaaaacctcccacacctccccctgaacctgaaacataaaatgaatgcaattgttgttgttaa |
| | | cttgtttattgcagcttataatggttacaaataaagcaatagcatcacaaatttcacaaataaagcatttttttcact |
| | | gcattctagttgtggtttgtccaaactcatcaatgtatcttatcatgtctggtaaccattctccaggttgagccaga |
| | | ccaatttgatggtagatttagcaaataaaaatacaggacacccagttaaatgtgaatttccgatgaacagcaaa |
| | | tacttttttagtattaaaaaagttcacatttaggctcacgcctgtaatcccagcactttgggaggccgaggcagg |
| | | cagatcacctgaggtcaggagttcgagaccagcctggccaacatggtgaaaccccatctccactaaaaatac |
| | | caaaaattagccaggcgtgctggtgggcacctgtagttccagctactcaggaggctaaggcaggagaattg |
| | | cttgaacctgggaggcagaggttgcagtgagctgagatcgcaccattgcactctagcctgggcgacaagaa |
| | | caaaactccatctcaaaaaaaaaaaaaaaaaaaaagttcacatttaactgggcattctgtatttaattggtaatct |
| | | gagatggcagggaacagcatcagcatggtgtgagggataggcattttttcattgtgtacagcttgtaaatcagt |
| | | atttttaaaactcaaagttaatggcttgggcatatttagaaaagagttgccgcacggacttgaaccctgtattcct |
| | | aaaatctaggatcttgttctgatggtctgcacaactggctgggggtgtccagccactgtccctcttgcctgggct |
| | | ccccagggcagttctgtcagcctctccatttccattcctgttccagcaaaacccaactgatagcacagcagcat |
| | | ttcagcctgtctacctctgtgcccacatacctggatgtctaccagccagaaaggtggcttagatttggttcctgt |
| | | gggtggattatggcccccagaacttccctgtgcttgctgggggtgtggagtggaaagagcaggaaatgggg |
| | | gaccctccgatactctatgggggtcctccaagtctctttgtgcaagttagggtaataatcaatatggagctaaga |
| | | aagagaaggggaactatgctttagaacaggacactgtgccaggagcattgcagaaattatatggttttcacga |
| | | cagttctttttggtaggtactgttattatcctcagtttgcagatgaggaaactgagacccagaaaggttaaataac |
| | | ttgctagggtcacacaagtcataactgacaaagcctgattcaaacccaggtctccctaacctttaaggtttctat |
| | | gacgccagctctcctagggagtttgtcttcagatgtcttggctctaggtgtcaaaaaaagacttggtgtcaggc |
| | | aggcataggttcaagtcccaactctgtcacttaccaactgtgactaggtgattgaactgaccatggaacctggt |
| | | cacatgcaggagcaggatggtgaagggttcttgaaggcacttaggcaggacatttaggcaggagagaaaa |
| | | cctggaaacagaagagctgtctccaaaaatacccactggggaagcaggttgtcatgtgggccatgaatggg |
| | | acctgttctggggtaaccacgtgcggaccgagcggccgcaggaacccctagtgatggagttggccactccc |
| | | tctctgcgcgctcgctcgctcactgaggccgggcgaccaaaggtcgcccgacgcccgggctttgcccggg |
| | | cggcctcagtgagcgagcgagcgcgcagctgcctgcaggggcgcctgatgcggtattttctccttacgcatc |
| | | tgtgcggtatttcacaccgcatacgtcaaagcaaccatagtacgcgccctgtagcggcgcattaagcgcggc |
| | | gggtgtggtggttacgcgcagcgtgaccgctacacttgccagcgccttagcgcccgctcctttcgctttcttcc |
| | | cttcctttctcgccacgttcgccggctttccccgtcaagctctaaatcgggggctccctttagggttccgatttag |
| | | tgctttacggcacctcgaccccaaaaaacttgatttgggtgatggttcacgtagtgggccatcgccctgataga |
| | | cggtttttcgccctttgacgttggagtccacgttctttaatagtggactcttgttccaaactggaacaacactcaa |
| | | ctctatctcgggctattcttttgatttataagggattttgccgatttcggtctattggttaaaaaatgagctg atttaa |
| | | caaaaatttaacgcgaattttaacaaaatattaacgtttacaattttatggtgcactctcagtacaatctgctctgat |
| | | gccgcatagttaagccagccccgacacccgccaacacccgctgacgcgccctgacgggcttgtctgctccc |
| | | ggcatccgcttacagacaagctgtgaccgtctccgggagctgcatgtgtcagaggttttcaccgtcatcaccg |
| | | aaacgcgcgagacgaaagggcctcgtgatacgcctatttttataggttaatgtcatgataataatggtttcttag |
| | | acgtcaggtggcacttttcggggaaatgtgcgcggaacccctatttgtttatttttctaaatacattcaaatatgta |
| | | tccgctcatgagacaataaccctgataaatgcttcaataatattgaaaaaggaagagtatgagtattcaacattt |
| | | ccgtgtcgcccttattcccttttttgcggcattttgccttcctgtttttgctcacccagaaacgctggtgaaagtaaa |
| | | agatgctgaagatcagttgggtgcacgagtgggttacatcgaactggatctcaacagcggtaagatccttga |
| | | gagttttcgccccgaagaacgttttccaatgatgagcacttttaaagttctgctatgtggcgcggtattatcccgt |
| | | attgacgccgggcaagagcaactcggtcgccgcatacactattctcagaatgacttggttgagtactcaccag |
| | | tcacagaaaagcatcttacggatggcatgacagtaagagaattatgcagtgctgccataaccatgagtgataa |
| | | cactgcggccaacttacttctgacaacgatcggaggaccgaaggagctaaccgcttttttgcacaacatggg |
| | | ggatcatgtaactcgccttgatcgttgggaaccggagctgaatgaagccataccaaacgacgagcgtgaca |
| | | ccacgatgcctgtagcaatggcaacaacgttgcgcaaactattaactggcgaactacttactctagcttcccg |
| | | gcaacaattaatagactggatggaggcggataaagttgcaggaccacttctgcgctcggcccttccggctgg |
| | | ctggtttattgctgataaatctggagccggtgagcgtgggtctcgcggtatcattgcagcactggggccagat |
| | | ggtaagccctcccgtatcgtagttatctacacgacggggagtcaggcaactatggatgaacgaaatagacag |
| | | atcgctgagataggtgcctcactgattaagcattggtaactgtcagaccaagtttactcatatatactttagattg |
| | | atttaaaacttcatttttaatttaaaaggatctaggtgaagatcctttttgataatctcatgaccaaaatcccttaacg |
| | | tgagttttcgttccactgagcgtcagaccccgtagaaaagatcaaaggatcttcttgaaatcctttttttctgcgc |
| | | gtaatctgctgcttgcaaacaaaaaaaccaccgctaccagcggtggtttgtttgccggatcaagagctaccaa |
| | | ctctttttccgaaggtaactggcttcagcagagcgcagataccaaatactgttcttctagtgtagccgtagttag |
| | | gccaccacttcaagaactctgtagcaccgcctacatacctcgctctgctaatcctgttaccagtggctgctgcc |
| | | agtggcgataagtcgtgtcttaccgggttggactcaagacgatagttaccggataaggcgcagcggtcggg |
| | | ctgaacggggggttcgtgcacacagcccagcttggagcgaacgacctacaccgaactgagatacctacag |
| | | cgtgagctatgagaaagcgccacgcttcccgaagggagaaaggcggacaggtatccggtaagcggcagg |
| | | gtcggaacaggagagcgcacgagggagcttccagggggaaacgcctggtatctttatagtcctgtcgggttt |
| | | cgccacctctgacttgagcgtcgatttttgtgatgctcgtcaggggggggagcctatggaaaaacgccagc |
| | | aacgcggcctttttacggttcctggccttttgctggccttttgctcacatgtcctgcaggcag |
| 35 | Plasmid AG006 | ctgcgcgctcgctcgctcactgaggccgcccgggcgtcgggcgacctttggtcgcccggcctcagtgagc |
| | | gagcgagcgcgcagagagggagtggccaactccatcactaggggttcctgcggccgcacgcgttacgtaa |
| | | ttctgtcattttactagggtgatgaaattcccaagcaacaccatccttttcagataagggcactgaggctgagag |
| | | aggagctgaaacctacccggcgtcaccacacacaggtggcaaggctgggaccagaaaccaggactgttg |
| | | actgcagcccggtattcattctttccatagcccacagggctgtcaaagaccccagggcctagtcagaggctc |
| | | ctccttcctggagagttcctggcacagaagttgaagctcagcacagccccctaacccccaactctctctgcaa |
| | | ggcctcaggggtcagaacactggtggagcagatcctttagcctctggattttagggccatggtagagggggt |
| | | gttgccctaaattccagccctggtctcagcccaacaccctccaagaagaaattagaggggccatggccagg |
| | | ctgtgctagccgttgcttctgagcagattacaagaagggactaagacaaggactcctttgtggaggtcctggc |
| | | ttagggagtcaagtgacggcggctcagcactcacgtgggcagtgccagcctctaagagtgggcaggggca |
| | | ctggccacagagtcccagggagtcccaccagcctagtcgccagaccgaattccccggggatcctctagagt |
| | | cgaaattcgccaccatggtgagcaagggcgaggagctgttcaccggggtggtgcccatcctggtcgagctg |
| | | gacggcgacgtaaacggccacaagttcagcgtgtccggcgagggcgagggcgatgccacctacggcaa |
| | | gctgaccctgaagttcatctgcaccaccggcaagctgcccgtgccctggcccaccctcgtgaccaccctga |
| | | cctacggcgtgcagtgcttcagccgctaccccgaccacatgaagcagcacgacttcttcaagtccgccatgc |
| | | ccgaaggctacgtccaggagcgcaccatcttcttcaaggacgacggcaactacaagacccgcgccgaggt |
| | | gaagttcgagggcgacaccctggtgaaccgcatcgagctgaagggcatcgacttcaaggaggacggcaa |
| | | catcctggggcacaagctggagtacaactacaacagccacaacgtctatatcatggccgacaagcagaaga |
| | | acggcatcaaggtgaacttcaagatccgccacaacatcgaggacggcagcgtgcagctcgccgaccacta |
| | | ccagcagaacacccccatcggcgacggccccgtgctgctgcccgacaaccactacctgagcacccagtcc |
| | | gccctgagcaaagaccccaacgagaagcgcgatcacatggtcctgctggagttcgtgaccgccgccggga |
| | | tcactctcggcatggacgagctgtacaagtaatagggtaccggtcgacctgcagaagcttgcctcgagcagc |
| | | gctgctcgagagatctggatcataatcagccataccacatttgtagaggttttacttgctttaaaaaacctcccac |
| | | acctccccctgaacctgaaacataaaatgaatgcaattgttgttgttaacttgtttattgcagcttataatggttaca |
| | | aataaagcaatagcatcacaaatttcacaaataaagcatttttttcactgcattctagttgtggtttgtccaaactca |
| | | tcaatgtatcttatcatgtctggtaaccattctccaggttgagccagaccaatttgatggtagatttagcaaataaa |
| | | aatacaggacacccagttaaatgtgaatttccgatgaacagcaaatacttttttagtattaaaaaagttcacattta |
| | | ggctcacgcctgtaatcccagcactttgggaggccgaggcaggcagatcacctgaggtcaggagttcgag |
| | | accagcctggccaacatggtgaaaccccatctccactaaaaataccaaaaattagccaggcgtgctggtgg |
| | | gcacctgtagttccagctactcaggaggctaaggcaggagaattgcttgaacctgggaggcagaggttgca |
| | | gtgagctgagatcgcaccattgcactctagcctgggcgacaagaacaaaactccatctcaaaaaaaaaaaa |
| | | aaaaaaaaagttcacatttaactgggcattctgtatttaattggtaatctgagatggcagggaacagcatcagc |
| | | atggtgtgagggataggcattttttcattgtgtacagcttgtaaatcagtatttttaaaactcaaagttaatggcttg |
| | | ggcatatttagaaaagagttgccgcacggacttgaaccctgtattcctaaaatctaggatcttgttctgatggtct |
| | | gcacaactggctgggggtgtccagccactgtccctcttgcctgggctccccagggcagttctgtcagcctctc |
| | | catttccattcctgttccagcaaaacccaactgatagcacagcagcatttcagcctgtctacctctgtgcccaca |
| | | tacctggatgtctaccagccagaaaggtggcttagatttggttcctgtgggtggattatggcccccagaacttc |
| | | cctgtgcttgctgggggtgtggagtggaaagagcaggaaatgggggaccctccgatactctatgggggtcc |
| | | tccaagtctctttgtgcaagttagggtaataatcaatatggagctaagaaagagaaggggaactatgctttaga |
| | | acaggacactgtgccaggagcattgcagaaattatatggttttcacgacagttctttttggtaggtactgttattat |
| | | cctcagtttgcagatgaggaaactgagacccagaaaggttaaataacttgctagggtcacacaagtcataact |
| | | gacaaagcctgattcaaacccaggtctccctaacctttaaggtttctatgacgccagctctcctagggagtttgt |
| | | cttcagatgtcttggctctaggtgtcaaaaaaagacttggtgtcaggcaggcataggttcaagtcccaactctg |
| | | tcacttaccaactgtgactaggtgattgaactgaccatggaacctggtcacatgcaggagcaggatggtgaa |
| | | gggttcttgaaggcacttaggcaggacatttaggcaggagagaaaacctggaaacagaagagctgtctcca |
| | | aaaatacccactggggaagcaggttgtcatgtgggccatgaatgggacctgttctggggtaaccacgtgcg |
| | | gaccgagcggccgcaggaacccctagtgatggagttggccactccctctctgcgcgctcgctcgctcactg |
| | | aggccgggcgaccaaaggtcgcccgacgcccgggctttgcccgggcggcctcagtgagcgagcgagcg |
| | | cgcagctgcctgcaggggcgcctgatgcggtattttctccttacgcatctgtgcggtatttcacaccgcatacg |
| | | tcaaagcaaccatagtacgcgccctgtagcggcgcattaagcgcggcgggtgtggtggttacgcgcagcgt |
| | | gaccgctacacttgccagcgccttagcgcccgctcctttcgctttcttcccttcctttctcgccacgttcgccgg |
| | | ctttccccgtcaagctctaaatcgggggctccctttagggttccgatttagtgctttacggcacctcgaccccaa |
| | | aaaacttgatttgggtgatggttcacgtagtgggccatcgccctgatagacggtttttcgccctttgacgttgga |
| | | gtccacgttctttaatagtggactcttgttccaaactggaacaacactcaactctatctcgggctattcttttgattt |
| | | ataagggattttgccgatttcggtctattggttaaaaaatgagctgatttaacaaaaatttaacgcgaattttaaca |
| | | aaatattaacgtttacaattttatggtgcactctcagtacaatctgctctgatgccgcatagttaagccagccccg |
| | | acacccgccaacacccgctgacgcgccctgacgggcttgtctgctcccggcatccgcttacagacaagctg |
| | | tgaccgtctccgggagctgcatgtgtcagaggttttcaccgtcatcaccgaaacgcgcgagacgaaagggc |
| | | ctcgtgatacgcctatttttataggttaatgtcatgataataatggtttcttagacgtcaggtggcacttttcgggg |
| | | aaatgtgcgcggaacccctatttgtttatttttctaaatacattcaaatatgtatccgctcatgagacaataaccct |
| | | gataaatgcttcaataatattgaaaaaggaagagtatgagtattcaacatttccgtgtcgcccttattcccttttttg |
| | | cggcattttgccttcctgtttttgctcacccagaaacgctggtgaaagtaaaagatgctgaagatcagttgggtg |
| | | cacgagtgggttacatcgaactggatctcaacagcggtaagatccttgagagttttcgccccgaagaacgtttt |
| | | ccaatgatgagcacttttaaagttctgctatgtggcgcggtattatcccgtattgacgccgggcaagagcaact |
| | | cggtcgccgcatacactattctcagaatgacttggttgagtactcaccagtcacagaaaagcatcttacggatg |
| | | gcatgacagtaagagaattatgcagtgctgccataaccatgagtgataacactgcggccaacttacttctgac |
| | | aacgatcggaggaccgaaggagctaaccgcttttttgcacaacatgggggatcatgtaactcgccttgatcgt |
| | | tgggaaccggagctgaatgaagccataccaaacgacgagcgtgacaccacgatgcctgtagcaatggcaa |
| | | caacgttgcgcaaactattaactggcgaactacttactctagcttcccggcaacaattaatagactggatggag |
| | | gcggataaagttgcaggaccacttctgcgctcggcccttccggctggctggtttattgctgataaatctggagc |
| | | cggtgagcgtgggtctcgcggtatcattgcagcactggggccagatggtaagccctcccgtatcgtagttatc |
| | | tacacgacggggagtcaggcaactatggatgaacgaaatagacagatcgctgagataggtgcctcactgatt |
| | | aagcattggtaactgtcagaccaagtttactcatatatactttagattgatttaaaacttcatttttaatttaaaagga |
| | | tctaggtgaagatcctttttgataatctcatgaccaaaatcccttaacgtgagttttcgttccactgagcgtcagac |
| | | cccgtagaaaagatcaaaggatcttcttgaaatcctttttttctgcgcgtaatctgctgcttgcaaaaaaaaaac |
| | | caccgctaccagcggtggtttgtttgccggatcaagagctaccaactctttttccgaaggtaactggcttcagc |
| | | agagcgcagataccaaatactgttcttctagtgtagccgtagttaggccaccacttcaagaactctgtagcacc |
| | | gcctacatacctcgctctgctaatcctgttaccagtggctgctgccagtggcgataagtcgtgtcttaccgggtt |
| | | ggactcaagacgatagttaccggataaggcgcagcggtcgggctgaacggggggttcgtgcacacagcc |
| | | cagcttggagcgaacgacctacaccgaactgagatacctacagcgtgagctatgagaaagcgccacgcttc |
| | | ccgaagggagaaaggcggacaggtatccggtaagcggcagggtcggaacaggagagcgcacgaggga |
| | | gcttccagggggaaacgcctggtatctttatagtcctgtcgggtttcgccacctctgacttgagcgtcgatttttg |
| | | tgatgctcgtcaggggggcggagcctatggaaaaacgccagcaacgcggcctttttacggttcctggcctttt |
| | | gctggccttttgctcacatgtcctgcaggcag |
| 36 | sc5′ ITR | ctgcgcgctcgctcgctcactgaggccgcccgggcaaagcccgggcgtcgggcgacctttggtcgcccg |
| | | gcctcagtgagcgagcgagcgcgcagagagggagtggccaactccatcactaggggttcct |
| 37 | Macaca mulatta | atgtcagaaggggtgggcacgttccgcatggtacctgaagaggaacaggagctccgtgcccaactggagc |
| | (Rhesus | agctcacaaccaaggaccatggacctgtctttggcccgtgcagccagctgccccgccacaccttgcagaag |
| | Monkey) RLBP1 | gccaaagatgagctgaatgagagagaggagacccgggaggaggcagtgcgagagctgcaggagatggt |
| | CDS | gcaggcgcaggcggcctcgggggaggagctggccgtggccgtggcggagagggtgcaagagaagga |
| | (XM 001091538 | cagcggcttcttcctgcgcttcatccgcgcgcgaaagttcaacgtgggccgtgcctatgagctgctcagagg |
| | A | ctatgtgaatttccggctgcagtaccctgagctctttgacagcctgtccccagaggctgtccgctgtaccattga |
| | | agctggctaccctggtgtcctctctagtcgggacaagtatggccgagtggtcatgctcttcaacattgagaact |
| | | ggcaaagtcaagaaatcaccttcgatgagatcttgcaggcatattgcttcatcctggagaagctgctggagaa |
| | | tgaggaaactcaaattaatggattctgcatcattgagaacttcaagggctttaccatgcagcaggctgctagtct |
| | | ccgcacttcagatctcaggaagatggtggacatgctccaggattccttcccagcccggttcaaagccatccac |
| | | ttcatccaccagccatggtacttcaccacgacctacaatgtggtcaagcccttcttgaagagcaagctgcttga |
| | | gagggtctttgtccacggggaggacctctctggtttctaccaggagattgatgagaacatcctgccctctgact |
| | | ttgggggcacgctgcccaagtatgatggcaaagctgttgctgagcagctctttggcccccgggcccaagctg |
| | | agaacacagccttctga |
| 38 | Macaca mulatta | MSEGVGTFRMVPEEEQELRAQLEQLTTKDHGPVFGPCSQLPRHTLQ |
| | (Rhesus | KAKDELNEREETREEAVRELQEMVQAQAASGEELAVAVAERVQEK |
| | Monkey) RLBP1 | DSGFFLRFIRARKFNVGRAYELLRGYVNFRLQYPELFDSLSPEAVRC |
| | gene product | TIEAGYPGVLSSRDKYGRVVMLFNIENWQSQEITFDEILQAYCFILEK |
| | (CRALBP) | LLENEETQINGFCIIENFKGFTMQQAASLRTSDLRKMVDMLQDSFPA |
| | | RFKAIHFIHQPWYFTTTYNVVKPFLKSKLLERVFVHGEDLSGFYQEID |
| | | ENILPSDFGGTLPKYDGKAVAEQLFGPRAQAENTAF |
| 39 | Bos taurus | atgtcagaggggggggcacgttccgcatggtccctgaagaggaacaggagctccgtgcccaactggag |
| | RLBP1 CDS | aggcttacgaccaaagaccatggacctgtctttggcccgtgcagccagctgccccgccacaccttgcagaa |
| | (NM_174451) | ggccaaggacgagctgaatgaaaaggaagagacccgggaagaggcagtgcgggagctacaggagctg |
| | | gtgcaggcggaggccgcctcggggcaggagctggccgtggccgtggcggagagggtgcagggaaaag |
| | | acagtgccttcttcctgcgcttcatccgcgcgcgcaagttccacgtggggcgcgcctacgagctgctcagag |
| | | gctacgtgaacttccggctgcagtacccagagctcttcgacagcctgtccccagaggctgtccgctgcaccg |
| | | ttgaggctggctaccctggtgtcctctccacgcgggacaagtatggccgagtggtcatgctcttcaatattgag |
| | | aactgggactctgaagaaatcacctttgatgagatcttgcaggcatactgcgtcatcctggagaagctactgg |
| | | agaatgaggagactcaaattaatggcttttgcatcattgagaacttcaagggcttcaccatgcagcaggctgc |
| | | cggacttcggccttccgatctcagaaagatggtggacatgctccaggattccttcccagctcggttcaaagcc |
| | | atccacttcatctaccagccctggtacttcaccaccacctacaacgtggtcaagcccttcttgaagagcaaatt |
| | | gctccagagggtatttgtccatggagaagacctctccagcttctaccaggagtttgacgaggacatcctgccc |
| | | tccgactttgggggtacactgcccaagtatgatggcaaggccgttgctgagcagctctttggtcctcgggacc |
| | | aaactgagaacacagccttctga |
| 40 | Bos taurus | MSEGAGTFRMVPEEEQELRAQLERLTTKDHGPVFGPCSQLPRHTLQ |
| | RLBP1 gene | KAKDELNEKEETREEAVRELQELVQAEAASGQELAVAVAERVQGK |
| | product | DSAFFLRFIRARKFHVGRAYELLRGYVNFRLQYPELFDSLSPEAVRC |
| | (CRALBP) | TVEAGYPGVLSTRDKYGRVVMLFNIENWDSEEITFDEILQAYCVILE |
| | | KLLENEETQINGFCIIENFKGFTMQQAAGLRPSDLRKMVDMLQDSFP |
| | | ARFKAIHFIYQPWYFTTTYNVVKPFLKSKLLQRVFVHGEDLSSFYQE |
| | | FDEDILPSDFGGTLPKYDGKAVAEQLFGPRDQTENTAF |
| 41 | Canis lupus | atgtcagaaggcgtgggcacattccgtgtggtccctgaagaggaacaggagctccgtgcccagctggagc |
| | familiaris | ggcttacaaccaaggaccatgggcctgtctttggcccttgcagccagctccctcgtcataccttacagaaggc |
| | RLBP1 CDS | caaggacgagctgaacgagagggaggagacccgggaggaggtggtgcgagagctgcaggagctggtg |
| | (XM_549634) | caggcacaggctgccaccgggcaggagctggccagggcggtggctgagagggtgcagggaagggaca |
| | | gtgccttcttcctgcgcttcatccgcgcgcggaagttccatgtggggcgtgcctacgagctgcttcgaggcta |
| | | cgtgaacttccggctgcagtacccagagctcttcgacagcctgtccctggaggctgtccgttgcaccgtcga |
| | | ggccggctatcctggggtcctccccagtcgggacaagtatggccgagtggtcatgctcttcaacatcgagaa |
| | | ctgggactccgaagaaatcaccttcgatgagatcttgcaggcatattgtttcatcctggagaagctactagaga |
| | | atgaggaaactcaaattaatggcttctgcattattgagaactttaagggctttaccatgcagcaggctgctggac |
| | | ttcgggcttccgatctcaggaagatggtggacatgctccaggattccttcccagcgcggttcaaagccatcca |
| | | cttcattcaccaaccatggtacttcaccaccacctacaacatggtcaagcccctcctgaagaacaagctgctc |
| | | caaagagtctttgtccatggagatgacctctctggcttcttccaggagattgatgaagacatactgcccgctga |
| | | ctttgggggcacactgcccaagtatgatggcaaggtggttgctgagcagctctttggcccccgggcccaagc |
| | | tgagaacacagccttctga |
| 42 | Canis lupus | MSEGVGTFRVVPEEEQELRAQLERLTTKDHGPVFGPCSQLPRHTLQK |
| | familiaris | AKDELNEREETREEVVRELQELVQAQAATGQELARAVAERVQGRD |
| | RLBPI gene | SAFFLRFIRARKFHVGRAYELLRGYVNFRLQYPELFDSLSLEAVRCT |
| | product | VEAGYPGVLPSRDKYGRVVMLFNIENWDSEEITFDEILQAYCFILEKL |
| | (CRALBP) | LENEETQINGFCIIENFKGFTMQQAAGLRASDLRKMVDMLQDSFPAR |
| | | FKAIHFIHQPWYFTTTYNMVKPLLKNKLLQRVFVHGDDLSGFFQEID |
| | | EDILPADFGGTLPKYDGKVVAEQLFGPRAQAENTAF |
| 43 | Rattus | atgtcagaggggggggcacattccgaatggtccctgaagaggagcaggagctccgggcacagctagaa |
| | norvegicus | cagctcacaaccaaggatcatggtcctgtctttggcccatgcagccagctgccccgccacactttgcagaag |
| | RLBP1 CDS | gctaaggatgagctgaatgaaagggaggaaacccgggatgaggcggtgagggagctacaggagctggtc |
| | (NM_001106274.1) | caggcacaggcagcttctggggaagagttggccgtggcagtggctgagagggtgcaggcaagagacagc |
| | | gccttcctcctgcgcttcatccgtgcccgaaagtttgatgtgggccgggcttatgagctgctcaaaggctatgt |
| | | gaacttccggctccagtaccctgaactcttcgatagcctatctatggaggctctccgctgcactatcgaggccg |
| | | gttaccctggtgtcctttccagtcgggacaagtatggtcgagtggttatgctcttcaacattgaaaactggcact |
| | | gtgaagaagtcacctttgatgagatcttacaggcatattgtttcattctggagaaactgctggagaacgaggaa |
| | | acccaaatcaacggcttctgtattgtggagaacttcaagggcttcaccatgcagcaggccgcgggactccgc |
| | | ccctccgatctcaagaagatggtggacatgctccaggattcattcccagccaggttcaaagctatccacttcat |
| | | ccaccaaccatggtacttcaccaccacttacaatgtggtcaagcccttcttgaagaacaagttgctacagagg |
| | | gtcttcgttcatggagatgacctggacggcttcttccaggagattgatgagaatatcttgcctgctgactttggg |
| | | ggtacactgcccaagtatgacggcaaagttgtcgctgagcagctcttcggtccccgggttgaggttgagaac |
| | | acagccttgtga |
| 44 | Rattus | MSEGVGTFRMVPEEEQELRAQLEQLTTKDHGPVFGPCSQLPRHTLQ |
| | norvegicus | KAKDELNEREETRDEAVRELQELVQAQAASGEELAVAVAERVQAR |
| | RLBP1 gene | DSAFLLRFIRARKFDVGRAYELLKGYVNFRLQYPELFDSLSMEALRC |
| | product | TIEAGYPGVLSSRDKYGRVVMLFNIENWHCEEVTFDEILQAYCFILE |
| | (CRALBP) | KLLENEETQINGFCIVENFKGFTMQQAAGLRPSDLKKMVDMLQDSF |
| | | PARFKAIHFIHQPWYFTTTYNVVKPFLKNKLLQRVFVHGDDLDGFFQ |
| | | EIDENILPADFGGTLPKYDGKVVAEQLFGPRVEVENTAL |
| 45 | Mus musculus | atgtcagacggggtgggcactttccgcatggttcctgaagaggagcaggagctccgagcacaactggagc |
| | RLBP1 CDS | agctcacaaccaaggatcatggtcctgtctttggcccatgcagccagctgccccgccacactttgcagaagg |
| | (NM_020599.2) | ccaaggatgagctgaatgaaaaggaggagacccgggaggaagcggtgagggagctacaggagctggta |
| | | caggcacaggcagcttctggcgaggaattggccctggcagtggctgagagggtgcaggcaagagacagc |
| | | gccttcctcctgcgcttcatccgtgcccgcaagttcgatgtgggtcgtgcttatgagctgctcaaaggctatgtg |
| | | aacttccgcctccagtaccctgaactcttcgatagtctctccatggaggctctccgctgcactatcgaggccgg |
| | | ataccctggtgtcctttccagtcgggacaagtatggtcgagtggttatgctcttcaacatcgaaaactggcact |
| | | gtgaagaagtgacctttgatgagatcttacaggcatattgtttcattttggagaaactgctggaaaatgaggaaa |
| | | cccaaatcaacggcttctgtattgttgagaacttcaagggcttcaccatgcagcaggcagcagggctccgcc |
| | | cctcggatctcaagaagatggtggacatgctccaggattcattcccagccaggttcaaagctatccacttcatc |
| | | caccagccatggtacttcaccaccacctataatgtggtcaagcccttcttgaagaacaagctgctacagaggg |
| | | tctttgttcacggagatgacctggatggcttcttccaggagattgatgagaacatcctgcctgctgactttgggg |
| | | gtacactgcccaagtacgacggcaaagttgttgctgagcagctctttggtccccgggctgaagttgagaaca |
| | | cagccttatga |
| 46 | Mus musculus | MSDGVGTFRMVPEEEQELRAQLEQLTTKDHGPVFGPCSQLPRHTLQ |
| | RLBP1 gene | KAKDELNEKEETREEAVRELQELVQAQAASGEELALAVAERVQAR |
| | product | DSAFLLRFIRARKFDVGRAYELLKGYVNFRLQYPELFDSLSMEALRC |
| | (CRALBP) | TIEAGYPGVLSSRDKYGRVVMLFNIENWHCEEVTFDEILQAYCFILE |
| | | KLLENEETQINGFCIVENFKGFTMQQAAGLRPSDLKKMVDMLQDSF |
| | | PARFKAIHFIHQPWYFTTTYNVVKPFLKNKLLQRVFVHGDDLDGFFQ |
| | | EIDENILPADFGGTLPKYDGKVVAEQLFGPRAEVENTAL |
| 47 | Gallus gallus | atgtctgctgttacgggcaccttccgcattgtctcggaagaggagcaggcgctgcgcaccaaactggagcg |
| | RLBP1 CDS | cctcaccaccaaggaccacggccctgtttttgggaggtgccagcagatcccccctcacaccctgcagaagg |
| | (NM_001024694 | caaaagatgagctgaatgagacggaggagcagagggaggcagcggtcaaagcgctgcgggagctggtg |
| | 1) | caggagcgggccggcagcgaggatgtctgcaaggcagtggcagagaagatgcaggggaaggacgattc |
| | | cttcttcctccgcttcatccgtgcccgcaagtttgacgtgcacagggcctacgacctgctgaaaggctatgtga |
| | | actttcgccagcaataccctgaactctttgacaacctgacccccgaggccgtgcgcagcaccatcgaggcg |
| | | ggctaccccggcatcctggccagcagggacaaatacggggggtagtgatgctcttcaacatcgagaactg |
| | | ggactacgaggagatcacctttgatgagatccttcgtgcctactgcgttatcttggagaagctgctggaaaac |
| | | gaagagacccagatcaatgggttctgcatcattgagaacttcaagggcttcaccatgcagcaggcatcaggg |
| | | atcaaaccctccgagctcaagaagatggtggacatgctacaggactccttcccagcgcggttcaaagctgtc |
| | | cacttcatccaccagccctggtacttcaccactacctacaacgtggtcaaaccgttcctgaagagcaagctgc |
| | | tggagagggtgtttgtgcacggcgaggagctggagtccttctaccaggagatcgatgctgacatactgccag |
| | | cagacttcggtggcaacctgcccaagtacgacggcaaagcaactgcagagcagctctttgggccccgcatt |
| | | gaggctgaagacacggcactttaa |
| 48 | Gallus gallus | MSAVTGTFRIVSEEEQALRTKLERLTTKDHGPVFGRCQQIPPHTLQK |
| | RLBP1 gene | AKDELNETEEQREAAVKALRELVQERAGSEDVCKAVAEKMQGKDD |
| | product | SFFLRFIRARKFDVHRAYDLLKGYVNFRQQYPELFDNLTPEAVRSTIE |
| | (CRALBP) | AGYPGILASRDKYGRVVMLFNIENWDYEEITFDEILRAYCVILEKLLE |
| | (NP_001019865.1) | NEETQINGFCIIENFKGFTMQQASGIKPSELKKMVDMLQDSFPARFK |
| | | AVHFIHQPWYFTTTYNVVKPFLKSKLLERVFVHGEELESFYQEIDADI |
| | | LPADFGGNLPKYDGKATAEQLFGPRIEAEDTAL |
| 49 | Kan-R bacterial | ctgcctgcagggttccatcccaatggcgcgtcaattcactggccgtcgttttacaacgtcgtgactgggaaaa |
| | backbone | ccctggcgttacccaacttaatcgccttgcagcacatccccctttcgccagctggcgtaatagcgaagaggcc |
| | | cgcaccgatcgcccttcccaacagttgcgcagcctgaatggcgaatggcgcctgatgcggtattttctcctta |
| | | cgcatctgtgcggtatttcacaccgcatatggtgcactctcagtacaatctgctctgatgccgcatagttaagcc |
| | | agccccgacacccgccaacacccgctgacgcgccctgacgggcttgtctgctcccggcatccgcttacaga |
| | | caagctgtgaccgtctccgggagctgcatgtgtcagaggttttcaccgtcatcaccgaaacgcgcgagacga |
| | | aagggcctcgtgatacgcctatttttataggttaatgtcatgataataatggtttcttagacgtcaggtggcactttt |
| | | cggggaaatgtgcgcggaacccctatttgtttatttttctaaatacattcaaatatgtatccgctcatgagacaata |
| | | accctgataaatgcttcaataatattgaaaaaggaagagtatgagccatattcaacgggaaacgtcttgctcta |
| | | ggccgcgattaaattccaacatggatgctgatttatatgggtataaatgggctcgcgataatgtcgggcaatca |
| | | ggtgcgacaatctatcgattgtatgggaagcccgatgcgccagagttgtttctgaaacatggcaaaggtagc |
| | | gttgccaatgatgttacagatgagatggtcagactaaactggctgacggaatttatgcctcttccgaccatcaa |
| | | gcattttatccgtactcctgatgatgcatggttactcaccactgcgatccctgggaaaacagcattccaggtatt |
| | | agaagaatatcctgattcaggtgaaaatattgttgatgcgctggcagtgttcctgcgccggttgcattcgattcc |
| | | tgtttgtaattgtccttttaacagcgatcgcgtatttcgtctcgctcaggcgcaatcacgaatgaataacggtttg |
| | | gttgatgcgagtgattttgatgacgagcgtaatggctggcctgttgaacaagtctggaaagaaatgcataaact |
| | | tttgccattctcaccggattcagtcgtcactcatggtgatttctcacttgataaccttatttttgacgaggggaaatt |
| | | aataggttgtattgatgttggacgagtcggaatcgcagaccgataccaggatcttgccatcctatggaactgcc |
| | | tcggtgagttttctccttcattacagaaacggctttttcaaaaatatggtattgataatcctgatatgaataaattgc |
| | | agtttcatttgatgctcgatgagtttttctaactgtcagaccaagtttactcatatatactttagattgatttaaaactt |
| | | catttttaatttaaaaggatctaggtgaagatcctttttgataatctcatgaccaaaatcccttaacgtgagttttcgt |
| | | tccactgagcgtcagaccccgtagaaaagatcaaaggatcttcttgagatcctttttttctgcgcgtaatctgctg |
| | | cttgcaaacaaaaaaaccaccgctaccagcggtggtttgtttgccggatcaagagctaccaactctttttccga |
| | | aggtaactggcttcagcagagcgcagataccaaatactgttcttctagtgtagccgtagttaggccaccacttc |
| | | aagaactctgtagcaccgcctacatacctcgctctgctaatcctgttaccagtggctgctgccagtggcgataa |
| | | gtcgtgtcttaccgggttggactcaagacgatagttaccggataaggcgcagcggtcgggctgaacggggg |
| | | gttcgtgcacacagcccagcttggagcgaacgacctacaccgaactgagatacctacagcgtgagctatga |
| | | gaaagcgccacgcttcccgaagggagaaaggcggacaggtatccggtaagcggcagggtcggaacagg |
| | | agagcgcacgagggagcttccagggggaaacgcctggtatctttatagtcctgtcgggtttcgccacctctg |
| | | acttgagcgtcgatttttgtgatgctcgtcaggggggcggagcctatggaaaaacgccagcaacgcggcctt |
| | | tttacggttcctggccttttgctggccttttgctcacatgttctttcctgcgttatcccctgattctgtggataaccgt |
| | | attaccgcctttgagtgagctgataccgctcgccgcagccgaacgaccgagcgcagcgagtcagtgagcg |
| | | aggaagcggaagagcgcccaatacgcaaaccgcctctccccgcgcgttggccgattcattaatgcagctgg |
| | | cacgacaggtttcccgactggaaagcgggcagtgagcgcaacgcaattaatgtgagttagctcactcattag |
| | | gcaccccaggctttacactttatgcttccggctcgtatgttgtgtggaattgtgagcggataacaatttcacaca |
| | | ggaaacagctatgaccatgattacgccaagctcggcgcgccattgggatggaaccctgcaggcag |
| 50 | Plasmid TM042 | ctgcgcgctcgctcgctcactgaggccgcccgggcaaagcccgggcgtcgggcgacctttggtcgcccg |
| | | gcctcagtgagcgagcgagcgcgcagagagggagtggggtaccacgcgtttgtcctctccctgcttggcct |
| | | taaccagccacatttctcaactgaccccactcactgcagaggtgaaaactaccatgccaggtcctgctggctg |
| | | ggggaggggtgggcaataggcctggatttgccagagctgccactgtagatgtagtcatatttacgatttccctt |
| | | cacctcttattaccctggtggtggtggtgggggggggggggtgctctctcagcaaccccaccccgggatctt |
| | | gaggagaaagagggcagagaaaagagggaatgggactggcccagatcccagccccacagccgggcttc |
| | | cacatggccgagcaggaactccagagcaggagcacacaaaggagggctttgatgcgcctccagccaggc |
| | | ccaggcctctcccctctcccctttctctctgggtcttcctttgccccactgagggcctcctgtgagcccgatttaa |
| | | cggaaactgtgggcggtgagaagttccttatgacacactaatcccaacctgctgaccggaccacgcctccag |
| | | cggagggaacctctagagctccaggacattcaggtaccaggtagccccaaggaggagctgccgaatcgat |
| | | ggatcgggaactgaaaaaccagaaagttaactggtaagtttagtctttttgtcttttatttcaggtcccggatccg |
| | | gtggtggtgcaaatcaaagaactgctcctcagtggatgttgcctttacttctaggcctgtacggaagtgttactt |
| | | ctgctctaaaagctgcggaattgtacccgccccgggatccatcgattgaattcgccaccatgtcagaaggggt |
| | | gggcacgttccgcatggtacctgaagaggaacaggagctccgtgcccaactggagcagctcacaaccaag |
| | | gaccatggacctgtctttggcccgtgcagccagctgccccgccacaccttgcagaaggccaaggatgagct |
| | | gaacgagagagaggagacccgggaggaggcagtgcgagagctgcaggagatggtgcaggcgcaggc |
| | | ggcctcgggggaggagctggcggtggccgtggcggagagggtgcaagagaaggacagcggcttcttcct |
| | | gcgcttcatccgcgcacggaagttcaacgtgggccgtgcctatgagctgctcagaggctatgtgaatttccg |
| | | gctgcagtaccctgagctctttgacagcctgtccccagaggctgtccgctgcaccattgaagctggctaccct |
| | | ggtgtcctctctagtcgggacaagtatggccgagtggtcatgctcttcaacattgagaactggcaaagtcaag |
| | | aaatcacctttgatgagatcttgcaggcatattgcttcatcctggagaagctgctggagaatgaggaaactcaa |
| | | atcaatggcttctgcatcattgagaacttcaagggctttaccatgcagcaggctgctagtctccggacttcagat |
| | | ctcaggaagatggtggacatgctccaggattccttcccagcccggttcaaagccatccacttcatccaccagc |
| | | catggtacttcaccacgacctacaatgtggtcaagcccttcttgaagagcaagctgcttgagagggtctttgtc |
| | | cacggggatgacctttctggtttctaccaggagatcgatgagaacatcctgccctctgacttcgggggcacgc |
| | | tgcccaagtatgatggcaaggccgttgctgagcagctctttggcccccaggcccaagctgagaacacagcc |
| | | ttctgaggatcgtaccggtcgacctgcagaagcttgcctcgagcagcgctgctcgagagatctggatcataat |
| | | cagccataccacatttgtagaggttttacttgctttaaaaaacctcccacacctccccctgaacctgaaacataa |
| | | aatgaatgcaattgttgttgttaacttgtttattgcagcttataatggttacaaataaagcaatagcatcacaaattt |
| | | cacaaataaagcatttttttcactgcattctagttgtggtttgtccaaactcatcaatgtatcttatcatgtctggtaa |
| | | ccacgtgcggaccgagcggccgcaggaacccctagtgatggagttggccactccctctctgcgcgctcgct |
| | | cgctcactgaggccgggcgaccaaaggtcgcccgacgcccgggctttgcccgggcggcctcagtgagcg |
| | | agcgagcgcgcagctgcctgcagggttccatcccaatggcgcgtcaattcactggccgtcgttttacaacgt |
| | | cgtgactgggaaaaccctggcgttacccaacttaatcgccttgcagcacatccccctttcgccagctggcgta |
| | | atagcgaagaggcccgcaccgatcgcccttcccaacagttgcgcagcctgaatggcgaatggcgcctgat |
| | | gcggtattttctccttacgcatctgtgcggtatttcacaccgcatatggtgcactctcagtacaatctgctctgatg |
| | | ccgcatagttaagccagccccgacacccgccaacacccgctgacgcgccctgacgggcttgtctgctcccg |
| | | gcatccgcttacagacaagctgtgaccgtctccgggagctgcatgtgtcagaggttttcaccgtcatcaccga |
| | | aacgcgcgagacgaaagggcctcgtgatacgcctatttttataggttaatgtcatgataataatggtttcttaga |
| | | cgtcaggtggcacttttcggggaaatgtgcgcggaacccctatttgtttatttttctaaatacattcaaatatgtat |
| | | ccgctcatgagacaataaccctgataaatgcttcaataatattgaaaaaggaagagtatgagccatattcaacg |
| | | ggaaacgtcttgctctaggccgcgattaaattccaacatggatgctgatttatatgggtataaatgggctcgcg |
| | | ataatgtcgggcaatcaggtgcgacaatctatcgattgtatgggaagcccgatgcgccagagttgtttctgaa |
| | | acatggcaaaggtagcgttgccaatgatgttacagatgagatggtcagactaaactggctgacggaatttatg |
| | | cctcttccgaccatcaagcattttatccgtactcctgatgatgcatggttactcaccactgcgatccctgggaaa |
| | | acagcattccaggtattagaagaatatcctgattcaggtgaaaatattgttg atgcgctggcagtgttcctgcgc |
| | | cggttgcattcgattcctgtttgtaattgtccttttaacagcgatcgcgtatttcgtctcgctcaggcgcaatcacg |
| | | aatgaataacggtttggttgatgcgagtgattttgatgacgagcgtaatggctggcctgttgaacaagtctgga |
| | | aagaaatgcataaacttttgccattctcaccggattcagtcgtcactcatggtgatttctcacttgataaccttattt |
| | | ttgacgaggggaaattaataggttgtattgatgttggacgagtcggaatcgcagaccgataccaggatcttgc |
| | | catcctatggaactgcctcggtgagttttctccttcattacagaaacggctttttcaaaaatatggtattgataatc |
| | | ctgatatgaataaattgcagtttcatttgatgctcgatgagtttttctaactgtcagaccaagtttactcatatatact |
| | | ttag attgatttaaaacttcatttttaatttaaaaggatctaggtgaagatcctttttgataatctcatgaccaaaatc |
| | | ccttaacgtgagttttcgttccactgagcgtcagaccccgtagaaaagatcaaaggatcttcttgagatccttttt |
| | | ttctgcgcgtaatctgctgcttgcaaacaaaaaaaccaccgctaccagcggtggtttgtttgccggatcaaga |
| | | gctaccaactctttttccgaaggtaactggcttcagcagagcgcagataccaaatactgttcttctagtgtagcc |
| | | gtagttaggccaccacttcaagaactctgtagcaccgcctacatacctcgctctgctaatcctgttaccagtgg |
| | | ctgctgccagtggcgataagtcgtgtcttaccgggttggactcaagacgatagttaccggataaggcgcagc |
| | | ggtcgggctgaacggggggttcgtgcacacagcccagcttggagcgaacgacctacaccgaactgagata |
| | | cctacagcgtgagctatgagaaagcgccacgcttcccgaagggagaaaggcggacaggtatccggtaag |
| | | cggcagggtcggaacaggagagcgcacgagggagcttccagggggaaacgcctggtatctttatagtcct |
| | | gtcgggtttcgccacctctgacttgagcgtcgatttttgtgatgctcgtcaggggggcggagcctatggaaaa |
| | | acgccagcaacgcggcctttttacggttcctggccttttgctggccttttgctcacatgttctttcctgcgttatcc |
| | | cctgattctgtggataaccgtattaccgcctttgagtgagctgataccgctcgccgcagccgaacgaccgagc |
| | | gcagcgagtcagtgagcgaggaagcggaagagcgcccaatacgcaaaccgcctctccccgcgcgttggc |
| | | cgattcattaatgcagctggcacgacaggtttcccgactggaaagcgggcagtgagcgcaacgcaattaatg |
| | | tgagttagctcactcattaggcaccccaggctttacactttatgcttccggctcgtatgttgtgtggaattgtgag |
| | | cggataacaatttcacacaggaaacagctatgaccatgattacgccaagctcggcgcgccattgggatggaa |
| | | ccctgcaggcag |
| 51 | Gene cassette of | cgcgctcgctcgctcactgaggccgcccgggcaaagcccgggcgtcgggcgacctttggtcgcccggcct |
| | plasmid TM017 | cagtgagcgagcgagcgcgcagagagggagtggggtaccacgcgtttgtcctctccctgcttggccttaac |
| | (occurs at bp 4 to | cagccacatttctcaactgaccccactcactgcagaggtgaaaactaccatgccaggtcctgctggctgggg |
| | 2330 of SEQ ID | gaggggtgggcaataggcctggatttgccagagctgccactgtagatgtagtcatatttacgatttcccttcac |
| | NO: 26) | ctcttattaccctggtggtggtggtgggggggggggggtgctctctcagcaaccccaccccgggatcttgag |
| | | gagaaagagggcagagaaaagagggaatgggactggcccagatcccagccccacagccgggcttccac |
| | | atggccgagcaggaactccagagcaggagcacacaaaggagggctttgatgcgcctccagccaggccca |
| | | ggcctctcccctctcccctttctctctgggtcttcctttgccccactgagggcctcctgtgagcccgatttaacgg |
| | | aaactgtgggcggtgagaagttccttatgacacactaatcccaacctgctgaccggaccacgcctccagcgg |
| | | agggaacctctagagctccaggacattcaggtaccaggtagccccaaggaggagctgccgaatcgatgga |
| | | tcgggaactgaaaaaccagaaagttaactggtaagtttagtctttttgtcttttatttcaggtcccggatccggtg |
| | | gtggtgcaaatcaaagaactgctcctcagtggatgttgcctttacttctaggcctgtacggaagtgttacttctgc |
| | | tctaaaagctgcggaattgtacccgccccgggatccatcgattgaattcgccaccatgtcagaaggggtggg |
| | | cacgttccgcatggtacctgaagaggaacaggagctccgtgcccaactggagcagctcacaaccaaggac |
| | | catggacctgtctttggcccgtgcagccagctgccccgccacaccttgcagaaggccaaggatgagctgaa |
| | | cgagagagaggagacccgggaggaggcagtgcgagagctgcaggagatggtgcaggcgcaggcggc |
| | | ctcgggggaggagctggcggtggccgtggcggagagggtgcaagagaaggacagcggcttcttcctgcg |
| | | cttcatccgcgcacggaagttcaacgtgggccgtgcctatgagctgctcagaggctatgtgaatttccggctg |
| | | cagtaccctgagctctttgacagcctgtccccagaggctgtccgctgcaccattgaagctggctaccctggtg |
| | | tcctctctagtcgggacaagtatggccgagtggtcatgctcttcaacattgagaactggcaaagtcaagaaat |
| | | cacctttgatgagatcttgcaggcatattgcttcatcctggagaagctgctggagaatgaggaaactcaaatca |
| | | atggcttctgcatcattgagaacttcaagggctttaccatgcagcaggctgctagtctccggacttcagatctca |
| | | ggaagatggtggacatgctccaggattccttcccagcccggttcaaagccatccacttcatccaccagccatg |
| | | gtacttcaccacgacctacaatgtggtcaagcccttcttgaagagcaagctgcttgagagggtctttgtccacg |
| | | gggatgacctttctggtttctaccaggagatcgatgagaacatcctgccctctgacttcgggggcacgctgcc |
| | | caagtatgatggcaaggccgttgctgagcagctctttggcccccaggcccaagctgagaacacagccttctg |
| | | aggatcgtaccggtcgacctgcagaagcttgcctcgagcagcgctgctcgagagatctggatcataatcagc |
| | | cataccacatttgtagaggttttacttgctttaaaaaacctcccacacctccccctgaacctgaaacataaaatg |
| | | aatgcaattgttgttgttaacttgtttattgcagcttataatggttacaaataaagcaatagcatcacaaatttcaca |
| | | aataaagcatttttttcactgcattctagttgtggtttgtccaaactcatcaatgtatcttatcatgtctggtaaccac |
| | | gtgcggaccgagcggccgcaggaacccctagtgatggagttggccactccctctctgcgcgctcgctcgct |
| | | cactgaggccgggcgaccaaaggtcgcccgacgcccgggctttgcccgggcggcctcagtgagcgagc |
| | | gagcgcgcag |
| 52 | Gene cassette of | ctgcgcgctcgctcgctcactgaggccgcccgggcgtcgggcgacctttggtcgcccggcctcagtgagc |
| | plasmid TM037 | gagcgagcgcgcagagagggagtggccaactccatcactaggggttcctgcggccgcacgcagcttttgt |
| | (occurs at bp 1 to | cctctccctgcttggccttaaccagccacatttctcaactgaccccactcactgcagaggtgaaaactaccatg |
| | 4711 of SEQ ID | ccaggtcctgctggctgggggaggggtgggcaataggcctggatttgccagagctgccactgtagatgtag |
| | NO: 27) | tcatatttacgatttcccttcacctcttattaccctggtggtggtggtgggggggggggggtgctctctcagcaa |
| | | ccccaccccgggatcttgaggagaaagagggcagagaaaagagggaatgggactggcccagatcccag |
| | | ccccacagccgggcttccacatggccgagcaggaactccagagcaggagcacacaaaggagggctttga |
| | | tgcgcctccagccaggcccaggcctctcccctctcccctttctctctgggtcttcctttgccccactgagggcct |
| | | cctgtgagcccgatttaacggaaactgtgggcggtgagaagttccttatgacacactaatcccaacctgctga |
| | | ccggaccacgcctccagcggagggaacctctagagctccaggacattcaggtaccaggtagccccaagg |
| | | aggagctgccgacctggcaggtaagtcaatacctggggcttgcctgggccagggagcccaggactggggt |
| | | gaggactcaggggagcagggagaccacgtcccaagatgcctgtaaaactgaaaccacctggccattctcc |
| | | aggttgagccagaccaatttgatggcagatttagcaaataaaaatacaggacacccagttaaatgtgaatttca |
| | | gatgaacagcaaatacttttttagtattaaaaaagttcacatttaggctcacgcctgtaatcccagcactttggga |
| | | ggccgaggcaggcagatcacctgaggtcaggagttcgagaccagcctggccaacatggtgaaaccccatc |
| | | tccactaaaaataccaaaaattagccaggcgtgctggtgggcacctgtagttccagctactcaggaggctaa |
| | | ggcaggagaattgcttgaacctgggaggcagaggttgcagtgagctgagatcgcaccattgcactctagcct |
| | | gggcgacaagaacaaaactccatctcaaaaaaaaaaaaaaaaaaaaagttcacatttaactgggcattctgta |
| | | tttaattggtaatctgagatggcagggaacagcatcagcatggtgtgagggataggcattttttcattgtgtaca |
| | | gcttgtaaatcagtatttttaaaactcaaagttaatggcttgggcatatttagaaaagagttgccgcacggacttg |
| | | aaccctgtattcctaaaatctaggatcttgttctgatggtctgcacaactggctgggggtgtccagccactgtcc |
| | | ctcttgcctgggctccccagggcagttctgtcagcctctccatttccattcctgttccagcaaaacccaactgat |
| | | agcacagcagcatttcagcctgtctacctctgtgcccacatacctggatgtctaccagccagaaaggtggctt |
| | | agatttggttcctgtgggtggattatggcccccagaacttccctgtgcttgctgggggtgtggagtggaaaga |
| | | gcaggaaatgggggaccctccgatactctatgggggtcctccaagtctctttgtgcaagttagggtaataatc |
| | | aatatggagctaagaaagagaaggggaactatgctttagaacaggacactgtgccaggagcattgcagaaa |
| | | ttatatggttttcacgacagttctttttggtaggtactgttattatcctcagtttgcagatgaggaaactgagaccca |
| | | gaaaggttaaataacttgctagggtcacacaagtcataactgacaaagcctgattcaaacccaggtctcccta |
| | | acctttaaggtttctatgacgccagctctcctagggagtttgtcttcagatgtcttggctctaggtgtcaaaaaaa |
| | | gacttggtgtcaggcaggcataggttcaagtcccaactctgtcacttaccaactgtgactaggtgattgaactg |
| | | accatggaacctggtcacatgcaggagcaggatggtgaagggttcttgaaggcacttaggcaggacatttag |
| | | gcaggagagaaaacctggaaacagaagagctgtctccaaaaatacccactggggaagcaggttgtcatgt |
| | | gggccatgaatgggacctgttctggtaaccaagcattgcttatgtgtccattacatttcataacacttccatccta |
| | | ctttacagggaacaaccaagactggggttaaatctcacagcctgcaagtggaagagaagaacttgaaccca |
| | | ggtccaacttttgcgccacagcaggctgcctcttggtcctgacaggaagtcacaacttgggtctgagtactgat |
| | | ccctggctattttttggctgtgttaccttggacaagtcacttattcctcctcccgtttcctcctatgtaaaatggaaat |
| | | aataatgttgaccctgggtctgagagagtggatttgaaagtacttagtgcatcacaaagcacagaacacacttc |
| | | cagtctcgtgattatgtacttatgtaactggtcatcacccatcttgagaatgaatgcattggggaaagggccatc |
| | | cactaggctgcgaagtttctgagggactccttcgggctggagaaggatggccacaggagggaggagagat |
| | | tgccttatcctgcagtgatcatgtcattgagaacagagccagattctttttttcctggcagggccaacttgttttaa |
| | | catctaaggactgagctatttgtgtctgtgccctttgtccaagcagtgtttcccaaagtgtagcccaagaaccat |
| | | ctccctcagagccaccaggaagtgctttaaattgcaggttcctaggccacagcctgcacctgcagagtcaga |
| | | atcatggaggttgggacccaggcacctgcgtttctaacaaatgcctcgggtgattctgatgcaattgaaagttt |
| | | gagatccacagttctgagacaataacagaatggtttttctaacccctgcagccctgacttcctatcctagggaa |
| | | ggggccggctggagaggccaggacagagaaagcagatcccttctttttccaaggactctgtgtcttccatag |
| | | gcaacgaattcgccaccatgtcagaagggggggcacgttccgcatggtacctgaagaggaacaggagct |
| | | ccgtgcccaactggagcagctcacaaccaaggaccatggacctgtctttggcccgtgcagccagctgcccc |
| | | gccacaccttgcagaaggccaaggatgagctgaacgagagagaggagacccgggaggaggcagtgcg |
| | | agagctgcaggagatggtgcaggcgcaggcggcctcgggggaggagctggcggtggccgtggcggag |
| | | agggtgcaagagaaggacagcggcttcttcctgcgcttcatccgcgcacggaagttcaacgtgggccgtgc |
| | | ctatgagctgctcagaggctatgtgaatttccggctgcagtaccctgagctctttgacagcctgtccccagagg |
| | | ctgtccgctgcaccattgaagctggctaccctggtgtcctctctagtcgggacaagtatggccgagtggtcat |
| | | gctcttcaacattgagaactggcaaagtcaagaaatcacctttgatgagatcttgcaggcatattgcttcatcct |
| | | ggagaagctgctggagaatgaggaaactcaaatcaatggcttctgcatcattgagaacttcaagggctttacc |
| | | atgcagcaggctgctagtctccggacttcagatctcaggaagatggtggacatgctccaggattccttcccag |
| | | cccggttcaaagccatccacttcatccaccagccatggtacttcaccacgacctacaatgtggtcaagcccttc |
| | | ttgaagagcaagctgcttgagagggtctttgtccacggggatgacctttctggtttctaccaggagatcgatga |
| | | gaacatcctgccctctgacttcgggggcacgctgcccaagtatgatggcaaggccgttgctgagcagctcttt |
| | | ggcccccaggcccaagctgagaacacagccttctgaggatcgtaccggtcgacctgcagaagcttgcctcg |
| | | agcagcgctgctcgagagatctggatcataatcagccataccacatttgtag aggttttacttgctttaaaaaac |
| | | ctcccacacctccccctgaacctgaaacataaaatgaatgcaattgttgttgttaacttgtttattgcagcttataa |
| | | tggttacaaataaagcaatagcatcacaaatttcacaaataaagcatttttttcactgcattctagttgtggtttgtc |
| | | caaactcatcaatgtatcttatcatgtctggtaaccacgtgcggaccgagcggccgcaggaacccctagtgat |
| | | ggagttggccactccctctctgcgcgctcgctcgctcactgaggccgggcgaccaaaggtcgcccgacgc |
| | | ccgggctttgcccgggcggcctcagtgagcgagcgagcgcgcag |
| 53 | Gene cassette of | ctgcgcgctcgctcgctcactgaggccgcccgggcgtcgggcgacctttggtcgcccggcctcagtgagc |
| | plasmid AG007 | gagcgagcgcgcagagagggagtggccaactccatcactaggggttcctgcggccgcacgcgttacgtaa |
| | (occurs at bp 1 to | tatttattgaagtttaatattgtgtttgtgatacagaagtatttgctttaattctaaataaaaattttatgcttttattgctg |
| | 4645 of SEQ ID | gtttaagaagatttggattatccttgtactttgaggagaagtttcttatttgaaatattttggaaacaggtcttttaatg |
| | NO: 28) | tggaaagatagatattaatctcctcttctattactctccaagatccaacaaaagtgattataccccccaaaatatg |
| | | atggtagtatcttatactaccatcattttataggcatagggctcttagctgcaaataatggaactaactctaataaa |
| | | gcagaacgcaaatattgtaaatattagagagctaacaatctctgggatggctaaaggatggagcttggaggct |
| | | acccagccagtaacaatattccgggctccactgttgaatggagacactacaactgccttggatgggcagaga |
| | | tattatggatgctaagccccaggtgctaccattaggacttctaccactgtccctaacgggtggagcccatcaca |
| | | tgcctatgccctcactgtaaggaaatgaagctactgttgtatatcttgggaagcacttggattaattgttatacagt |
| | | tttgttgaagaagacccctagggtaagtagccataactgcacactaaatttaaaattgttaatgagtttctcaaaa |
| | | aaaatgttaaggttgttagctggtatagtatatatcttgcctgttttccaaggacttctttgggcagtaccttgtctgt |
| | | gctggcaagcaactgagacttaatgaaagagtattggagatatgaatgaattgatgctgtatactctcagagtg |
| | | ccaaacatataccaatggacaagaaggtgaggcagagagcagacaggcattagtgacaagcaaagatatg |
| | | cagaatttcattctcagcaaatcaaaagtcctcaacctggttggaagaatattggcactgaatggtatcaataag |
| | | gttgctagagagggttagaggtgcacaatgtgcttccataacattttatacttctccaatcttagcactaatcaaa |
| | | catggttgaatactttgtttactataactcttacagagttataagatctgtgaagacagggacagggacaatacc |
| | | catctctgtctggttcataggtggtatgtaatagatatttttaaaaataagtgagttaatgaatgagggtgagaatg |
| | | aaggcacagaggtattagggggaggtgggccccagagaatggtgccaaggtccagtggggtgactggga |
| | | tcagctcaggcctgacgctggccactcccacctagctcctttctttctaatctgttctcattctccttgggaaggat |
| | | tgaggtctctggaaaacagccaaacaactgttatgggaacagcaagcccaaataaagccaagcatcaggg |
| | | ggatctgagagctgaaagcaacttctgttccccctccctcagctgaaggggggggaagggctcccaaagc |
| | | cataactccttttaagggatttagaaggcataaaaaggcccctggctgagaacttccttcttcattctgcagttgg |
| | | tgaattcgccaccatgtcagaagggggggcacgttccgcatggtacctgaagaggaacaggagctccgtg |
| | | cccaactggagcagctcacaaccaaggaccatggacctgtctttggcccgtgcagccagctgccccgccac |
| | | accttgcagaaggccaaggatgagctgaacgagagagaggagacccgggaggaggcagtgcgagagct |
| | | gcaggagatggtgcaggcgcaggcggcctcgggggaggagctggcggtggccgtggcggagagggtg |
| | | caagagaaggacagcggcttcttcctgcgcttcatccgcgcacggaagttcaacgtgggccgtgcctatga |
| | | gctgctcagaggctatgtgaatttccggctgcagtaccctgagctctttgacagcctgtccccagaggctgtcc |
| | | gctgcaccattgaagctggctaccctggtgtcctctctagtcgggacaagtatggccgagtggtcatgctcttc |
| | | aacattgagaactggcaaagtcaagaaatcacctttgatgagatcttgcaggcatattgcttcatcctggagaa |
| | | gctgctggagaatgaggaaactcaaatcaatggcttctgcatcattgagaacttcaagggctttaccatgcag |
| | | caggctgctagtctccggacttcagatctcaggaagatggtggacatgctccaggattccttcccagcccggt |
| | | tcaaagccatccacttcatccaccagccatggtacttcaccacgacctacaatgtggtcaagcccttcttgaag |
| | | agcaagctgcttgagagggtctttgtccacggggatgacctttctggtttctaccaggagatcgatgagaacat |
| | | cctgccctctgacttcgggggcacgctgcccaagtatgatggcaaggccgttgctgagcagctctttggccc |
| | | ccaggcccaagctgagaacacagccttctgaggatctaccggtcgacctgcagaagcttgcctcgagcagc |
| | | gctgctcgagagatctggatcataatcagccataccacatttgtagaggttttacttgctttaaaaaacctcccac |
| | | acctccccctgaacctgaaacataaaatgaatgcaattgttgttgttaacttgtttattgcagcttataatggttaca |
| | | aataaagcaatagcatcacaaatttcacaaataaagcatttttttcactgcattctagttgtggtttgtccaaactca |
| | | tcaatgtatcttatcatgtctggtaaccattctccaggttgagccagaccaatttgatggtagatttagcaaataaa |
| | | aatacaggacacccagttaaatgtgaatttccgatgaacagcaaatacttttttagtattaaaaaagttcacattta |
| | | ggctcacgcctgtaatcccagcactttgggaggccgaggcaggcagatcacctgaggtcaggagttcgag |
| | | accagcctggccaacatggtgaaaccccatctccactaaaaataccaaaaattagccaggcgtgctggtgg |
| | | gcacctgtagttccagctactcaggaggctaaggcaggagaattgcttgaacctgggaggcagaggttgca |
| | | gtgagctgagatcgcaccattgcactctagcctgggcgacaagaacaaaactccatctcaaaaaaaaaaaa |
| | | aaaaaaaaagttcacatttaactgggcattctgtatttaattggtaatctgagatggcagggaacagcatcagc |
| | | atggtgtgagggataggcattttttcattgtgtacagcttgtaaatcagtatttttaaaactcaaagttaatggcttg |
| | | ggcatatttagaaaagagttgccgcacggacttgaaccctgtattcctaaaatctaggatcttgttctgatggtct |
| | | gcacaactggctgggggtgtccagccactgtccctcttgcctgggctccccagggcagttctgtcagcctctc |
| | | catttccattcctgttccagcaaaacccaactgatagcacagcagcatttcagcctgtctacctctgtgcccaca |
| | | tacctggatgtctaccagccagaaaggtggcttagatttggttcctgtgggtggattatggcccccagaacttc |
| | | cctgtgcttgctgggggtgtggagtggaaagagcaggaaatgggggaccctccgatactctatgggggtcc |
| | | tccaagtctctttgtgcaagttagggtaataatcaatatggagctaagaaagagaaggggaactatgctttaga |
| | | acaggacactgtgccaggagcattgcagaaattatatggttttcacgacagttctttttggtaggtactgttattat |
| | | cctcagtttgcagatgaggaaactgagacccagaaaggttaaataacttgctagggtcacacaagtcataact |
| | | gacaaagcctgattcaaacccaggtctccctaacctttaaggtttctatgacgccagctctcctagggagtttgt |
| | | cttcagatgtcttggctctaggtgtcaaaaaaagacttggtgtcaggcaggcataggttcaagtcccaactctg |
| | | tcacttaccaactgtgactaggtgattgaactgaccatggaacctggtcacatgcaggagcaggatggtgaa |
| | | gggttcttgaaggcacttaggcaggacatttaggcaggagagaaaacctggaaacagaagagctgtctcca |
| | | aaaatacccactggggaagcaggttgtcatgtgggccatgaatgggacctgttctggggtaaccacgtgcg |
| | | gaccgagcggccgcaggaacccctagtgatggagttggccactccctctctgcgcgctcgctcgctcactg |
| | | aggccgggcgaccaaaggtcgcccgacgcccgggctttgcccgggcggcctcagtgagcgagcgagcg |
| | | cgcag |
| 54 | Gene cassette of | ctgcgcgctcgctcgctcactgaggccgcccgggcgtcgggcgacctttggtcgcccggcctcagtgagc |
| | plasmid TM039 | gagcgagcgcgcagagagggagtggccaactccatcactaggggttcctgcggccgcacgcgtactagtt |
| | (occurs at bp 1 to | attaatagtaatcaattacggggtcattagttcatagcccatatatggagttccgcgttacataacttacggtaaat |
| | 4702 of SEQ ID | ggcccgcctggctgaccgcccaacgacccccgcccattgacgtcaataatgacgtatgttcccatagtaacg |
| | NO: 29) | ccaatagggactttccattgacgtcaatgggtggagtatttacggtaaactgcccacttggcagtacatcaagt |
| | | gtatcatatgccaagtacgccccctattgacgtcaatgacggtaaatggcccgcctggcattatgcccagtac |
| | | atgaccttatgggactttcctacttggcagtacatctacgtattagtcatcgctattaccatggtcgaggtgagcc |
| | | ccacgttctgcttcactctccccatctcccccccctccccacccccaattttgtatttatttattttttaattattttgtg |
| | | cagcgatgggggcggggggggggggggggcgcgcgccaggcggggcggggcggggcgaggggcg |
| | | gggcggggcgaggcggagaggtgcggcggcagccaatcagagcggcgcgctccgaaagtttccttttat |
| | | ggcgaggcggcggcggcggcggccctataaaaagcgaagcgcgcgggggcggggagtcgctgcga |
| | | cgctgccttcgccccgtgccccgctccgccgccgcctcgcgccgcccgccccggctctgactgaccgcgtt |
| | | actcccacaggtgagcggggggacggcccttctcctccgggctgtaattagcgcttggtttaatgacggctt |
| | | gtttcttttctgtggctgcgtgaaagccttgaggggctccgggagggccctttgtgcggggggagcggctcg |
| | | gggggtgcgtgcgtgtgtgtgtgcgtggggagcgccgcgtgcggctccgcgctgcccggcggctgtgag |
| | | cgctgcgggcgcggcgcggggctttgtgcgctccgcagtgtgcgcgaggggagcgcggccgggggcg |
| | | gtgccccgcggtgcggggggggctgcgaggggaacaaaggctgcgtgcggggtgtgtgcgtgggggg |
| | | gtgagcagggggtgtgggcgcgtcggtcgggctgcaaccccccctgcacccccctccccgagttgctgag |
| | | cacggcccggcttcgggtgcggggctccgtacggggcgtggcgcggggctcgccgtgccgggcgggg |
| | | ggtggcggcaggtgggggtgccgggcggggggggccgcctcgggccggggagggctcgggggag |
| | | gggcgcggcggcccccggagcgccggcggctgtcgaggcgcggcgagccgcagccattgccttttatgg |
| | | taatcgtgcgagagggcgcagggacttcctttgtcccaaatctgtgcggagccgaaatctgggaggcgccg |
| | | ccgcaccccctctagcgggcgcggggcgaagcggtgcggcgccggcaggaaggaaatgggcgggga |
| | | gggccttcgtgcgtcgccgcgccgccgtccccttctccctctccagcctcggggctgtccgcggggggacg |
| | | gctgccttcgggggggacggggcagggcggggttcggcttctggcgtgtgaccggcggcatcgattgaat |
| | | tcgccaccatgtcagaagggggggcacgttccgcatggtacctgaagaggaacaggagctccgtgccca |
| | | actggagcagctcacaaccaaggaccatggacctgtctttggcccgtgcagccagctgccccgccacacct |
| | | tgcagaaggccaaggatgagctgaacgagagagaggagacccgggaggaggcagtgcgagagctgca |
| | | ggagatggtgcaggcgcaggcggcctcgggggaggagctggcggtggccgtggcggagagggtgcaa |
| | | gagaaggacagcggcttcttcctgcgcttcatccgcgcacggaagttcaacgtgggccgtgcctatgagctg |
| | | ctcagaggctatgtgaatttccggctgcagtaccctgagctctttgacagcctgtccccagaggctgtccgctg |
| | | caccattgaagctggctaccctggtgtcctctctagtcgggacaagtatggccgagtggtcatgctcttcaaca |
| | | ttgagaactggcaaagtcaagaaatcacctttgatgagatcttgcaggcatattgcttcatcctggagaagctg |
| | | ctggagaatgaggaaactcaaatcaatggcttctgcatcattgagaacttcaagggctttaccatgcagcagg |
| | | ctgctagtctccggacttcagatctcaggaagatggtggacatgctccaggattccttcccagcccggttcaa |
| | | agccatccacttcatccaccagccatggtacttcaccacgacctacaatgtggtcaagcccttcttgaagagc |
| | | aagctgcttgagagggtctttgtccacggggatgacctttctggtttctaccaggagatcgatgagaacatcct |
| | | gccctctgacttcgggggcacgctgcccaagtatgatggcaaggccgttgctgagcagctctttggccccca |
| | | ggcccaagctgagaacacagccttctgaggatcgtaccggtcgacctgcagaagcttgcctcgagcagcg |
| | | ctgctcgagagatctggatcataatcagccataccacatttgtagaggttttacttgctttaaaaaacctcccaca |
| | | cctccccctgaacctgaaacataaaatgaatgcaattgttgttgttaacttgtttattgcagcttataatggttacaa |
| | | ataaagcaatagcatcacaaatttcacaaataaagcatttttttcactgcattctagttgtggtttgtccaaactcat |
| | | caatgtatcttatcatgtctggtactagggttaccccagaacaggtcccattcatggcccacatgacaacctgct |
| | | tccccagtgggtatttttggagacagctcttctgtttccaggttttctctcctgcctaaatgtcctgcctaagtgcct |
| | | tcaagaacccttcaccatcctgctcctgcatgtgaccaggttccatggtcagttcaatcacctagtcacagttgg |
| | | taagtgacagagttgggacttgaacctatgcctgcctgacaccaagtctttttttgacacctagagccaagaca |
| | | tctgaagacaaactccctaggagagctggcgtcatagaaaccttaaaggttagggagacctgggtttgaatc |
| | | aggctttgtcagttatgacttgtgtgaccctagcaagttatttaacctttctgggtctcagtttcctcatctgcaaac |
| | | tgaggataataacagtacctaccaaaaagaactgtcgtgaaaaccatataatttctgcaatgctcctggcacag |
| | | tgtcctgttctaaagcatagttccccttctctttcttagctccatattgattattaccctaacttgcacaaagagactt |
| | | ggaggacccccatagagtatcggagggtcccccatttcctgctctttccactccacacccccagcaagcaca |
| | | gggaagttctgggggccataatccacccacaggaaccaaatctaagccacctttctggctggtagacatcca |
| | | ggtatgtgggcacagaggtagacaggctgaaatgctgctgtgctatcagttgggttttgctggaacaggaatg |
| | | gaaatggagaggctgacagaactgccctggggagcccaggcaagagggacagtggctggacaccccca |
| | | gccagttgtgcagaccatcagaacaagatcctagattttaggaatacagggttcaagtccgtgcggcaactct |
| | | tttctaaatatgcccaagccattaactttgagttttaaaaatactgatttacaagctgtacacaatgaaaaaatgcc |
| | | tatccctcacaccatgctgatgctgttccctgccatctcagattaccaattaaatacagaatgcccagttaaatgt |
| | | gaactttttttttttttttttttttgagatggagttttgttcttgtcgcccaggctagagtgcaatggtgcgatctcagct |
| | | cactgcaacctctgcctcccaggttcaagcaattctcctgccttagcctcctgagtagctggaactacaggtgc |
| | | ccaccagcacgcctggctaatttttggtatttttagtggagatggggtttcaccatgttggccaggctggtctcg |
| | | aactcctgacctcaggtgatctgcctgcctcggcctcccaaagtgctgggattacaggcgtgagcctaaatgt |
| | | gaacttttttaatactaaaaaagtatttgctgttcatcggaaattcacatttaactgggtgtcctgtatttttatttgcta |
| | | aatctaccatcaaattggtctggctcaacctggagaatggttaccctaggtaaccacgtgcggaccgagcgg |
| | | ccgcaggaacccctagtgatggagttggccactccctctctgcgcgctcgctcgctcactgaggccgggcg |
| | | accaaaggtcgcccgacgcccgggctttgcccgggcggcctcagtgagcgagcgagcgcgcag |
| 55 | Gene cassette of | ctgcgcgctcgctcgctcactgaggccgcccgggcgtcgggcgacctttggtcgcccggcctcagtgagc |
| | plasmid TM040 | gagcgagcgcgcagagagggagtggccaactccatcactaggggttcctgcggccgcacgcgtttgtcct |
| | (occurs at bp 1 to | ctccctgcttggccttaaccagccacatttctcaactgaccccactcactgcagaggtgaaaactaccatgcca |
| | 3873 of SEQ ID | ggtcctgctggctgggggaggggtgggcaataggcctggatttgccagagctgccactgtagatgtagtcat |
| | NO: 30) | atttacgatttcccttcacctcttattaccctggtggtggtggtgggggggggggggtgctctctcagcaaccc |
| | | caccccgggatcttgaggagaaagagggcagagaaaagagggaatgggactggcccagatcccagccc |
| | | cacagccgggcttccacatggccgagcaggaactccagagcaggagcacacaaaggagggctttgatgc |
| | | gcctccagccaggcccaggcctctcccctctcccctttctctctgggtcttcctttgccccactgagggcctcct |
| | | gtgagcccgatttaacggaaactgtgggcggtgagaagttccttatgacacactaatcccaacctgctgaccg |
| | | gaccacgcctccagcggagggaacctctagagctccaggacattcaggtaccaggtagccccaaggagg |
| | | agctgccgaatcgatggatcgggaactgaaaaaccagaaagttaactggtaagtttagtctttttgtcttttatttc |
| | | aggtcccggatccggtggtggtgcaaatcaaagaactgctcctcagtggatgttgcctttacttctaggcctgt |
| | | acggaagtgttacttctgctctaaaagctgcggaattgtacccgccccgggatccatcgattgaattcgccacc |
| | | atgtcagaaggggtgggcacgttccgcatggtacctgaagaggaacaggagctccgtgcccaactggagc |
| | | agctcacaaccaaggaccatggacctgtctttggcccgtgcagccagctgccccgccacaccttgcagaag |
| | | gccaaggatgagctgaacgagagagaggagacccgggaggaggcagtgcgagagctgcaggagatgg |
| | | tgcaggcgcaggcggcctcgggggaggagctggcggtggccgtggcggagagggtgcaagagaagga |
| | | cagcggcttcttcctgcgcttcatccgcgcacggaagttcaacgtgggccgtgcctatgagctgctcagagg |
| | | ctatgtgaatttccggctgcagtaccctgagctctttgacagcctgtccccagaggctgtccgctgcaccattg |
| | | aagctggctaccctggtgtcctctctagtcgggacaagtatggccgagtggtcatgctcttcaacattgagaac |
| | | tggcaaagtcaagaaatcacctttgatgagatcttgcaggcatattgcttcatcctggagaagctgctggagaa |
| | | tgaggaaactcaaatcaatggcttctgcatcattgagaacttcaagggctttaccatgcagcaggctgctagtc |
| | | tccggacttcagatctcaggaagatggtggacatgctccaggattccttcccagcccggttcaaagccatcca |
| | | cttcatccaccagccatggtacttcaccacgacctacaatgtggtcaagcccttcttgaagagcaagctgcttg |
| | | agagggtctttgtccacggggatgacctttctggtttctaccaggagatcgatgagaacatcctgccctctgac |
| | | ttcgggggcacgctgcccaagtatgatggcaaggccgttgctgagcagctctttggcccccaggcccaagc |
| | | tgagaacacagccttctgaggatcgtaccggtcgacctgcagaagcttgcctcgagcagcgctgctcgaga |
| | | gatctggatcataatcagccataccacatttgtagaggttttacttgctttaaaaaacctcccacacctccccctg |
| | | aacctgaaacataaaatgaatgcaattgttgttgttaacttgtttattgcagcttataatggttacaaataaagcaat |
| | | agcatcacaaatttcacaaataaagcatttttttcactgcattctagttgtggtttgtccaaactcatcaatgtatctt |
| | | atcatgtctggtactagggttaccccagaacaggtcccattcatggcccacatgacaacctgcttccccagtg |
| | | ggtatttttggagacagctcttctgtttccaggttttctctcctgcctaaatgtcctgcctaagtgccttcaagaacc |
| | | cttcaccatcctgctcctgcatgtgaccaggttccatggtcagttcaatcacctagtcacagttggtaagtgaca |
| | | gagttgggacttgaacctatgcctgcctgacaccaagtctttttttgacacctagagccaagacatctgaagac |
| | | aaactccctaggagagctggcgtcatagaaaccttaaaggttagggagacctgggtttgaatcaggctttgtc |
| | | agttatgacttgtgtgaccctagcaagttatttaacctttctgggtctcagtttcctcatctgcaaactgaggataat |
| | | aacagtacctaccaaaaagaactgtcgtgaaaaccatataatttctgcaatgctcctggcacagtgtcctgttct |
| | | aaagcatagttccccttctctttcttagctccatattgattattaccctaacttgcacaaagagacttggaggaccc |
| | | ccatagagtatcggagggtcccccatttcctgctctttccactccacacccccagcaagcacagggaagttct |
| | | gggggccataatccacccacaggaaccaaatctaagccacctttctggctggtagacatccaggtatgtggg |
| | | cacagaggtagacaggctgaaatgctgctgtgctatcagttgggttttgctggaacaggaatggaaatggag |
| | | aggctgacagaactgccctggggagcccaggcaagagggacagtggctggacacccccagccagttgtg |
| | | cagaccatcagaacaagatcctagattttaggaatacagggttcaagtccgtgcggcaactcttttctaaatatg |
| | | cccaagccattaactttgagttttaaaaatactgatttacaagctgtacacaatgaaaaaatgcctatccctcaca |
| | | ccatgctgatgctgttccctgccatctcagattaccaattaaatacagaatgcccagttaaatgtgaacttttttttt |
| | | ttttttttttttgagatggagttttgttcttgtcgcccaggctagagtgcaatggtgcgatctcagctcactgcaacc |
| | | tctgcctcccaggttcaagcaattctcctgccttagcctcctgagtagctggaactacaggtgcccaccagca |
| | | cgcctggctaatttttggtatttttagtggagatggggtttcaccatgttggccaggctggtctcgaactcctgac |
| | | ctcaggtgatctgcctgcctcggcctcccaaagtgctgggattacaggcgtgagcctaaatgtgaactttttta |
| | | atactaaaaaagtatttgctgttcatcggaaattcacatttaactgggtgtcctgtatttttatttgctaaatctaccat |
| | | caaattggtctggctcaacctggagaatggttaccctaggtaaccacgtgcggaccgagcggccgcaggaa |
| | | cccctagtgatggagttggccactccctctctgcgcgctcgctcgctcactgaggccgggcgaccaaaggtc |
| | | gcccgacgcccgggctttgcccgggggcctcagtgagcgagcgagcgcgcag |
| 56 | Gene cassette of | cgcgctcgctcgctcactgaggccgcccgggcaaagcccgggcgtcgggcgacctttggtcgcccggcct |
| | plasmid TM016 | cagtgagcgagcgagcgcgcagagagggagtggggtaccacgcgtttgtcctctccctgcttggccttaac |
| | (occurs at bp 1 to | cagccacatttctcaactgaccccactcactgcagaggtgaaaactaccatgccaggtcctgctggctgggg |
| | 2119 of SEQ ID | gaggggtgggcaataggcctggatttgccagagctgccactgtagatgtagtcatatttacgatttcccttcac |
| | NO: 31) | ctcttattaccctggtggtggtggtgggggggggggggtgctctctcagcaaccccaccccgggatcttgag |
| | | gagaaagagggcagagaaaagagggaatgggactggcccagatcccagccccacagccgggcttccac |
| | | atggccgagcaggaactccagagcaggagcacacaaaggagggctttgatgcgcctccagccaggccca |
| | | ggcctctcccctctcccctttctctctgggtcttcctttgccccactgagggcctcctgtgagcccgatttaacgg |
| | | aaactgtgggcggtgagaagttccttatgacacactaatcccaacctgctgaccggaccacgcctccagcgg |
| | | agggaacctctagagctccaggacattcaggtaccaggtagccccaaggaggagctgccgaatcgatgga |
| | | tcgggaactgaaaaaccagaaagttaactggtaagtttagtctttttgtcttttatttcaggtcccggatccggtg |
| | | gtggtgcaaatcaaagaactgctcctcagtggatgttgcctttacttctaggcctgtacggaagtgttacttctgc |
| | | tctaaaagctgcggaattgtacccgccccgggatccatcgattgaattccccggggatcctctagagtcgaaa |
| | | ttcgccaccatggtgagcaagggcgaggagctgttcaccggggtggtgcccatcctggtcgagctggacg |
| | | gcgacgtaaacggccacaagttcagcgtgtccggcgagggcgagggcgatgccacctacggcaagctga |
| | | ccctgaagttcatctgcaccaccggcaagctgcccgtgccctggcccaccctcgtgaccaccctgacctacg |
| | | gcgtgcagtgcttcagccgctaccccgaccacatgaagcagcacgacttcttcaagtccgccatgcccgaa |
| | | ggctacgtccaggagcgcaccatcttcttcaaggacgacggcaactacaagacccgcgccgaggtgaagtt |
| | | cgagggcgacaccctggtgaaccgcatcgagctgaagggcatcgacttcaaggaggacggcaacatcct |
| | | ggggcacaagctggagtacaactacaacagccacaacgtctatatcatggccgacaagcagaagaacggc |
| | | atcaaggtgaacttcaagatccgccacaacatcgaggacggcagcgtgcagctcgccgaccactaccagc |
| | | agaacacccccatcggcgacggccccgtgctgctgcccgacaaccactacctgagcacccagtccgccct |
| | | gagcaaagaccccaacgagaagcgcgatcacatggtcctgctggagttcgtgaccgccgccgggatcact |
| | | ctcggcatggacgagctgtacaagtaatagggtaccggtcgacctgcagaagcttgcctcgagcagcgctg |
| | | ctcgagagatctggatcataatcagccataccacatttgtagaggttttacttgctttaaaaaacctcccacacct |
| | | ccccctgaacctgaaacataaaatgaatgcaattgttgttgttaacttgtttattgcagcttataatggttacaaata |
| | | aagcaatagcatcacaaatttcacaaataaagcatttttttcactgcattctagttgtggtttgtccaaactcatcaa |
| | | tgtatcttatcatgtctggtaaccacgtgcggaccgagcggccgcaggaacccctagtgatggagttggcca |
| | | ctccctctctgcgcgctcgctcgctcactgaggccgggcgaccaaaggtcgcccgacgcccgggctttgcc |
| | | cgggcggcctcagtgagcgagcgagcgcgcag |
| 57 | Gene cassette of | ctgcgcgctcgctcgctcactgaggccgcccgggcgtcgggcgacctttggtcgcccggcctcagtgagc |
| | plasmid TM035 | gagcgagcgcgcagagagggagtggccaactccatcactaggggttcctgcggccgcacgcagcttttgt |
| | (occurs at bp 1 to | cctctccctgcttggccttaaccagccacatttctcaactgaccccactcactgcagaggtgaaaactaccatg |
| | 4503 of SEQ ID | ccaggtcctgctggctgggggaggggtgggcaataggcctggatttgccagagctgccactgtagatgtag |
| | NO: 32) | tcatatttacgatttcccttcacctcttattaccctggtggtggtggtgggggggggggggtgctctctcagcaa |
| | | ccccaccccgggatcttgaggagaaagagggcagagaaaagagggaatgggactggcccagatcccag |
| | | ccccacagccgggcttccacatggccgagcaggaactccagagcaggagcacacaaaggagggctttga |
| | | tgcgcctccagccaggcccaggcctctcccctctcccctttctctctgggtcttcctttgccccactgagggcct |
| | | cctgtgagcccgatttaacggaaactgtgggcggtgagaagttccttatgacacactaatcccaacctgctga |
| | | ccggaccacgcctccagcggagggaacctctagagctccaggacattcaggtaccaggtagccccaagg |
| | | aggagctgccgacctggcaggtaagtcaatacctggggcttgcctgggccagggagcccaggactggggt |
| | | gaggactcaggggagcagggagaccacgtcccaagatgcctgtaaaactgaaaccacctggccattctcc |
| | | aggttgagccagaccaatttgatggcagatttagcaaataaaaatacaggacacccagttaaatgtgaatttca |
| | | gatgaacagcaaatacttttttagtattaaaaaagttcacatttaggctcacgcctgtaatcccagcactttggga |
| | | ggccgaggcaggcagatcacctgaggtcaggagttcgagaccagcctggccaacatggtgaaaccccatc |
| | | tccactaaaaataccaaaaattagccaggcgtgctggtgggcacctgtagttccagctactcaggaggctaa |
| | | ggcaggagaattgcttgaacctgggaggcagaggttgcagtgagctgagatcgcaccattgcactctagcct |
| | | gggcgacaagaacaaaactccatctcaaaaaaaaaaaaaaaaaaaaagttcacatttaactgggcattctgta |
| | | tttaattggtaatctgagatggcagggaacagcatcagcatggtgtgagggataggcattttttcattgtgtaca |
| | | gcttgtaaatcagtatttttaaaactcaaagttaatggcttgggcatatttagaaaagagttgccgcacggacttg |
| | | aaccctgtattcctaaaatctaggatcttgttctgatggtctgcacaactggctgggggtgtccagccactgtcc |
| | | ctcttgcctgggctccccagggcagttctgtcagcctctccatttccattcctgttccagcaaaacccaactgat |
| | | agcacagcagcatttcagcctgtctacctctgtgcccacatacctggatgtctaccagccagaaaggtggctt |
| | | agatttggttcctgtgggtggattatggcccccagaacttccctgtgcttgctgggggtgtggagtggaaaga |
| | | gcaggaaatgggggaccctccgatactctatgggggtcctccaagtctctttgtgcaagttagggtaataatc |
| | | aatatggagctaagaaagagaaggggaactatgctttagaacaggacactgtgccaggagcattgcagaaa |
| | | ttatatggttttcacgacagttctttttggtaggtactgttattatcctcagtttgcagatgaggaaactgagaccca |
| | | gaaaggttaaataacttgctagggtcacacaagtcataactgacaaagcctgattcaaacccaggtctcccta |
| | | acctttaaggtttctatgacgccagctctcctagggagtttgtcttcagatgtcttggctctaggtgtcaaaaaaa |
| | | gacttggtgtcaggcaggcataggttcaagtcccaactctgtcacttaccaactgtgactaggtgattgaactg |
| | | accatggaacctggtcacatgcaggagcaggatggtgaagggttcttgaaggcacttaggcaggacatttag |
| | | gcaggagagaaaacctggaaacagaagagctgtctccaaaaatacccactggggaagcaggttgtcatgt |
| | | gggccatgaatgggacctgttctggtaaccaagcattgcttatgtgtccattacatttcataacacttccatccta |
| | | ctttacagggaacaaccaagactggggttaaatctcacagcctgcaagtggaagagaagaacttgaaccca |
| | | ggtccaacttttgcgccacagcaggctgcctcttggtcctgacaggaagtcacaacttgggtctgagtactgat |
| | | ccctggctattttttggctgtgttaccttggacaagtcacttattcctcctcccgtttcctcctatgtaaaatggaaat |
| | | aataatgttgaccctgggtctgagagagtggatttgaaagtacttagtgcatcacaaagcacagaacacacttc |
| | | cagtctcgtgattatgtacttatgtaactggtcatcacccatcttgagaatgaatgcattggggaaagggccatc |
| | | cactaggctgcgaagtttctgagggactccttcgggctggagaaggatggccacaggagggaggagagat |
| | | tgccttatcctgcagtgatcatgtcattgagaacagagccagattctttttttcctggcagggccaacttgttttaa |
| | | catctaaggactgagctatttgtgtctgtgccctttgtccaagcagtgtttcccaaagtgtagcccaagaaccat |
| | | ctccctcagagccaccaggaagtgctttaaattgcaggttcctaggccacagcctgcacctgcagagtcaga |
| | | atcatggaggttgggacccaggcacctgcgtttctaacaaatgcctcgggtgattctgatgcaattgaaagttt |
| | | gagatccacagttctgagacaataacagaatggtttttctaacccctgcagccctgacttcctatcctagggaa |
| | | ggggccggctggagaggccaggacagagaaagcagatcccttctttttccaaggactctgtgtcttccatag |
| | | gcaacgaattccccggggatcctctagagtcgaaattcgccaccatggtgagcaagggcgaggagctgttc |
| | | accggggggtgcccatcctggtcgagctggacggcgacgtaaacggccacaagttcagcgtgtccggcg |
| | | agggcgagggcgatgccacctacggcaagctgaccctgaagttcatctgcaccaccggcaagctgcccgt |
| | | gccctggcccaccctcgtgaccaccctgacctacggcgtgcagtgcttcagccgctaccccgaccacatga |
| | | agcagcacgacttcttcaagtccgccatgcccgaaggctacgtccaggagcgcaccatcttcttcaaggacg |
| | | acggcaactacaagacccgcgccgaggtgaagttcgagggcgacaccctggtgaaccgcatcgagctga |
| | | agggcatcgacttcaaggaggacggcaacatcctggggcacaagctggagtacaactacaacagccacaa |
| | | cgtctatatcatggccgacaagcagaagaacggcatcaaggtgaacttcaagatccgccacaacatcgagg |
| | | acggcagcgtgcagctcgccgaccactaccagcagaacacccccatcggcgacggccccgtgctgctgc |
| | | ccgacaaccactacctgagcacccagtccgccctgagcaaagaccccaacgagaagcgcgatcacatggt |
| | | cctgctggagttcgtgaccgccgccgggatcactctcggcatggacgagctgtacaagtaatagggtaccg |
| | | gtcgacctgcagaagcttgcctcgagcagcgctgctcgagagatctggatcataatcagccataccacatttg |
| | | tagaggttttacttgctttaaaaaacctcccacacctccccctgaacctgaaacataaaatgaatgcaattgttgt |
| | | tgttaacttgtttattgcagcttataatggttacaaataaagcaatagcatcacaaatttcacaaataaagcatttttt |
| | | tcactgcattctagttgtggtttgtccaaactcatcaatgtatcttatcatgtctggtaaccacgtgcggaccgag |
| | | cggccgcaggaacccctagtgatggagttggccactccctctctgcgcgctcgctcgctcactgaggccgg |
| | | gcgaccaaaggtcgcccgacgcccgggctttgcccgggcggcctcagtgagcgagcgagcgcgcag |
| 58 | Insert of plasmid | ctgcgcgctcgctcgctcactgaggccgcccgggcgtcgggcgacctttggtcgcccggcctcagtgagc |
| | AG012 (occurs at | gagcgagcgcgcagagagggagtggccaactccatcactaggggttcctgcggccgcacgcgtgacgtc |
| | bp 1 to 4543 of | gtttaaacgggccccggtgttatctcattcttttttctcctctgtaagttgacatgtgatgtgggaacaaaggggat |
| | SEQ ID NO: 33) | aaagtcattattttgtgctaaaatcgtaattggagaggacctcctgttagctgggctttcttctatttattgtggtggt |
| | (negative control) | tactggagttccttcttctagttttaggatatatatatatattttttttttttctttccctgaagatataataatatatatact |
| | | tctgaagattgagatttttaaattagttgtattgaaaactagctaatcagcaatttaaggctagcttgagacttatgt |
| | | cttgaatttgtttttgtaggctccaaaaccaaggagggagtggtgcatggtgtggcaacaggtaagctccattg |
| | | tgcttatatccaaagatgatatttaaagtatctagtgattagtgtggcccagtattcaagattcctatgaaattgtaa |
| | | aacaatcactgagcattctaagaacatatcagtcttattgaaactgaattctttataaagtatttttaaaaaggtaaa |
| | | tattgattataaataaaaaatatacttgccaagaataatgagggctttgaattgataagctatgtttaatttatagta |
| | | agtgggcatttaaatattctgaccaaaaatgtattgacaaactgctgacaaaaataaaatgtgaatattgccata |
| | | attttaaaaaaagagtaaaatttctgttgattacagtaaaatattttgaccttaaattatgttgattacaatattcctttg |
| | | ataattcagagtgcatttcaggaaacacccttggacagtcagtaaattgtttattgtatttatctttgtattgttatgg |
| | | tatagctatttgtacaaatattattgtgcaattattacatttctgattatattattcatttggcctaaatttaccaagaatt |
| | | tgaacaagtcaattaggtttacaatcaagaaatatcaaaaatgatgaaaaggatgataatcatcatcagatgttg |
| | | aggaagatgacgatgagagtgccagaaatagagaaatcaaaggagaaccaaaatttaacaaattaaaagcc |
| | | cacagacttgctgtaattaagttttctgttgtaagtactccacgtttcctggcagatgtggtgaagcaaaagatat |
| | | aatcagaaatataatttatatgatcggaaagcattaaacacaatagtgcctatacaaataaaatgttcctatcact |
| | | gacttctaaaatggaaatgaggacaatgatatgggaatcttaatacagtgttgtggataggactaaaaacaca |
| | | ggagtcagatcttcttggttcaacttcctgcttactccttaccagctgtgtgttttttgcaaggttcttcacctctatg |
| | | tgatttagcttcctcatctataaaataattcagtgaattaatgtacacaaaacatctggaaaacaaaagcaaaca |
| | | atatgtattttataagtgttacttatagttttatagtgaactttcttgtgcaacatttttacaactagtggagaaaaatat |
| | | ttctttaaatgaatacttttgatttaaaaatcagagtgtaaaaataaaacagactcctttgaaactagttctgttaga |
| | | agttaattgtgcacctttaatgggctctgttgcaatccaacagagaagtagttaagtaagtggactatgatggctt |
| | | ctagggacctcctataaatatgatattgtgaagcatgattataataagaactagataacagacaggtggagact |
| | | ccactatctgaagagggtcaacctagatgaatggtgttccatttagtagttgaggaagaacccatgaggtttag |
| | | aaagcagacaagcatgtggcaagttctggagtcagtggtaaaaattaaagaacccaactattactgtcaccta |
| | | atgatctaatggagactgtggagatgggctgcatttttttaatcttctccagaatgccaaaatgtaaacacatatc |
| | | tgtgtgtgtgtgtgtgtgtgtgtgtgtgtgtgagagagagagagagagagagagagactgaagtttgtacaatt |
| | | agacattttataaaatgttttctgaaggacagtggctcacaatcttaagtttctaacattgtacaatgttgggagac |
| | | tttgtatactttattttctctttagcatattaaggaatctgagatgtcctacagtaaagaaatttgcattacatagttaa |
| | | aatcagggttattcaaactttttgattattgaaacctttcttcattagttactagggttgaatgaaactagtgttccac |
| | | agaaaactatgggaaatgttgctaggcagtaaggacatggtgatttcagcatgtgcaatatttacagcgattgc |
| | | acccatggaccaccctggcagtagtgaaataaccaaaaatgctgtcataactagtatggctatgagaaacac |
| | | attgggcagaagcttgcctcgagcagcgctgctcgagagatctggatcataatcagccataccacatttgtag |
| | | aggttttacttgctttaaaaaacctcccacacctccccctgaacctgaaacataaaatgaatgcaattgttgttgtt |
| | | aacttgtttattgcagcttataatggttacaaataaagcaatagcatcacaaatttcacaaataaagcatttttttca |
| | | ctgcattctagttgtggtttgtccaaactcatcaatgtatcttatcatgtctggtaaccattctccaggttgagccag |
| | | accaatttgatggtagatttagcaaataaaaatacaggacacccagttaaatgtgaatttccgatgaacagcaa |
| | | atacttttttagtattaaaaaagttcacatttaggctcacgcctgtaatcccagcactttgggaggccgaggcag |
| | | gcagatcacctgaggtcaggagttcgagaccagcctggccaacatggtgaaaccccatctccactaaaaat |
| | | accaaaaattagccaggcgtgctggtgggcacctgtagttccagctactcaggaggctaaggcaggagaat |
| | | tgcttgaacctgggaggcagaggttgcagtgagctgagatcgcaccattgcactctagcctgggcgacaag |
| | | aacaaaactccatctcaaaaaaaaaaaaaaaaaaaaagttcacatttaactgggcattctgtatttaattggtaat |
| | | ctgagatggcagggaacagcatcagcatggtgtgagggataggcattttttcattgtgtacagcttgtaaatca |
| | | gtatttttaaaactcaaagttaatggcttgggcatatttagaaaagagttgccgcacggacttgaaccctgtattc |
| | | ctaaaatctaggatcttgttctgatggtctgcacaactggctgggggtgtccagccactgtccctcttgcctggg |
| | | ctccccagggcagttctgtcagcctctccatttccattcctgttccagcaaaacccaactgatagcacagcagc |
| | | atttcagcctgtctacctctgtgcccacatacctggatgtctaccagccagaaaggtggcttagatttggttcct |
| | | gtgggtggattatggcccccagaacttccctgtgcttgctgggggtgtggagtggaaagagcaggaaatgg |
| | | gggaccctccgatactctatgggggtcctccaagtctctttgtgcaagttagggtaataatcaatatggagcta |
| | | agaaagagaaggggaactatgctttagaacaggacactgtgccaggagcattgcagaaattatatggttttca |
| | | cgacagttctttttggtaggtactgttattatcctcagtttgcagatgaggaaactgagacccagaaaggttaaat |
| | | aacttgctagggtcacacaagtcataactgacaaagcctgattcaaacccaggtctccctaacctttaaggtttc |
| | | tatgacgccagctctcctagggagtttgtcttcagatgtcttggctctaggtgtcaaaaaaagacttggtgtcag |
| | | gcaggcataggttcaagtcccaactctgtcacttaccaactgtgactaggtgattgaactgaccatggaacctg |
| | | gtcacatgcaggagcaggatggtgaagggttcttgaaggcacttaggcaggacatttaggcaggagagaaa |
| | | acctggaaacagaagagctgtctccaaaaatacccactggggaagcaggttgtcatgtgggccatgaatgg |
| | | gacctgttctggggtaaccacgtgcggaccgagcggccgcaggaacccctagtgatggagttggccactc |
| | | cctctctgcgcgctcgctcgctcactgaggccgggcgaccaaaggtcgcccgacgcccgggctttgcccg |
| | | ggcggcctcagtgagcgagcgagcgcgcag |
| 59 | Gene cassette of | ctgcgcgctcgctcgctcactgaggccgcccgggcgtcgggcgacctttggtcgcccggcctcagtgagc |
| | plasmid AG004 | gagcgagcgcgcagagagggagtggccaactccatcactaggggttcctgcggccgcacgcgttacgtaa |
| | (occurs at bp 1 to | tatttattgaagtttaatattgtgtttgtgatacagaagtatttgctttaattctaaataaaaattttatgcttttattgctg |
| | 4438 of SEQ ID | gtttaagaagatttggattatccttgtactttgaggagaagtttcttatttgaaatattttggaaacaggtcttttaatg |
| | NO: 34) | tggaaagatagatattaatctcctcttctattactctccaagatccaacaaaagtgattataccccccaaaatatg |
| | | atggtagtatcttatactaccatcattttataggcatagggctcttagctgcaaataatggaactaactctaataaa |
| | | gcagaacgcaaatattgtaaatattagagagctaacaatctctgggatggctaaaggatggagcttggaggct |
| | | acccagccagtaacaatattccgggctccactgttgaatggagacactacaactgccttggatgggcagaga |
| | | tattatggatgctaagccccaggtgctaccattaggacttctaccactgtccctaacgggtggagcccatcaca |
| | | tgcctatgccctcactgtaaggaaatgaagctactgttgtatatcttgggaagcacttggattaattgttatacagt |
| | | tttgttgaagaagacccctagggtaagtagccataactgcacactaaatttaaaattgttaatgagtttctcaaaa |
| | | aaaatgttaaggttgttagctggtatagtatatatcttgcctgttttccaaggacttctttgggcagtaccttgtctgt |
| | | gctggcaagcaactgagacttaatgaaagagtattggagatatgaatgaattgatgctgtatactctcagagtg |
| | | ccaaacatataccaatggacaagaaggtgaggcagagagcagacaggcattagtgacaagcaaagatatg |
| | | cagaatttcattctcagcaaatcaaaagtcctcaacctggttggaagaatattggcactgaatggtatcaataag |
| | | gttgctagagagggttagaggtgcacaatgtgcttccataacattttatacttctccaatcttagcactaatcaaa |
| | | catggttgaatactttgtttactataactcttacagagttataagatctgtgaagacagggacagggacaatacc |
| | | catctctgtctggttcataggtggtatgtaatagatatttttaaaaataagtgagttaatgaatgagggtgagaatg |
| | | aaggcacagaggtattagggggaggtgggccccagagaatggtgccaaggtccagtggggtgactggga |
| | | tcagctcaggcctgacgctggccactcccacctagctcctttctttctaatctgttctcattctccttgggaaggat |
| | | tgaggtctctggaaaacagccaaacaactgttatgggaacagcaagcccaaataaagccaagcatcaggg |
| | | ggatctgagagctgaaagcaacttctgttccccctccctcagctgaaggggggggaagggctcccaaagc |
| | | cataactccttttaagggatttagaaggcataaaaaggcccctggctgagaacttccttcttcattctgcagttgg |
| | | tgaattccccggggatcctctagagtcgaaattcgccaccatggtgagcaagggcgaggagctgttcaccg |
| | | gggtggtgcccatcctggtcgagctggacggcgacgtaaacggccacaagttcagcgtgtccggcgaggg |
| | | cgagggcgatgccacctacggcaagctgaccctgaagttcatctgcaccaccggcaagctgcccgtgccct |
| | | ggcccaccctcgtgaccaccctgacctacggcgtgcagtgcttcagccgctaccccgaccacatgaagcag |
| | | cacgacttcttcaagtccgccatgcccgaaggctacgtccaggagcgcaccatcttcttcaaggacgacggc |
| | | aactacaagacccgcgccgaggtgaagttcgagggcgacaccctggtgaaccgcatcgagctgaagggc |
| | | atcgacttcaaggaggacggcaacatcctggggcacaagctggagtacaactacaacagccacaacgtcta |
| | | tatcatggccgacaagcagaagaacggcatcaaggtgaacttcaagatccgccacaacatcgaggacggc |
| | | agcgtgcagctcgccgaccactaccagcagaacacccccatcggcgacggccccgtgctgctgcccgac |
| | | aaccactacctgagcacccagtccgccctgagcaaagaccccaacgagaagcgcgatcacatggtcctgc |
| | | tggagttcgtgaccgccgccgggatcactctcggcatggacgagctgtacaagtaatagggtaccggtcga |
| | | cctgcagaagcttgcctcgagcagcgctgctcgagagatctggatcataatcagccataccacatttgtagag |
| | | gttttacttgctttaaaaaacctcccacacctccccctgaacctgaaacataaaatgaatgcaattgttgttgttaa |
| | | cttgtttattgcagcttataatggttacaaataaagcaatagcatcacaaatttcacaaataaagcatttttttcact |
| | | gcattctagttgtggtttgtccaaactcatcaatgtatcttatcatgtctggtaaccattctccaggttgagccaga |
| | | ccaatttgatggtagatttagcaaataaaaatacaggacacccagttaaatgtgaatttccgatgaacagcaaa |
| | | tacttttttagtattaaaaaagttcacatttaggctcacgcctgtaatcccagcactttgggaggccgaggcagg |
| | | cagatcacctgaggtcaggagttcgagaccagcctggccaacatggtgaaaccccatctccactaaaaatac |
| | | caaaaattagccaggcgtgctggtgggcacctgtagttccagctactcaggaggctaaggcaggagaattg |
| | | cttgaacctgggaggcagaggttgcagtgagctgagatcgcaccattgcactctagcctgggcgacaagaa |
| | | caaaactccatctcaaaaaaaaaaaaaaaaaaaaagttcacatttaactgggcattctgtatttaattggtaatct |
| | | gagatggcagggaacagcatcagcatggtgtgagggataggcattttttcattgtgtacagcttgtaaatcagt |
| | | atttttaaaactcaaagttaatggcttgggcatatttagaaaagagttgccgcacggacttgaaccctgtattcct |
| | | aaaatctaggatcttgttctgatggtctgcacaactggctgggggtgtccagccactgtccctcttgcctgggct |
| | | ccccagggcagttctgtcagcctctccatttccattcctgttccagcaaaacccaactgatagcacagcagcat |
| | | ttcagcctgtctacctctgtgcccacatacctggatgtctaccagccagaaaggtggcttagatttggttcctgt |
| | | gggtggattatggcccccagaacttccctgtgcttgctgggggtgtggagtggaaagagcaggaaatgggg |
| | | gaccctccgatactctatgggggtcctccaagtctctttgtgcaagttagggtaataatcaatatggagctaaga |
| | | aagagaaggggaactatgctttagaacaggacactgtgccaggagcattgcagaaattatatggttttcacga |
| | | cagttctttttggtaggtactgttattatcctcagtttgcagatgaggaaactgagacccagaaaggttaaataac |
| | | ttgctagggtcacacaagtcataactgacaaagcctgattcaaacccaggtctccctaacctttaaggtttctat |
| | | gacgccagctctcctagggagtttgtcttcagatgtcttggctctaggtgtcaaaaaaagacttggtgtcaggc |
| | | aggcataggttcaagtcccaactctgtcacttaccaactgtgactaggtgattgaactgaccatggaacctggt |
| | | cacatgcaggagcaggatggtgaagggttcttgaaggcacttaggcaggacatttaggcaggagagaaaa |
| | | cctggaaacagaagagctgtctccaaaaatacccactggggaagcaggttgtcatgtgggccatgaatggg |
| | | acctgttctggggtaaccacgtgcggaccgagcggccgcaggaacccctagtgatggagttggccactccc |
| | | tctctgcgcgctcgctcgctcactgaggccgggcgaccaaaggtcgcccgacgcccgggctttgcccggg |
| | | cggcctcagtgagcgagcgagcgcgcag |
| 60 | Gene cassette of | ctgcgcgctcgctcgctcactgaggccgcccgggcgtcgggcgacctttggtcgcccggcctcagtgagc |
| | plasmid AG006 | gagcgagcgcgcagagagggagtggccaactccatcactaggggttcctgcggccgcacgcgttacgtaa |
| | (occurs at bp 1 to | ttctgtcattttactagggtgatgaaattcccaagcaacaccatccttttcagataagggcactgaggctgagag |
| | 3481 of SEQ ID | aggagctgaaacctacccggcgtcaccacacacaggtggcaaggctgggaccagaaaccaggactgttg |
| | NO: 35) | actgcagcccggtattcattctttccatagcccacagggctgtcaaagaccccagggcctagtcagaggctc |
| | | ctccttcctggagagttcctggcacagaagttgaagctcagcacagccccctaacccccaactctctctgcaa |
| | | ggcctcaggggtcagaacactggtggagcagatcctttagcctctggattttagggccatggtagagggggt |
| | | gttgccctaaattccagccctggtctcagcccaacaccctccaagaagaaattagaggggccatggccagg |
| | | ctgtgctagccgttgcttctgagcagattacaagaagggactaagacaaggactcctttgtggaggtcctggc |
| | | ttagggagtcaagtgacggcggctcagcactcacgtgggcagtgccagcctctaagagtgggcaggggca |
| | | ctggccacagagtcccagggagtcccaccagcctagtcgccagaccgaattccccggggatcctctagagt |
| | | cgaaattcgccaccatggtgagcaagggcgaggagctgttcaccggggtggtgcccatcctggtcgagctg |
| | | gacggcgacgtaaacggccacaagttcagcgtgtccggcgagggcgagggcgatgccacctacggcaa |
| | | gctgaccctgaagttcatctgcaccaccggcaagctgcccgtgccctggcccaccctcgtgaccaccctga |
| | | cctacggcgtgcagtgcttcagccgctaccccgaccacatgaagcagcacgacttcttcaagtccgccatgc |
| | | ccgaaggctacgtccaggagcgcaccatcttcttcaaggacgacggcaactacaagacccgcgccgaggt |
| | | gaagttcgagggcgacaccctggtgaaccgcatcgagctgaagggcatcgacttcaaggaggacggcaa |
| | | catcctggggcacaagctggagtacaactacaacagccacaacgtctatatcatggccgacaagcagaaga |
| | | acggcatcaaggtgaacttcaagatccgccacaacatcgaggacggcagcgtgcagctcgccgaccacta |
| | | ccagcagaacacccccatcggcgacggccccgtgctgctgcccgacaaccactacctgagcacccagtcc |
| | | gccctgagcaaagaccccaacgagaagcgcgatcacatggtcctgctggagttcgtgaccgccgccggga |
| | | tcactctcggcatggacgagctgtacaagtaatagggtaccggtcgacctgcagaagcttgcctcgagcagc |
| | | gctgctcgagagatctggatcataatcagccataccacatttgtagaggttttacttgctttaaaaaacctcccac |
| | | acctccccctgaacctgaaacataaaatgaatgcaattgttgttgttaacttgtttattgcagcttataatggttaca |
| | | aataaagcaatagcatcacaaatttcacaaataaagcatttttttcactgcattctagttgtggtttgtccaaactca |
| | | tcaatgtatcttatcatgtctggtaaccattctccaggttgagccagaccaatttgatggtagatttagcaaataaa |
| | | aatacaggacacccagttaaatgtgaatttccgatgaacagcaaatacttttttagtattaaaaaagttcacattta |
| | | ggctcacgcctgtaatcccagcactttgggaggccgaggcaggcagatcacctgaggtcaggagttcgag |
| | | accagcctggccaacatggtgaaaccccatctccactaaaaataccaaaaattagccaggcgtgctggtgg |
| | | gcacctgtagttccagctactcaggaggctaaggcaggagaattgcttgaacctgggaggcagaggttgca |
| | | gtgagctgagatcgcaccattgcactctagcctgggcgacaagaacaaaactccatctcaaaaaaaaaaaa |
| | | aaaaaaaaagttcacatttaactgggcattctgtatttaattggtaatctgagatggcagggaacagcatcagc |
| | | atggtgtgagggataggcattttttcattgtgtacagcttgtaaatcagtatttttaaaactcaaagttaatggcttg |
| | | ggcatatttagaaaagagttgccgcacggacttgaaccctgtattcctaaaatctaggatcttgttctgatggtct |
| | | gcacaactggctgggggtgtccagccactgtccctcttgcctgggctccccagggcagttctgtcagcctctc |
| | | catttccattcctgttccagcaaaacccaactgatagcacagcagcatttcagcctgtctacctctgtgcccaca |
| | | tacctggatgtctaccagccagaaaggtggcttagatttggttcctgtgggtggattatggcccccagaacttc |
| | | cctgtgcttgctgggggtgtggagtggaaagagcaggaaatgggggaccctccgatactctatgggggtcc |
| | | tccaagtctctttgtgcaagttagggtaataatcaatatggagctaagaaagagaaggggaactatgctttaga |
| | | acaggacactgtgccaggagcattgcagaaattatatggttttcacgacagttctttttggtaggtactgttattat |
| | | cctcagtttgcagatgaggaaactgagacccagaaaggttaaataacttgctagggtcacacaagtcataact |
| | | gacaaagcctgattcaaacccaggtctccctaacctttaaggtttctatgacgccagctctcctagggagtttgt |
| | | cttcagatgtcttggctctaggtgtcaaaaaaagacttggtgtcaggcaggcataggttcaagtcccaactctg |
| | | tcacttaccaactgtgactaggtgattgaactgaccatggaacctggtcacatgcaggagcaggatggtgaa |
| | | gggttcttgaaggcacttaggcaggacatttaggcaggagagaaaacctggaaacagaagagctgtctcca |
| | | aaaatacccactggggaagcaggttgtcatgtgggccatgaatgggacctgttctggggtaaccacgtgcg |
| | | gaccgagcggccgcaggaacccctagtgatggagttggccactccctctctgcgcgctcgctcgctcactg |
| | | aggccgggcgaccaaaggtcgcccgacgcccgggctttgcccgggcggcctcagtgagcgagcgagcg |
| | | cgcag |
| 61 | Gene cassette of | cgcgctcgctcgctcactgaggccgcccgggcaaagcccgggcgtcgggcgacctttggtcgcccggcct |
| | plasmid TM042 | cagtgagcgagcgagcgcgcagagagggagtggggtaccacgcgtttgtcctctccctgcttggccttaac |
| | (occurs at bp 4 to | cagccacatttctcaactgaccccactcactgcagaggtgaaaactaccatgccaggtcctgctggctgggg |
| | 2330 of SEQ ID | gagggggggcaataggcctggatttgccagagctgccactgtagatgtagtcatatttacgatttcccttcac |
| | NO: 50) | ctcttattaccctggtggtggtggtgggggggggggggtgctctctcagcaaccccaccccgggatcttgag |
| | | gagaaagagggcagagaaaagagggaatgggactggcccagatcccagccccacagccgggcttccac |
| | | atggccgagcaggaactccagagcaggagcacacaaaggagggctttgatgcgcctccagccaggccca |
| | | ggcctctcccctctcccctttctctctgggtcttcctttgccccactgagggcctcctgtgagcccgatttaacgg |
| | | aaactgtgggcggtgagaagttccttatgacacactaatcccaacctgctgaccggaccacgcctccagcgg |
| | | agggaacctctagagctccaggacattcaggtaccaggtagccccaaggaggagctgccgaatcgatgga |
| | | tcgggaactgaaaaaccagaaagttaactggtaagtttagtctttttgtcttttatttcaggtcccggatccggtg |
| | | gtggtgcaaatcaaagaactgctcctcagtggatgttgcctttacttctaggcctgtacggaagtgttacttctgc |
| | | tctaaaagctgcggaattgtacccgccccgggatccatcgattgaattcgccaccatgtcagaaggggtggg |
| | | cacgttccgcatggtacctgaagaggaacaggagctccgtgcccaactggagcagctcacaaccaaggac |
| | | catggacctgtctttggcccgtgcagccagctgccccgccacaccttgcagaaggccaaggatgagctgaa |
| | | cgagagagaggagacccgggaggaggcagtgcgagagctgcaggagatggtgcaggcgcaggcggc |
| | | ctcgggggaggagctggcggtggccgtggcggagagggtgcaagagaaggacagcggcttcttcctgcg |
| | | cttcatccgcgcacggaagttcaacgtgggccgtgcctatgagctgctcagaggctatgtgaatttccggctg |
| | | cagtaccctgagctctttgacagcctgtccccagaggctgtccgctgcaccattgaagctggctaccctggtg |
| | | tcctctctagtcgggacaagtatggccgagtggtcatgctcttcaacattgagaactggcaaagtcaagaaat |
| | | cacctttgatgagatcttgcaggcatattgcttcatcctggagaagctgctggagaatgaggaaactcaaatca |
| | | atggcttctgcatcattgagaacttcaagggctttaccatgcagcaggctgctagtctccggacttcagatctca |
| | | ggaagatggtggacatgctccaggattccttcccagcccggttcaaagccatccacttcatccaccagccatg |
| | | gtacttcaccacgacctacaatgtggtcaagcccttcttgaagagcaagctgcttgagagggtctttgtccacg |
| | | gggatgacctttctggtttctaccaggagatcgatgagaacatcctgccctctgacttcgggggcacgctgcc |
| | | caagtatgatggcaaggccgttgctgagcagctctttggcccccaggcccaagctgagaacacagccttctg |
| | | aggatcgtaccggtcgacctgcagaagcttgcctcgagcagcgctgctcgagagatctggatcataatcagc |
| | | cataccacatttgtagaggttttacttgctttaaaaaacctcccacacctccccctgaacctgaaacataaaatg |
| | | aatgcaattgttgttgttaacttgtttattgcagcttataatggttacaaataaagcaatagcatcacaaatttcaca |
| | | aataaagcatttttttcactgcattctagttgtggtttgtccaaactcatcaatgtatcttatcatgtctggtaaccac |
| | | gtgcggaccgagcggccgcaggaacccctagtgatggagttggccactccctctctgcgcgctcgctcgct |
| | | cactgaggccgggcgaccaaaggtcgcccgacgcccgggctttgcccgggcggcctcagtgagcgagc |
| | | gagcgcgcag |
| 62 | Reverse | agacatgataagatacattgatgagtttggacaaaccacaactagaatgcagtgaaaaaaatgctttatttgtga |
| | complement of | aatttgtgatgctattgctttatttgtaaccattataagctgcaataaacaagttaacaacaacaattgcattcatttt |
| | SV40 polyA | atgtttcaggttcagggggaggtgtgggaggttttttaaagcaagtaaaacctctacaaatgtggtatggctgat |
| | (SEQ ID NO: 8) | tatgatc |
| 63 | Reverse | tcagaaggctgtgttctcagcttgggcctgggggccaaagagctgctcagcaacggccttgccatcatacttg |
| | complement of | ggcagcgtgcccccgaagtcagagggcaggatgttctcatcgatctcctggtagaaaccagaaaggtcatc |
| | Human RLBP1 | cccgtggacaaagaccctctcaagcagcttgctcttcaagaagggcttgaccacattgtaggtcgtggtgaa |
| | CDS (SEQ ID | gtaccatggctggtggatgaagtggatggctttgaaccgggctgggaaggaatcctggagcatgtccaccat |
| | NO: 7) | cttcctgagatctgaagtccggagactagcagcctgctgcatggtaaagcccttgaagttctcaatgatgcag |
| | | aagccattgatttgagtttcctcattctccagcagcttctccaggatgaagcaatatgcctgcaagatctcatcaa |
| | | aggtgatttcttgactttgccagttctcaatgttgaagagcatgaccactcggccatacttgtcccgactagaga |
| | | ggacaccagggtagccagcttcaatggtgcagcggacagcctctggggacaggctgtcaaagagctcagg |
| | | gtactgcagccggaaattcacatagcctctgagcagctcataggcacggcccacgttgaacttccgtgcgcg |
| | | gatgaagcgcaggaagaagccgctgtccttctcttgcaccctctccgccacggccaccgccagctcctccc |
| | | ccgaggccgcctgcgcctgcaccatctcctgcagctctcgcactgcctcctcccgggtctcctctctctcgttc |
| | | agctcatccttggccttctgcaaggtgtggcggggcagctggctgcacgggccaaagacaggtccatggtc |
| | | cttggttgtgagctgctccagttgggcacggagctcctgttcctcttcaggtaccatgcggaacgtgcccacc |
| | | ccttctgacat |
| 64 | Reverse | ggtggc |
| | complement of | |
| | Kozak sequence | |
| | (SEQ ID NO: 5) | |
| 65 | Reverse | ggatcccggggcgggtacaattccgcagcttttagagcagaagtaacacttccgtacaggcctagaagtaaa |
| | complement of | ggcaacatccactgaggagcagttctttgatttgcaccaccaccggatccgggacctgaaataaaagacaaa |
| | modified SV40 | aag actaaacttaccagttaactttctggtttttcagtt |
| | intron (SEQ ID | |
| | NO: 4) | |
| 66 | Reverse | tcggcagctcctccttggggctacctggtacctgaatgtcctggagctctagaggttccctccgctggaggcg |
| | complement of | tggtccggtcagcaggttgggattagtgtgtcataaggaacttctcaccgcccacagtttccgttaaatcgggc |
| | Human RLBP1 | tcacaggaggccctcagtggggcaaaggaagacccagagagaaaggggagaggggagaggcctgggc |
| | promoter (short) | ctggctggaggcgcatcaaagccctcctttgtgtgctcctgctctggagttcctgctcggccatgtggaagcc |
| | (SEQ ID NO: 3) | cggctgtggggctgggatctgggccagtcccattccctcttttctctgccctctttctcctcaagatcccggggt |
| | | ggggttgctgagagagcacccccccccccccaccaccaccaccagggtaataagaggtgaagggaaatc |
| | | gtaaatatgactacatctacagtggcagctctggcaaatccaggcctattgcccacccctcccccagccagca |
| | | ggacctggcatggtagttttcacctctgcagtgagtggggtcagttgagaaatgtggctggttaaggccaagc |
| | | agggagaggacaa |
| 67 | Reverse | ttacttgtacagctcgtccatgccgagagtgatcccggcggcggtcacgaactccagcaggaccatgtgatc |
| | complement of | gcgcttctcgttggggtctttgctcagggcggactgggtgctcaggtagtggttgtcgggcagcagcacggg |
| | eGFP (SEQ ID | gccgtcgccgatgggggtgttctgctggtagtggtcggcgagctgcacgctgccgtcctcgatgttgtggcg |
| | NO: 24) | gatcttgaagttcaccttgatgccgttcttctgcttgtcggccatgatatagacgttgtggctgttgtagttgtactc |
| | | cagcttgtgccccaggatgttgccgtcctccttgaagtcgatgcccttcagctcgatgcggttcaccagggtgt |
| | | cgccctcgaacttcacctcggcgcgggtcttgtagttgccgtcgtccttgaagaagatggtgcgctcctggac |
| | | gtagccttcgggcatggcggacttgaagaagtcgtgctgcttcatgtggtcggggtagcggctgaagcactg |
| | | cacgccgtaggtcagggtggtcacgagggtgggccagggcacgggcagcttgccggtggtgcagatgaa |
| | | cttcagggtcagcttgccgtaggtggcatcgccctcgccctcgccggacacgctgaacttgtggccgtttacg |
| | | tcgccgtccagctcgaccaggatgggcaccaccccggtgaacagctcctcgcccttgctcaccat |
| 68 | AAV2 capsid | MAPGKKRPVEHSPVEPDSSSGTGKAGQQPARKRLNFGQTGDADSVP |
| | protein sequence | DPQPLGQPPAAPSGLGTNTMATGSGAPMADNNEGADGVGNSSGNW |
| | (VP2) | HCDSTWMGDRVITTSTRTWALPTYNNHLYKQISSQSGASNDNHYFG |
| | | YSTPWGYFDFNRFHCHFSPRDWQRLINNNWGFRPKRLNFKLFNIQV |
| | | KEVTQNDGTTTIANNLTSTVQVFTDSEYQLPYVLGSAHQGCLPPFPA |
| | | DVFMVPQYGYLTLNNGSQAVGRSSFYCLEYFPSQMLRTGNNFTFSY |
| | | TFEDVPFHSSYAHSQSLDRLMNPLIDQYLYYLSRTNTPSGTTTQSRLQ |
| | | FSQAGASDIRDQSRNWLPGPCYRQQRVSKTSADNNNSEYSWTGATK |
| | | YHLNGRDSLVNPGPAMASHKDDEEKFFPQSGVLIFGKQGSEKTNVDI |
| | | EKVMITDEEEIRTTNPVATEQYGSVSTNLQRGNRQAATADVNTQGV |
| | | LPGMVWQDRDVYLQGPIWAKIPHTDGHFHPSPLMGGFGLKHPPPQI |
| | | LIKNTPVPANPSTTFSAAKFASFITQYSTGQVSVEIEWELQKENSKRW |
| | | NPEIQYTSNYNKSVNVDFTVDTNGVYSEPRPIGTRYLTRNL |
| 69 | AAV2 capsid | MATGSGAPMADNNEGADGVGNSSGNWHCDSTWMGDRVITTSTRT |
| | protein sequence | WALPTYNNHLYKQISSQSGASNDNHYFGYSTPWGYFDFNRFHCHFS |
| | (VP3) | PRDWQRLINNNWGFRPKRLNFKLFNIQVKEVTQNDGTTTIANNLTST |
| | | VQVFTDSEYQLPYVLGSAHQGCLPPFPADVFMVPQYGYLTLNNGSQ |
| | | AVGRSSFYCLEYFPSQMLRTGNNFTFSYTFEDVPFHSSYAHSQSLDR |
| | | LMNPLIDQYLYYLSRTNTPSGTTTQSRLQFSQAGASDIRDQSRNWLP |
| | | GPCYRQQRVSKTSADNNNSEYSWTGATKYHLNGRDSLVNPGPAMA |
| | | SHKDDEEKFFPQSGVLIFGKQGSEKTNVDIEKVMITDEEEIRTTNPVA |
| | | TEQYGSVSTNLQRGNRQAATADVNTQGVLPGMVWQDRDVYLQGPI |
| | | WAKIPHTDGHFHPSPLMGGFGLKHPPPQILIKNTPVPANPSTTFSAAK |
| | | FASFITQYSTGQVSVEIEWELQKENSKRWNPEIQYTSNYNKSVNVDF |
| | | TVDTNGVYSEPRPIGTRYLTRNL |
| 70 | AAV8 capsid | MAPGKKRPVEPSPQRSPDSSTGIGKKGQQPARKRLNFGQTGDSESVP |
| | protein sequence | DPQPLGEPPAAPSGVGPNTMAAGGGAPMADNNEGADGVGSSSGNW |
| | (VP2) | HCDSTWLGDRVITTSTRTWALPTYNNHLYKQISNGTSGGATNDNTY |
| | | FGYSTPWGYFDFNRFHCHFSPRDWQRLINNNWGFRPKRLSFKLFNIQ |
| | | VKEVTQNEGTKTIANNLTSTIQVFTDSEYQLPYVLGSAHQGCLPPFP |
| | | ADVFMIPQYGYLTLNNGSQAVGRSSFYCLEYFPSQMLRTGNNFQFT |
| | | YTFEDVPFHSSYAHSQSLDRLMNPLIDQYLYYLSRTQTTGGTANTQT |
| | | LGFSQGGPNTMANQAKNWLPGPCYRQQRVSTTTGQNNNSNFAWTA |
| | | GTKYHLNGRNSLANPGIAMATHKDDEERFFPSNGILIFGKQNAARDN |
| | | ADYSDVMLTSEEEIKTTNPVATEEYGIVADNLQQQNTAPQIGTVNSQ |
| | | GALPGMVWQNRDVYLQGPIWAKIPHTDGNFHPSPLMGGFGLKHPPP |
| | | QILIKNTPVPADPPTTFNQSKLNSFITQYSTGQVSVEIEWELQKENSKR |
| | | WNPEIQYTSNYYKSTSVDFAVNTEGVYSEPRPIGTRYLTRNL |
| 71 | AAV8 capsid | MAAGGGAPMADNNEGADGVGSSSGNWHCDSTWLGDRVITTSTRT |
| | protein sequence | WALPTYNNHLYKQISNGTSGGATNDNTYFGYSTPWGYFDFNRFHC |
| | (VP3) | HFSPRDWQRLINNNWGFRPKRLSFKLFNIQVKEVTQNEGTKTIANNL |
| | | TSTIQVFTDSEYQLPYVLGSAHQGCLPPFPADVFMIPQYGYLTLNNG |
| | | SQAVGRSSFYCLEYFPSQMLRTGNNFQFTYTFEDVPFHSSYAHSQSL |
| | | DRLMNPLIDQYLYYLSRTQTTGGTANTQTLGFSQGGPNTMANQAK |
| | | NWLPGPCYRQQRVSTTTGQNNNSNFAWTAGTKYHLNGRNSLANPG |
| | | IAMATHKDDEERFFPSNGILIFGKQNAARDNADYSDVMLTSEEEIKT |
| | | TNPVATEEYGIVADNLQQQNTAPQIGTVNSQGALPGMVWQNRDVY |
| | | LQGPIWAKIPHTDGNFHPSPLMGGFGLKHPPPQILIKNTPVPADPPTTF |
| | | NQSKLNSFITQYSTGQVSVEIEWELQKENSKRWNPEIQYTSNYYKST |
| | | SVDFAVNTEGVYSEPRPIGTRYLTRNL |
| 72 | Human RPE65 | atgtctatccaggttgagcatcctgctggtggttacaagaaactgtttgaaactgtggaggaactgtcctcgcc |
| | coding sequence | gctcacagctcatgtaacaggcaggatccccctctggctcaccggcagtctccttcgatgtgggccaggact |
| | (Gene ID: 6121; | ctttgaagttggatctgagccattttaccacctgtttgatgggcaagccctcctgcacaagtttgactttaaagaa |
| | CCDS643.1) | ggacatgtcacataccacagaaggttcatccgcactgatgcttacgtacgggcaatgactgagaaaaggatc |
| | | gtcataacagaatttggcacctgtgctttcccagatccctgcaagaatatattttccaggtttttttcttactttcgag |
| | | gagtagaggttactgacaatgcccttgttaatgtctacccagtgggggaagattactacgcttgcacagagac |
| | | caactttattacaaagattaatccagagaccttggagacaattaagcaggttgatctttgcaactatgtctctgtc |
| | | aatggggccactgctcacccccacattgaaaatgatggaaccgtttacaatattggtaattgctttggaaaaaat |
| | | ttttcaattgcctacaacattgtaaagatcccaccactgcaagcagacaaggaagatccaataagcaagtcag |
| | | agatcgttgtacaattcccctgcagtgaccgattcaagccatcttacgttcatagttttggtctgactcccaactat |
| | | atcgtttttgtggagacaccagtcaaaattaacctgttcaagttcctttcttcatggagtctttggggagccaacta |
| | | catggattgttttgagtccaatgaaaccatgggggtttggcttcatattgctgacaaaaaaaggaaaaagtacct |
| | | caataataaatacagaacttctcctttcaacctcttccatcacatcaacacctatgaagacaatgggtttctgattg |
| | | tggatctctgctgctggaaaggatttgagtttgtttataattacttatatttagccaatttacgtgagaactgggaa |
| | | gaggtgaaaaaaaatgccagaaaggctccccaacctgaagttaggagatatgtacttcctttgaatattgaca |
| | | aggctgacacaggcaagaatttagtcacgctccccaatacaactgccactgcaattctgtgcagtgacgaga |
| | | ctatctggctggagcctgaagttctcttttcagggcctcgtcaagcatttgagtttcctcaaatcaattaccagaa |
| | | gtattgtgggaaaccttacacatatgcgtatggacttggcttgaatcactttgttccagataggctctgtaagctg |
| | | aatgtcaaaactaaagaaacttgggtttggcaagagcctgattcatacccatcagaacccatctttgtttctcac |
| | | ccagatgccttggaagaagatgatggtgtagttctgagtgtggtggtgagcccaggagcaggacaaaagcc |
| | | tgcttatctcctgattctgaatgccaaggacttaagtgaagttgcccgggctgaagtggagattaacatccctgt |
| | | cacctttcatggactgttcaaaaaatcttga |
| 73 | Human RPE65 | MSIQVEHPAGGYKKLFETVEELSSPLTAHVTGRIPLWLTGSLLRCGP |
| | amino acid | GLFEVGSEPFYHLFDGQALLHKFDFKEGHVTYHRRFIRTDAYVRAM |
| | sequence | TEKRIVITEFGTCAFPDPCKNIFSRFFSYFRGVEVTDNALVNVYPVGE |
| | (Uniprot ID: | DYYACTETNFITKINPETLETIKQVDLCNYVSVNGATAHPHIENDGT |
| | Q16518; | VYNIGNCFGKNFSIAYNIVKIPPLQADKEDPISKSEIVVQFPCSDRFKP |
| | CCDS643.1) | SYVHSFGLTPNYIVFVETPVKINLFKFLSSWSLWGANYMDCFESNET |
| | | MGVWLHIADKKRKKYLNNKYRTSPFNLFHHINTYEDNGFLIVDLCC |
| | | WKGFEFVYNYLYLANLRENWEEVKKNARKAPQPEVRRYVLPLNID |
| | | KADTGKNLVTLPNTTATAILCSDETIWLEPEVLFSGPRQAFEFPQINY |
| | | QKYCGKPYTYAYGLGLNHFVPDRLCKLNVKTKETWVWQEPDSYPS |
| | | EPIFVSHPDALEEDDGVVLSVVVSPGAGQKPAYLLILNAKDLSEVAR |
| | | AEVEINIPVTFHGLFKKS |
| 74 | Human LRAT | atgaagaaccccatgctggaggtggtgtctttactactggagaagctgctcctcatctccaacttcacgctcttt |
| | coding sequence | agttcgggcgccgcgggcgaagacaaagggaggaacagtttttatgaaaccagctctttccaccgaggcga |
| | (Genbank Gene | cgtgctggaggtgccccggacccacctgacccactatggcatctacctaggagacaaccgtgttgcccacat |
| | ID: 9227; | gatgcccgacatcctgttggccctgacagacgacatggggcgcacgcagaaggtggtctccaacaagcgt |
| | CCDS3789.1) | ctcatcctgggcgttattgtcaaagtggccagcatccgcgtggacacagtggaggacttcgcctacggagct |
| | | aacatcctggtcaatcacctggacgagtccctccagaaaaaggcactgctcaacgaggaggtggcgcgga |
| | | gggctgaaaagctgctgggctttaccccctacagcctgctgtggaacaactgcgagcacttcgtgacctact |
| | | gcagatatggcaccccgatcagtccccagtccgacaagttttgtgagactgtgaagataattattcgtgatcag |
| | | agaagtgttcttgcttcagcagtcttgggattggcgtctatagtctgtacgggcttggtatcatacactacccttc |
| | | ctgcaatttttattccattcttcctatggatggctggctaa |
| 75 | Human LRAT | MKNPMLEVVSLLLEKLLLISNFTLFSSGAAGEDKGRNSFYETSSFHR |
| | amino acid | GDVLEVPRTHLTHYGIYLGDNRVAHMMPDILLALTDDMGRTQKVV |
| | sequence | SNKRLILGVIVKVASIRVDTVEDFAYGANILVNHLDESLQKKALLNE |
| | (Uniprot ID: | EVARRAEKLLGFTPYSLLWNNCEHFVTYCRYGTPISPQSDKFCETVK |
| | O95237; | IIIRDQRSVLASAVLGLASIVCTGLVSYTTLPAIFIPFFLWMAG |
| | CCDS3789.1) | |
| 76 | Human RDH5 | atgtggctgcctcttctgctgggtgccttactctgggcagtgctgtggttgctcagggaccggcagagcctgc |
| | coding sequence | ccgccagcaatgcctttgtcttcatcaccggctgtgactcaggctttgggcgccttctggcactgcagctggac |
| | (Gene ID: 5959; | cagagaggcttccgagtcctggccagctgcctgaccccctccggggccgaggacctgcagcgggtggcct |
| | CCDS31829.1) | cctcccgcctccacaccaccctgttggatatcactgatccccagagcgtccagcaggcagccaagtgggtg |
| | | gagatgcacgttaaggaagcagggctttttggtctggtgaataatgctggtgtggctggtatcatcggaccca |
| | | caccatggctgacccgggacgatttccagcgggtgctgaatgtgaacacaatgggtcccatcggggtcacc |
| | | cttgccctgctgcctctgctgcagcaagcccggggccgggtgatcaacatcaccagcgtcctgggtcgcct |
| | | ggcagccaatggtgggggctactgtgtctccaaatttggcctggaggccttctctgacagcctgaggcggga |
| | | tgtagctcattttgggatacgagtctccatcgtggagcctggcttcttccgaacccctgtgaccaacctggaga |
| | | gtctggagaaaaccctgcaggcctgctgggcacggctgcctcctgccacacaggcccactatgggggggc |
| | | cttcctcaccaagtacctgaaaatgcaacagcgcatcatgaacctgatctgtgacccggacctaaccaaggt |
| | | gagccgatgcctggagcatgccctgactgctcgacacccccgaacccgctacagcccaggttgggatgcc |
| | | aagctgctctggctgcctgcctcctacctgccagccagcctggtggatgctgtgctcacctgggtccttccca |
| | | agcctgcccaagcagtctactga |
| 77 | Human RDH5 | MWLPLLLGALLWAVLWLLRDRQSLPASNAFVFITGCDSGFGRLLAL |
| | amino acid | QLDQRGFRVLASCLTPSGAEDLQRVASSRLHTTLLDITDPQSVQQAA |
| | sequence | KWVEMHVKEAGLFGLVNNAGVAGIIGPTPWLTRDDFQRVLNVNTM |
| | (UniProtKB - | GPIGVTLALLPLLQQARGRVINITSVLGRLAANGGGYCVSKFGLEAF |
| | Q92781; | SDSLRRDVAHFGIRVSIVEPGFFRTPVTNLESLEKTLQACWARLPPAT |
| | CCDS31829.1) | QAHYGGAFLTKYLKMQQRIMNLICDPDLTKVSRCLEHALTARHPRT |
| | | RYSPGWDAKLLWLPASYLPASLVDAVLTWVLPKPAQAVY |

In one aspect, the present disclosure is related to a single-stranded AAV vector genome comprising, in the 5′ to 3′ direction: (i) a 5′ ITR, (ii) a recombinant nucleotide sequence comprising a CRALBP coding sequence, and (iii) a 3′ ITR. In one aspect, a recombinant nucleotide sequence comprises in the 5′ to 3′ direction: (i) a promoter, (ii) a CRALBP coding sequence, and (iii) an SV40 poly(A) sequence. In one aspect, a promoter can be an RLBP1 (short) promoter, an RLBP1 (long) promoter, or a truncated promoter of RLBP1. In one aspect, a 5′ ITR comprises a nucleic acid sequence set forth in SEQ ID NO: 2. In another aspect, a 5′ ITR comprises a nucleic acid sequence as set forth in SEQ ID NO: 16 or 17. In one aspect, a 3′ ITR comprises a nucleic acid sequence as set forth in SEQ ID NO: 9.

In one aspect, an AAV vector comprises an AAV2 capsid (encoded by SEQ ID NO: 18) and a vector genome comprising in the 5′ to 3′ direction nucleotide sequences selected from the following: a) SEQ ID NO: 2, 10, 5, 6, 8, and 9; b) SEQ ID NO: 2, 11, 5, 6, 8, 14, and 9; c) SEQ ID NO: 2, 22, 5, 6, 8, 23, and 9; and d) SEQ ID NO: 2, 3, 4, 5, 6, 8, 23, and 9. In one aspect, an AAV2 capsid comprises capsid proteins VP1, VP2, and VP3 having an amino acid sequence of SEQ ID NO: 19, 68, and 69, respectively. In another aspect, an AAV2 capsid comprises sub-combinations of capsid proteins VP1, VP2, and/or VP3.

In another aspect, an AAV vector comprises an AAV8 capsid (encoded by SEQ ID NO: 20) and a vector genome comprising in the 5′ to 3′ direction nucleotide sequences selected from the following: a) SEQ ID NO: 2, 10, 5, 6, 8, and 9; b) SEQ ID NO: 2, 11, 5, 6, 8, 14, and 9; c) SEQ ID NO: 2, 22, 5, 6, 8, 23, and 9; and d) SEQ ID NO: 2, 3, 4, 5, 6, 8, 23, and 9. In one aspect, an AAV8 capsid comprises capsid proteins VP1, VP2, and VP3 having an amino acid sequence of SEQ ID NO: 21, 70, and 71, respectively. In another aspect, the AAV8 capsid may comprise sub-combinations of capsid proteins VP1, VP2, and/or VP3.

An AAV vector of the present disclosure can comprise a self-complementary genome. Self-complementary AAV vectors have been previously described in the art and can be adapted for use in the present disclosure. See U.S. Pat. Nos. 7,465,583 and 9,163,259, McCarty 2008, which are all incorporated by reference in their entirety. A self-complementary genome comprises a 5′ ITR and a 3′ ITR (i.e., resolvable ITR or wild-type ITR) at either end of the genome and a non-resolvable ITR (e.g., ΔITR, as set forth in SEQ ID NO: 1) interposed between the 5′ and 3′ ITRs. Each portion of the genome (i.e., between each resolvable ITR and non-resolvable ITR) comprises a recombinant nucleotide sequence, wherein each half (i.e., the first recombinant nucleotide sequence and the second recombinant nucleotide sequence) is complementary to the other, or self-complementary. In other words, a self-complementary vector genome is essentially an inverted repeat with the two halves joined by the non-resolvable ITR. In one aspect the present disclosure is related to a self-complementary vector genome comprising, in the 5′ to 3′ direction, (i) a 5′ ITR, (ii) a first recombinant nucleotide sequence, (iii) a non-resolvable ITR, (iv) a second recombinant nucleotide sequence, and (v) a 3′ ITR. In a certain aspect of the present disclosure the second recombinant nucleotide sequence of the vector genome comprises, an RLBP1 promoter, a CRALBP-coding sequence, and an SV40 poly(A) sequence and the first recombinant nucleotide sequence is self-complementary to the second nucleotide sequence.

In one aspect, an RLBP1 promoter has the nucleotide sequence of SEQ ID NO: 3 or a functional portion thereof. In one aspect of the present disclosure, a second recombinant nucleotide sequence comprises nucleic acid sequences in the 5′ to 3′ direction of SEQ ID NO: 3, 4, 5, 6, and 8 and the first recombinant nucleotide sequence comprises sequences that are self-complementary to, or the reverse complement of, the second recombinant sequence, for example, SEQ ID NOs: 62, 63, 64, 65, and 66. It is also contemplated that the viral vector of the present disclosure can comprise a self-complementary genome wherein the first recombinant nucleotide sequence of the vector genome comprises, an RLBP1 promoter, an RLBP1 coding sequence, and an SV40 polyA sequence and the second recombinant nucleotide sequence is self-complementary to the first recombinant nucleotide sequence.

In one aspect, a self-complementary viral vector comprises an AAV2 capsid (encoded by SEQ ID NO: 18) and a vector genome comprising a nucleotide sequence comprising sequences, in the 5′ to 3′ direction, SEQ ID NOs: 36, 62, 63, 64, 65, 66, 1, 3, 4, 5, 6, 8, and 9. In one aspect, an AAV2 capsid comprises capsid proteins VP1, VP2, and VP3 having an amino acid sequence of SEQ ID NO: 19, 68, and 69, respectively. In certain other aspects, an AAV2 capsid can comprise sub-combinations of capsid proteins VP1, VP2, and/or VP3.

In one aspect, a self-complementary viral vector comprises an AAV8 capsid (encoded by SEQ ID NO: 20) and a vector genome comprising a nucleotide sequence comprising sequences in the 5′ to 3′ direction SEQ ID NOs: 36, 62, 63, 64, 65, 66, 1, 3, 4, 5, 6, 8, and 9. In one aspect, an AAV8 capsid comprises capsid proteins VP1, VP2, and VP3 having an amino acid sequence of SEQ ID NO: 21, 70, and 71. In certain other aspects, an AAV8 capsid can comprise sub-combinations of capsid proteins VP1, VP2, and/or VP3.

AAV vectors of the present disclosure can be used to express CRALBP protein in RPE cells and Müller cells of the retina in a subject suffering from eye diseases or blindness.

Methods for generating viral vectors are well known in the art and are described in U.S. Pat. No. 9,163,259 B2, which is incorporated by reference in its entirety. The plasmids used in U.S. Pat. No. 9,163,259 B2 are summarized in Table 2 and the AAV vectors generated therefrom are described in Table 3 in Example 1 below.

The genetic elements as described in Table 2 are in the context of a circular plasmid, but one of skill in the art will appreciated that a DNA substrate may be provided in any form known in the art, including but not limited to, a plasmid, naked DNA vector, bacterial artificial chromosome (BAC), yeast artificial chromosome (YAC), or a viral vector (e.g., adenovirus, herpesvirus, Epstein-Barr Virus, AAV, baculoviral, retroviral vectors, and the like). Alternatively, the genetic elements in Table 2 necessary to produce the viral vectors described herein may be stably incorporated into the genome of a packaging cell.

In one aspect, an AAV vector of the present disclosure can be produced by providing to a cell permissive for parvovirus replication: (a) an AAV-ITR-containing plasmid comprising a heterologous gene encoding a CRALBP protein; (b) an AAV-Rep-Cap-containing plasmid; (c) a helper plasmid.

Any method of introducing a nucleotide sequence carrying a CRALBP-coding sequence into a cellular host for replication and packaging may be employed, including but not limited to, electroporation, calcium phosphate precipitation, microinjection, cationic or anionic liposomes, and liposomes in combination with a nuclear localization signal.

AAV vectors described herein can be produced using methods known in the art, such as, for example, triple transfection or baculovirus mediated virus production. Any suitable permissive or packaging cell known in the art may be employed to produce the vectors. Mammalian cells are preferred. Also preferred are trans-complementing packaging cell lines that provide functions deleted from a replication-defective helper virus, e.g., HEK293 cells or other Ela trans-complementing cells.

A nucleotide sequence containing a gene of interest can contain some or all of the AAV Cap and/or Rep genes. Preferably, however, some or all of the Cap and Rep functions are provided in trans by introducing a packaging vector(s) encoding the capsid and/or Rep proteins into the cell. Most preferably, the nucleotide sequence containing a gene of interest does not encode the capsid or Rep proteins. Alternatively, a packaging cell line is used that is stably transformed to express the Cap and/or Rep genes.

In addition, helper virus functions are provided for an AAV vector to propagate new virus particles. Both adenovirus and herpes simplex virus may serve as helper viruses for AAV. Exemplary helper plasmid viruses include, but are not limited to, Herpes simplex (HSV) varicella zoster, cytomegalovirus, and Epstein-Barr virus. The multiplicity of infection (MOI) and the duration of the infection will depend on the type of virus used and the packaging cell line employed. Any suitable helper vector may be employed. Preferably, a vector is a plasmid. The vector can be introduced into the packaging cell by any suitable method known in the art, as described above.

In summary, a gene cassette containing a gene of interest (e.g., CRALBP) to be replicated and packaged, AAV capsid and Rep genes, and helper functions are provided to a cell (e.g., a permissive or packaging cell) to produce AAV particles carrying the gene of interest. The combined expression of the Rep and Cap genes encoded by the gene cassette and/or the packaging vector(s) and/or the stably transformed packaging cell results in the production of an AAV vector particle in which an AAV vector capsid packages an AAV vector according to the present disclosure. Single stranded or self-complementary AAV vectors are allowed to assemble within the cell, and may then be recovered by any method known by those of skill in the art and described in the examples. For example, viral vectors may be purified by standard CsCl centrifugation methods or by various methods of column chromatography known to the skilled artisan.

Reagents and methods disclosed herein can be employed to produce high titer stocks of AAV vectors, preferably at essentially wild-type titers. It is also preferred that the parvovirus stock has a titer of at least about 10⁵ transducing units (tu)/ml, more preferably at least about 10⁶ tu/ml, more preferably at least about 10⁷ tu/ml, yet more preferably at least about 10⁸ tu/ml, yet more preferably at least about 10⁹ tu/ml, still yet more preferably at least about 10¹⁰ to/ml, still more preferably at least about 10¹¹ tu/ml or more.

An AAV vector produced as described in the present disclosure can be contacted with a cell to produce a cell lysate in a method for measuring CRALBP activity. In one aspect, an amount of about 500 to about 5×10⁶ of an AAV vector per cell can be used. In another aspect, an amount of about 1,000 to about 1×10⁶ of an AAV vector per cell can be used. In yet another aspect, an amount of about 2,000 to about 5×10⁵ of an AAV vector per cell can be used.

### Nucleic Acids Used in Generating an AAV Vector

In one aspect of the present disclosure, nucleic acids useful for the generation of AAV vectors of the present disclosure can be in the form of plasmids. Plasmids useful for the generation of viral vectors, also referred to as a viral vector plasmid, may contain a gene cassette. At a minimum, a gene cassette of a viral vector plasmid contains: a heterologous gene and its regulatory elements (e.g., promoter, enhancer, and/or introns, etc.), and 5′ and 3′ AAV inverted terminal repeats (ITRs).

In one aspect, a heterologous gene in the present disclosure comprises a CRALBP-encoding sequence. In one aspect, a CRALBP-coding sequence comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6. In another aspect, a CRALBP-coding sequence encodes a protein that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7. In another aspect, a CRALBP-coding sequence comprises a nucleic acid sequence selected from the group consisting of SEQ ID NOs: 37, 39, 41, 43, 45, and 47. In another aspect, a recombinant CRALBP-coding sequence encodes a protein that comprises an amino acid sequence selected from the group consisting of SEQ ID NOs: 38, 40, 42, 44, 46, and 48.

In addition to the heterologous gene, a gene cassette may include regulatory elements operably linked to the heterologous gene. These regulatory elements may include appropriate transcription initiation, termination, promoter and enhancer sequences, efficient RNA processing signals such as splicing and polyadenylation (polyA) signals; sequences that stabilize cytoplasmic mRNA; sequences that enhance translation efficiency; sequences that enhance protein stability; and when desired, sequences that enhance secretion of the encoded product. A great number of regulatory sequences, including promoters which are native, constitutive, inducible, and/or tissue-specific, are known in the art and may be utilized. In one aspect, a recombinant CRALBP-coding sequence is operably linked to a promoter sequence selected from the group consisting of SEQ ID NOs: 3, 10, 11, 12, 22, and a functional portion thereof. In another aspect, a recombinant CRALBP-coding sequence is operably linked to a regulatory element selected from the group consisting of SEQ ID NO: 3, 4, 5, 8, 10, 11, 12, 22, and a functional portion thereof.

In one aspect, a promoter with a nucleic acid sequence of SEQ ID NO: 3 or 10 is operably linked to a heterologous gene. In particular, a RLBP1 short promoter (SEQ ID NO: 3) is operably linked to a CRALBP-coding sequence as set forth in SEQ ID NO: 6. In another aspect, a RLBP1 short promoter (SEQ ID NO: 3) is operably linked to a CRALBP-coding sequence selected from the group consisting of SEQ ID NOs: 37, 39, 41, 43, 45, and 47. Alternatively, a RLBP1 long promoter (SEQ ID NO: 10) is operably linked to a CRALBP-coding sequence as set forth in SEQ ID NO: 6. In another aspect, a RLBP1 long promoter (SEQ ID NO: 10) is operably linked to a CRALBP-coding sequence selected from the group consisting of SEQ ID NOs: 37, 39, 41, 43, 45, and 47.

It is contemplated that ITRs of AAV serotype 2 can be used (e.g., SEQ ID NO: 2, 9, 16, 17, or 36). However, ITRs from other suitable serotypes can be selected from among any AAV serotype known in the art, as described herein. These ITRs or other AAV components can be readily isolated using techniques available to those of skill in the art from any AAV serotype known, or yet to be identified serotypes.

In one aspect of the present disclosure, one ITR can be a modified ITR, or non-resolvable ITR, i.e., a sequence without the terminal resolution site (TRS). During replication of a gene cassette comprising a non-resolvable ITR, the inability of Rep protein to resolve the non-resolvable ITRs will result in a dimeric inverted repeat sequence (i.e., self-complementary) with a non-resolvable ITR (e.g., ΔITR) in the middle and a wild-type ITR at each end. The resulting sequence is a self-complementary viral genome sequence such that the genome is capable of forming a hairpin structure upon release from the capsid. A non-resolvable ITR may be produced by any method known in the art. For example, insertion into the ITR will displace the TRS and result in a non-resolvable ITR. In one aspect, the insertion is in the region of the TRS site. In one aspect, an ITR can be rendered non-resolvable by deletion of the TRS site, resulting in a ΔITR as set forth in SEQ ID NO: 1.

In one aspect, a nucleic acid sequence of the present disclosure comprises, in the 5′ to 3′ direction, nucleic acid sequences selected from the group consisting of: a) SEQ ID NOs: 2, 10, 5, 6, 8, and 9; b) SEQ ID NOs: 2, 11, 5, 6, 8, 14 and 9; c) SEQ ID NOs: 2, 22, 5, 6, 8, 23 and 9; d) SEQ ID NOs: 2, 3, 4, 5, 6, 8, 23 and 9; e) SEQ ID NOs: 2, 10, 5, 24, 8, and 9; f) SEQ ID NOs: 2, 11, 24, 8, 14, and 9; and g) SEQ ID NOs: 2, 12, 24, 8, 14, and 9. In one aspect, a nucleic acid sequence comprising a gene cassette can be a plasmid. In particular, the sequence of the plasmid may have a sequence selected from SEQ ID NOs: 27, 28, 29, 30, 32, 33, 34 and 35.

In another aspect, a nucleic acid sequence of the present disclosure comprises, in the 5′ to 3′ direction, nucleic acid sequences selected from the group consisting of: a) SEQ ID NOs: 1, 5, 6, 8, and 9; and b) SEQ ID NOs: 1, 3, 4, 5, 6, 8, and 9. In one aspect, a nucleic acid sequence comprising a gene cassette can be a plasmid. In particular, the sequence of the plasmid may have a sequence selected from SEQ ID NOs: 26, 31, and 50.

Viral vectors as described herein, can be used at a therapeutically useful concentration for the treatment of eye related diseases, by administering to a subject in need thereof, an effective amount of the viral vectors of the present disclosure.

### CRALBP Activity Assay

The present disclosure provides a method for measuring activity of CRALBP or potency of an AAV vector comprising a CRALBP coding sequence for expressing a CRALBP protein. The method comprises a) contacting a cell with an adeno-associated viral (AAV) vector comprising a heterologous gene encoding a CRALBP protein, whereby a transduced cell expressing the CRALBP protein is generated; b) lysing the transduced cell to produce a cell extract thereof; c) incubating the cell extract with a composition comprising a substrate of the vision cycle, under conditions wherein the substrate is converted to a reaction product in the presence of CRALBP protein; and d) determining the reaction product, whereby the amount of the reaction product reflects the activity of the CRALBP protein. Also provided in the present disclosure is a kit for use in measuring activity of CRALBP comprising: a) an AAV-ITR-containing plasmid comprising a heterologous gene encoding a CRALBP protein; b) an AAV-Rep-Cap-containing plasmid; c) an helper plasmid; and d) a composition comprising a substrate. In one aspect, a kit further comprises a cell expressing a protein having LRAT activity. In another aspect, a kit further comprises a protein having LRAT activity.

Methods for preparing a cell extract for the present disclosure is known in the art. A DNA sequence encoding LRAT can be introduced into an expression vector appropriate for expression in a host cell. Potential host-vector systems include, but are not limited to, mammalian cell systems transfected with expression plasmids or infected with virus (e.g., vaccinia virus, adenovirus, AAV, herpes virus, etc.); insect cell systems infected with virus (e.g., baculovirus); microorganisms such as yeast containing yeast vectors; or bacteria transformed with bacteriophage, DNA, plasmid DNA, or cosmid DNA.

In one aspect, a method of the present disclosure comprises contacting an AAV vector with a cell expressing a protein having LRAT activity. In one aspect, a cell expressing a protein having LRAT activity is a mammalian cell. In another aspect, a cell expressing a protein having LRAT activity is a human cell. In one aspect, a cell extract comprising a protein having LRAT activity is obtained from a cell stably expressing LRAT. In one aspect, a cell stably expressing LRAT is an HEK293 cell. In another aspect, a cell stably expressing LRAT is a HeLa cell. In another aspect, a cell extract comprising a protein having LRAT activity is obtained from a cell transiently expressing LRAT. In another aspect, a cell transiently expressing LRAT is an HEK293 cell. In another aspect, a cell transiently expressing LRAT is a HeLa cell

A wide variety of cell lines for use in the presently disclosed methods are known in the art. Examples of cell lines include, but are not limited to, C8161, CCRF-CEM, MOLT, mIMCD-3, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts, 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, Hepa1c1c7, HMEC, HT-29, Jurkat, JY cells, K562 cells, Ku812, KCL22, KG1, KYO1, LNCap, Ma-MeI 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (See, e.g., the American Type Culture Collection (ATCC) (Manassas, Va.)).

In some aspects, a cell extract comprising a protein having LRAT activity is obtained from a cell transduced with an AAV vector comprising a LRAT-coding sequence. In another aspect, a cell extract comprising a protein having LRAT activity is obtained from a cell transduced with a baculovirus-based expression system. In yet another aspect, a cell extract comprising a protein having LRAT activity is obtained from a cell transduced with a herpes virus-based expression system.

In one aspect, a protein having LRAT activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 74. In another aspect, a protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75.

A first DNA sequence encoding LRAT and second DNA sequence encoding RPE65 can be co-introduced into a host cell by using standard methods known in the art. A cell lysate produced therefrom can be used in an assay for measuring activity of CRALBP. In one aspect, a first DNA sequence encoding LRAT and second DNA sequence encoding RPE65 are stably or transiently expressed from a mammalian cell. In one aspect, the mammalian cell is an HEK293 cell. In another aspect, the mammalian cell is a HeLa cell. In one aspect, an HEK293 cell is transduced with an AAV vector containing a LRAT-coding sequence and an RPE65-coding sequence. In another aspect, a HeLa cell is transduced with an AAV vector containing a LRAT-coding sequence and an RPE65-coding sequence. In yet another aspect, an mammalian cell is transduced with a herpes virus vector containing a LRAT-coding sequence and an RPE65-coding sequence. In one aspect, a cell lysate is prepared by lysing a host, transduced cell. In one aspect, the lysing comprises freeze-thawing, sonication, or a combination thereof. In one aspect, after lysing the host, transduced cell the cell lysate is diluted in a salt buffer. In another aspect, the salt buffer is a sodium chloride buffer.

In one aspect, a protein having LRAT and/or RPE65 activity can be isolated from a host cell and added to a cell lysate in the presence of CRALBP and one or more substrate in a method for measuring CRALBP activity. Recombinant protein having LRAT and/or RPE65 activity can be tagged with an N- or C-terminal tag, including HA, His, GST, FLAG or other suitable tags, and be purified using standard methods in the art. Recombinant protein having LRAT and/or RPE65 protein can also be purified by using methods based on size, affinity, and/or polarity/hydrophobicity, which include, but are not limited to, size exclusion chromatograph, hydrophobic interaction chromatography, ion exchange chromatography, free-flow-electrophoresis, affinity chromatography, metal binding, immuno-affinity chromatography, HPLC, and reverse-phase chromatography.

In one aspect, an RPE65 protein or a protein having RPE65 activity is a mammalian or a human RPE65. In one aspect, an RPE65 protein or a protein having RPE65 activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 72. In another aspect, an RPE65 protein or a protein having RPE65 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 73.

A cell lysate containing a protein having LRAT activity and CRALBP is incubated with a composition comprising a protein having RPE65 activity and a substrate in an assay for measuring the amount of a reaction product which reflects the CRALBP activity. The incubation is performed in the dark, under dim light, or under dim yellow light. In one aspect, the incubation is at a temperature from about 30° C. to about 40° C. In one aspect, the incubation is from about 30 minutes to about 240 minutes. In another aspect, the incubation is from about 6 hours to about 96 hours. The incubation is then quenched or stopped. In one aspect, an alcohol is added to quench or stop the reaction. A reaction product is extracted with an organic solvent for purification and/or quantification. In one aspect, an organic solvent is hexane.

A composition comprising a substrate is added to a cell lysate. In one aspect, a substrate is all-trans retinyl ester and a reaction product is 11-cis retinol. Without being bound by any theory, all-trans retinyl ester can be converted to 11-cis retinol by RPE65. Without being bound by any theory, the presence of CRALBP increases the conversion from all-trans retinyl ester to 11-cis retinol. See e.g., WO 2017/190081 A1. In another aspect, a substrate comprises a precursor to the substrate. In one aspect, a precursor to a substrate is all-trans retinol and a reaction product is 11-cis retinol. Without being bound by any theory, all-trans retinol can be converted by LRAT to all-trans retinyl ester, which can be in turn converted to 11-cis retinol by RPE65 in the presence of CRALBP. In one aspect, all-trans retinol is mixed with an at least 10% solution of dimethylformamide (DMF). In one aspect, all-trans retinol is added such that the final concentration is about 1 mM to about 20 mM. The amount of the reaction product, 11-cis retinol, can be measured as described in the present disclosure which reflects the activity of the CRALBP protein.

In another aspect, a protein having RDH5 activity can be added to a cell lysate containing proteins having LRAT and RPE65 activity and CRALBP together with a substrate, wherein the substrate is all-trans retinol or all-trans retinyl ester and a reaction product is 11-cis retinal. Without being bound by any theory, all-trans retinol or all-trans retinyl ester can be converted to 11-cis retinol which can be in turn converted to 11-cis retinal by a protein having RDH5 activity. The amount of the reaction product, 11-cis retinal, can be measured as described in the present disclosure which reflects the activity of the CRALBP protein.

In one aspect, a protein having RDH5 activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 76. In another aspect, a protein having RDH5 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 77.

Methods for isolating and purifying a reaction product of the present disclosure are known in the art. In one aspect, the purification of a reaction product comprises subjecting the reaction product to column chromatography, thereby producing a column chromatography purified reaction product. In one aspect, a column chromatography comprises a reverse-phase chromatography. In another aspect, a column chromatography comprises a reverse-phase stationary phase. In one aspect, a method for measuring CRALBP activity comprises subjecting the column chromatography purified reaction product to mass spectrometry, thereby quantifying the reaction product.

### EMBODIMENTS

The following are exemplary embodiments of the present specification.

Embodiment 1. A method for measuring activity of cellular retinaldehyde-binding protein (CRALBP) comprising:
a. contacting a cell with an adeno-associated viral (AAV) vector comprising a heterologous gene encoding a CRALBP protein, whereby a transduced cell expressing the CRALBP protein is generated;
b. lysing the transduced cell to produce a cell extract thereof;
c. incubating the cell extract with a composition comprising a substrate of the vision cycle, under conditions wherein the substrate is converted to a reaction product in the presence of CRALBP protein; and
d. determining the reaction product, whereby the amount of the reaction product reflects the activity of the CRALBP protein.

Embodiment 2. A method for measuring potency of a composition comprising an AAV vector comprising a CRALBP coding sequence for expressing a CRALBP protein, the method comprising:
a. contacting a cell with the AAV vector, whereby a transduced cell expressing the CRALBP protein is generated;
b. lysing the transduced cell to produce a cell extract thereof;
c. incubating the cell extract with a composition comprising a substrate of the vision cycle, wherein the substrate is converted to a reaction product in the presence of CRALBP protein; and
d. determining the reaction product, whereby the amount of the reaction product reflects the activity of the CRALBP protein.

Embodiment 3. The method of embodiment 1 or 2, wherein the cell expresses a protein having lecithin retinol acyltransferase (LRAT) activity.

Embodiment 4. The method of embodiment 1 or 2, wherein the composition further comprises a protein having LRAT activity.

Embodiment 5. The method of any one of embodiments 1 to 4, wherein the substrate in step (c) is all-trans retinyl ester or all-trans retinol.

Embodiment 6. The method of any one of embodiments 1 to 5, wherein the reaction product is 11-cis retinol.

Embodiment 7. The method of embodiment 6, wherein the composition in step (c) further comprises a protein having retinal pigment epithelium-specific protein 65-KD (RPE65) activity.

Embodiment 8. The method of embodiment 7, wherein the protein having RPE65 activity is a mammalian RPE65.

Embodiment 9. The method of embodiment 7, wherein the protein having RPE65 activity is a human RPE65.

Embodiment 10. The method of any one of embodiments 7 to 9, wherein the protein having RPE65 activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 72.

Embodiment 11. The method of any one of embodiments 7 to 9, wherein the protein having RPE65 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 73.

Embodiment 12. The method of any one of embodiments 1 to 11, wherein the reaction product comprises 11-cis retinal.

Embodiment 13. The method of embodiment 12, wherein the composition in step (c) further comprises a protein having RPE65 activity and a protein having 11-cis retinol dehydrogenase 5 (RDH5) activity.

Embodiment 14. The method of embodiment 13, wherein the protein having RDH5 activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 76.

Embodiment 15. The method of embodiment 13, wherein the protein having RDH5 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 77.

Embodiment 16. The method of any one of embodiments 1 to 15, wherein the AAV vector comprises in the 5′ to 3′ direction:
a. a 5′ inverted terminal repeat (ITR);
b. a recombinant CRALBP-coding sequence; and
c. a 3′ ITR.

Embodiment 17. The method of embodiment 16, wherein the recombinant CRALBP-coding sequence is operably linked to a promoter sequence selected from the group consisting of SEQ ID NOs: 3, 10, 11, 12, and 22.

Embodiment 18. The method of embodiment 17, wherein the recombinant CRALBP-coding sequence comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6.

Embodiment 19. The method of embodiment 17, wherein the recombinant CRALBP-coding sequence encodes a protein that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7.

Embodiment 20. The method of embodiment 17, wherein the recombinant CRALBP-coding sequence comprises a nucleic acid sequence selected from the group consisting of SEQ ID NOs: 37, 39, 41, 43, 45, and 47.

Embodiment 21. The method of embodiment 17, wherein the recombinant CRALBP-coding sequence encodes a protein that comprises an amino acid sequence selected from the group consisting of SEQ ID NOs: 38, 40, 42, 44, 46, and 48.

Embodiment 22. The method of any one of embodiments 16 to 21, wherein the 5′ ITR comprises a nucleic acid sequence set forth in SEQ ID NO: 2.

Embodiment 23. The method of any one of embodiments 16 to 21, wherein the 5′ ITR comprises a nucleic acid sequence as set forth in SEQ ID NO: 16 or 17.

Embodiment 24. The method of any one of embodiments 16 to 23, wherein the AAV vector comprises a nucleic acid sequence, in the 5′ to 3′ direction, selected from the group consisting of:
a. SEQ ID NOs: 2, 10, 5, 6, 8, and 9;
b. SEQ ID NOs: 2, 11, 5, 6, 8, 14, and 9;
c. SEQ ID NOs: 2, 22, 5, 6, 8, 23, and 9; and
d. SEQ ID NOs: 2, 3, 4, 5, 6, 8, 23, and 9.

Embodiment 25. The method of any one of embodiments 16 to 21, wherein the 5′ ITR comprises a non-resolvable ITR.

Embodiment 26. The method of embodiment 25, wherein the non-resolvable ITR comprises a nucleic acid sequence as set forth in SEQ ID NO: 1.

Embodiment 27. The method of embodiment 26, wherein the recombinant CRALBP-coding sequence comprises a nucleic acid sequence as set forth in SEQ ID NO: 6.

Embodiment 28. The method of embodiment 27, wherein the AAV vector comprises a nucleic acid sequence, in the 5′ to 3′ direction, of SEQ ID NOs: 1, 5, 6, 8, and 9.

Embodiment 29. The method of embodiment 28, wherein the AAV vector comprises a nucleic acid sequence, in the 5′ to 3′ direction, of SEQ ID NOs: 1, 3, 4, 5, 6, 8, and 9.

Embodiment 30. The method of embodiment 29, wherein the AAV vector comprises a nucleic acid sequence, in the 5′ to 3′ direction, of SEQ ID NOs: 36, 62, 63, 64, 65, 66, 1, 3, 4, 5, 6, 8, and 9.

Embodiment 31. The method of any one of embodiments 16 to 30, wherein the AAV vector comprises an AAV serotype 2 capsid.

Embodiment 32. The method of embodiment 31, wherein the AAV serotype 2 capsid is encoded by a nucleic acid sequence of SEQ ID NO: 18.

Embodiment 33. The method of any one of embodiments 16 to 30, wherein the AAV vector comprises an AAV serotype 8 capsid.

Embodiment 34. The method of embodiment 33, wherein the AAV serotype 8 capsid is encoded by a nucleic acid sequence of SEQ ID NO: 20.

Embodiment 35. The method of any one of embodiments 16 to 30, wherein the AAV vector comprises an AAV serotype 5 capsid.

Embodiment 36. The method of any one of embodiments 1 to 35, wherein the cell expressing a protein having LRAT activity is a mammalian cell.

Embodiment 37. The method of any one of embodiments 1 to 35, wherein the cell expressing a protein having LRAT activity is a human cell.

Embodiment 38. The method of embodiment 37, wherein the cell expressing a protein having LRAT activity is a HeLa cell.

Embodiment 39. The method of embodiment 37, wherein the cell expressing a protein having LRAT activity is a human embryonic kidney (HEK) 293 cell.

Embodiment 40. The method of any one of embodiments 1 to 39, wherein the cell expresses a protein having LRAT activity stably.

Embodiment 41. The method of any one of embodiments 1 to 39, wherein the cell expresses a protein having LRAT activity transiently.

Embodiment 42. The method of any one of embodiments 1 to 41, wherein the protein having LRAT activity is encoded by a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 74.

Embodiment 43. The method of any one of embodiments 1 to 41, wherein the protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75.

Embodiment 44. The method of any one of embodiments 1 to 43, wherein step (c) comprises adding a precursor of the substrate to the cell extract, whereby the precursor is converted to the substrate.

Embodiment 45. The method of embodiment 44, wherein the precursor comprises all-trans retinol.

Embodiment 46. The method of embodiment 45, wherein the precursor is mixed with an at least 10% solution of dimethylformamide (DMF).

Embodiment 47. The method of embodiment 45, wherein the all-trans retinol is added such that the final concentration is about 1 mM to about 20 mM.

Embodiment 48. The method of any one of embodiments 1 to 47, wherein the contacting in step (a) is with an amount of about 500 to about 5×10⁶ of the AAV vector per cell.

Embodiment 49. The method of embodiment 48, wherein the contacting in step (a) is with an amount of about 1,000 to about 1×10⁶ of the AAV vector per cell.

Embodiment 50. The method of embodiment 49, wherein the contacting in step (a) is with an amount of about 2,000 to about 5×10⁵ of the AAV vector per cell.

Embodiment 51. The method of any one of embodiments 1 to 50, wherein the lysing in step (b) comprises freeze-thawing, sonication, or a combination thereof.

Embodiment 52. The method of embodiment 51, wherein after the lysing in step (b) the transduced cell is diluted in a salt buffer.

Embodiment 53. The method of embodiment 52, wherein the salt buffer is a sodium chloride buffer.

Embodiment 54. The method of any one of embodiments 1 to 53, wherein steps (c) and (d) are performed in the dark, under dim light, or under dim yellow light.

Embodiment 55. The method of any one of embodiments 1 to 54, wherein the incubating in step (c) is from about 30 minutes to about 240 minutes.

Embodiment 56. The method of any one of embodiments 1 to 54, wherein the incubating in step (c) is from about 6 hours to about 96 hours.

Embodiment 57. The method of any one of embodiments 1 to 56, wherein the incubating in step (c) is at a temperature from about 30° C. to about 40° C.

Embodiment 58. The method of embodiment 57, wherein after step (c) but before step (d) the reaction is quenched or stopped.

Embodiment 59. The method of embodiment 58, wherein after step (c) but before step (d) an alcohol is added.

Embodiment 60. The method of any one of embodiments 1 to 59, wherein the reaction product is extracted with an organic solvent.

Embodiment 61. The method of embodiment 60, wherein said organic solvent is hexane.

Embodiment 62. The method of any one of embodiments 1 to 61, wherein the determining in step (d) comprises subjecting the reaction product to column chromatography, thereby producing a column chromatography purified reaction product.

Embodiment 63. The method of embodiment 62, wherein the column chromatography comprises a reverse-phase chromatography.

Embodiment 64. The method of embodiment 62, wherein the column chromatography comprises a reverse-phase stationary phase.

Embodiment 65. The method of embodiment 62, wherein step (d) comprises subjecting the column chromatography purified reaction product to mass spectrometry, thereby quantifying the reaction product.

Embodiment 66. A kit for use in measuring activity of CRALBP comprising:
a. an AAV-ITR-containing plasmid comprising a heterologous gene encoding a CRALBP protein;
b. an AAV-Rep-Cap-containing plasmid;
c. a helper plasmid; and
d. a composition comprising a substrate of the vision cycle.

Embodiment 67. The kit of embodiment 66, further comprising a cell expressing a protein having LRAT activity.

Embodiment 68. The kit of embodiment 66, further comprising a protein having LRAT activity.

Embodiment 69. The kit of any one of embodiments 66 to 68, wherein the composition further comprises a protein having RPE65 activity.

Embodiment 70. The kit of any one of embodiments 66 to 69, wherein the helper plasmid is an Adeno-helper plasmid.

Embodiment 71. The kit of embodiment 67, wherein the cell expressing a protein having LRAT activity is a human embryonic kidney (HEK) 293 cell.

Embodiment 72. The kit of any one of embodiments 67, 68, and 71, wherein the protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75.

Embodiment 73. The kit of any one of embodiments 66 to 72, wherein the recombinant CRALBP-coding sequence comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6.

Embodiment 74. The kit of any one of embodiments 66 to 72, wherein the recombinant CRALBP-coding sequence comprises a nucleic acid sequence selected from the group consisting of SEQ ID NOs: 37, 39, 41, 43, 45, and 47.

Embodiment 75. The kit of any one of embodiments 66 to 74, wherein the AAV-ITR-containing plasmid comprises a nucleic acid sequence selected from the group consisting of SEQ ID NOs: 26, 27, 28, 29, 30, and 50.

Embodiment 76. The kit of embodiment 75, wherein the AAV-ITR-containing plasmid comprises a nucleic acid sequence in the 5′ to 3′ direction, selected from the group consisting of:
a. SEQ ID NOs: 2, 10, 5, 6, 8, and 9;
b. SEQ ID NOs: 2, 11, 5, 6, 8, 14, and 9;
c. SEQ ID NOs: 2, 22, 5, 6, 8, 23, and 9;
d. SEQ ID NOs: 2, 3, 4, 5, 6, 8, 23, and 9; and
e. SEQ ID NOs: 1, 5, 6, 8, and 9.

Embodiment 77. The kit of any one of embodiments 66 to 76, wherein the AAV-Rep-Cap-containing plasmid encodes an AAV serotype 2 capsid.

Embodiment 78. The kit of embodiment 77, wherein the AAV serotype 2 capsid is encoded by a nucleic acid sequence of SEQ ID NO: 18.

Embodiment 79. The kit of any one of embodiments 66 to 76, wherein the AAV-Rep-Cap-containing plasmid encodes an AAV serotype 8 capsid.

Embodiment 80. The kit of embodiment 79, wherein the AAV serotype 8 capsid is encoded by a nucleic acid sequence of SEQ ID NO: 20.

Embodiment 81. The kit of any one of embodiments 66 to 80, wherein the substrate comprises all-trans retinyl ester or all-trans retinol.

Embodiment 82. The kit of embodiment 81, wherein the protein having RPE65 activity is a human RPE65.

Embodiment 83. The kit of embodiment 82, wherein the protein having RPE65 activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 73.

Embodiment 84. A cell for use in a method for measuring activity of CRALBP, wherein the cell recombinantly expresses a protein having LRAT activity and a protein having CRALBP activity.

Embodiment 85. The cell for use in a method for measuring activity of CRALBP of embodiment 84, wherein the protein having LRAT activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 75.

Embodiment 86. The cell for use in a method for measuring activity of CRALBP of embodiment 84, wherein the protein having CRALBP activity comprises an amino acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 7.

Embodiment 87. The cell for use in a method for measuring activity of CRALBP of any one of embodiments 84 to 86, wherein the cell comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 74.

Embodiment 88. The cell for use in a method for measuring activity of CRALBP of any one of embodiments 84 to 87, wherein the cell comprises a nucleic acid sequence that is at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical or complementary to SEQ ID NO: 6.

Embodiment 89. The cell for use in a method for measuring activity of CRALBP of any one of embodiments 84 to 88, wherein the cell is an HEK293 cell.

Embodiment 90. The cell for use in a method for measuring activity of CRALBP of any one of embodiments 84 to 88, wherein the cell is a HeLa cell.

### EXAMPLES

### Example 1. CRALBP Binding Assay

Binding of 11-cis-retinol to human CRALBP protein was assessed for affinity determinations using Biacore. Kinetic rate constants was performed via surface plasmon resonance (SPR) using the Biacore T200 instrument (Cytiva, formerly GE Healthcare Lifesciences) as described below. The proteinA/G capture method was utilized in order to determine kinetics for 11-cis-retinol.

Recombinant proteinA/G (PIERCE, Cat #21186) was immobilized on the chip surface by using amino-coupling procedure according to the supplier's instruction (Cytiva, BR-1000-50). This immobilized proteinA/G captured commercial anti-CRALBP mouse IgG (Sigma, WH0006017M1, lot #11319-1H7), which then captured human CARLBP protein (GeneTex, GTX109228-pro, lot #42226) on chip surface. The 11-cis-retinol (Biosynth Carbosynth, FR163659) flowed over as analyte.

The 11-cis-retinol concentration started at 20004 and was serially diluted at one part to one part for five levels of concentration. Regeneration was performed at the end of each cycle using Glycine-HCl pH2.0 (Cytiva, BR-1003-55). The sample dilution step and Biacore experiment were performed either under ambient light or dark condition in which the Biacore sample compartment door was covered by aluminum foil.

Double reference subtraction was completed to generate final data. Kinetic rate constants was obtained by applying 1:1 binding model with Biacore T200 evaluation 3.0 software, wherein the Rmax values were fit locally. Binding was assess under dark and ambient light conditions.

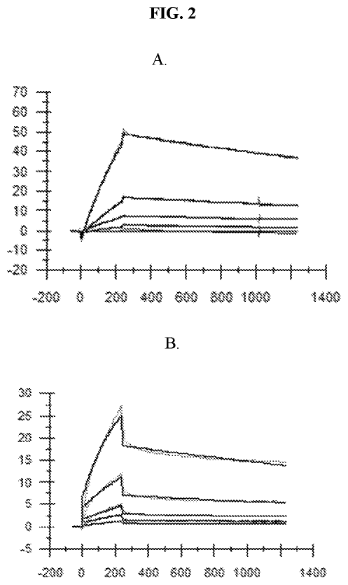

As shown in FIG. 2, 11-cis-retinol was able to bind to human CRALBP in both ambient light (FIG. 2A) and dark (FIG. 2B) conditions. The unit of the X-axis in FIG. 2 is seconds. Kinetic rate constants were measured and summarized in Table 2 below.

TABLE 2
Kinetic rate constants as measured in FIG. 2

| | Ambient light | Dark |
|---|---|---|
| Captured | 100 | 220 |
| huCRALBP | | |
| (RU) | | |
| Ka (1/Ms) | 12.69 | 21.03 |
| Kd (1/s) | 2.89E−04 | 2.85E−04 |
| KD (μM) | 22.8 | 13.6 |

Overall affinities were comparable in both conditions but increased binding signal was observed under ambient light conditions.

Alternatively, binding between CRALBP and 11-cis-retinal can be assessed for affinity determination as described above, e.g., in J. Biol. Chem., 273: 20712-20720, 1998, which is incorporated by reference in its entirety.

### Example 2. Cloning and Preparation of AAV Vectors

AAV vectors for delivering an RLBP1 gene are known in the art. See e.g., US 2019/0071681 A1, US 2016/0194374 A1, and US 2004/0208847 A1, each one of which is incorporated by reference in its entirety. Sequences of AAV-ITR-containing plasmids for generating AAV vectors are described in U.S. Pat. Nos. 9,163,259 B2 and 9,803,217 B2, and are summarized in Table 3 below:

TABLE 3
Summary of AAV plasmids

| | Plasmid | |
|---|---|---|
| | SEQ ID | |
| Plasmid | NO | Component SEQ ID NOS |
| TM017 | 26 | 1, 3, 4, 5, 6, 8, 9, 15, 51, |
| TM037 | 27 | 2, 10, 5, 6, 8, 9, 15, 52 |
| AG007 | 28 | 2, 11, 5, 6, 8, 14, 9, 15, 53 |
| TM039 | 29 | 2, 22, 5, 6, 8, 23, 9, 15, 54 |
| TM040 | 30 | 2, 3, 4, 5, 6, 8, 23, 9, 15, 55 |
| TM016 | 31 | 1, 3, 4, 5, 24, 8, 9, 15, 56 |
| TM035 | 32 | 2, 10, 5, 24, 8, 9, 15, 57 |
| AG012 | 33 | 2, 13, 8, 14, 9, 15, 58 |
| AG004 | 34 | 2, 11, 5, 24, 8, 14, 9, 15, 59 |
| AG006 | 35 | 2, 12, 5, 24, 8, 14, 9, 15, 60 |
| TM042 | 50 | 1, 3, 4, 5, 6, 8, 9, 49, 61 |

AAV vectors of the present disclosure are generated by triple transfection. Methods for triple transfection are known in the art. Briefly, AAV-ITR-containing plasmids (described in Table 3), AAV-RepCap containing plasmid (carrying Rep2 and Cap2 or Cap8) and Adeno-helper plasmid (carrying genes that assist in completing AAV replication cycle) were co-transfected into HEK293 cells. The transfected HEK293 cells were cultured for four days. At the end of the culture period the cells are lysed and the vectors in the culture supernatant and in the cell lysate are purified by a standard CsCl gradient centrifugation method. The purified viral vectors are described in U.S. Pat. No. 9,163,259 B2, and are summarized in Table 4 below.

TABLE 4
Summary of AAV vectors

| | | Component | |
|---|---|---|---|
| AAV | | SEQ ID NOs | Capsid protein |
| vector | Generated from | from 5′ to 3′ | SEQ ID NOs |
| NVS1 | TM017 or | 36, 62, 63, 64, 65, 66, | 19, 68, 69 |
| | TM042 and AAV | 1, 3, 4, 5, 6, 8, 9, | (encoded by 18) |
| | Rep2/Cap2 | | |
| | plasmid | | |
| NVS2 | TM017 or | 36, 62, 63, 64, 65, 66, | 21, 70, 71 |
| | TM042 and AAV | 1, 3, 4, 5, 6, 8, 9 | (encoded by 20) |
| | Rep2/Cap8 | | |
| | plasmid | | |
| NVS3 | TM037 and AAV | 2, 10, 5, 6, 8, 9 | 19, 68, 69 |
| | Rep2/Cap2 | | (encoded by 18) |
| | plasmid | | |
| NVS4 | TM037 and AAV | 2, 10, 5, 6, 8, 9 | 21, 70, 71 |
| | Rep2/Cap8 | | (encoded by 20) |
| | plasmid | | |
| NVS5 | AG007 and AAV | 2, 11, 5, 6, 8, 14, 9 | 19, 68, 69 |
| | Rep2/Cap2 | | (encoded by 18) |
| | plasmid | | |
| NVS6 | AG007 and AAV | 2, 11, 5, 6, 8, 14, 9 | 21, 70, 71 |
| | Rep2/Cap8 | | (encoded by 20) |
| | plasmid | | |
| NVS7 | TM039 and AAV | 2, 22, 5, 6, 8, 23, 9 | 19, 68, 69 |
| | Rep2/Cap2 | | (encoded by 18) |
| | plasmid | | |
| NVS8 | TM039 and AAV | 2, 22, 5, 6, 8, 23, 9 | 21, 70, 71 |
| | Rep2/Cap8 | | (encoded by 20) |
| | plasmid | | |
| NVS9 | TM040 and AAV | 2, 3, 4, 5, 6, 8, 23, 9 | 19, 68, 69 |
| | Rep2/Cap2 | | (encoded by 18) |
| | plasmid | | |
| NVS10 | TM040 and AAV | 2, 3, 4, 5, 6, 8, 23, 9 | 21, 70, 71 |
| | Rep2/Cap2 | | (encoded by 20) |
| | plasmid | | |
| scAAV8- | TM016 and AAV | 36, 62, 67, 64, 65, 66, | 21, 70, 71 |
| pRLBP1 | Rep2/Cap8 | 1, 3, 4, 5, 24, 8, 9 | (encoded by 20) |
| (short)-eGFP | plasmid | | |
| AAV8- | TM035 and AAV | 2, 10, 5, 24, 8, 9 | 21, 70, 71 |
| pRLBP1 | Rep2/Cap8 | | (encoded by 20) |
| (long)-eGFP | plasmid | | |
| AAV8- | AG004 | 2, 11, 5, 24, 8, 14, 9 | 21, 70, 71 |
| pRPE65- | and | | (encoded by 20) |
| eGFP | AAVRep2/Cap8 | | |
| | plasmid | | |
| AAV8- | AG006 and AAV | 2, 12, 5, 24, 8, 14, 9 | 21, 70, 71 |
| pVMD2- | Rep2/Cap8 | | (encoded by 20) |
| eGFP | plasmid | | |
| NVS 11 | AG012 and | 2, 13, 8, 14, 9 | 21, 70, 71 |
| | AAVRep2/Cap8 | | (encoded by 20) |
| | plasmid | | |

Alternatively, GMP-like AAV vectors are generated by cell transfection and culture methods described in the art. The harvested cell culture material is then processed by column chromatography based on methods described by Lock M. et al. (2010), Smith R. H. et al. (2009) and Vadenberghe L. H. et al. (2010).

### Example 3. Transduction of Cells with AAV Vectors

Cells overexpressing lecithin retinol acyltransferase (LRAT) are described in the art, e.g., in WO 2017/190081 A1, US 2017/226490 A1, and US 2009/326074 A1, each of which is incorporated by reference in its entirety. Specifically, HEK293 cells overexpressing LRAT, stably or transiently, (“HEK293 LRAT”) are grown in culture before being plated and allowed to grow for one to five days prior to transduction. See e.g., On the day of transduction, one well of HEK293 LRAT cells is counted to determine cell count. The virus requirements for the transduction are calculated based on the cell count and desired multiplicity of infection (MOI). Appropriate volume of AAV vectors, e.g., from one or more of NVS1 to NVS10, are added to HEK293 LRAT cells to produced transduced HEK293 cells overexpressing LRAT and CRALBP (“HEK293 LRAT/CRALBP”). Pictures are taken on a microscope to show cell viability after transduction.

Alternatively, HeLa cells overexpressing lecithin retinol acyltransferase (“HeLa LRAT”), stably or transiently, are grown in culture before being plated and allowed to grow for one to five days prior to transduction. On the day of transduction, one well of HeLa LRAT cells is counted to determine cell count. The virus requirements for the transduction are calculated based on the cell count and desired multiplicity of infection (MOI). Appropriate volume of AAV vectors, e.g., from one or more of NVS1 to NVS10, are added to HeLa LRAT cells to produce transduced HeLa cells overexpressing LRAT and CRALBP (“HeLa LRAT/CRALBP”). Pictures are taken on a microscope to show cell viability after transduction.

In another example, HEK293 cells overexpressing both LRAT and RPE65 proteins, stably or transiently, (“HEK293 LRAT/RPE65”) are grown in culture before being plated and allowed to grow for one to five days prior to transduction. On the day of transduction, one well of HEK293 LRAT/RPE65 cells is counted to determine cell count. The virus requirements for the transduction are calculated based on the cell count and desired multiplicity of infection (MOI). Appropriate volume of AAV vectors, e.g., from one or more of NVS1 to NVS10, are added to HEK293 LRAT/RPE65 cells to produce transduced HEK293 cells overexpressing LRAT, RPE65, and CRALBP (“HEK293 LRAT/RPE65/CRALBP”). Pictures are taken on a microscope to show cell viability after transduction.

In yet another example, HeLa cells overexpressing both LRAT and RPE65 proteins, stably or transiently, (“HeLa LRAT/RPE65”) are grown in culture before being plated and allowed to grow for one to five days prior to transduction. On the day of transduction, one well of HeLa LRAT/RPE65 cells is counted to determine cell count. The virus requirements for the transduction are calculated based on the cell count and desired multiplicity of infection (MOI). Appropriate volume of AAV vectors, e.g., from one or more of NVS1 to NVS10, are added to HeLa LRAT/RPE65 cells to produce transduced HeLa cells overexpressing LRAT, RPE65, and CRALBP (“HeLa LRAT/RPE65/CRALBP”). Pictures are taken on a microscope to show cell viability after transduction.

In another example, appropriate volume of AAV vectors, e.g., from one or more of NVS1 to NVS10, is added to HEK293 cells to produce transduced cells overexpressing CRALBP. A cell lysate thereof is prepared and added with recombinantly-expressed-and-purified LRAT to produce a cell lysate containing CRALBP and recombinant LRAT (“HEK293 rLRAT/CRALBP lysate”).

In another example, appropriate volume of AAV vectors, e.g., from one or more of NVS1 to NVS10, is added to HeLa cells to produce transduced cells overexpressing CRALBP. A cell lysate thereof is prepared and added with recombinantly-expressed-and-purfied LRAT to produce a cell lysate containing CRALBP and recombinant LRAT (“HeLa rLRAT/CRALBP lysate”).

After transduction, the cells are incubated for one to three days before the cells are harvested for analysis. Once the cells are harvested, pellets are homogenized in 100 μl reaction buffer (10 mM BTP, pH 8.0 adjusted with 1 ON HCl, 100 mM NaCl) and the protein concentration is ascertained by the Bradford assay. The volume of lysate needed to obtain 100 μg of total protein is calculated and the final volume is brought up to 200 μl by adding BTP (pH 8.0), NaCl, BSA, and water.

### Example 4. CRALBP Potency Assay

Protected from light from this point on, all-trans retinol (prepared in at least 10% DMF) is added to the cell lysate prepared from HEK293 LRAT/CRALBP or HeLa LRAT/CRALBP cells. Also added is cell lysate containing RPE65 protein prepared from HEK293 cells transduced with AAV vectors containing RPE65-coding sequences. See e.g., WO 2017/190081 A1, herein incorporated by reference in its entirety. Alternatively, all-trans retinol (prepared in at least 10% DMF) is added to the cell lysate prepared from HEK293 LRAT/RPE65/CRALBP or HeLa LRAT/RPE65/CRALBP cells. In another example, all-trans retinol (prepared in at least 10% DMF) and the cell lysate containing RPE65 protein are added to the HEK293 rLRAT/CRALBP lysate or HeLa rLRAT/CRALBP lysate. Alternatively, all-trans retinol (prepared in at least 10% DMF) and the cell lysate containing RPE65 protein are added to a cell lysate prepared from cells recombinantly expressing LRAT and CRALBP proteins.

The samples are incubated at 37° C. for 2 hr. The reaction is then stopped (quenched) by adding 300 μl 10 mM butylated hydroxytoluene (BHT) in methanol and vortexed for 1 min. The resulting reaction product, i.e., 11-cis retinol is then extracted with hexane and analyzed.

Alternatively, 11-cis retinol dehydrogenase 5 (RDH5) is isolated from HEK293 cells overexpressing RDH5 or HEK293 cells transduced with AAV vectors containing a RDH5-coding sequence and added to any one of the cell lysates described above in the presence of RPE65 and all-trans retinol. The samples are incubated at 37° C. for 2 hr. The reaction is then stopped (quenched) by adding 300 μl 10 mM BHT in methanol and vortexed for 1 min. The resulting reaction product, i.e., 11-cis retinal, is then extracted with hexane and analyzed.

### Example 5. Purification and Quantification of Reaction Products

A LC-MS/MS method is developed for the analysis of 11-cis-retinol and/or 11-cis retinal in the reaction. Samples are prepared by using liquid-liquid extraction (LLE). A 200 μl aliquot of reaction matrix is mixed well with 300 of MeOH with 10 mM BHT, 20 μl of STD or QC working solutions, 20 μl of internal standard working solution, and 300 μl of hexane. The sample is vortexed vigorously and centrifuged. The upper organic layer is carefully transferred to a clean 96-well plate, and evaporated to dryness under a gentle N₂ flow. The sample is reconstituted with 75 μl of Reconstitution Solution (MeOH with 10 mM BHT:water, 3:2 v/v). The analysis is performed using UPLC-MS/MS system by injecting 10 μl of the LLE-processed sample. All sample preparations are under dim yellow light.

A 200 μl aliquot of the reaction matrix is mixed with 200 ul of PBS/Ethanol (50:50, v:v) containing internal standard and 40 mM hydroxyl amine. The mixture was vortexed for 5 minutes, then allowed to shake for 30 minutes at 500 RPM. 1.5 ml of hexane is added the mixture, and the mixture is vortexed for 5 minutes, then centrifuged for 10 minutes at 4000 RPM at 4° C. 1 ml of the hexane is transferred to a new tube, dried down under N₂ then reconstituted in 250 μl of hexane for analysis. All samples are prepared under dim red lights or dark conditions.

The chromatography is performed on a Waters Acquity BEH C18, 1.7 μm, 2.1×100 mm column and analyzed by atmospheric pressure chemical ionization (APCI) mass spectrometry in the positive ion mode. An isocratic condition is used to elute the analytes using acetonitrile: methanol: isopropyl alcohol:water (45:20:5:30, v/v/v/v) as the mobile phase. Sample analysis is conducted with an Agilent 1290 InfinityII, equipped with a Supelcosil LC-SI 4.6×250 mm, 5 um column. The analytes are separated using a gradient mobile phase consisting of mobile phase A (hexane) and mobile phase B (1,4-Dioxane) at 2 ml/min flow. The gradient is as follows: 0.0 min is 99.6% A, at 5.0 min is 99.6% mobile phase A, at 20 min 90% A, at 20.1 min 80% A, at 25 min 80% A, at 25.1 min 99.6% A, and at 30 min, 99.6% A. The mobile phase is post column modified with 10 mM ammonium formate in isopropanol at 200 μl/min, and eluted to a Sciex 6500 QTrap with an APCI source operating in MRM mode. Under these conditions, 11-cis retinol and all-trans retinol are separated and 11-cis retinal and all-trans retinal are separated.

The reaction products, i.e., 11-cis retinol and/or 11-cis retinal, elute separately from all-trans-retinol and the internal standards. The concentrations of the eluted reaction products are measured by using assays known in the art and they reflect the activity of the CRALBP protein.

Having described the present disclosure in detail, it will be apparent that modifications, variations, and equivalent aspects are possible without departing from the spirit and scope of the present disclosure as described herein and in the appended claims. Furthermore, it should be appreciated that all examples in the present disclosure are provided as non-limiting examples.

1. A method for measuring activity of cellular retinaldehyde-binding protein (CRALBP) comprising:
a. contacting a cell with an adeno-associated viral (AAV) vector comprising a heterologous gene encoding a CRALBP protein, whereby a transduced cell expressing the CRALBP protein is generated;
b. lysing the transduced cell to produce a cell extract thereof;
c. incubating the cell extract with a composition comprising a substrate of the vision cycle, under conditions wherein the substrate is converted to a reaction product in the presence of CRALBP protein; and
d. determining the reaction product, whereby the amount of the reaction product reflects the activity of the CRALBP protein.

2. A method for measuring potency of a composition comprising an AAV vector comprising a CRALBP coding sequence for expressing a CRALBP protein, the method comprising:
a. contacting a cell with the AAV vector, whereby a transduced cell expressing the CRALBP protein is generated;
b. lysing the transduced cell to produce a cell extract thereof;
c. incubating the cell extract with a composition comprising a substrate of the vision cycle, wherein the substrate is converted to a reaction product in the presence of CRALBP protein; and
d. determining the reaction product, whereby the amount of the reaction product reflects the activity of the CRALBP protein.

3. The method of claim 1 or 2, wherein the cell or the composition expresses a protein having lecithin retinol acyltransferase (LRAT) activity, and wherein the substrate in step (c) is all-trans retinyl ester or all-trans retinol.

4. The method of any one of claims 1 to 3, wherein the reaction product is 11-cis retinol.

5. The method of claim 4, wherein the composition in step (c) further comprises a protein having retinal pigment epithelium-specific protein 65-KD (RPE65) activity comprising an amino acid sequence that is at least 90% identical to SEQ ID NO: 73.

6. The method of any one of claims 1 to 5, wherein the reaction product comprises 11-cis retinal.

7. The method of claim 6, wherein the composition in step (c) further comprises a protein having RPE65 activity and a protein having 11-cis retinol dehydrogenase 5 (RDH5) activity, wherein the protein having RDH5 activity comprises an amino acid sequence that is at least 90% identical to SEQ ID NO: 77.

8. The method of any one of claims 1 to 7, wherein the AAV vector comprises in the 5′ to 3′ direction:
a. a 5′ inverted terminal repeat (ITR);
b. a recombinant CRALBP-coding sequence; and
c. a 3′ ITR.

9. The method of claim 8, wherein the recombinant CRALBP-coding sequence encodes a protein that is at least 90% identical to SEQ ID NO: 7.

10. The method of claim 8 or 9, wherein the AAV vector comprises a nucleic acid sequence, in the 5′ to 3′ direction, selected from the group consisting of:
a. SEQ ID NOs: 2, 10, 5, 6, 8, and 9;
b. SEQ ID NOs: 2, 11, 5, 6, 8, 14, and 9;
c. SEQ ID NOs: 2, 22, 5, 6, 8, 23, and 9; and
d. SEQ ID NOs: 2, 3, 4, 5, 6, 8, 23, and 9.

11. The method of claim 8 or 9, wherein the 5′ ITR comprises a non-resolvable ITR comprising a nucleic acid sequence as set forth in SEQ ID NO: 1.

12. The method of claim 11, wherein the AAV vector comprises a nucleic acid sequence, in the 5′ to 3′ direction, selected from the group consisting of:
a. SEQ ID NOs: 1, 5, 6, 8, and 9;
b. SEQ ID NOs: 1, 3, 4, 5, 6, 8, and 9; and
c. SEQ ID NOs: 36, 62, 63, 64, 65, 66, 1, 3, 4, 5, 6, 8, and 9.

13. The method of claim 12, wherein the AAV vector comprises an AAV serotype 2 capsid encoded by a nucleic acid sequence of SEQ ID NO: 18.

14. The method of claim 12, wherein the AAV vector comprises an AAV serotype 8 capsid encoded by a nucleic acid sequence of SEQ ID NO: 20.

15. The method of any one of claims 1 to 14, wherein step (c) comprises adding a precursor of the substrate to the cell extract, whereby the precursor is converted to the substrate, and wherein the precursor comprises all-trans retinol.

16. A kit for use in measuring activity of CRALBP comprising:
a. an AAV-ITR-containing plasmid comprising a heterologous gene encoding a CRALBP protein;
b. an AAV-Rep-Cap-containing plasmid;
c. a helper plasmid; and
d. a composition comprising a substrate of the vision cycle.

17. The kit of claim 16, wherein the AAV-ITR-containing plasmid comprises a nucleic acid sequence selected from the group consisting of SEQ ID NOs: 26, 27, 28, 29, 30, and 50.

18. The kit of claim 17, wherein the AAV-ITR-containing plasmid comprises a nucleic acid sequence in the 5′ to 3′ direction, selected from the group consisting of:
a. SEQ ID NOs: 2, 10, 5, 6, 8, and 9;
b. SEQ ID NOs: 2, 11, 5, 6, 8, 14, and 9;
c. SEQ ID NOs: 2, 22, 5, 6, 8, 23, and 9;
d. SEQ ID NOs: 2, 3, 4, 5, 6, 8, 23, and 9; and
e. SEQ ID NOs: 1, 5, 6, 8, and 9.

19. The kit of any one of claims 16 to 18, wherein the AAV-Rep-Cap-containing plasmid encodes an AAV serotype 8 capsid encoded by a nucleic acid sequence of SEQ ID NO: 20.

20. The kit of any one of claims 16 to 19, wherein the substrate comprises all-trans retinyl ester or all-trans retinol.
